(19) United States
(12) Patent Application Publication
(10) Pub. No.: US 2010/0069380 A1
(43) Pub. Date: Mar. 18, 2010

(54) HETEROCYCLIC INHIBITORS OF BACTERIAL PEPTIDYL TRNA HYDROLASE AND USES THEREOF

(76) Inventors: Soongyu Choi, Skillman, NJ (US); Arthur Branstrom, East Windsor, NJ (US); Scott A. Gothe, Morris Plains, NJ (US); Richard Lipman, Monmouth Junction, NJ (US); Tamil Arasu, Edison, NJ (US); Richard G. Wilde, Somerville, NJ (US)

Correspondence Address: JONES DAY, 222 EAST 41ST ST, NEW YORK, NY, 10017, US

(21) Appl. No.: 12/441,929

(22) PCT Filed: Sep. 21, 2007
(86) PCT No.: PCT/US07/20461

§ 371 Date: Oct. 6, 2009

Related U.S. Application Data
(60) Provisional application No. 60/846,799, filed on Sep. 22, 2006.

(51) Int. Cl. C07D 491/052; A61K 31/5377; A61K 31/4741; C07D 491/22; C07D 491/16; A61P 31/04; A61K 31/4743; A61K 31/506; C07D 491/147; C07D 491/153; A61K 31/4375

(52) U.S. Cl. 514/232.5; 435/18; 514/232.8; 514/274; 514/278; 514/285; 514/287; 514/288; 514/291; 514/293; 514/300; 544/80; 544/126; 544/317; 546/18; 546/62; 546/65; 546/66; 546/83; 546/89; 546/123

(57) ABSTRACT

Provided herein are compounds that modulate the activity of a bacterial peptidyl tRNA hydrolase, including compositions and dosage forms comprising the compounds. Further provided herein are methods for screening and identifying compounds that modulate the activity of a bacterial peptidyl tRNA hydrolase. In particular, provided herein are assays for the identification of compounds that inhibit or reduce the activity of a bacterial peptidyl tRNA hydrolase. The methods provided herein provide a simple, sensitive assay for high-throughput screening of libraries of compounds to identify pharmaceutical leads useful for preventing, treating, and managing a bacterial infection or one or more symptoms thereof. Further provided herein are methods for preventing or inhibiting bacterial proliferation as well as methods for preventing, treating, and/or managing a bacterial infection using such compounds and compositions.

This application claims the benefit of U.S. provisional application No. 60/846,799, filed Sep. 22, 2006, which is incorporated by reference herein in its entirety.

### 1. FIELD

Provided herein are compounds that modulate the activity of a bacterial peptidyl tRNA hydrolase, including compositions and dosage forms comprising the compounds. Further provided herein are methods for screening and identifying compounds that modulate the activity of a bacterial peptidyl tRNA hydrolase. In particular, provided herein are assays for the identification of compounds that inhibit or reduce the activity of a bacterial peptidyl tRNA hydrolase. Further provided herein are methods for preventing or inhibiting bacterial proliferation as well as methods for preventing, treating, and/or managing a bacterial infection using such compounds and compositions.

### 2. BACKGROUND

Therapeutic challenge to pathogens creates selective pressure to evolve or acquire resistance, and the emergence and spread of bacterial resistance to every antibiotic available has been documented. Antibiotic resistance will certainly remain as a major issue for treating bacterial infections. Additional factors, such as treatment of non-susceptible infections and poor compliance with recommended dosage regimens accelerate the frequency and spread of resistance. Further, environmental dissemination of pathogenic strains as an act of bioterrorism is an important concern. Several organisms, including Francisella tularensis, Yersinia pestis, Brucella spp., Coxiella burnetii, Bacillus anthracis, and Mycobacterium tuberculosis are of concern as potential agents for biowarfare and bioterrorism. New antibiotics are thus necessary for the treatment of bacterial infections. In particular, it is advantageous to develop antibacterials that inhibit a novel molecular target, one that is different from the targets of currently available antibacterial compounds, because such a compound would not encounter preexisting resistance.

Peptidyl tRNA hydrolase (“Pth”) recycles tRNA from peptidyl-tRNAs that prematurely dissociate from the ribosome during translation (Kramer, B. et al. 1999 Proteins 37:228-241; Menez, J., et al. 2002 Mol. Microbiol. 45:123-129; Menninger, J. R. 1979 J. Bacteriol. 137:694-696; Menninger, J. R., et al. 1973. Mol. Gen. Genet. 121:307-324). Protein synthesis involves the concerted effort of a number of factors, including the ribosome, mRNA, tRNA, and assorted protein factors. In bacteria, it is estimated that only 76% of the initiated protein chains will actually complete the synthesis of the polypeptide (Jorgensen, F. and C. G. Kurland 1990 J. Mol. Biol. 215:511-521). To process these prematurely dissociated products, Pth cleaves the ester bond between the peptide and the tRNA (Kossel, H. 1970 Biochim. Biophys. Acta. 204:191-202; Shiloach, J., et al. 1975 Nucleic Acids Res. 2:1941-1950) and restores the tRNA portion of the peptidyl-tRNA for aminoacylation. This dissociation is believed to be the result of a general editing function to prevent the expression of mutant proteins resulting from the incorporation of incorrect amino acids due to improper codon-anticodon pairing, where the rate of mis-incorporation of amino acids in translation is estimated to occur once per 90 peptide elongation steps (Menninger, J. R. 1976 J. Biol. Chem. 251:3392-3398). Importantly, accumulated peptidyl-tRNA is toxic to the cell and must be cleared by Pth activity. This was demonstrated when an E. coli strain containing a temperature-sensitive mutation within the Pth enzyme was isolated (Atherly, A. G. and J. R. Menninger 1972 Nat. New Biol. 240:245-246). When grown under non-permissive conditions, the mutant cells accumulate peptidyl-tRNA, which reduces the availability of acylatable tRNAs, thus inhibiting protein synthesis and leading to cell death (Menninger, J. R. 1979 J. Bacteriol. 137:694-696). In fact, the bacterial peptidyl-tRNA hydrolase enzyme has been shown in genetic studies to be essential in E. coli (Heurgue-Hamard, V., et al. 1996 EMBO J. 15:2826-2833; Menninger, J. R. 1979 J. Bacteriol. 137:694-696) and Bacillus subtilis (Menez, J., R. H. et al. 2002 Mol. Microbiol. 45:123-129).

### 3. SUMMARY

The present embodiments are based, in part, on the use of bacterial Pth as a novel target for the identification and development of new antibacterial compounds. Pth is an attractive target for several reasons. First, the Pth gene is highly conserved among bacteria so that inhibitors of Pth activity may be used as a broad-spectrum antibacterial agent. Second, Pth is not targeted by currently available antibacterial compounds. Thus, a Pth inhibitor would be useful against strains of bacteria that have demonstrated resistance to currently available antibiotics. Third, Pth is an essential enzyme in bacteria but is nonessential in eukaryotes. Thus, Pth inhibitors demonstrate bactericidal activity while maintaining low cytotoxicity in mammalian cells. Fourth, Pth inhibitors have an advantage over antibiotics which target the ribosome and inhibit protein synthesis. This is because of the relatively small number of Pth enzyme molecules present in a bacterial cell, compared, for example, to the number of ribosomes. The number of Pth molecules per cell has been estimated to be at least one or two orders of magnitude less than the number of ribosomes (Cruz-Vera, L. R., et al. 2000. J. Bacteriol. 182:1523-1528; Dutka, S., et al. 1993 Nucleic Acids Res. 21:4025-4030). This means that Pth inhibitors have a stoichiometric advantage over currently available protein synthesis inhibitors that target the ribosome because a Pth inhibitor has fewer potential targets with which to interact. This also means that Pth inhibitors should be effective at lower concentrations compared to conventional antibiotics that target the ribosome. Fifth, Pth inhibitors are likely to be highly selective for inhibition of the bacterial enzyme versus mammalian homologs, permitting the use of lower doses and leading to fewer side effects. This is because the primary structure of human Pth active site differs somewhat from that of bacterial Pth, therefore inhibitors of bacterial Pth should not inhibit the eukaryotic enzyme. Likewise, bacterial Pth inhibitors should not inhibit the mammalian phosphodiesterase-type enzyme which has demonstrated peptidyl-tRNA hydrolase activity. Finally, the availability of the crystal structure of the E. coli enzyme, which was solved at a resolution of 1.2 Å (Schmitt, E., et al. 1997 Proteins 28:135-136; Schmitt, E., et al. 1997 EMBO J. 16:4760-4769) and which includes mapping of key amino acid residues, makes possible a comprehensive approach to identify inhibitory compounds through modeling of additional bacterial and eukaryotic peptidyl tRNA hydrolase enzymes and through de novo structure-based drug design of small molecule peptidyl tRNA hydrolase inhibitors.

In certain embodiments, provided herein are compounds having the formulas IA, IB and IC:

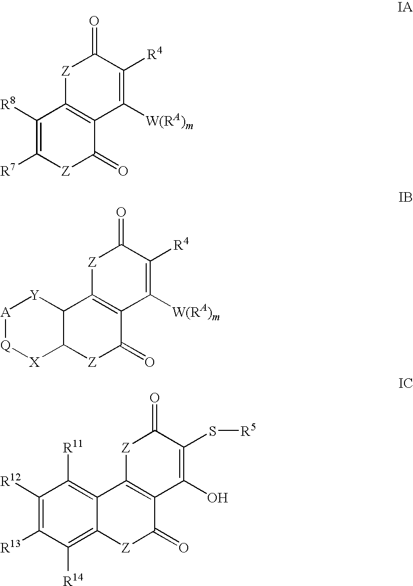

wherein R^{A}, R⁴, R⁵, R⁷, R⁸, R¹¹, R¹², R¹³, R¹⁴, W, Z, Y, A, Q and X are as set forth below. In addition, compounds provided herein include the compounds set forth in Table 1.

In another embodiment, provided herein are pharmaceutical compositions comprising an effective amount of a compound provided herein and a pharmaceutically acceptable carrier, excipient or diluent.

In another embodiment, provided herein are methods of preventing or inhibiting replication of a bacterial organism, comprising contacting the microorganism with an effective amount of a compound provided herein.

In another embodiment, provided herein are methods of preventing, treating or managing a bacterial infection, comprising administering to a subject in need thereof (e.g., a subject having a bacterial infection) an effective amount of a compound provided herein.

In one embodiment, provided herein are methods for identifying a compound that inhibits the activity of a peptidyl tRNA hydrolase enzyme, said methods comprising:

(a) contacting a compound or a pool of compounds with a peptidyl tRNA hydrolase enzyme and a substrate for the enzyme under conditions permitting the cleavage of the substrate by the enzyme; and

(b) measuring the amount of substrate cleaved by the enzyme, wherein a compound that inhibits peptidyl tRNA hydrolase enzyme activity is identified if the amount of substrate cleaved by the enzyme in the presence of the compound is reduced compared to the amount of substrate cleaved in the absence of the compound.

In another embodiment, provided herein are methods for identifying a compound having antibacterial activity, said methods comprising:

(a) contacting a compound or a pool of compounds with a peptidyl tRNA hydrolase enzyme and a substrate for the enzyme under conditions permitting the cleavage of the substrate by the enzyme; and

(b) measuring the amount of substrate cleaved by the enzyme,

wherein a compound that has antibacterial activity is identified if the amount of substrate cleaved by the enzyme in the presence of the compound is reduced compared to the amount of substrate cleaved in the absence of the compound.

In certain embodiments, provided herein are compounds identified by these methods.

### 4. BRIEF DESCRIPTION OF THE FIGURES

FIG. 1: Schematic showing the amino acid sequence of peptidyl tRNA hydrolase is highly conserved among bacteria.

FIG. 2: Schematic of the fluorescence polarization assay.

FIG. 3: Initial results of library screening for compounds having inhibitory activity against peptidyl tRNA hydrolase, showing hit compounds falling within a 95% confidence interval (at 2 Std. Dev.) which were selected for further analysis.

FIG. 4: Minimal Inhibitory Concentrations (MIC) for selected compounds identified using E. coli peptidyl tRNA hydrolase in an assay for antiproliferative activity, showing compounds having antibacterial activity combined with low cytotoxicity in mammalian cells (Huh7 cells).

FIG. 5: Minimal Inhibitory Concentrations (MIC) for selected compounds identified using E. coli. peptidyl tRNA hydrolase in an assay for antiproliferative activity, showing inhibition of vancomycin-resistant (VRE), methicillin-resistant (MRSA), or multi-drug resistant (MDR) bacteria.

FIG. 6: Bactericidal Curves (in vitro), showing a 3 log reduction in bacterial load at 18 hours for the tested peptidyl tRNA hydrolase inhibitors (series II and series I). S. epidermidis 12228 was used as the prototype bacteria in this assay.

FIG. 7. Sequence alignment of the loop region of peptidyl tRNA hydrolase from various bacteria.

FIGS. 8A-8C: Non-limiting list of bacteria that cause infections which can be reduced, inhibited, prevented, treated or managed in accordance with the invention.

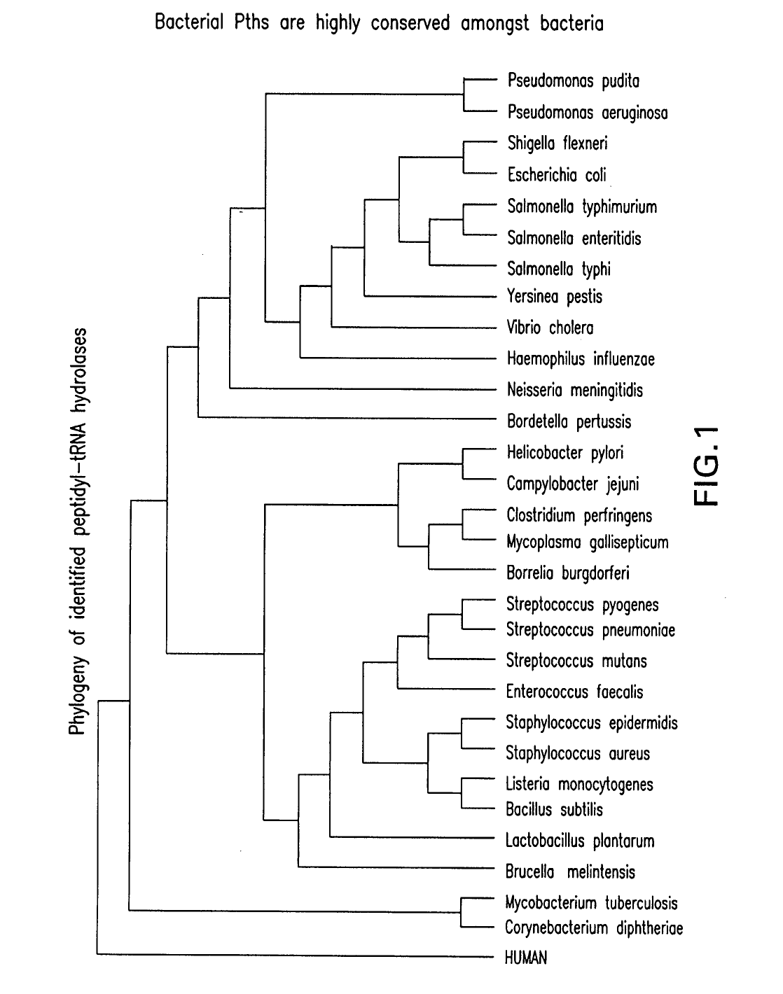

### 5. DETAILED DESCRIPTION

### 5.1 Terminology

As used herein, the terms “about” or “approximately” in the context of a numerical value refers to a number within 10% of the numerical value recited.

As used herein, the terms “compound” and “compounds provided herein” refer to any agent that is being tested for its ability to inhibit the activity of a peptidyl tRNA hydrolase or has been identified as inhibiting the activity of a peptidyl tRNA hydrolase, including the compounds provided herein, such as in Section 5.2 and Table 1, and pharmaceutically acceptable salts, solvates, hydrates, prodrugs and stereoisomers thereof.

As used herein, the term “effective amount” refers to the amount (e.g., of a compound, identified in accordance with the methods provided herein, including the compounds described in Section 5.2 and Table 1, infra) which is sufficient to (1) reduce, ameliorate, or prevent the progression of a bacterial infection; (2) reduce or inhibit bacterial replication and/or bacterial viability; (3) reduce or inhibit a bacterial infection; (4) reduce or inhibit the spread of a bacterial infection; (5) reduce or ameliorate the severity and/or duration of a bacterial infection or one or more symptoms thereof; (6) prevent the recurrence, development or onset of a bacterial infection or one or more symptoms thereof; (7) reduce or inhibit protein synthesis; and/or (8) enhance or improve the prophylactic and/or therapeutic effect(s) of another therapy.

As used herein, the term “in combination” refers to the use of more than one therapy (e.g., prophylactic and/or therapeutic agents). The use of the term “in combination” does not restrict the order in which therapies (e.g., prophylactic and/or therapeutic agents) are administered to a subject with a bacterial infection. A first therapy (e.g., a prophylactic or therapeutic agent, such as a compound identified in accordance with the methods provided herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to a subject with a bacterial infection.

As used herein, the term “infection” means the invasion by and presence of a bacterial cell in a subject. In one embodiment, an infection is an “active” infection, i.e., one in which the bacteria are proliferating in the subject. Such an infection is characterized by the spread of bacteria to other cells, tissues, and organs, from the cells, tissues, or organs initially infected by the bacteria. An infection may also be a latent infection, i.e., one in which the bacteria are not proliferating. In one embodiment, an infection refers to the pathological state resulting from the presence of the bacteria in the body or by the invasion of the body by bacteria.

As used herein, the term “library” refers to a plurality of compounds. A library can be a combinatorial library, e.g., a collection of compounds synthesized using combinatorial chemistry techniques, or a collection of unique chemicals of low molecular weight (less than 1000 daltons).

As used herein, the terms “manage,” “managing” and “management” in the context of the administration of a therapy to a subject, refer to the beneficial effects that a subject derives from a therapy (e.g., a prophylactic or therapeutic agent), which does not result in the eradication of the infection. In certain embodiments, a subject is administered one or more therapies to manage an infection so as to prevent the progression or worsening of the infection.

As used herein, the terms “non-responsive” and refractory” describe patients treated with a currently available therapy (e.g., a prophylactic or therapeutic agent) for a bacterial infection, which is not clinically adequate to eradicate such infection, and/or relieve one or more symptoms thereof. Typically, such patients suffer from severe, persistently active bacterial infection and require additional therapy to ameliorate the symptoms associated with the infection.

As used herein, the term “pool” in the context of a “pool of compounds,” i.e., for use in a high throughput assay, refers to a number of compounds in excess of one compound. In certain embodiments, a pool of compounds is a number of compounds in the range of 1-5, 5-10, 10-25, 25-50, 50-100, 100-150, 150-200, 250-300, 350-400, 200-2,000, 500-2,000, 1,000-5,000 compounds.

As used herein, the terms “prevent,” “preventing,” and “prevention” in the context of the administration of a therapy to a subject, refer to the prevention of the development, recurrence or onset of a bacterial infection or one or more symptoms thereof, resulting from the administration of one or more compounds identified in accordance the methods provided herein, including the compounds described in Section 5.2 and Table 1, or the administration of a combination of such a compound and another therapy for a bacterial infection.

As used herein, the term “previously determined reference range” refers to a reference range for the readout of a particular assay. In a specific embodiment, the term refers to a reference range for the activity of a peptidyl tRNA hydrolase in an assay described in Section 5.8, infra. In some embodiments, each laboratory establishes its own reference range for each particular assay. In one embodiment, at least one positive control and at least one negative control are included in each batch of compounds analyzed.

As used herein, the terms “prophylactic agent” and “prophylactic agents” refer to any agent(s) which can be used in the prevention of a bacterial infection. In certain embodiments, the term “prophylactic agent” refers to a compound identified in the screening assays described herein, or a compound described in Section 5.2 and Table 1. In certain other embodiments, the term “prophylactic agent” refers to an agent other than a compound identified in the screening assays described herein, or a compound described in Section 5.2 and Table 1, which is known to be useful for, or has been or is currently being used to prevent or impede the onset, development, progression, replication, spread, and/or severity of a bacterial infection or one or more symptoms thereof.

As used herein, the phrase “prophylactically effective amount” refers to the amount of a therapy (e.g., a prophylactic agent) which is sufficient to result in the prevention of the development, recurrence or onset of a bacterial infection or one or more symptoms thereof.

As used herein, the term “purified,” in the context of a compound, (e.g., a compound identified in accordance with the methods provided herein, or a compound described in Section 5.2 and Table 1, infra), refers to a compound that is substantially free of chemical precursors or other chemicals when chemically synthesized. In a specific embodiment, the compound is 60%, 65%, 70%, 75%, 80%, 85%, 90%, or 99% free of other, different compounds. In one embodiment, a compound is purified.

As used herein, the term “purified,” in the context of a proteinaceous agent (e.g., a peptide, polypeptide, or protein) refers to a proteinaceous agent which is substantially free of cellular material or contaminating proteins from the cell or tissue source from which it is derived, or substantially free of chemical precursors or other chemicals when chemically synthesized. The language “substantially free of cellular material” includes preparations of a proteinaceous agent in which the proteinaceous agent is separated from cellular components of the cells from which it is isolated or recombinantly produced. Thus, a proteinaceous agent that is substantially free of cellular material includes preparations of a proteinaceous agent having less than about 30%, 20%, 10%, or 5% (by dry weight) of heterologous protein, polypeptide, peptide, or antibody (also referred to as a “contaminating protein”). When the proteinaceous agent is recombinantly produced, it is also preferably substantially free of culture medium, i.e., culture medium represents less than about 20%, 10%, or 5% of the volume of the protein preparation. When the proteinaceous agent is produced by chemical synthesis, it is preferably substantially free of chemical precursors or other chemicals, i.e., it is separated from chemical precursors or other chemicals which are involved in the synthesis of the proteinaceous agent. Accordingly, such preparations of a proteinaceous agent have less than about 30%, 20%, 10%, 5% (by dry weight) of chemical precursors or compounds other than the proteinaceous agent of interest. In one embodiment, proteinaceous agents disclosed herein are purified.

As used herein, the term “small molecules” and analogous terms include, but are not limited to, peptides, peptidomimetics, amino acids, amino acid analogs, polynucleotides, polynucleotide analogs, nucleotides, nucleotide analogs, other organic and inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, organic or inorganic compounds having a molecular weight less than about 100 grams per mole, and salts, esters, and other pharmaceutically acceptable forms of such compounds. Salts, esters, and other pharmaceutically acceptable forms of such compounds are also encompassed.

As used herein, the terms “subject” and “patient” are used interchangeably herein. The terms “subject” and “subjects” refer to an animal, such as a mammal, including non-primates (e.g., cow, pig, horse, cat, dog, rat or mouse) and primates (e.g., monkey or human), and in one embodiment a human. In one embodiment, the subject is a farm animal (e.g., horse, cow, pig) or a pet (e.g., dog or cat). In one embodiment, the subject is a human. In certain embodiments, the subject is refractory or non-responsive to current therapies for a bacterial infection. In one embodiment, the subject is a premature human infant. In one embodiment, the subject is a human infant. In one embodiment, the subject is a human adult. In one embodiment, the subject is a human child. In one embodiment, the subject is an elderly human. In one embodiment, the subject is immunosuppressed or immunocompromised.

As used herein, the term “premature human infant” refers to a human infant born at less than 37 weeks of gestational age.

As used herein, the term “human infant” refers to a newborn to 1 year old year human.

As used herein, the term “human child” refers to a human that is 1 year to 18 years old.

As used herein, the term “human adult” refers to a human that is 18 years or older.

As used herein, the term “elderly human” refers to a human 65 years or older.

As used herein, the term “synergistic” refers to a combination of a compound identified using one of the methods described herein or a compound described herein, and another therapy (e.g., a prophylactic or therapeutic agent), which is more effective than the additive effects of the agents. In a specific embodiment, a synergistic effect of a combination of therapies permits the use of lower dosages of one or more of the therapies and/or less frequent administration of the therapies to a subject with a bacterial infection. The ability to utilize lower dosages of a therapy and/or to administer the therapy less frequently reduces the toxicity associated with the administration of the therapy to a subject without reducing the efficacy of the therapy in the prevention, treatment, management or amelioration of the bacterial infection or one or more symptoms thereof. In another embodiment, a synergistic effect results in improved efficacy of therapies in the prevention, treatment, and/or management of a bacterial infection or one or more symptoms thereof. In another embodiment, a synergistic effect of a combination of therapies may avoid or reduce adverse or unwanted side effects associated with the use of either therapy alone.

As used herein, the terms “therapeutic agent” and “therapeutic agents” refer to any agent(s) which can be used in the prevention, treatment, and/or management of a bacterial infection or one or more symptoms thereof. In certain embodiments, the term “therapeutic agent” refers to a compound provided herein. In other embodiments, the term “therapeutic agent” refers to an agent other than a compound provided herein (e.g., a compound described in Section 5.2 and Table 1). In a specific embodiment, such a therapeutic agent is known to be useful for, or has been or is currently being used for the prevention, treatment, and/or management of a bacterial infection or one or more symptoms thereof.

As used herein, the term “therapeutically effective amount” refers to that amount of the therapy (e.g., a therapeutic agent) sufficient to (1) reduce or inhibit bacterial cell proliferation; (2) reduce or inhibit the viability of bacteria; (3) reduce or inhibit the spread of bacteria from one tissue or organ to another tissue or organ, and/or from one subject to another subject; (4) reduce the severity of a bacterial infection; (5) reduce the duration of a bacterial infection; (6) ameliorate one or more symptoms of a bacterial infection; (7) prevent advancement of a bacterial infection; and/or (8) enhance or improve the therapeutic effect(s) of another therapy. In a specific embodiment, a therapeutically effective amount refers to the amount of a therapy (e.g., therapeutic agent) that inhibits or reduces the replication and/or viability of bacterial cells, inhibits or reduces the onset, development or progression of a bacterial infection or one or more symptoms thereof, or inhibits or reduces the spread of a bacterial infection from one tissue, organ or cell to another tissue, organ or cell. In another specific embodiment, a therapeutically effective amount of a therapy (e.g., a therapeutic agent) reduces the replication of bacterial cells by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%, relative to a negative control, such as PBS.

As used herein, the terms “therapy” and “therapies” refer to any protocol(s), method(s) and/or agent(s) that can be used in the prevention, treatment, management or amelioration of a bacterial infection or one or more symptoms thereof. In certain embodiments, the terms “therapy” and “therapies” refer to antibacterial therapy, supportive therapy and/or other therapies useful in the prevention, treatment, management or amelioration of a bacterial infection or one or more symptoms thereof known to skilled medical personnel.

As used herein, the terms “treat,” “treatment,” and “treating” in the context of the administration of a therapy to a subject, refer to (1) the reduction or inhibition of bacterial cell proliferation; (2) the reduction or inhibition of bacterial viability; (3) the reduction or inhibition of a bacterial infection; (4) the reduction or amelioration of the progression, severity and/or duration of a bacterial infection or one or more symptoms thereof, (5) the amelioration of a symptom of a bacterial infection; and/or (6) the reduction or inhibition of the spread of the bacteria from one organ, tissue or cell to another organ, tissue or cell, resulting from the administration of one or more therapies (e.g., one or more compounds provided herein), or a combination of therapies. In specific embodiments, such terms refer to the inhibition or reduction in the replication and/or viability of bacterial cells.

### ABBREVIATION

Pth Peptidyl tRNA hydrolase

HTS High-throughput Screen

FP Fluorescence Polarization

FRET Fluorescence Resonance Energy Transfer

HPLC High-Performance Liquid Chromatography

FPLC Fast Performance Liquid Chromatography

A “C₁₋₈alkyl” group is a saturated straight chain or branched non-cyclic hydrocarbon having from 1 to 8 carbon atoms. Representative —(C₁₋₈alkyls) include -methyl, -ethyl, -n-propyl, -n-butyl, -n-pentyl, -n-hexyl, -n-heptyl and -n-octyl; while saturated branched alkyls include -isopropyl, -sec-butyl, -isobutyl, -tert-butyl, -isopentyl, 2-methylpentyl, 3-methylpentyl, 4-methylpentyl, 2,3-dimethylbutyl and the like. A —(C₁₋₈alkyl) group can be substituted or unsubstituted.

A “C₁₋₈alkoxy” group is an —O—C₁₋₈alkyl group, wherein C₁₋₈alkyl is as defined above. Representative —(C₁₋₈alkyls) include —O-methyl, —O-ethyl, —O-n-propyl, —O-n-butyl, —O-n-pentyl, —O-n-hexyl, —O-n-heptyl and —O-n-octyl, —O-isopropyl, —O-sec-butyl, —O-isobutyl, —O-tert-butyl, —O-isopentyl, —O-2-methylpentyl, —O-3-methylpentyl, —O-4-methylpentyl, —O-2,3-dimethylbutyl and the like. A C₁₋₈alkoxy group can be substituted or unsubstituted.

A “C₁₋₈alkylsulfoxide” group is an —S(O)—C₁₋₈alkyl group, wherein C₁₋₈alkyl is as defined above. Representative C₁₋₈alkylsulfoxide groups are shown in the compounds provided herein. A C₁₋₈alkylsulfoxide group can be substituted or unsubstituted.

The terms “halogen” and “halo” mean fluorine, chlorine, bromine and iodine.

An “aryl” group is an unsaturated aromatic carbocyclic group of from 6 to 14 carbon atoms having a single ring (e.g., phenyl) or multiple condensed rings (e.g., naphthyl or anthryl). Particular aryls include phenyl, biphenyl, naphthyl and the like. An aryl group can be substituted or unsubstituted.

A “heteroaryl” group is an aryl ring system having one to four heteroatoms (e.g., O, S or N) as ring atoms in a heteroaromatic ring system, wherein the remainder of the atoms are carbon atoms. Suitable heteroatoms include oxygen, sulfur and nitrogen. In certain embodiments, the heterocyclic ring system is monocyclic or bicyclic. Non-limiting examples include aromatic groups selected from the following:

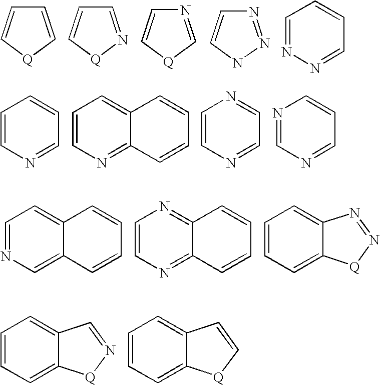

wherein Q is CH₂, CH═CH, O, S or NH. Further representative examples of heteroaryl groups include, but are not limited to, benzofuranyl, benzothienyl, indolyl, benzopyrazolyl, coumarinyl, furanyl, isothiazolyl, imidazolyl, isoxazolyl, thiazolyl, triazolyl, tetrazolyl, thiophenyl, pyrimidinyl, isoquinolinyl, quinolinyl, pyridinyl, pyrrolyl, pyrazolyl, 1H-indolyl, 1H-indazolyl, benzo[d]thiazolyl and pyrazinyl. Further representative examples of heteroaryl groups included those of the compounds disclosed herein. Heteroaryls can be bonded at any ring atom (i.e., at any carbon atom or heteroatom of the heteroaryl ring). A heteroaryl group can be substituted or unsubstituted. In one embodiment, the heteroaryl group is a C₃₋₁₀heteroaryl group.

A “cycloalkyl” group is a saturated or unsaturated non-aromatic carbocyclic ring. Representative cycloalkyl groups include, but are not limited to, cyclopropyl, cyclobutyl, cyclopentyl, cyclopentadienyl, cyclohexyl, cyclohexenyl, 1,3-cyclohexadienyl, 1,4-cyclohexadienyl, cycloheptyl, 1,3-cycloheptadienyl, 1,3,5-cycloheptatrienyl, cyclooctyl, and cyclooctadienyl. Further representative examples of cycloalkyl groups included those of the compounds disclosed herein. A cycloalkyl group can be substituted or unsubstituted. In one embodiment, the cycloalkyl group is a C₃₋₈cycloalkyl group.

A “heterocycloalkyl” group is a non-aromatic cycloalkyl in which one to four of the ring carbon atoms are independently replaced with a heteroatom from the group consisting of O, S and N. Representative examples of a heterocycloalkyl group include, but are not limited to, morpholinyl, pyrrolyl, pyrrolidinyl, thienyl, furanyl, thiazolyl, imidazolyl, pyrazolyl, triazolyl, piperizinyl, isothiazolyl, isoxazolyl, (1,4)-dioxane, (1,3)-dioxolane, 4,5-dihydro-1H-imidazolyl and tetrazolyl. Further representative examples of heterocycloalkyl groups included those of the compounds disclosed herein. Heterocycloalkyls can also be bonded at any ring atom (i.e., at any carbon atom or heteroatom of the Heteroaryl ring). A heterocycloalkyl group can be substituted or unsubstituted. In one embodiment, the heterocycloalkyl is a 3-7 membered heterocycloalkyl.

An “amido” group is —N(R)C(O)substituted or unsubstituted C₁₋₈alkyl, wherein R is H or substituted or unsubstituted C₁₋₈alkyl.

An “amino” group is —N(R)₂, wherein each R is independently H or substituted or unsubstituted C₁₋₈alkyl.

When the groups described herein are said to be “substituted or unsubstituted,” when substituted, they may be substituted with one or more of any substituent. Examples of substituents are those found in the exemplary compounds and embodiments disclosed herein, as well as halo (e.g., chloro, iodo, bromo, or fluoro); C₁₋₈ alkyl; C₂₋₈ alkenyl; C₂₋₈ alkynyl; hydroxyl; C₁₋₈ alkoxyl; amino; nitro; thiol; thioether; imine; cyano; amido; phosphonato; phosphine; carboxyl; carbamoyl; carbamate; acetal; urea; thiocarbonyl; sulfonyl; sulfonamide; ketone; aldehyde; ester; acetyl; acetoxy; oxygen (═O); haloalkyl (e.g., trifluoromethyl); substituted aminoacyl and aminoalkyl; carbocyclic cycloalkyl, which may be monocyclic or fused or non-fused polycyclic (e.g., cyclopropyl, cyclobutyl, cyclopentyl, or cyclohexyl), or a heterocycloalkyl, which may be monocyclic or fused or non-fused polycyclic (e.g., pyrrolidinyl, piperidinyl, piperazinyl, morpholinyl, furanyl, or thiazinyl); carbocyclic or heterocyclic, monocyclic or fused or non-fused polycyclic aryl (e.g., phenyl, naphthyl, pyrrolyl, indolyl, furanyl, thienyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothienyl, or benzofuranyl); amino (primary, secondary, or tertiary); —O-lower alkyl; —O-aryl; aryl; aryl-lower alkyl; CO₂CH₃; CONH₂; OCH₂CONH₂; NH₂; N(C₁₋₄alkyl)₂; NHC(O)C₁₋₄alkyl; SO₂NH₂; SO₂C₁₋₄alkyl; OCHF₂; CF₃; OCF₃; and such moieties may also be optionally substituted by a fused-ring structure or bridge, for example —OCH₂O— or —O-lower alkylene-O—. These substituents may optionally be further substituted with a substituent selected from such groups.

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the compounds include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art, see for example, Remington's Pharmaceutical Sciences, 18^{th} eds., Mack Publishing, Easton Pa. (1990) or Remington: The Science and Practice of Pharmacy, 19^{th} eds., Mack Publishing, Easton Pa. (1995).

As used herein and unless otherwise indicated, the term “hydrate” means a compound, or a salt thereof, that further includes a stoichiometric or non-stoichiometric amount of water bound by non-covalent intermolecular forces.

As used herein and unless otherwise indicated, the term “solvate” means a compound, or a salt thereof, that further includes a stoichiometric or non-stoichiometric amount of a solvent bound by non-covalent intermolecular forces.

As used herein and unless otherwise indicated, the term “prodrug” means a compound derivative that can hydrolyze, oxidize, or otherwise react under biological conditions (in vitro or in vivo) to provide an active compound, particularly a compound provided herein. Examples of prodrugs include, but are not limited to, derivatives and metabolites of a compound that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. In certain embodiments, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh).

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can have chiral centers and can occur as racemates, individual enantiomers or diastereomers, and mixtures thereof. All such isomeric forms are included within the embodiments disclosed herein, including mixtures thereof.

Various compounds contain one or more chiral centers, and can exist as racemic mixtures of enantiomers, mixtures of diastereomers or enantiomerically or optically pure compounds. The use of stereomerically pure forms of such compound, as well as the use of mixtures of those forms are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, Ind., 1972).

It should also be noted the compounds can include E and Z isomers, or a mixture thereof, and cis and trans isomers or a mixture thereof. In certain embodiments, the compounds are isolated as either the E or Z isomer. In other embodiments, the compounds are a mixture of the E and Z isomers.

Concentrations, amounts, cell counts, percentages and other numerical values may be presented herein in a range format. It is to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

### 5.2 Compounds

In one embodiment, provided herein are compounds of formula IA:

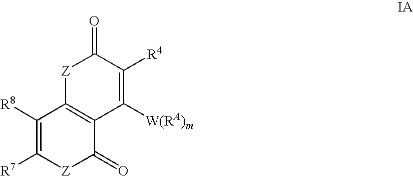

or a pharmaceutically acceptable salt thereof, wherein:

each occurrence of Z is independently O or NR⁶;

W is O, S or N or a direct bond, wherein m is 1 when W is O, S or a direct bond and m is 2 when W is N;

R^{A} is H, (C₁₋₈)alkyl, substituted or unsubstituted aryl, substituted or unsubstituted C(O)—(C₁₋₈)alkyl, C(O)-amino, or substituted or unsubstituted C(O)-aryl;

R⁴ is H, halo, NO₂, CN, substituted or unsubstituted (C₁₋₈)alkyl, substituted or unsubstituted aryl, substituted or unsubstituted heteroaryl, substituted or unsubstituted heterocycloalkyl, substituted or unsubstituted aryl(C₁₋₈)alkyl, OR⁵, S—C(O)—R⁵ or S(O)ₙ—R⁵, wherein n is 0, 1 or 2;

R⁵ is (C₁₋₈)alkyl, amino, CN, substituted or unsubstituted aryl, substituted or unsubstituted heteroaryl or substituted or unsubstituted aryl(C₁₋₈)alkyl;

R⁶ is independently at each occurrence H, substituted or unsubstituted (C₁₋₈)alkyl, substituted or unsubstituted C₂₋₈alkenyl, substituted or unsubstituted cycloalkyl, substituted or unsubstituted aryl, substituted or unsubstituted heteroaryl, substituted or unsubstituted aryl(C₁₋₈)alkyl, or substituted or unsubstituted heteroaryl(C₁₋₈)alkyl;

R⁷ is H, halo, hydroxyl, (C₁₋₈)alkyl, (C₁₋₈)alkoxy, trihalomethyl, substituted or unsubstituted aryl, or substituted or unsubstituted heteroaryl;

or R⁶ and R⁷ together with the atoms to which they are attached form a moiety of the following formula:

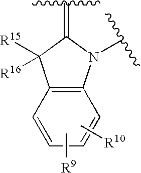

wherein R⁹ and R¹⁰ are independently H, halo, hydroxy, substituted or unsubstituted (C₁₋₈)alkyl, substituted or unsubstituted (C₁₋₈)alkoxy, or trihalomethyl;

wherein R¹⁵ and R¹⁶ are independently (C₁₋₄)alkyl or (C₁₋₂)perfluoroalkyl, or R¹⁵ and R¹⁶ together with the carbon atom to which they are attached form a 3-7 membered cycloalkyl ring, wherein one CH₂ ring member may be optionally replaced by O; and

R⁸ is H, (C₁₋₈)alkyl, halo, hydroxyl, (C₁₋₈)alkoxy, trihalomethyl, S-aryl, substituted or unsubstituted aryl or substituted or unsubstituted heteroaryl; or

R⁷ and R⁸ together with the carbons to which they are attached form a 5, 6 or 7-membered ring optionally containing 1-2 nitrogen atoms, 1-3 double bonds and 1-2 carbonyl groups; and said 5, 6 or 7-membered ring being optionally substituted by one to four substituents selected from halo, hydroxy, (C₁₋₈)alkyl, (C₁₋₈)alkoxy, piperonyl, (C₁₋₈)alkylsulfoxide, or trihalomethyl or said 5, 6 or 7-membered ring being optionally fused to a substituted or unsubstituted phenyl ring; or

R⁶, R⁷ and R⁸ together with the atoms to which they are attached form a 8, 9 or 10-membered bicyclic ring containing 1-3 nitrogen atoms and 1-3 double bonds; and said 8, 9 or 10-membered ring being optionally substituted by one to four substituents selected from halo, hydroxy, (C₁₋₈)alkyl, (C₁₋₈)alkoxy, piperonyl, (C₁₋₈)alkylsulfoxide, or trihalomethyl; or

R⁵ and one of R^{A} together with the atoms to which they are attached form a substituted or unsubstituted heteroaryl ring; or

R⁴ and one of R^{A} together with the atoms to which they are attached form a substituted or unsubstituted heteroaryl ring or a substituted or unsubstituted heterocycloalkyl ring.

In certain embodiments of the compounds having the formula IA, R^{A} is H.

In certain embodiments of the compounds having the formula IA, Z is O and the other is NR⁶.

In certain embodiments of the compounds having the formula IA, R⁴ is S—R⁵.

In certain embodiments of the compounds having the formula IA, W(R_{A})ₘ is OH.

In certain embodiments of the compounds having the formula IA, R⁷ and R⁸ together with the carbons to which they are attached form a substituted or unsubstituted phenyl ring.

In certain embodiments of the compounds having the formula IA, R⁶ is unsubstituted aryl(C₁₋₈)alkyl or aryl(C₁₋₈)alkyl substituted with one or more of halo, hydroxy, C₁₋₈alkyl, C₁₋₈alkoxy, C₁₋₈alkylsulfoxide, or trihalomethyl. In certain embodiments of the compounds having the formula IA, the substituted or unsubstituted aryl(C₁₋₈)alkyl is substituted or unsubstituted benzyl.

In certain embodiments of the compounds having the formula IA, R⁵ is unsubstituted aryl or aryl substituted with one or more of halo, hydroxy, C₁₋₈alkyl, C₁₋₈alkoxy, C₁₋₈alkylsulfoxide, or trihalomethyl. In certain embodiments of the compounds having the formula IA, the substituted or unsubstituted aryl is substituted or unsubstituted phenyl.

In certain embodiments of the compounds having the formula IA, R⁶ is unsubstituted aryl(C₁₋₈)alkyl or aryl(C₁₋₈)alkyl substituted with one or more of halo, hydroxy, C₁₋₈alkyl, C₁₋₈alkoxy, C₁₋₈alkylsulfoxide, or trihalomethyl; and R⁵ is unsubstituted aryl or aryl substituted with one or more of halo, hydroxy, C₁₋₈alkyl, C₁₋₈alkoxy, C₁₋₈alkylsulfoxide, or trihalomethyl.

In certain embodiments, the compounds of formula IA do not include 6-benzyl-4-hydroxy-3-(2,4,5-trichlorophenylsulfonyl)-2H-pyrano[3,2-c]quinoline-2,5(6H)-dione.

In one embodiment, provided herein are compounds of formula IA1:

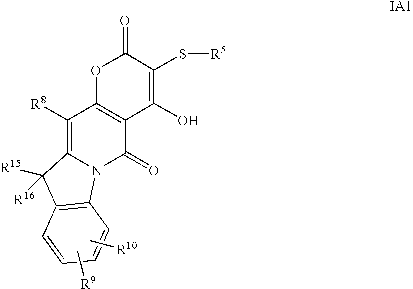

or a pharmaceutically acceptable salt thereof, wherein:

R⁵ is (C₁₋₈)alkyl, CN, substituted or unsubstituted aryl, substituted or unsubstituted heteroaryl or substituted or unsubstituted aryl(C₁₋₈)alkyl;

R⁸ is H, halo, amino, hydroxy, substituted or unsubstituted (C₁₋₈)alkyl, substituted or unsubstituted (C₁₋₈)alkoxy, (C₁₋₈)alkylsulfoxide, substituted or unsubstituted amido, trihalomethyl, O-aryl, O-heteroaryl, substituted or unsubstituted heterocycloalkyl, substituted or unsubstituted S-aryl or substituted or unsubstituted S-alkyl;

R⁹ and R¹⁰ are independently H, halo, hydroxy, substituted or unsubstituted (C₁₋₈)alkyl, substituted or unsubstituted (C₁₋₈)alkoxy, trihalomethyl or (C₁₋₃)alkyl-S(O)ₙ; wherein n can be 0, 1 or 2; and

R¹⁵ and R¹⁶ are independently H, (C₁₋₄)alkyl or (C₁₋₂)perfluoroalkyl, or R¹⁵ and R¹⁶ together with the carbon atom to which they are attached form a 3-7 membered cycloalkyl ring.

In certain embodiments of compounds having formula IA1, R⁵ is substituted or unsubstituted aryl, substituted or unsubstituted heteroaryl, or substituted or unsubstituted aryl(C₁₋₈)alkyl.

In certain embodiments of compounds having formula IA1, R⁵ is substituted or unsubstituted aryl or substituted or unsubstituted heteroaryl.

In certain embodiments of compounds having formula IA1, R⁵ is substituted or unsubstituted aryl.

In certain embodiments of compounds having formula IA1, R⁵ is substituted or unsubstituted aryl; R⁸ is H; R¹⁵ and R¹⁶ are independently H, (C₁₋₄)alkyl, or R¹⁵ and R¹⁶ together with the carbon atom to which they are attached form a 3-7 membered cycloalkyl ring. In one embodiment, R¹⁵ and R¹⁶ are both methyl or together with the carbon atom to which they are attached form a cyclohexyl ring.

In one embodiment, R⁵ is a substituted or unsubstituted phenyl; R⁸ is H; and R¹⁵ and R¹⁶ are independently H, (C₁₋₄)alkyl or R¹⁵ and R¹⁶ together with the carbon atom to which they are attached form a 3-7 membered cycloalkyl ring. In one embodiment, R¹⁵ and R¹⁶ are both methyl or together with the carbon atom to which they are attached form a cyclohexyl ring; and R⁹ and R¹⁰ are independently H, halo, substituted or unsubstituted (C₁₋₈)alkyl, substituted or unsubstituted (C₁₋₈)alkoxy, trihalomethyl, (C₁₋₃)alkyl-S(O)ₙ; wherein n can be 0, 1 or 2.

In another embodiment, provided herein are compounds of formula IA2:

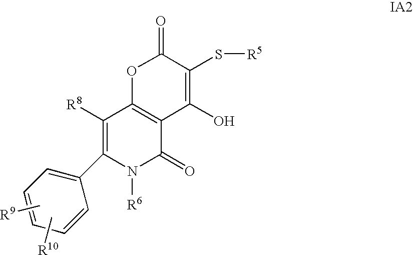

or a pharmaceutically acceptable salt thereof, wherein:

R⁵ is (C₁₋₈)alkyl, CN, substituted or unsubstituted aryl, substituted or unsubstituted heteroaryl or substituted or unsubstituted aryl(C₁₋₈)alkyl;

R⁶ is substituted or unsubstituted aryl or (C₁₋₈)alkyl;

R⁸ is H, halo, hydroxy, (C₁₋₈)alkyl, (C₁₋₈)alkoxy, piperonyl, (C₁₋₈)alkylsulfoxide, or trihalomethyl; and

R⁹ and R¹⁰ are independently H, halo, amino, hydroxy, substituted or unsubstituted (C₁₋₈)alkyl, substituted or unsubstituted (C₁₋₈)alkoxy, or trihalomethyl.

In a further embodiment of the compounds having formula IA2, R⁵ is substituted or unsubstituted aryl, substituted or unsubstituted heteroaryl or substituted or unsubstituted aryl(C₁₋₈)alkyl.

In a further embodiment of the compounds having formula IA2, R⁵ is a substituted or unsubstituted aryl or substituted or unsubstituted heteroaryl. In one embodiment, R⁵ is substituted or unsubstituted phenyl.

In a further embodiment of the compounds having formula IA2, R⁵ is a substituted or unsubstituted aryl; wherein one or more substituents carried by R⁵ are at each occurrence independently hydroxyl, halo, trihalomethyl, substituted or unsubstituted (C₁₋₈)alkyl, substituted or unsubstituted (C₁₋₈)alkoxy. In a further embodiment of the compounds having formula IA2, R⁵ is a substituted or unsubstituted aryl; wherein one or more substituents carried by R⁵ are at each occurrence independently hydroxyl, halo, trihalomethyl, substituted or unsubstituted (C₁₋₈)alkyl, substituted or unsubstituted (C₁₋₈)alkoxy; or R⁸ is H or (C₁₋₃)alkyl. In one embodiment, R⁸ is methyl.

In another embodiment, provided herein are compounds of formula IA2 wherein R⁶ is (C₁₋₈)alkyl. In one embodiment, R⁶ is isopropyl.

In another embodiment, provided herein are compounds of formula IA2 wherein R⁶ is a substituted or unsubstituted aryl group. In one embodiment, R⁶ is substituted or unsubstituted phenyl.

In another embodiment, provided herein are compounds of formula IA3:

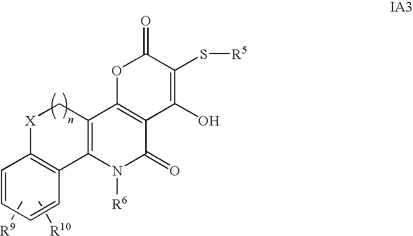

or a pharmaceutically acceptable salt thereof, wherein:

X is O or CH₂;

n is 0, 1 or 2; and

R⁵ is (C₁₋₈)alkyl, CN, substituted or unsubstituted aryl, substituted or unsubstituted heteroaryl or substituted or unsubstituted aryl(C₁₋₈)alkyl;

R⁶ is substituted or unsubstituted aryl group; and

R⁹ and R¹⁰ are independently H, halo, amino, hydroxy, substituted or unsubstituted (C₁₋₈)alkyl, substituted or unsubstituted (C₁₋₈)alkoxy or trihalomethyl.

In a further embodiment of the compounds having formula IA3, R⁵ is substituted or unsubstituted aryl, substituted or unsubstituted heteroaryl or substituted or unsubstituted aryl(C₁₋₈)alkyl.

In a further embodiment of the compounds having formula IA3, R⁵ is substituted or unsubstituted aryl or substituted or unsubstituted heteroaryl. In one embodiment, R⁵ is substituted or unsubstituted phenyl or benzoxazole-2-yl.

In a further embodiment of the compounds having formula IA3, R⁵ is substituted or unsubstituted aryl; wherein one or more substituents carried by R⁵ are at each occurrence independently hydroxyl, halo, trihalomethyl, substituted or unsubstituted (C₁₋₈)alkyl or substituted or unsubstituted (C₁₋₈)alkoxy

In a further embodiment of the compounds having formula IA3, R⁵ is substituted or unsubstituted aryl or substituted or unsubstituted heteroaryl.

In another embodiment, provided herein are compounds of formula IA3 wherein X is O and n is 2.

In another embodiment, provided herein are compounds of formula IA3 wherein X is CH₂ and n is 1.

In another embodiment, provided herein are compounds of formula IA3 wherein X is CH₂ and n is 0.

In one embodiment, provided herein are compounds of formula IA4:

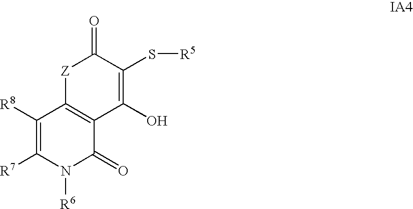

or a pharmaceutically acceptable salt thereof, wherein:

Z is O or NR^{6′};

R⁵ is (C₁₋₈)alkyl, amino, CN, substituted or unsubstituted aryl, substituted or unsubstituted heteroaryl or substituted or unsubstituted aryl(C₁₋₈)alkyl;

R⁶ and R^{6′} are independently H, substituted or unsubstituted (C₁₋₈)alkyl, substituted or unsubstituted (C₂₋₈)alkenyl, substituted or unsubstituted cycloalkyl, substituted or unsubstituted aryl, substituted or unsubstituted heteroaryl, substituted or unsubstituted aryl(C₁₋₈)alkyl, or substituted or unsubstituted heteroaryl(C₁₋₈)alkyl; and

R⁷ and R⁸ are independently H, halo, hydroxyl, (C₁₋₈)alkyl, (C₁₋₈)alkoxy, trihalomethyl, substituted or unsubstituted aryl, or substituted or unsubstituted heteroaryl.

In certain embodiments of the compounds having formula IA4, R⁶ is substituted or unsubstituted aryl, substituted or unsubstituted heteroaryl, substituted or unsubstituted aryl(C₁₋₈)alkyl, or substituted or unsubstituted heteroaryl(C₁₋₈)alkyl.

In an additional embodiment of the compounds having formula IA4, R⁶ is substituted or unsubstituted aryl(C₁₋₈)alkyl. In one embodiment, R⁶ is substituted or unsubstituted benzyl or substituted or unsubstituted 1-phenylethyl.

In a further embodiment of the compounds having formula IA4, R⁶ is substituted or unsubstituted aryl(C₁₋₈)alkyl; and R⁵ is substituted or unsubstituted aryl, substituted or unsubstituted heteroaryl or substituted or unsubstituted aryl(C₁₋₈)alkyl.

In a further embodiment of the compounds having formula IA4, R⁶ is substituted or unsubstituted aryl(C₁₋₈)alkyl; and R⁵ is substituted or unsubstituted aryl, substituted or unsubstituted heteroaryl.

In a further embodiment of the compounds having formula IA4, R⁶ is substituted or unsubstituted aryl(C₁₋₈)alkyl; and R⁵ is substituted or unsubstituted aryl.

In a further embodiment of the compounds having formula IA4, R⁶ is substituted or unsubstituted aryl(C₁₋₈)alkyl; and R⁵ is substituted or unsubstituted aryl, wherein the one or more substituents carried by R⁵ and R⁶ are independently H, hydroxyl, halo, trihalomethyl, substituted or unsubstituted (C₁₋₈)alkyl or substituted or unsubstituted (C₁₋₈)alkoxy; and R⁷ and R⁸ are independently H or (C₁₋₃)alkyl. In one embodiment, R⁷ or R⁸ are methyl.

In one embodiment, provided herein are compounds of formula IA4 wherein Z is O.

In one embodiment, provided herein are compounds of formula IA4 wherein Z is NR^{6′}.

In another embodiment, provided herein are compounds of formula IB:

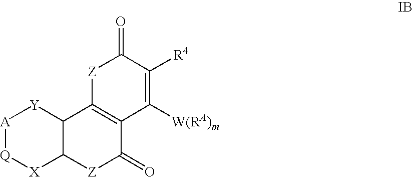

or a pharmaceutically acceptable salt thereof, wherein:

each occurrence of Z is independently O or NR⁶;

W is O, S or N or a direct bond, wherein m is 1 when W is O, S or a direct bond and

m is 2 when W is N;

—Y-A-Q-X— taken together form —C(R¹¹)═C(R¹²)—C(R¹³)═C(R¹⁴)—, —CH₂—CH₂—C(R¹³)═C(R¹⁴)—, —CH₂—CH₂—CH₂—C(R¹³)═C(R¹⁴)—, —CH₂—C(R¹³)═C(R¹⁴)—, or —C(R¹¹)═C(R¹²)—C(R¹³)═N—;

R^{A} is H, (C₁₋₈)alkyl, substituted or unsubstituted aryl, substituted or unsubstituted C(O)—(C₁₋₈)alkyl, C(O)-amino, or substituted or unsubstituted C(O)-aryl;

R⁴ is H, halo, NO₂, CN, substituted or unsubstituted (C₁₋₈)alkyl, substituted or unsubstituted aryl, substituted or unsubstituted heteroaryl, substituted or unsubstituted heterocycloalkyl, substituted or unsubstituted aryl(C₁₋₈)alkyl, OR⁵ or S(O)ₙ—R⁵, wherein n is 0, 1 or 2;

R⁵ is (C₁₋₈)alkyl, amino, CN, substituted or unsubstituted aryl, substituted or unsubstituted heteroaryl or substituted or unsubstituted aryl(C₁₋₈)alkyl;

R⁶ is H, substituted or unsubstituted (C₁₋₈)alkyl, substituted or unsubstituted cycloalkyl, substituted or unsubstituted aryl, substituted or unsubstituted heteroaryl, substituted or unsubstituted aryl(C₁₋₈)alkyl, or substituted or unsubstituted heteroaryl(C₁₋₈)alkyl; and

R¹¹, R¹², R¹³, and R¹⁴ are independently H, halo, amino, hydroxy, substituted or unsubstituted (C₁₋₈)alkyl, substituted or unsubstituted (C₁₋₈)alkoxy, (C₁₋₈)alkylsulfoxide, substituted or unsubstituted amido, trihalomethyl, O-aryl, O-heteroaryl, substituted or unsubstituted heterocycloalkyl, substituted or unsubstituted S-aryl or substituted or unsubstituted S-alkyl; or

R¹³ and R¹⁴ together with the carbon atoms to which they are attached form a substituted or unsubstituted aryl ring, a substituted or unsubstituted 5-6 membered heteroaryl ring containing 0-3 nitrogen atoms, 0-1 oxygen atoms and 0-1 sulfur atoms; or

R⁶ and R¹⁴ together with the atoms to which they are attached form a substituted or unsubstituted heterocycloalkyl; or

R¹² and R¹³ together with the atoms to which they are attached form a substituted or unsubstituted heterocycloalkyl; or

R¹¹ and R¹² together with the atoms to which they are attached form a substituted or unsubstituted heterocycloalkyl.

In certain embodiments of the compounds having the formula IB, R^{A} is H.

In certain embodiments, the compounds of formula IB do not include 6-benzyl-4-hydroxy-3-(2,4,5-trichlorophenylsulfonyl)-2H-pyrano[3,2-c]quinoline-2,5(6H)-dione.

In another embodiment, provided herein are compounds of formula IB1:

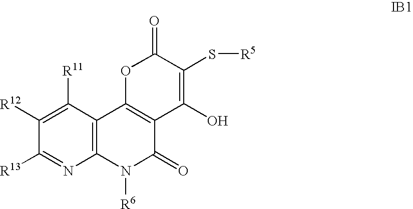

or a pharmaceutically acceptable salt thereof, wherein:

R⁵ is (C₁₋₈)alkyl, amino, CN, substituted or unsubstituted aryl, substituted or unsubstituted heteroaryl or substituted or unsubstituted aryl(C₁₋₈)alkyl;

R⁶ is H, substituted or unsubstituted (C₁₋₈)alkyl, substituted or unsubstituted cycloalkyl, substituted or unsubstituted aryl, substituted or unsubstituted heteroaryl, substituted or unsubstituted aryl(C₁₋₈)alkyl, or substituted or unsubstituted heteroaryl(C₁₋₈)alkyl; and

R¹¹, R¹², and R¹³ are independently H, halo, amino, hydroxy, substituted or unsubstituted (C₁₋₈)alkyl, substituted or unsubstituted (C₁₋₈)alkoxy, (C₁₋₈)alkylsulfoxide, substituted or unsubstituted amido, trihalomethyl, O-aryl, O-heteroaryl, substituted or unsubstituted heterocycloalkyl, substituted or unsubstituted S-aryl or substituted or unsubstituted S-alkyl; or

R¹² and R¹³ together with the atoms to which they are attached form a substituted or unsubstituted heterocycloalkyl; or

R¹¹ and R¹² together with the atoms to which they are attached form a substituted or unsubstituted heterocycloalkyl.

In certain embodiments of the compounds having formula IB1, R⁶ is substituted or unsubstituted aryl, substituted or unsubstituted heteroaryl, substituted or unsubstituted aryl(C₁₋₈)alkyl, or substituted or unsubstituted heteroaryl(C₁₋₈)alkyl.

In an additional embodiment of the compounds having formula IB1, R⁶ is substituted or unsubstituted aryl or substituted or unsubstituted aryl(C₁₋₈)alkyl.

In a further embodiment of the compounds having formula IB1, R⁶ is substituted or unsubstituted aryl or substituted or unsubstituted arylmethyl.

In a further embodiment of the compounds having formula IB1, R⁶ is substituted or unsubstituted aryl or substituted or unsubstituted arylmethyl; R⁵ is substituted or unsubstituted aryl, substituted or unsubstituted heteroaryl or substituted or unsubstituted aryl(C₁₋₈)alkyl.

In a further embodiment of the compounds having formula IB1, R⁶ is substituted or unsubstituted aryl or substituted or unsubstituted arylmethyl; R⁵ is substituted or unsubstituted aryl or substituted or unsubstituted heteroaryl.

In a further embodiment of the compounds having formula IB1, R⁶ is substituted or unsubstituted aryl or substituted or unsubstituted arylmethyl; R⁵ is substituted or unsubstituted aryl or substituted or unsubstituted heteroaryl; wherein the one or more substituents carried by R⁵ and R⁶ are at each occurrence independently, hydroxyl, halo, trihalomethyl, substituted or unsubstituted (C₁₋₈)alkyl, substituted or unsubstituted (C₁₋₈)alkoxy, or a carboxylic acid (C₁₋₃)alkyl ester; and R¹¹, R¹², and R¹³ are independently H, halo, hydroxy, substituted or unsubstituted (C₁₋₈)alkyl, substituted or unsubstituted (C₁₋₈)alkoxy, (C₁₋₈)alkylsulfone, substituted or unsubstituted S—(C₁₋₈)alkyl, substituted or unsubstituted (C₁₋₈)alkylamino or (C₁₋₈)dialkylamino, trihalomethyl, or substituted or unsubstituted heterocycloalkyl; or R¹² and R¹³ together with the atoms to which they are attached form a substituted or unsubstituted heterocycloalkyl.

An another embodiment, provided herein are compounds of formula IB2:

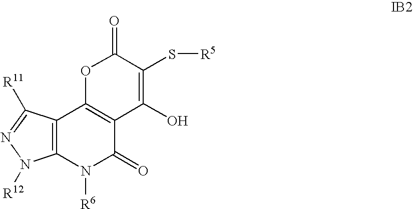

or a pharmaceutically acceptable salt thereof, wherein:

R⁵ is (C₁₋₈)alkyl, amino, CN, substituted or unsubstituted aryl, substituted or unsubstituted heteroaryl or substituted or unsubstituted aryl(C₁₋₈)alkyl;

R⁶ is H, substituted or unsubstituted (C₁₋₈)alkyl, substituted or unsubstituted cycloalkyl, substituted or unsubstituted aryl, substituted or unsubstituted heteroaryl, substituted or unsubstituted aryl(C₁₋₈)alkyl, or substituted or unsubstituted heteroaryl(C₁₋₈)alkyl;

R¹¹ is H, halo, amino, hydroxy, substituted or unsubstituted (C₁₋₈)alkyl, substituted or unsubstituted (C₁₋₈)alkoxy, (C₁₋₈)alkylsulfoxide, substituted or unsubstituted amido, trihalomethyl, substituted or unsubstituted heterocycloalkyl, substituted or unsubstituted S-aryl or substituted or unsubstituted S-alkyl; and

R¹² is H or substituted or unsubstituted (C₁₋₈)alkyl.

In certain embodiments of the compounds having formula IB2, R⁶ is substituted or unsubstituted aryl, substituted or unsubstituted heteroaryl, substituted or unsubstituted aryl(C₁₋₈)alkyl, or substituted or unsubstituted heteroaryl(C₁₋₈)alkyl.

In an additional embodiment of the compounds having formula IB2, R⁶ is substituted or unsubstituted aryl, or substituted or unsubstituted aryl(C₁₋₈)alkyl. In one embodiment, R⁶ is substituted or unsubstituted phenyl or substituted or unsubstituted benzyl.

In a further embodiment of the compounds having formula IB2, R⁶ is substituted or unsubstituted aryl, or substituted or unsubstituted arylmethyl.

In a further embodiment of the compounds having formula IB2, R⁶ is substituted or unsubstituted aryl or substituted or unsubstituted arylmethyl; R⁵ is a substituted or unsubstituted aryl, substituted or unsubstituted heteroaryl or substituted or unsubstituted aryl(C₁₋₈)alkyl.

In a further embodiment of the compounds having formula IB2, R⁶ is substituted or unsubstituted aryl or substituted or unsubstituted arylmethyl; R⁵ is substituted or unsubstituted aryl or substituted or unsubstituted heteroaryl. In one embodiment, R⁵ is substituted or unsubstituted phenyl.

In a further embodiment of the compounds having formula IB2, R⁶ is substituted or unsubstituted aryl or substituted or unsubstituted arylmethyl; R⁵ is substituted or unsubstituted aryl, substituted or unsubstituted heteroaryl; wherein the one or more substituents carried by R⁵ and R⁶ are independently H, hydroxyl, halo, trihalomethyl, substituted or unsubstituted (C₁₋₈)alkyl, substituted or unsubstituted (C₁₋₈)alkoxy, or a carboxylic acid (C₁₋₃)alkyl ester; and R¹¹ and R¹² are independently H or substituted or unsubstituted (C₁₋₈)alkyl. In one embodiment, R¹¹ and R¹² are methyl.

In another embodiment, provided herein are compounds of formula IC:

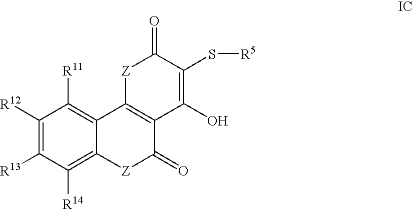

or a pharmaceutically acceptable salt thereof, wherein:

each occurrence of Z is independently O or NR⁶;

R⁵ is C(O)-amino, CN, (C₁₋₈)alkyl, substituted or unsubstituted aryl, substituted or unsubstituted heteroaryl or substituted or unsubstituted aryl(C₁₋₈)alkyl;

R⁶ is H, substituted or unsubstituted (C₁₋₈)alkyl, substituted or unsubstituted cycloalkyl, substituted or unsubstituted aryl, substituted or unsubstituted heteroaryl, substituted or unsubstituted aryl(C₁₋₈)alkyl, or substituted or unsubstituted heteroaryl(C₁₋₈)alkyl; and

R¹¹, R¹², R¹³, and R¹⁴ are independently H, halo, amino, hydroxy, substituted or unsubstituted (C₁₋₈)alkyl, substituted or unsubstituted (C₁₋₈)alkoxy, (C₁₋₈)alkylsulfoxide, substituted or unsubstituted amido, trihalomethyl, O-aryl, O-heteroaryl, substituted or unsubstituted heterocycloalkyl, substituted or unsubstituted S-aryl or substituted or unsubstituted S-alkyl; or

R⁶ and R¹⁴ together with the atoms to which they are attached form a substituted or unsubstituted heterocycloalkyl; or

R¹² and R¹³ together with the atoms to which they are attached form a substituted or unsubstituted heterocycloalkyl; or

R¹¹ and R¹² together with the atoms to which they are attached form a substituted or unsubstituted heterocycloalkyl.

In certain embodiments of the compounds having the formula IC, R⁶ is substituted or unsubstituted aryl(C₁₋₈)alkyl, wherein the substitutions of the substituted aryl(C₁₋₈)alkyl are one or more halo, hydroxy, (C₁₋₈)alkyl, (C₁₋₈)alkoxy, (C₁₋₈)alkylsulfoxide, or trihalomethyl. In one embodiment, the substituted or unsubstituted aryl(C₁₋₈)alkyl can be substituted or unsubstituted benzyl.

In certain embodiments of the compounds having the formula IC, R⁵ is substituted or unsubstituted aryl, wherein the substitutions of the substituted aryl are one or more halo, hydroxy, (C₁₋₈)alkyl, (C₁₋₈)alkoxy, (C₁₋₈)alkylsulfoxide, or trihalomethyl. In one embodiment, R⁵ is be substituted or unsubstituted phenyl.

In certain embodiments of the compounds having the formula IC, R¹¹ is H. In other embodiments R¹⁴ is H. In other embodiments R¹¹ and R¹⁴ are both H.

In certain embodiments of the compounds having the formula IC, R⁶ is substituted or unsubstituted aryl(C₁₋₈)alkyl, wherein the substitutions of the substituted aryl(C₁₋₈)alkyl are one or more halo, hydroxy, (C₁₋₈)alkyl, (C₁₋₈)alkoxy, (C₁₋₈)alkylsulfoxide, or trihalomethyl; R⁵ is substituted or unsubstituted aryl, wherein the substitutions of the substituted aryl are one or more halo, hydroxy, (C₁₋₈)alkyl, (C₁₋₈)alkoxy, (C₁₋₈)alkylsulfoxide, or trihalomethyl; and R¹¹ and R¹⁴ are H.

In another embodiment, provided herein are compounds of formula IC1:

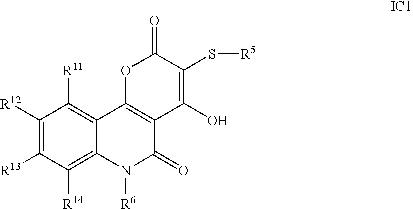

or a pharmaceutically acceptable salt thereof, wherein:

R⁵ is C(O)-amino, CN, (C₁₋₈)alkyl, substituted or unsubstituted aryl, substituted or unsubstituted heteroaryl or substituted or unsubstituted aryl(C₁₋₈)alkyl;

R⁶ is H, substituted or unsubstituted (C₁₋₈)alkyl, substituted or unsubstituted cycloalkyl, substituted or unsubstituted aryl, substituted or unsubstituted heteroaryl, substituted or unsubstituted aryl(C₁₋₈)alkyl, or substituted or unsubstituted heteroaryl(C₁₋₈)alkyl; and

R¹¹, R¹², R¹³, and R¹⁴ are independently H, halo, amino, hydroxy, substituted or unsubstituted (C₁₋₈)alkyl, substituted or unsubstituted (C₁₋₈)alkoxy, (C₁₋₈)alkylsulfoxide, substituted or unsubstituted amido, trihalomethyl, O-aryl, O-heteroaryl, substituted or unsubstituted heterocycloalkyl, substituted or unsubstituted S-aryl or substituted or unsubstituted S-alkyl; or

R¹¹ and R¹² together with the atoms to which they are attached form a substituted or unsubstituted heterocycloalkyl; or

R¹² and R¹³ together with the atoms to which they are attached form a substituted or unsubstituted heterocycloalkyl; or

R¹³ and R¹⁴ together with the atoms to which they are attached form a substituted or unsubstituted heterocycloalkyl.

In a further embodiment of the compounds having formula IC1, R⁵ is a substituted or unsubstituted aryl, substituted or unsubstituted heteroaryl or substituted or unsubstituted aryl(C₁₋₈)alkyl. In one embodiment, R⁵ is phenyl or benzoxazol-2-yl.

In a further embodiment of the compounds having formula IC1, R⁵ is substituted or unsubstituted aryl or substituted or unsubstituted heteroaryl.

In a further embodiment of the compounds having formula IC1, R⁵ is a substituted or unsubstituted aryl, substituted or unsubstituted heteroaryl; wherein the one or more substituents carried by R⁵ and R⁶ are independently H, hydroxyl, halo, nitro, trihalomethyl, substituted or unsubstituted (C₁₋₈)alkyl, substituted or unsubstituted (C₁₋₈)alkoxy, or a carboxylic acid (C₁₋₃)alkylester; R¹¹ and R¹⁴ are H; or R¹² and R¹³ are independently halo, hydroxy, substituted or unsubstituted (C₁₋₈)alkyl, substituted or unsubstituted (C₁₋₈)alkoxy, (C₁₋₈)alkylsulfone, substituted or unsubstituted (C₁₋₈)alkyl-mercaptane, substituted or unsubstituted (C₁₋₈)alkylamino or (C₁₋₈)dialkylamino, trihalomethyl or substituted or unsubstituted heterocycloalkyl (e.g. morpholine or piperidine); and R¹² and R¹³ together with the atoms to which they are attached form a substituted or unsubstituted heterocycloalkyl, such as a 1,3-dioxolane ring.

In another embodiment, provided herein are compounds of formula IC1 wherein R⁶ is substituted or unsubstituted aryl-(C₁₋₈)alkyl. In one embodiment, R⁶ is substituted or unsubstituted benzyl.

In another embodiment, provided herein are compounds of formula IC1 wherein R⁶ is substituted or unsubstituted (C₁₋₈)alkyl or substituted or unsubstituted cycloalkyl. In one embodiment, R⁶ is methyl, ethyl, vinyl, butyl, cyclopropyl, 1-cyclopropylethyl or cyclopentyl.

In a further embodiment of the compounds having formula IC1, R⁵ is substituted or unsubstituted aryl, substituted or unsubstituted heteroaryl or substituted or unsubstituted aryl(C₁₋₈)alkyl. In one embodiment, R⁵ is substituted or unsubstituted phenyl or benzoxazol-2-yl.

In a further embodiment of the compounds having formula IC1, R⁵ is substituted or unsubstituted aryl or substituted or unsubstituted heteroaryl.

In a further embodiment of the compounds having formula IC1, R⁵ is a substituted or unsubstituted aryl, substituted or unsubstituted heteroaryl; wherein the one or more substituents carried by R⁵ are independently H, hydroxyl, halo, nitro, trihalomethyl, substituted or unsubstituted (C₁₋₈)alkyl, substituted or unsubstituted (C₁₋₈)alkoxy, or a carboxylic acid (C₁₋₃)alkylester; or R¹¹ and R¹⁴ are H; or R¹² and R¹³ are independently halo, hydroxy, substituted or unsubstituted (C₁₋₈)alkyl, substituted or unsubstituted (C₁₋₈)alkoxy, (C₁₋₈)alkylsulfone, substituted or unsubstituted (C₁₋₈)alkyl-mercaptane, substituted or unsubstituted (C₁₋₈)alkylamino or (C₁₋₈)dialkylamino, trihalomethyl or substituted or unsubstituted heterocycloalkyl (e.g. morpholine or piperidine); and R¹² and R¹³ together with the atoms to which they are attached form a substituted or unsubstituted heterocycloalkyl, such as a 1,3-dioxolane ring.

In another embodiment, provided herein are compounds of formula IC1 wherein R⁶ is substituted or unsubstituted aryl. In one embodiment, R⁶ is phenyl, R⁵ is C(O)-amino, CN, (C₁₋₈)alkyl, substituted or unsubstituted aryl, substituted or unsubstituted heteroaryl or substituted or unsubstituted aryl(C₁₋₈)alkyl; and R¹¹, R¹², R¹³, and R¹⁴ are independently H, halo, amino, hydroxy, substituted or unsubstituted (C₁₋₈)alkyl, substituted or unsubstituted (C₁₋₈)alkoxy, (C₁₋₈)alkylsulfoxide, substituted or unsubstituted amido, trihalomethyl, O-aryl, O-heteroaryl, substituted or unsubstituted heterocycloalkyl, substituted or unsubstituted S-aryl or substituted or unsubstituted S-alkyl; or R¹¹ and R¹² together with the atoms to which they are attached form a substituted or unsubstituted heterocycloalkyl; or R¹² and R¹³ together with the atoms to which they are attached form a substituted or unsubstituted heterocycloalkyl; or R¹³ and R¹⁴ together with the atoms to which they are attached form a substituted or unsubstituted heterocycloalkyl.

In another embodiment, provided herein are compounds of formula IC2:

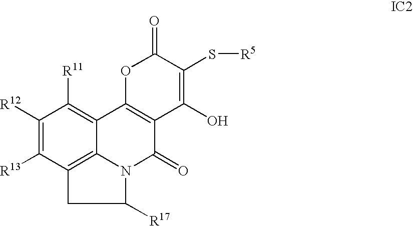

or a pharmaceutically acceptable salt thereof, wherein:

R⁵ is C(O)-amino, CN, (C₁₋₈)alkyl, substituted or unsubstituted aryl, substituted or unsubstituted heteroaryl or substituted or unsubstituted aryl(C₁₋₈)alkyl;

R¹¹, R¹² and R¹³ are independently H, halo, amino, hydroxy, substituted or unsubstituted (C₁₋₈)alkyl, substituted or unsubstituted (C₁₋₈)alkoxy, (C₁₋₈)alkylsulfoxide, substituted or unsubstituted amido, trihalomethyl, O-aryl, O-heteroaryl, substituted or unsubstituted heterocycloalkyl, substituted or unsubstituted S-aryl or substituted or unsubstituted S-alkyl; and

R¹⁷ is substituted or unsubstituted aryl or (C₁₋₈)alkyl; or

R¹¹ and R¹² together with the atoms to which they are attached form a substituted or unsubstituted heterocycloalkyl; or

R¹² and R¹³ together with the atoms to which they are attached form a substituted or unsubstituted heterocycloalkyl.

In a further embodiment of the compounds having formula IC2, R⁵ is substituted or unsubstituted aryl, substituted or unsubstituted heteroaryl or substituted or unsubstituted aryl(C₁₋₈)alkyl. In one embodiment, R⁵ is phenyl or benzoxazol-2-yl.

In a further embodiment of the compounds having formula IC2, R⁵ is substituted or unsubstituted aryl or substituted or unsubstituted heteroaryl.

In a further embodiment of the compounds having formula IC2, R⁵ is substituted or unsubstituted aryl or substituted or unsubstituted heteroaryl; wherein the one or more substituents carried by R⁵ and R¹⁷ being a substituted phenyl are independently H, hydroxyl, halo, nitro, trihalomethyl, substituted or unsubstituted (C₁₋₈)alkyl, substituted or unsubstituted (C₁₋₈)alkoxy; R¹¹, R¹² and R¹³ are independently halo, hydroxy, substituted or unsubstituted (C₁₋₈)alkyl, substituted or unsubstituted (C₁₋₈)alkoxy, (C₁₋₈)alkylsulfone, substituted or unsubstituted (C₁₋₈)alkyl-mercaptane, substituted or unsubstituted (C₁₋₈)alkylamino or (C₁₋₈)dialkylamino, trihalomethyl or substituted or unsubstituted heterocycloalkyl (e.g. morpholine or piperidine); and R¹² and R¹³ together with the atoms to which they are attached form a substituted or unsubstituted heterocycloalkyl, such as a 1,3-dioxolane ring.

Representative compounds are set forth in Table 1.

Lengthy table referenced here
US20100069380A1-20100318-T00001
Please refer to the end of the specification for access instructions.

0.1-1 μg/ml=5 stars
1.1-10 μg/ml=4 stars
10.1-20 μg/ml=3 stars
20.1-50 μg/ml=2 stars
>50 μg/ml=1 star

Any of the compounds disclosed herein can be used in the uses described in section 5.5.

In one embodiment, provided herein are the disclosed compounds being isotopically-labelled (i.e., having one or more atoms replaced by an atom having a different atomic mass or mass number). Examples of isotopes that can be incorporated into the disclosed compounds include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

### 5.3 Methods for Making Compounds

Compounds disclosed herein can be made by one skilled in the art using conventional organic syntheses and commercially available materials. By way of example and not limitation, compounds disclosed herein can be prepared as outlined in Schemes 1-30 shown below, as well as in the accompanying description. It should be noted that one skilled in the art can modify the procedures set forth in the illustrative schemes and examples to obtain the desired product.

Aryl substituents R and R′ (see Compound D in Scheme 3) and substituent Ar can be any suitable substitution as illustrated in Section 5.2 or Table 1.

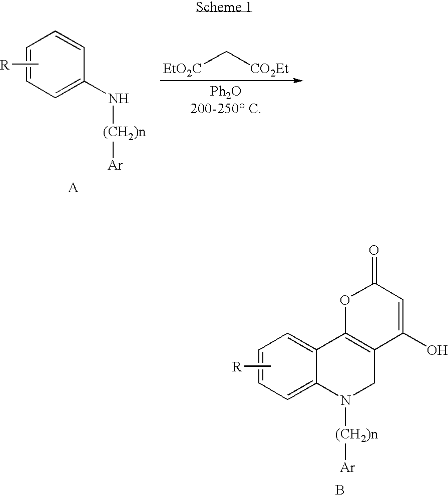

A solution of the aniline compound A (1.0 eq.) and diethyl malonate (2.1 eq.) in diphenyl ether (1 M) is heated to >200° C. in a roundbottom flask equipped with a thermometer and a short-path stillhead (also with a thermometer). As the reaction proceeds, the distillate (ethanol) is collected and the temperature of the vapor monitored. The reaction mixture is allowed to cool to ambient temperature, and is poured into ether or 1:1 ether-hexane. The resulting solid (compound B) is collected by filtration, washed with more solvent, and dried under vacuum.

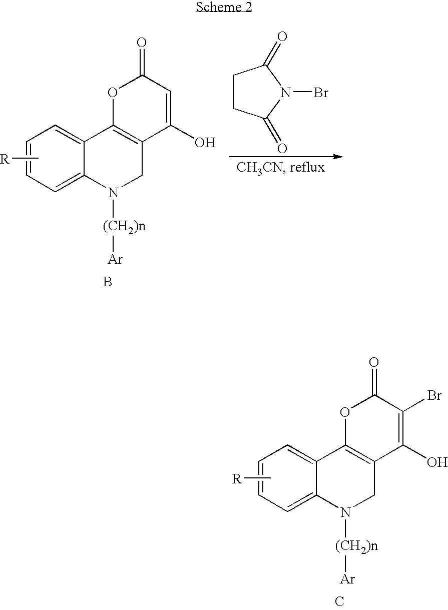

A suspension of compound B and N-bromosuccinimide (1.05 eq.) in acetonitrile (0.25 M) is heated to reflux overnight. The mixture is cooled, filtered and washed with a small volume of acetonitrile, then ether. The solid product (compound C) is dried under vacuum.

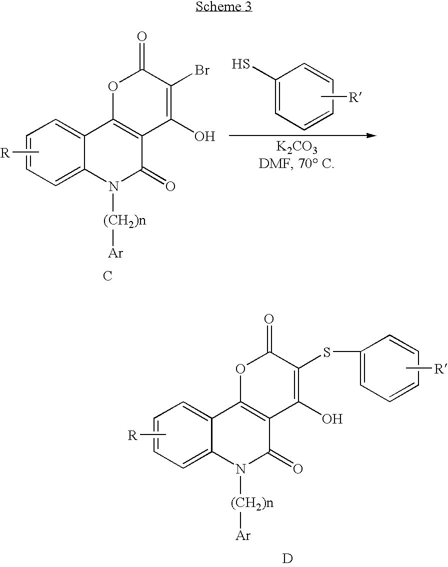

A suspension of bromide compound C, thiophenol (3-4 eq.) and potassium carbonate (2 eq.) in dimethylformamide (0.5 M) is heated to 70° C. overnight. The mixture is cooled, and partitioned between 4 volumes each of 0.25 N aq. HCl and ethyl acetate or diethyl ether. In the event that a precipitate forms that fails to dissolve in either phase, the mixture is filtered and the solid is washed with diethyl ether and dried under vacuum (compound D). If no precipitate forms, the organic phase is washed twice more with water and once with satd. aq. brine, dried over anhydrous magnesium sulfate, filtered and evaporated. The resulting solid (D) is then washed with 1:1 ether-hexane and dried under vacuum.

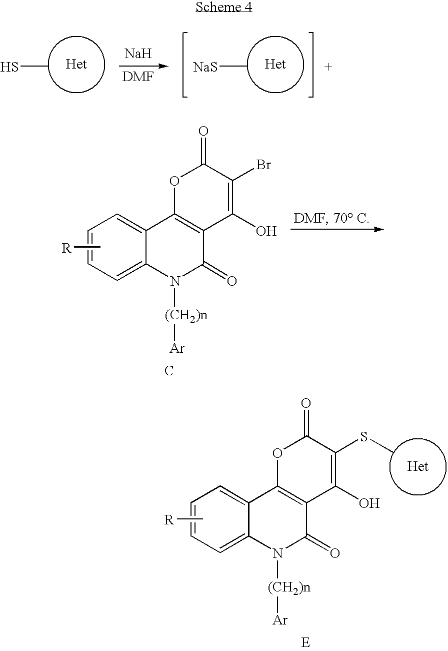

A solution of the desired mercaptoheterocycle in dimethylformamide is treated with sodium hydride suspension in mineral oil, and the mixture is allowed to stir for 30 minutes. This is treated with compound C, and the resulting solution is heated to 70° C. overnight. The workup then proceeds as described above, to afford product E.

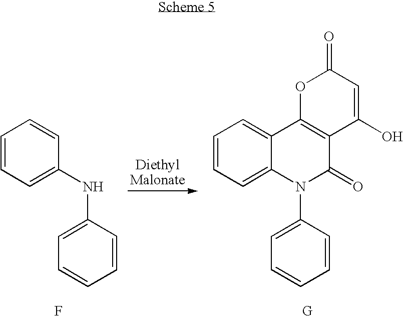

Compound G can be prepared from Compound F using the following methods:

Method 1: diethyl malonate in diphenyl ether at 250° C.

Method 2: malonyl dicholoride in dichloromethane.

Method 3: meldrum's acid in dichloromethane.

Compound F, if not commercially available, can be prepared by the following methods 4 and 5.

Method 4:

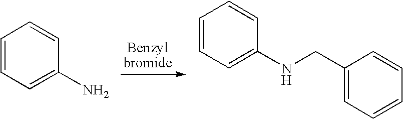

Method 5: Reductive amination from benzaldehyde with sodium triacetoxyborohydride

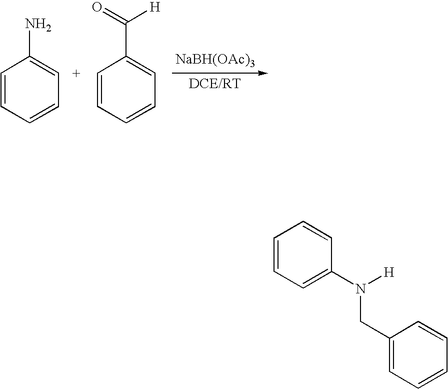

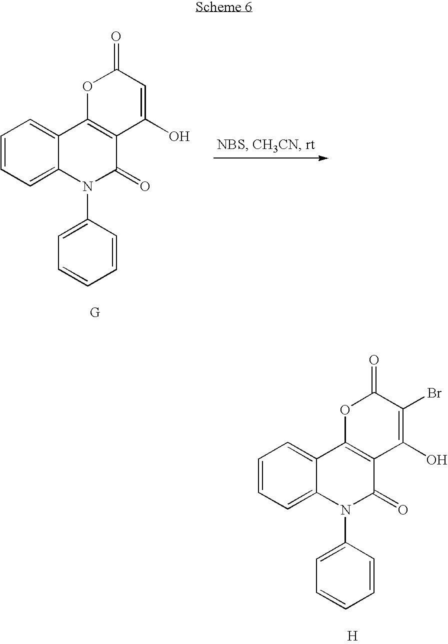

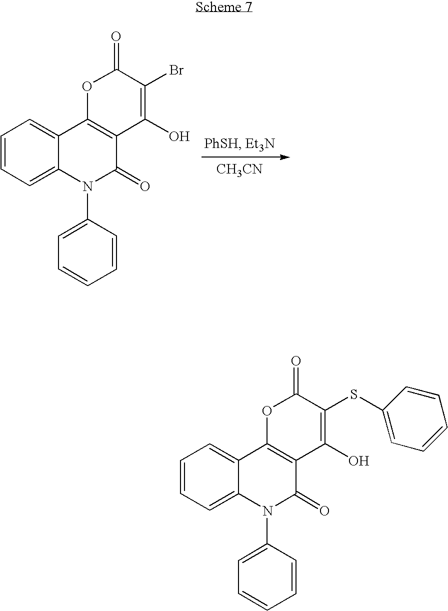

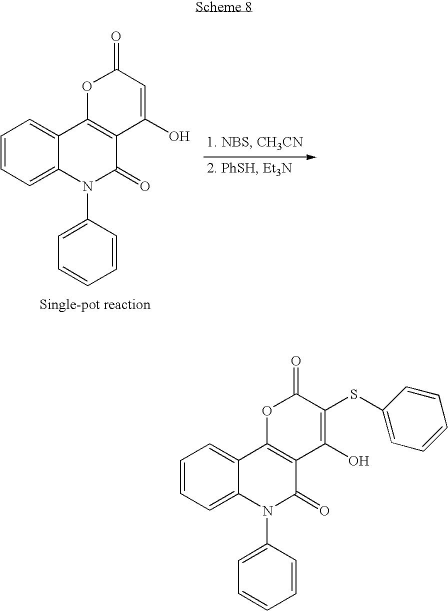

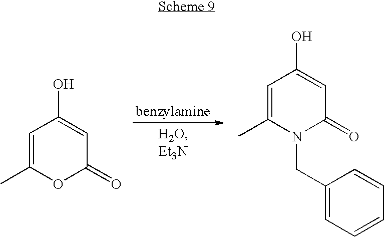

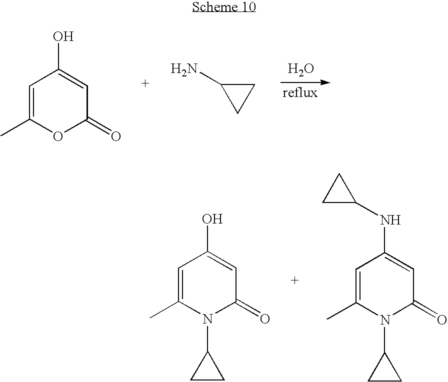

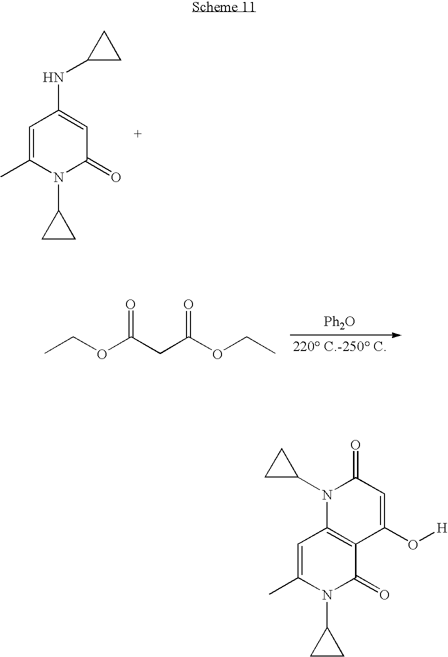

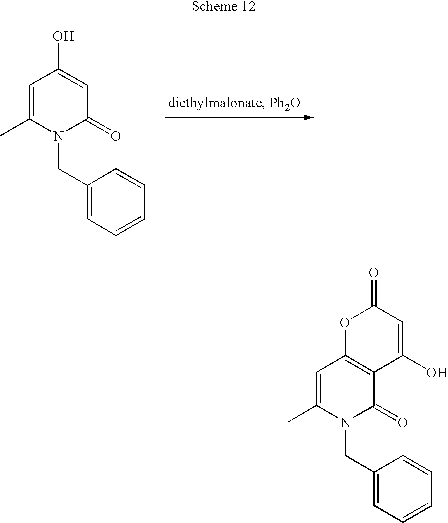

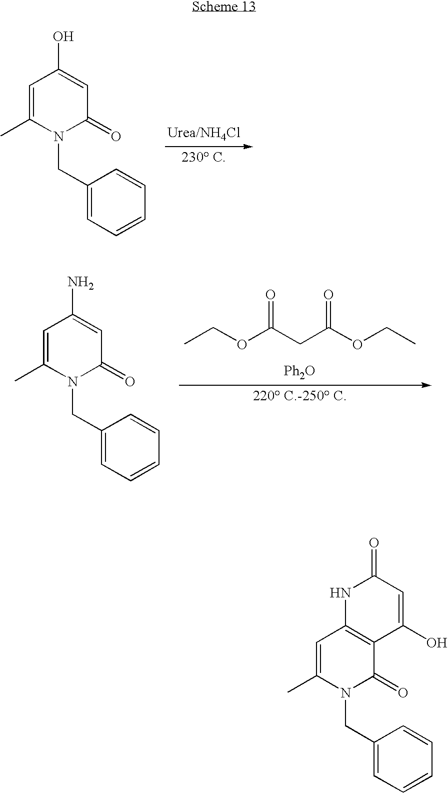

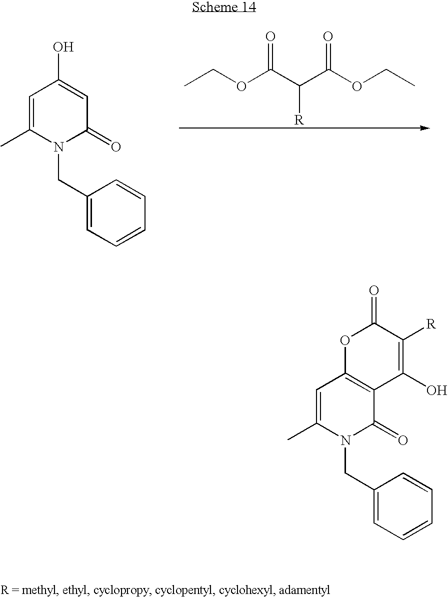

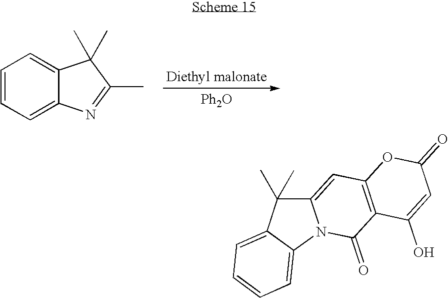

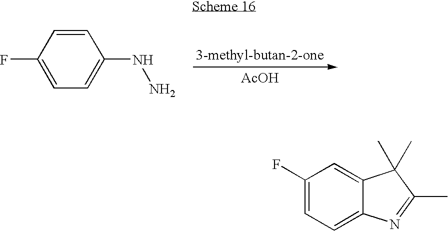

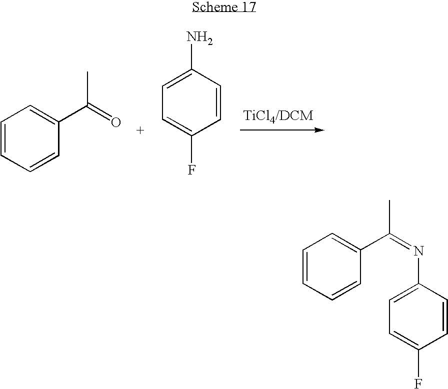

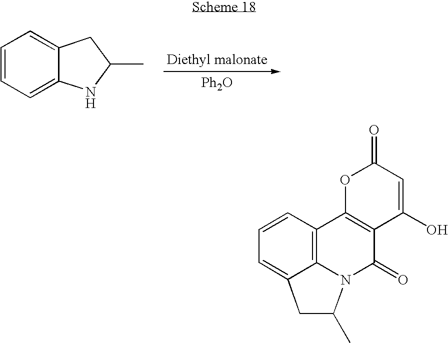

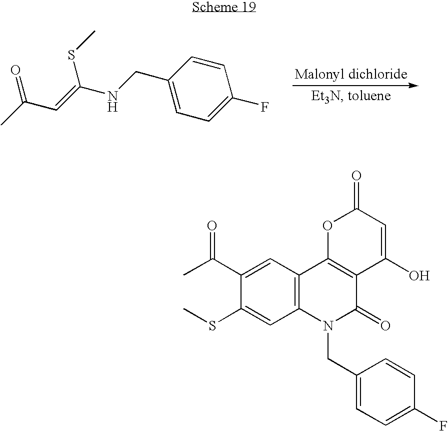

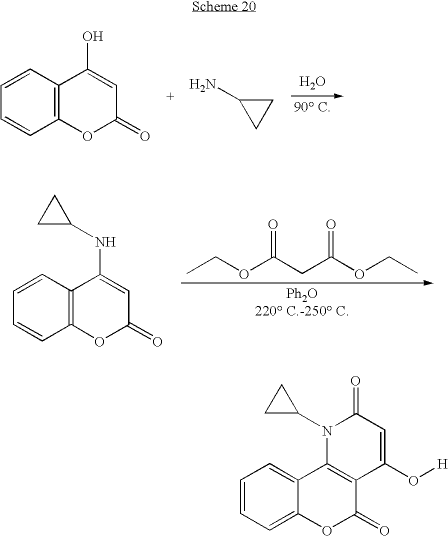

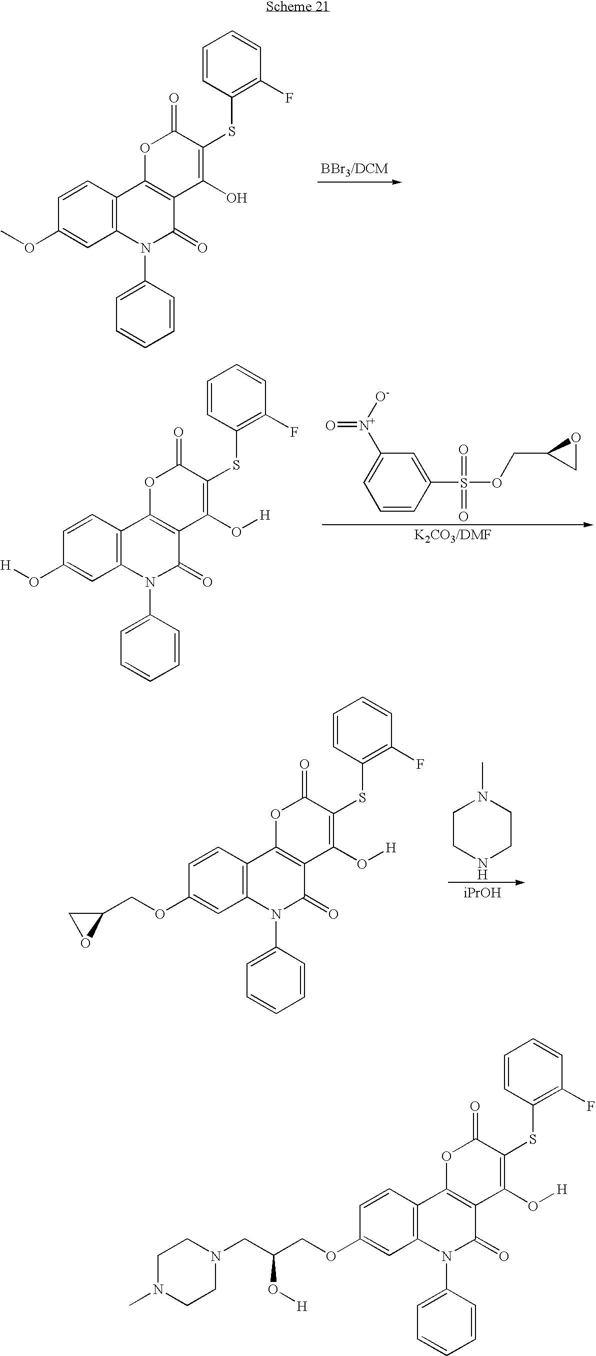

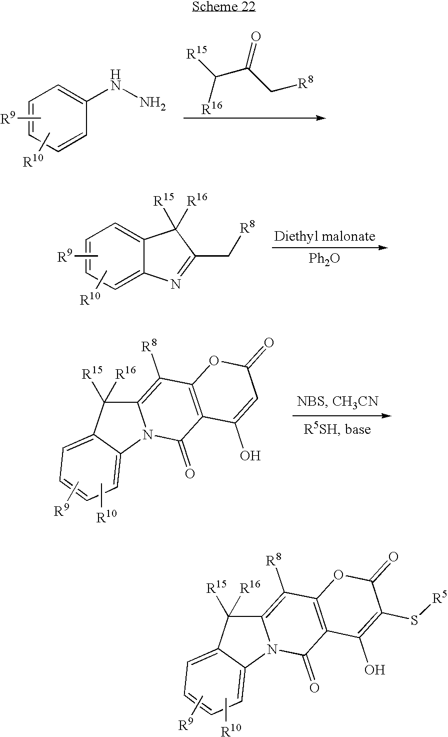

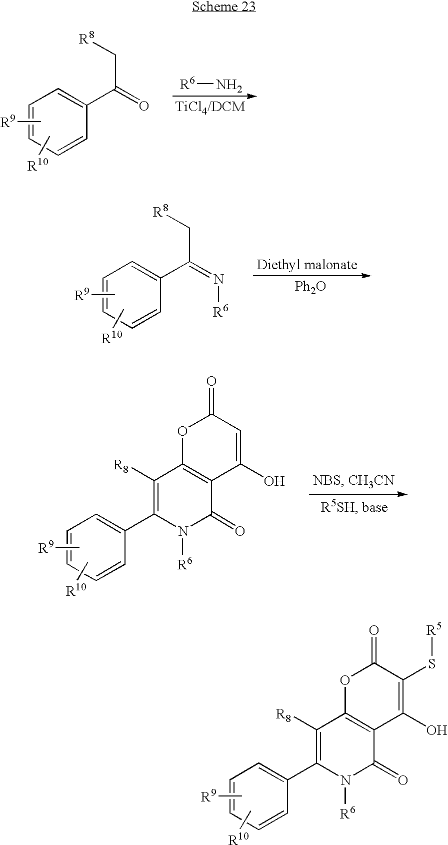

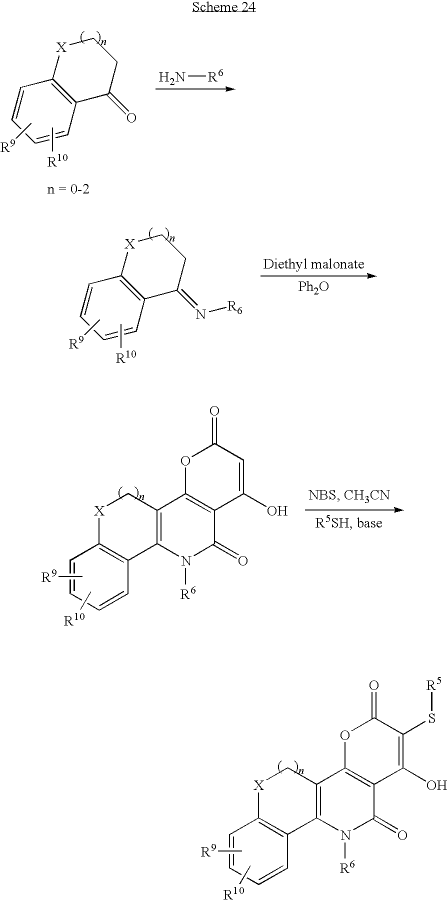

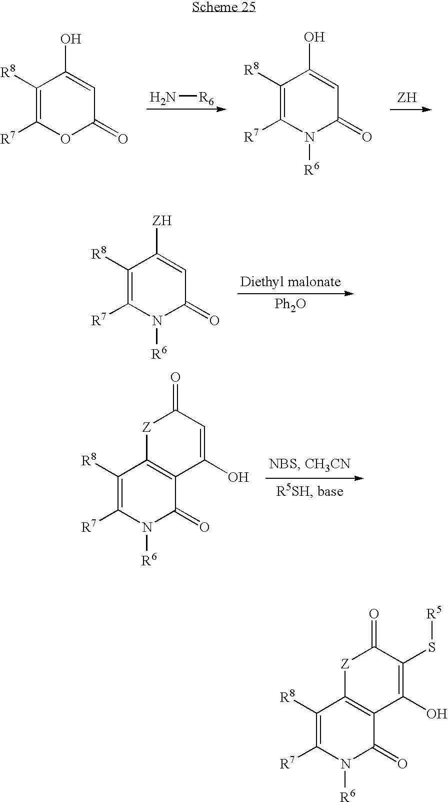

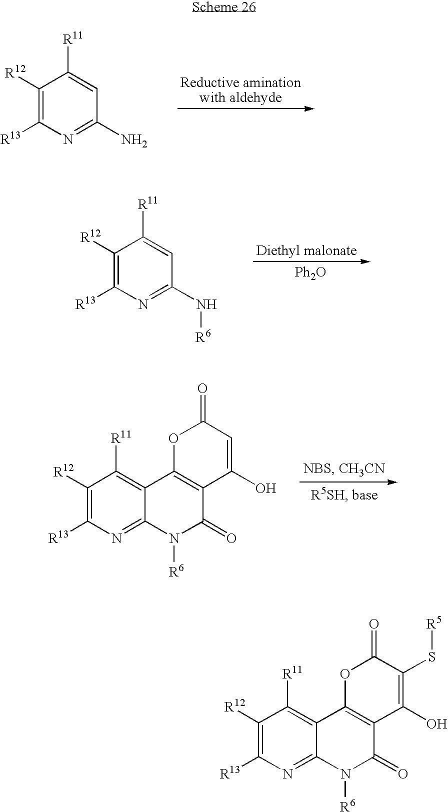

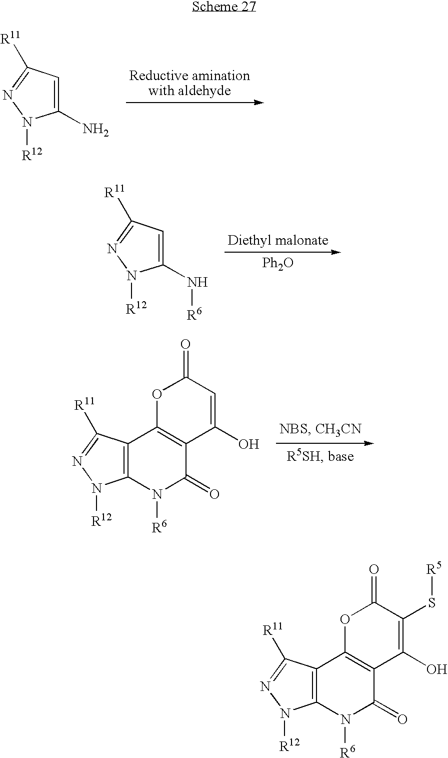

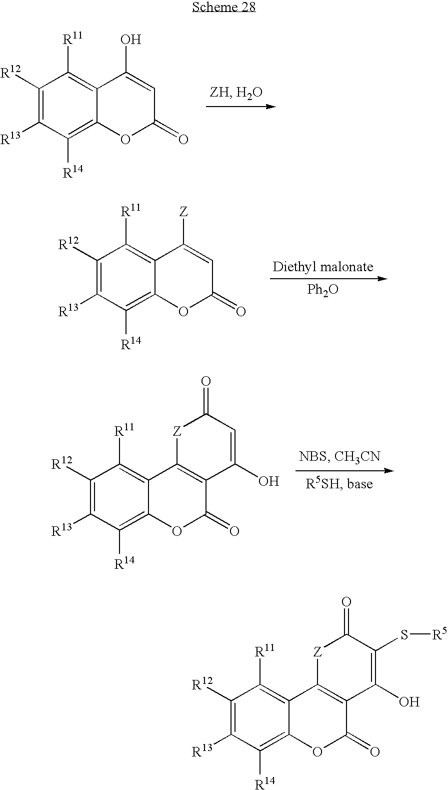

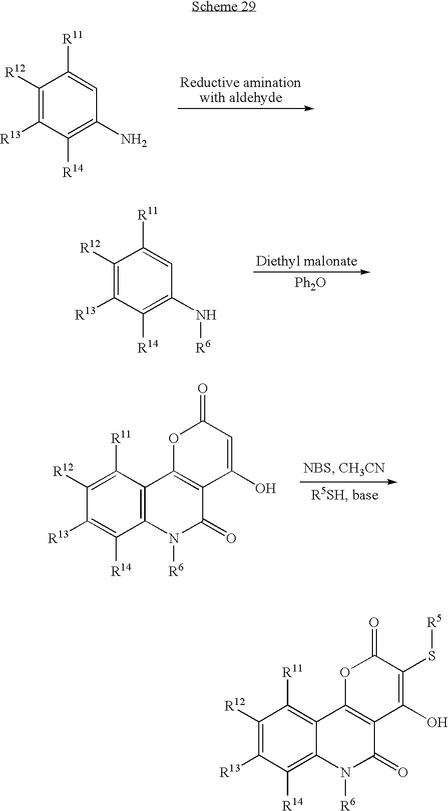

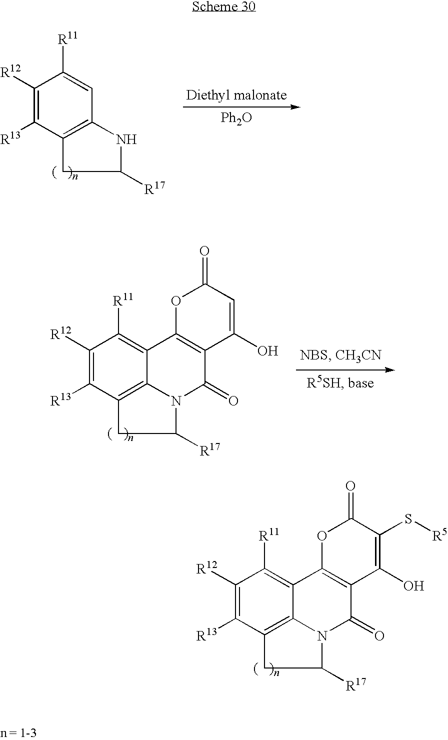

Pharmaceutically acceptable salts of the compounds provided herein can be formed by conventional and known techniques, such as by reacting a compound provided herein with a suitable acid or base.

### 5.4 Compositions

Any of the compounds provided herein, including the compounds described in Section 5.2 and Table 1, can optionally be in the form of a composition comprising the compound or its pharmaceutically acceptable salt, solvate, hydrate, prodrug or stereoisomer thereof.

In some embodiments, provided herein are compositions (including pharmaceutical compositions) comprising a compound and a pharmaceutically acceptable carrier, excipient, or diluent.

In certain embodiments, provided herein are pharmaceutical compositions comprising an effective amount of a compound and a pharmaceutically acceptable carrier, excipient, or diluent. The pharmaceutical compositions are suitable for veterinary and/or human administration.

The pharmaceutical compositions provided herein can be in any form that allows for the composition to be administered to a subject, said subject being an animal in one embodiment, including, but not limited to a human, or non-human animal.

In a specific embodiment and in this context, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is an exemplary carrier when the pharmaceutical composition is administered intravenously. Saline solutions and aqueous dextrose and glycerol solutions can also be employed as liquid carriers, particularly for injectable solutions. Examples of suitable pharmaceutical carriers are described in “Remington's Pharmaceutical Sciences” by E. W. Martin.

Typical compositions and dosage forms comprise one or more excipients. Suitable excipients are well-known to those skilled in the art of pharmacy, and non limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a patient and the specific active ingredients in the dosage form. The composition or single unit dosage form, if desired, can also contain minor amounts of wetting or emulsifying agents, or pH buffering agents.

In certain specific embodiments provided herein, the compositions are in oral, injectable, or transdermal dosage forms. In one specific embodiment, the compositions are in oral dosage forms.

### 5.5 Uses of Compounds

Compounds provided herein are useful generally as inhibitors of protein synthesis. Specifically, compounds provided herein are useful as inhibitors of a peptidyl tRNA hydrolase, in one embodiment a bacterial peptidyl hydrolase. In certain embodiments, the compounds provided herein exhibit specificity for bacterial peptidyl tRNA hydrolase enzymes compared to eukaryotic peptidyl tRNA hydrolase enzymes and in particular, mammalian peptidyl tRNA hydrolase enzymes. In a specific embodiment, a compound provided herein is an inhibitor of bacterial cell proliferation. In another embodiment, a compound provided herein is cytotoxic to bacterial cells and has comparatively low cytotoxicity in eukaryotic cells, in one embodiment mammalian cells. In alternative embodiment, a compound provided herein is cytostatic to bacterial cells and has comparatively low cytotoxicity in eukaryotic cells, in one embodiment mammalian cells.

As used in this context, the term low toxicity refers to a therapeutic window between effective dose whereby bacterial growth is inhibited, and non-specific cytotoxicity is observed having a detrimental effect on mammalian cell growth. The difference targeted for hit-to-lead molecules are greater than 5 fold between MIC and CC540. Development candidates are greater than 50 fold.

In one embodiment, a compound provided herein reduces or inhibits a bacterial infection. In a specific embodiment, a compound eliminates or reduces the amount of bacteria by 75%, 80%, 85%, 90%, 95%, 98%, 99%, 75-99.5%, 85-99.5%, or 90-99.8% in a subject as determined by an assay described herein or known to one of skill in the art. Accordingly, compounds provided herein are useful in methods of preventing, treating and/or managing bacterial infections. In a particular embodiment, a compound provided herein is useful in preventing, treating and/or managing a bacterial infection caused by a strain of bacteria that exhibits resistance to other antibacterial agents.

In certain embodiments, a compound provided herein inhibits or reduces bacterial protein synthesis by at least 20% to 25%, at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% as measured by a standard assay (e.g., an in vitro protein translation assay, or other inhibition assay) known to one of skill in the art, or an assay described herein.

In some embodiments, a compound provided herein inhibits or reduces bacterial proliferation by at least 20% to 25%, at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% as measured by an assay to determine the minimal inhibitory concentration (e.g., by microbroth dilution or agar diffusion) known to one of skill in the art, or an assay described herein.

In some embodiments, a compound provided herein eliminates or reduces the amount of bacteria by at least 20% to 25%, at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% as measured by bacterial assays known to one of skill in the art, or an assay described herein.

Bacterial infections reduced, inhibited, prevented, treated, and/or managed in accordance with the methods provided herein include infections caused by gram negative bacteria and gram positive bacteria. In a specific embodiment, the bacterial infection reduced, inhibited, prevented, treated, and/or managed is caused by an intracellular bacteria. In another embodiment, the bacterial infections reduced, inhibited, prevented, treated, and/or managed are resistant to one or more currently available antibiotics. Nonlimiting examples of bacteria which can cause bacterial infections that can be reduced, inhibited, prevented, treated, and/or managed in accordance with the methods provided herein include, but are not limited to Streptococcus pyogenes, Streptococcus pneumoniae, Neisseria gonorrhoea, Neisseria meningitidis, Corynebacterium diphtheriae, Clostridium botulinum, Clostridium perfringens, Clostridium tetani, Haemophilus influenzae, Klebsiella pneumoniae, Klebsiella ozaenae, Klebsiella rhinoscleromotis, Staphylococcus aureus, Vibrio cholerae, Escherichia coli, Pseudomonas aeruginosa, Campylobacter (Vibrio) fetus, Campylobacter jejuni, Aeromonas hydrophila, Bacillus cereus, Edwardsiella tarda, Yersinia enterocolitica, Yersinia pestis, Yersinia pseudotuberculosis, Shigella dysenteriae, Shigella flexneri, Shigella sonnei, Salmonella typhimurium, Treponema pallidum, Treponema pertenue, Treponema carateneum, Borrelia vincentii, Borrelia burgdorferi, Leptospira icterohemorrhagiae, Mycobacterium tuberculosis, Toxoplasma gondii, Pneumocystis carinii, Francisella tularensis, Brucella abortus, Brucella suis, Brucella melitensis, Mycoplasma spp., Rickettsia prowazeki, Rickettsia tsutsugumushi, Chlamydia spp., and Helicobacter pylori.

In certain embodiments, a compound provided herein reduces or inhibits a bacterial infection caused by one or more bacteria selected from the group consisting of Brucella, Bacillus, Yersinia, Coxiella, Francisella, Mycobacterium, Shigella, Salmonella, Vibrio, and Campylobacter.

In certain embodiments, a compound provided herein reduces or inhibits a bacterial infection caused by one or more bacteria selected from the group consisting of Haemophilus influenzae, Neisseria meningitidis, and Streptococcus pneumoniae.

In some embodiments, a compound provided herein reduces or inhibits a bacterial infection caused by one or more bacteria selected from the group consisting of Staphylococcus aureus, Staphylococcus epidermidis, Enterococcus faecium, Enterococcus faecalis, and Pseudomonas aeruginosa.

5.5.1 Prophylactic and Therapeutic Methods

Provided herein are methods of preventing, treating and/or managing a bacterial infection, said methods comprising administering to a subject in need thereof one or more compounds provided herein, such as a compound identified in accordance with the methods provided herein. In one embodiment, provided herein are methods of preventing, treating/and or managing a bacterial infection, said methods comprising administering to a subject having a bacterial infection a dose of a prophylactically or therapeutically effective amount of one or more compounds provided herein.

Further provided herein are methods of preventing, treating and/or managing a bacterial infection, said methods comprising administering to a subject in need thereof one or more compounds provided herein, and one or more other therapies (e.g., prophylactic or therapeutic agents). In a specific embodiment, the other therapies are currently being used, have been used or are known to be useful in the prevention, treatment and/or management of a bacterial infection. Non-limiting examples of such prophylactic or therapeutics are provided in §5.6, infra.

The combination therapies provided herein can be administered sequentially or concurrently. In one embodiment, the combination therapies provided herein comprise a compound provided herein and at least one other therapy which has the same mechanism of action. In another embodiment, the combination therapies provided herein comprise a compound provided herein and at least one other therapy which has a different mechanism of action than the compound.

In a specific embodiment, the combination therapies provided herein improve the prophylactic and/or therapeutic effect of a compound provided herein by functioning together with the compound to have an additive or synergistic effect. In another embodiment, the combination therapies provided herein reduce the side effects associated with each therapy taken alone.

The prophylactic or therapeutic agents of the combination therapies can be administered to a subject in the same pharmaceutical composition. Alternatively, the prophylactic or therapeutic agents of the combination therapies can be administered concurrently to a subject in separate pharmaceutical compositions. The prophylactic or therapeutic agents may be administered to a subject by the same or different routes of administration.

In certain embodiments, provided herein are methods for treating and/or managing a bacterial infection, in a subject refractory to conventional therapies for such an infection, the methods comprising administering to said subject a dose of a prophylactically or therapeutically effective amount of a compound provided herein. An infection may be determined to be refractory to a therapy means when at least some significant portion of the bacterial cells are not killed or their cell division arrested in response to the therapy. Such a determination can be made either in vivo or in vitro by any method known in the art for assaying the effectiveness of treatment on bacterial cells, using the art-accepted meanings of “refractory” in such a context.

5.5.2 Use as Disinfectant

Further provided herein are methods for the use of the compounds provided herein as active ingredients in products having antibacterial properties or in products in which it is desirable to have antibacterial activity. In one embodiment, one or more of the compounds provided herein is used as an additive in a cosmetic product, a personal hygiene product, or a household or industrial cleaning product. In another embodiment, one or more of the compounds provided herein is used as an additive in an antibacterial ointment or cream. In another embodiment one or more compounds provided herein is used as an additive to soap.

### 5.6 Agents Useful in Combination with Compounds

Therapeutic or prophylactic agents that can be used in combination with the compounds provided herein for the prevention, treatment and/or management of a bacterial infection include, but are not limited to, small molecules, synthetic drugs, peptides (including cyclic peptides), polypeptides, proteins, nucleic acids (e.g., DNA and RNA nucleotides including, but not limited to, antisense nucleotide sequences, triple helices, RNAi, and nucleotide sequences encoding biologically active proteins, polypeptides or peptides), antibodies, synthetic or natural inorganic molecules, mimetic agents, and synthetic or natural organic molecules. Specific examples of such agents include, but are not limited to, immunomodulatory agents (e.g., interferon), anti-inflammatory agents (e.g., adrenocorticoids, corticosteroids (e.g., beclomethasone, budesonide, flunisolide, fluticasone, triamcinolone, methylprednisolone, prednisolone, prednisone, hydrocortisone), glucocorticoids, steroids, and non-steriodal anti-inflammatory drugs (e.g., aspirin, ibuprofen, diclofenac, and COX-2 inhibitors), pain relievers, leukotreine antagonists (e.g., montelukast, methyl xanthines, zafirlukast, and zileuton), beta2-agonists (e.g., albuterol, biterol, fenoterol, isoetharie, metaproterenol, pirbuterol, salbutamol, terbutalin formoterol, salmeterol, and salbutamol terbutaline), anticholinergic agents (e.g., ipratropium bromide and oxitropium bromide), sulphasalazine, penicillamine, dapsone, antihistamines, anti-malarial agents (e.g., hydroxychloroquine), anti-viral agents (e.g., nucleoside analogs (e.g., zidovudine, acyclovir, gangcyclovir, vidarabine, idoxuridine, trifluridine, and ribavirin), foscarnet, amantadine, rimantadine, saquinavir, indinavir, ritonavir, and AZT) and antibiotics (e.g., dactinomycin (formerly actinomycin), bleomycin, erythomycin, penicillin, mithramycin, and anthramycin (AMC)).

Any therapy which is known to be useful, or which has been used or is currently being used for the prevention, management, and/or treatment of a bacterial infection or can be used in combination with the compounds provided herein in accordance with the invention described herein. See, e.g., Gilman et al., Goodman and Gilman's: The Pharmacological Basis of Therapeutics, 10th ed., McGraw-Hill, New York, 2001; The Merck Manual of Diagnosis and Therapy, Berkow, M. D. et al. (eds.), 17th Ed., Merck Sharp & Dohme Research Laboratories, Rahway, N.J., 1999; Cecil Textbook of Medicine, 20th Ed., Bennett and Plum (eds.), W.B. Saunders, Philadelphia, 1996 for information regarding therapies (e.g., prophylactic or therapeutic agents) which have been or are currently being used for preventing, treating and/or managing bacterial infections.

5.6.1 Antibacterial Agents

Antibacterial agents, including antibiotics, that can be used in combination with the compounds provided herein include, but are not limited to, aminoglycoside antibiotics, glycopeptides, amphenicol antibiotics, ansamycin antibiotics, cephalosporins, cephamycins oxazolidinones, penicillins, quinolones, streptogamins, tetracyclins, and analogs thereof.

In a specific embodiment, the compounds provided herein are used in combination with other protein synthesis inhibitors, including but not limited to, streptomycin, neomycin, erythromycin, carbomycin, and spiramycin.

In one embodiment, the antibacterial agent is selected from the group consisting of ampicillin, amoxicillin, ciprofloxacin, gentamycin, kanamycin, neomycin, penicillin G, streptomycin, sulfanilamide, and vancomycin. In another embodiment, the antibacterial agent is selected from the group consisting of azithromycin, cefonicid, cefotetan, cephalothin, cephamycin, chlortetracycline, clarithromycin, clindamycin, cycloserine, dalfopristin, doxycycline, erythromycin, linezolid, mupirocin, oxytetracycline, quinupristin, rifampin, spectinomycin, and trimethoprim

Additional, non-limiting examples of antibacterial agents for use in combination with the compounds provided herein include the following: aminoglycoside antibiotics (e.g., apramycin, arbekacin, bambermycins, butirosin, dibekacin, neomycin, neomycin, undecylenate, netilmicin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and imipenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cefinetazole, and cefminox), folic acid analogs (e.g., trimethoprim), glycopeptides (e.g., vancomycin), lincosamides (e.g., clindamycin, and lincomycin), macrolides (e.g., azithromycin, carbomycin, clarithomycin, dirithromycin, erythromycin, and erythromycin acistrate), monobactams (e.g., aztreonam, carumonam, and tigemonam), nitrofurans (e.g., furaltadone, and furazolium chloride), oxacephems (e.g., flomoxef, and moxalactam), oxazolidinones (e.g., linezolid), penicillins (e.g., amdinocillin, amdinocillin pivoxil, amoxicillin, bacampicillin, benzylpenicillinic acid, benzylpenicillin sodium, epicillin, fenbenicillin, floxacillin, penamccillin, penethamate hydriodide, penicillin o benethamine, penicillin 0, penicillin V, penicillin V benzathine, penicillin V hydrabamine, penimepicycline, and phencihicillin potassium), quinolones and analogs thereof (e.g., cinoxacin, ciprofloxacin, clinafloxacin, flumequine, grepagloxacin, levofloxacin, and moxifloxacin), streptogramins (e.g., quinupristin and dalfopristin), sulfonamides (e.g., acetyl sulfamethoxypyrazine, benzylsulfamide, noprylsulfamide, phthalylsulfacetamide, sulfachrysoidine, and sulfacytine), sulfones (e.g., diathymosulfone, glucosulfone sodium, and solasulfone), and tetracyclines (e.g., apicycline, chlortetracycline, clomocycline, and demeclocycline). Additional examples include cycloserine, mupirocin, tuberin amphomycin, bacitracin, capreomycin, colistin, enduracidin, enviomycin, and 2,4 diaminopyrimidines (e.g., brodimoprim).

5.6.2 Antiviral Agents

Antiviral agents that can be used in combination with the compounds provided herein include, but are not limited to, non-nucleoside reverse transcriptase inhibitors, nucleoside reverse transcriptase inhibitors, protease inhibitors, and fusion inhibitors. In one embodiment, the antiviral agent is selected from the group consisting of amantadine, oseltamivir phosphate, rimantadine, and zanamivir. In another embodiment, the antiviral agent is a non-nucleoside reverse transcriptase inhibitor selected from the group consisting of delavirdine, efavirenz, and nevirapine. In another embodiment, the antiviral agent is a nucleoside reverse transcriptase inhibitor selected from the group consisting of abacavir, didanosine, emtricitabine, emtricitabine, lamivudine, stavudine, tenofovir DF, zalcitabine, and zidovudine. In another embodiment, the antiviral agent is a protease inhibitor selected from the group consisting of amprenavir, atazanavir, fosamprenav, indinavir, lopinavir, nelfinavir, ritonavir, and saquinavir. In another embodiment, the antiviral agent is a fusion inhibitor such as enfuvirtide.

Additional, non-limiting examples of antiviral agents for use in combination with the compounds provided herein include the following: rifampicin, nucleoside reverse transcriptase inhibitors (e.g., AZT, ddI, ddC, 3TC, d4T), non-nucleoside reverse transcriptase inhibitors (e.g., delavirdine efavirenz, nevirapine), protease inhibitors (e.g., aprenavir, indinavir, ritonavir, and saquinavir), idoxuridine, cidofovir, acyclovir, ganciclovir, zanamivir, amantadine, and palivizumab. Other examples of anti-viral agents include but are not limited to acemannan; acyclovir; acyclovir sodium; adefovir; alovudine; alvircept sudotox; amantadine hydrochloride (SYMMETREL™); aranotin; arildone; atevirdine mesylate; pyridine; cidofovir; cipamfylline; cytarabine hydrochloride; delavirdine mesylate; desciclovir; didanosine; disoxaril; edoxudine; enviradene; enviroxime; famciclovir; famotine hydrochloride; fiacitabine; fialuridine; fosarilate; foscamet sodium; fosfonet sodium; ganciclovir; ganciclovir sodium; idoxuridine; kethoxal; lamivudine; lobucavir; memotine hydrochloride; methisazone; nevirapine; oseltamivir phosphate (TAMIFLU™); penciclovir; pirodavir; ribavirin; rimantadine hydrochloride (FLUMADINE™); saquinavir mesylate; somantadine hydrochloride; sorivudine; statolon; stavudine; tilorone hydrochloride; trifluridine; valacyclovir hydrochloride; vidarabine; vidarabine phosphate; vidarabine sodium phosphate; viroxime; zalcitabine; zanamivir (RELENZA™); zidovudine; and zinviroxime.

### 5.7 Dosages and Frequency

The amount of a compound provided herein, or the amount of a composition comprising the compound, that will be effective in the prevention, treatment and/or management of a bacterial infection can be determined by standard clinical techniques. In vitro or in vivo assays may optionally be employed to help identify optimal dosage ranges. The precise dose to be employed will also depend, e.g., on the route of administration, the type of infection, and the seriousness of the infection, and should be decided according to the judgment of the practitioner and each patient's circumstances.

Exemplary doses of the compounds or compositions provided herein include milligram or microgram amounts per kilogram of subject or sample weight (e.g., about 1 microgram per kilogram to about 500 milligrams per kilogram, about 5 micrograms per kilogram to about 100 milligrams per kilogram, or about 1 microgram per kilogram to about 50 micrograms per kilogram). In specific embodiments, a daily dose is at least 50 mg, 75 mg, 100 mg, 150 mg, 250 mg, 500 mg, 750 mg, or at least 1 g.

In one embodiment, the dosage is a concentration of 0.01 to 5000 mM, 1 to 300 mM, 10 to 100 mM and 10 mM to 1 M. In another embodiment, the dosage is a concentration of at least 5 μM, at least 10 μM, at least 50 μM, at least 100 μM, at least 500 μM, at least 1 mM, at least 5 mM, at least 10 mM, at least 50 mM, at least 100 mM, or at least 500 mM.

In a specific embodiment, the dosage is 0.25 μg/kg or more, 0.5 μg/kg or more, 1 μg/kg or more, 2 μg/kg or more, 3 μg/kg or more, 4 μg/kg or more, 5 μg/kg or more, 6 μg/kg or more, 7 μg/kg or more, 8 μg/kg or more, 9 μg/kg or more, or 10 μg/kg or more, 25 μg/kg or more, 50 μg/kg or more, 100 μg/kg or more, 250 μg/kg or more, 500 μg/kg or more, 1 mg/kg or more, 5 mg/kg or more, 6 mg/kg or more, 7 mg/kg or more, 8 mg/kg or more, 9 mg/kg or more, or 10 mg/kg or more of a patient's body weight.

The dosages of prophylactic or therapeutic agents other than a compound provided herein or composition provided herein which have been or are currently being used for the prevention, treatment and/or management of a bacterial infection can be determined using references available to a clinician such as, e.g., the Physicians' Desk Reference (55th ed. 2001). In one embodiment, dosages lower than those which have been or are currently being used to prevent, treat and/or manage the infection are utilized in combination with one or more compounds or compositions provided herein.

The above-described administration schedules are provided for illustrative purposes only and should not be considered limiting. A person of ordinary skill in the art will readily understand that all doses are within the scope of the embodiments provided herein.

### 5.8 Assays to Identify Compounds

The methods provided herein provide assays designed to identify novel, broad spectrum antibacterial compounds. In particular, the methods provided herein identify compounds having inhibitory activity against a bacterial peptidyl tRNA hydrolase (“Pth”). Pth inhibitors are further screened in a series of secondary assays designed to select for the ability to specifically inhibit bacterial cell proliferation. The methods provided herein further provide for the synthesis of novel compounds based on the identified Pth inhibitors. The novel compounds are designed using structure activity relationship analyses combined with molecular modeling approaches. The novel compounds represent compounds optimized for their ability to inhibit bacterial cell proliferation while maintaining low toxicity with respect to eukaryotic cells, in one embodiment mammalian cells. The novel compounds are also optimized for their ability to minimize the emergence of bacterial resistance. In a specific embodiment, compounds for use in the prevention, treatment and/or management of bacterial infections include those having a 50% inhibitory concentration of less than 1 micromolar against bacterial Pth, a minimal inhibitory concentration (“MIC”) of less than 1 micromolar, preferably, less than 0.80, 0.75, 0.50, 0.25, or 0.15 micromolar in assays of bacterial cell proliferation, a fifty to one hundred fold therapeutic window between the MIC value and cytoxicity, less than 90% binding to serum proteins, and sustained serum protein levels at least 4-fold above the MIC value.

Various in vitro assays can be used to identify and verify compounds having the desired antibacterial activity. Such assays include, for example, assays which measure the ability of a compound to inhibit Pth activity, inhibit bacterial protein synthesis, inhibit bacterial cell proliferation, or promote cytotoxicity in bacterial cells. Multiple in vitro assays can be performed simultaneously or sequentially to assess the antibacterial activity of a compound or a pool of compounds. In a specific embodiment, the in vitro assays described herein are performed in a high-throughput assay format.

5.8.1 Fluorescence Polarization Assay

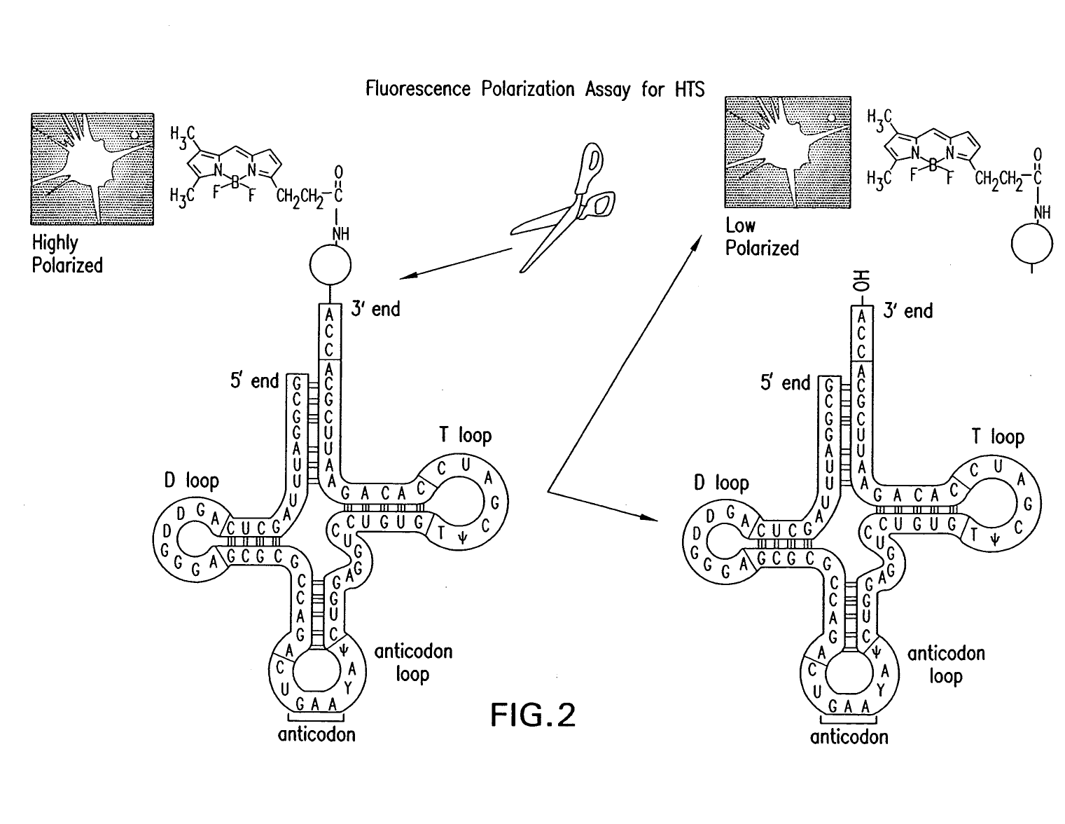

A compound, or a pool of compounds, can be tested for the ability to enhance or inhibit the activity of a peptidyl tRNA hydrolase using a cell-free fluorescence polarization assay. A substrate of the peptidyl tRNA hydrolase is labeled such that cleavage by the peptidyl tRNA hydrolase results in a decrease of size of the labeled portion of the substrate and thus, in a change of fluorescence polarization. The labeled substrate of the peptidyl tRNA hydrolase is incubated with a bacterial extract comprising peptidyl tRNA hydrolase or a purified peptidyl tRNA hydrolase and a compound to be tested. A compound that enhances the activity of the peptidyl tRNA hydrolase will result in an increase in cleavage, thus resulting in a change in the fluorescence polarization relative to a negative control or the absence of the compound, which will result in more of the light emitted being depolarized. In contrast, a compound that reduces the activity of the peptidyl tRNA hydrolase will decrease the amount of fluor tag released from the substrate relative to a negative control or the absence of the compound which will result in the emitted light remaining polarized. See, e.g., FIG. 2 for a schematic of the fluorescence polarization assay.

In such an assay, a fluorescently labeled substrate for a peptidyl tRNA hydrolase is contacted with a bacterial extract containing peptidyl tRNA hydrolase or a purified peptidyl tRNA hydrolase and a compound or a pool of compounds; and the fluorescently polarized light emitted is measured. An important aspect of this assay is that the size of the substrate used in the assay is large enough to distinguish a change in fluorescent polarized light emitted following cleavage of the substrate. The peptidyl tRNA hydrolase will cleave the substrate and result in a change in intensity of emitted polarized light. Fluorescently labeled substrates when excited with plane polarized light will emit light in a fixed plane only if they do not rotate during the period between excitation and emission. The extent of depolarization of the emitted light depends upon the amount of rotation of the substrate, which is dependent on the size of the substrate. Small substrates rotate more than larger substrates between the time they are excited and the time they emit fluorescent light. A small fluorescently labeled substrate rotates rapidly and the emitted light is depolarized. A large fluorescently labeled substrate rotates more slowly and results in the emitted light remaining polarized. A compound that inhibits or reduces the activity of the peptidyl tRNA hydrolase will inhibit or reduce the cleavage of the substrate relative to a negative control (e.g., PBS or DMSO), which will result in the emitted light remaining polarized. A compound that enhances the activity of the peptidyl tRNA hydrolase will enhance the cleavage of the substrate relative to a negative control (e.g., PBS or DMSO), which will result in more of the emitted light being depolarized.

The light intensities are measured in planes 90° apart and are conventionally designated the horizontal and vertical intensities. In some instruments the excitation filter is moveable while the emission filter is fixed. In certain other machines the horizontal and vertical intensities are measured simultaneously via fiber optics. Research grade fluorescence polarization instruments are commercially available from, e.g., Pan Vera, BMG Lab Technologies, and LJL Biosystems. Abbott provides clinical laboratory instrumentation. The value of fluorescence polarization is determined by the following equation:

$$polarization = \frac{{intensity}_{vertical} - {intensity}_{horizontal}}{{intensity}_{vertical} + {intensity}_{horizontal}}$$

Fluorescence polarization values are most often divided by 1000 and expressed as millipolarization units (mP).

The homogeneous assay format of fluorescence polarization allows for kinetic measurements and is sensitive, making it ideal for high throughput target screening. The assay design is compatible with e.g., 36-well, 64-well, 96-well and 384-well plate screening. Liquid handling systems are known in the art for transferring compounds and reagents (e.g., PlateMate Plus systems from Matrix (Hudson, N.H.) and PerkinElmer systems, the MiniTrak and the Multiprobe II HTEX). In a specific embodiment, each screening plate contains a set of standards comprising 64 wells: 16 wells representing totals (no compound, only compound solvent, e.g., DMSO), 16 wells of blanks (high inhibitor concentration, i.e., mature tRNA), and an 8 point dose response curve using mature tRNA in 8×4 wells. Data can be collected using commercially available imaging systems such as the ViewLux Imaging System (PerkinElmer). In a specific embodiment, simultaneous screening is performed with E. coli Pth using labeled substrate containing a different wavelength emitting fluorescent tag to eliminate false positives due to compound fluorescence.

5.8.2 FRET Assays

Fluorescence resonance energy transfer (“FRET”) can be used to detect alterations in the activity of a peptidyl tRNA hydrolase. In the FRET assays described herein, a substrate of the peptidyl tRNA hydrolase can be labeled with fluorophores using methods conventionally available in the art. See, e.g., Qin & Pyle, 1999, “Site-Specific Labeling of RNA with Fluorophores and Other Structural Probes,” in Methods: A Companion to Methods in Enzymology 18:60-70, which is hereby incorporated by reference in its entirety. In a specific embodiment, a substrate of peptidyl tRNA hydrolase is labeled with fluorophores.

Fluorescence resonance energy transfer (“FRET”) assays can be used to detect alterations in the activity of a peptidyl tRNA hydrolase, such as a bacterial peptidyl tRNA hydrolase. FRET based assays rely for signal generation on fluorescence resonance energy transfer, according to which a change in fluorescence is caused by a change in the distance separating a first fluorophore from an interacting resonance energy acceptor, either another fluorophore (a donor) or a quencher. Combinations of a fluorophore and an interacting molecule or moiety, including quenching molecules or moieties, are known as “FRET pairs” and are known to those skilled in the art.

The mechanism of FRET pair interaction requires that the absorption spectrum of one member of the pair overlaps the emission spectrum of the other member. If the interacting molecule or moiety is a quencher, its absorption spectrum must overlap the emission spectrum of the fluorophore (See, Stryer, L., Ann. Rev. Biochem. 1978, 47: 819 846; BIOPHYSICAL CHEMISTRY part II, Techniques for the Study of Biological Structure and Function, C. R. Cantor and P. R. Schimmel, pages 448 455 (W. H. Freeman and Co., San Francisco, U.S.A., 1980); Selvin, P. R., 1995, Methods in Enzymology 246: 300 335; all of which are incorporated herein by reference). Efficient, or a substantial degree of, FRET interaction requires that the absorption and emission spectra of the pair have a large degree of overlap. The efficiency of FRET interaction is linearly proportional to that overlap (Haugland et al., P.N.A.S. U.S.A. 63: 24 30 (1969). To obtain a large magnitude of signal, a high degree of overlap is required. FRET pairs, including fluorophore quencher pairs, have been chosen on that basis.

In order to obtain FRET between the fluorescent moiety and a quencher, the two moieties have to be in spatial proximity with each other. Thus, in certain embodiments, a substrate for a peptidyl tRNA hydrolase is labeled such that the fluorescent moiety a quencher are at most 0.5 nm, at most 1 nm, at most 5 nm, at most 10 nm, at most 20 nm, at most 30 nm, at most 40 nm, at most 50 m or at most 100 nm apart from each other.

The FRET assays may be conducted by contacting a substrate for peptidyl tRNA hydrolase with the enzyme and a compound provided herein, wherein the substrate is labeled at the 5′ end with a fluorophore or labeled internally and at the 3′ end with a quencher or, alternatively, the substrate is labeled at the 3′ end with a fluorophore and labeled internally or at the 5′ end with a quencher, and measuring the fluorescence of the substrate in, e.g., a fluorescence emission detector such as a Viewlux or Analyst. The peptidyl tRNA hydrolase will cleave the substrate and result in the production of a detectable fluorescent signal. A compound that inhibits or reduces the activity of the peptidyl tRNA hydrolase will inhibit or reduce the cleavage of the substrate and thus, inhibit or reduce the production of a detectable fluorescent signal relative to a negative control (e.g., PBS). A compound that enhances the activity of the peptidyl tRNA hydrolase will enhance the cleavage of the substrate and thus, increase the production of a detectable signal relative to a negative control (e.g., PBS).

In a FRET-based assay, excitation of the donor leads in enhanced fluorescence emission from the acceptor in the absence of the enzyme. In the presence of the enzyme, cleavage induces a separation between donor and acceptor reducing the acceptor emission. Thus, a compound that inhibits or reduces the activity of the peptidyl tRNA hydrolase will inhibit or reduce the cleavage of the substrate and thus, reduce the production of a detectable acceptor fluorescent signal relative to a negative control (e.g., PBS).

In a fluorescence quench assay, the substrate fluorescence is reduced due to the proximity of the fluorophore and the quencher. In the presence of the enzyme, cleavage induces a separation between the quencher and the fluorophore, increasing the fluorescence from the fluorophore. Thus, a compound that inhibits or reduced the activity of the peptidyl tRNA hydrolase will inhibit or reduce the cleavage of the substrate and thus, reduce the production of a detectable fluorescent signal relative to a negative control (e.g., PBS).

5.8.3 Radioassay

A radioactive assay can be used to measure the activity of a peptidyl tRNA hyrolasae enzyme. For example, the assay utilize a 96-well filter plate (Millipore Multiscreen FB). For kinetic assays, each enzyme and tRNA reaction aliquot (10 μl) is quenched by adding an excess (250 μl) of 5% trichloroacetic acid to precipitate tRNA in each well of the 96-well filter plate (Millipore Multiscreen FB). The assay is quantified by measuring the tritium signal of substrate remaining after filtration to remove the diacetyl-[³H]lysine cleavage product. Tritium is measured by liquid scintillation using a Wallac MicroBeta scintillation counter (Perkin-Elmer).

5.8.4 Substrates for Pth

Any known substrate of a peptidyl-tRNA hydrolase enzyme can be used in the methods provided herein. The substrate can be purified. The substrate may be purified from a bacterial cell or a eukaryotic cell, or the substrate may be chemically synthesized. In certain embodiments, the substrate is labeled with a detectable marker, such as a radiolabel or a fluorescent label. Such labels are well known in the art. Examples of radiolabels that may be incorporated into as substrate include isotopes of hydrogen, carbon, nitrogen, oxygen, phosphorous, fluorine and chlorine, such as 2H, 3H, 13C, 14C, 15N, 18O, 17O, 31P, 32P, 35S, 18F, and 36Cl. Preferably, a radiolabel is a 3H, 14C, 32P, or 35S label. In one embodiment, the substrate is labeled with a marker that is detectable upon cleavage of the substrate by a peptidyl tRNA hydrolase enzyme.

The following substrates are particularly suitable for use in a fluorescence polarization assay as described herein. In one embodiment, the substrate is an N-blocked aminoacylated tRNA, for example, with lysine or phenylalanine. In one embodiment, the N-blocked aminoacylated tRNA is aminoacylated with lysine. Lysine tRNA synthetase is required to specifically aminoacylate tRNALys (Sigma) with lysine to generate lys-tRNALys. In a specific embodiment, E. coli and S. aureus lysine tRNA synthetases (LysRS) containing a His6 tag are constructed in the pQE-70 and pQE-60 vectors, respectively (Qiagen, Valencia, Calif.). The enzymes are expressed in the E. coli expression strain M15-[pREP4] (Qiagen) and purified by metal affinity chromatography (Talon resin, Clontech, Inc.). The purified enzyme can be RNAase-free. In a specific embodiment, radioactive lysine-tRNALys is generated using tritium-labeled lysine (Amersham, Piscataway, N.J.) to a specific activity of 5,500 DPM/pmol and a catalytic efficiency of 5.24×106 M⁻¹ sec⁻¹ for tRNA. The lysine alpha-amino group on lys-tRNALys is further modified to generate the di-acetyl-lysine-tRNALys substrate according to published procedures.

In another embodiment, the substrate is an N-acylaminoacylated tRNA minihelix. In a specific embodiment, the substrate is an N-acylaminoacylated tRNA tyrosine minihelix from Methanoccus jannaschii. In one embodiment, the 3′ puromycin tRNA minihelix comprises a helix bound at its 3′ end to puromycin, the helix represented by the sequence 5′-CCGGCGGGCUGGUUCAAAUCCGGCCCGCCGGACC-3′. The 3′ puromycin tRNA minihelix is particularly useful as a substrate in the FRET assay. In another embodiment, the 3′ puromycin minihelix is used as a competitive inhibitor for peptidyl tRNA hydrolase in the assays described herein.

5.8.5 Compounds to be Tested

The compounds identified by the methods provided herein may be from libraries which comprise a variety of types of compounds or may be compounds that have been synthesized de novo. In one embodiment, a library is used for an initial screen of many compounds to identify promising candidate structures for further characterization and optimization. In one embodiment, the library is a library of small molecules.

Examples of libraries that can be screened in accordance with the methods provided herein include, but are not limited to: peptoids; random biooligomers; diversomers such as hydantoins, benzodiazepines and dipeptides; vinylogous polypeptides; nonpeptidal peptidomimetics; oligocarbamates; peptidyl phosphonates; peptide nucleic acid libraries; antibody libraries; carbohydrate libraries; and small molecule libraries (such as small organic molecule libraries). In some embodiments, the compounds in the libraries screened are nucleic acid or peptide molecules. In a non-limiting example, peptide molecules can exist in a phage display library. In other embodiments, the types of compounds include, but are not limited to, peptide analogs including peptides comprising non-naturally occurring amino acids, e.g., D-amino acids, phosphorous analogs of amino acids, such as α-amino phosphoric acids, or amino acids having non-peptide linkages, nucleic acid analogs such as phosphorothioates and PNAs, hormones, antigens, synthetic or naturally occurring drugs, opiates, dopamine, serotonin, catecholamines, thrombin, acetylcholine, prostaglandins, organic molecules, pheromones, adenosine, sucrose, glucose, lactose and galactose. Libraries of polypeptides or proteins can also be used in the assays provided herein.

In certain embodiments, the compound is a small molecule.

### 5.9 Cloning, Expression and Characterization of Pth for Use in Screening Assays

Techniques for practicing this specific aspect will employ, unless otherwise indicated, conventional techniques of molecular biology, microbiology, and recombinant DNA manipulation and production, which are routinely practiced by one of skill in the art. See, e.g., Sambrook, 1989, Molecular Cloning, A Laboratory Manual, Second Edition; DNA Cloning, Volumes I and II (Glover, Ed. 1985); Oligonucleotide Synthesis (Gait, Ed. 1984); Nucleic Acid Hybridization (Hames & Higgins, Eds. 1984); Transcription and Translation (Hames & Higgins, Eds. 1984); Animal Cell Culture (Freshney, Ed. 1986); Immobilized Cells and Enzymes (IRL Press, 1986); Perbal, A Practical Guide to Molecular Cloning (1984); Gene Transfer Vectors for Mammalian Cells (Miller & Calos, Eds. 1987, Cold Spring Harbor Laboratory); Methods in Enzymology, Volumes 154 and 155 (Wu & Grossman, and Wu, Eds., respectively), (Mayer & Walker, Eds., 1987); Immunochemical Methods in Cell and Molecular Biology (Academic Press, London, Scopes, 1987), Expression of Proteins in Mammalian Cells Using Vaccinia Viral Vectors in Current Protocols in Molecular Biology, Volume 2 (Ausubel et al., Eds., 1991).

5.9.1 Cloning and Expression of Pth Genes

The nucleotide sequences of various bacterial and eukaryotic peptidyl tRNA hydrolase genes are known in the art and these sequences can be cloned into an expression vector for making the peptidyl tRNA hydrolase enzyme for use in the methods provided herein. Examples of such sequences can be found, e.g., in public sequence databases such as GENBANK, the EMBL and NCBI database (e.g., Accession No. POA7D1 for the S. aureus Pth, Accession No. Q86Y79 for the human Pth, Accession No. B1204 for the E. coli Pth, and Accession No. RV1014C for Mycobacterium tuberculosis. Examples of sequences include, but are not limited to SAS0459 and SAS0503 from Staphylococcus aureus; BA0050, Bacillus anthracis, CBU1841, Coxiella burnetti, BR1536, Brucella suis; Rv1014c, Mycobacterium tuberculosis etc. The peptidyl tRNA hydrolase genes can be cloned into a suitable expression vector using techniques commonly known in the art of molecular biology. For example, oligonucleotide primers which hybridize to the coding sequence of a peptidyl tRNA hydrolase gene can be designed using routine skill. Such primers are then used to amplify the gene using a polymerase chain reaction. The amplified gene product is purified using routine methods and subsequently cloned into a suitable vector. The peptidyl tRNA hydrolase genes from various organisms, including E. coli, S. aureus, B. subtilis, and M. tuberculosis, as well as both human peptidyl tRNA hydrolase genes (Pth and Pth2), can be used to produce peptidyl tRNA hydrolase enzyme for use in the methods provided herein. In a specific embodiment, the peptidyl tRNA hydrolase gene of E. coli serves as the prototype for a Gram-negative organism and the peptidyl tRNA hydrolase gene of B. subtilis serves as the prototype for a Gram-positive organism.

In a specific embodiment, the peptidyl tRNA hydrolase genes are PCR-amplified and cloned into a plasmid vector engineered to express the protein with a C-terminal hexahistidine tag (His6). In a specific embodiment, the vector is pET-27b and the enzyme is then expressed in an E. coli host, such as BL21(DE3).

5.9.1.1 Expression Constructs

A variety of host-vector systems may be utilized to express a peptidyl tRNA hydrolase enzyme. Such relevant host-vector systems include, but are not limited to, mammalian cell systems infected with virus (e.g., vaccinia virus, adenovirus, etc.); insect cell systems infected with virus (e.g., baculovirus); microorganisms such as yeast containing yeast vectors, or bacteria transformed with bacteriophage, DNA, plasmid DNA, or cosmid DNA; and stable cell lines generated by transformation using a selectable marker. The expression elements of vectors vary in their strengths and specificities. Depending on the host-vector system utilized, any one of a number of suitable transcription and translation elements may be used.

Any of the methods known in the art for the insertion of DNA fragments into a vector may be used to construct expression vectors containing a chimeric nucleic acid consisting of appropriate transcriptional/translational control signals and the protein coding sequences. These methods may include in vitro recombinant DNA and synthetic techniques and in vivo recombinants (genetic recombination). Expression of the peptidyl tRNA hydrolase may be regulated by a second nucleic acid sequence so that the peptidyl tRNA hydrolase is expressed in a host transformed with the recombinant DNA molecule. For example, expression of a gene construct may be controlled by any promoter/enhancer element known in the art, such as a constitutive promoter, a tissue-specific promoter, or an inducible promoter. Specific examples of promoters which may be used to control gene expression include, but are not limited to, the SV40 early promoter region (Bernoist & Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42); prokaryotic expression vectors such as the β-lactamase promoter (Villa-Kamaroff et al., 1978, Proc. Natl. Acad. Sci. U.S.A. 75:3727-3731), or the tac promoter (DeBoer et al., 1983, Proc. Natl. Acad. Sci. U.S.A. 80:21-25); see also “Useful proteins from recombinant bacteria” in Scientific American, 1980, 242:74-94; plant expression vectors comprising the nopaline synthetase promoter region (Herrera-Estrella et al., Nature 303:209-213) or the cauliflower mosaic virus 35S RNA promoter (Gardner et al., 1981, Nucl. Acids Res. 9:2871), and the promoter of the photosynthetic enzyme ribulose biphosphate carboxylase (Herrera-Estrella et al., 1984, Nature 310:115-120); promoter elements from yeast or other fungi such as the Gal 4 promoter, the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I gene control region, which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); insulin gene control region, which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region, which is active in testicular, breast, lymphoid and mast cells (Leder et al., 1986, Cell 45:485-495), albumin gene control region, which is active in liver (Pinkert et al., 1987, Genes and Devel. 1:268-276), alpha-fetoprotein gene control region, which is active in liver (Krumlauf et al., 1985, Mol. Cell. Biol. 5:1639-1648; Hammer et al., 1987, Science 235:53-58; alpha 1-antitrypsin gene control region, which is active in the liver (Kelsey et al., 1987, Genes and Devel. 1:161-171), beta-globin gene control region, which is active in myeloid cells (Mogram et al., 1985, Nature 315:338-340; Kollias et al., 1986, Cell 46:89-94; myelin basic protein gene control region which is active in oligodendrocyte cells in the brain (Readhead et al., 1987, Cell 48:703-712); myosin light chain-2 gene control region which is active in skeletal muscle (Sani, 1985, Nature 314:283-286), and gonadotropic releasing hormone gene control region which is active in the hypothalamus (Mason et al., 1986, Science 234:1372-1378).

In a specific embodiment, a vector is used that comprises a promoter operably linked to a peptidyl tRNA hydrolase, one or more origins of replication, and, optionally, one or more selectable markers (e.g., an antibiotic resistance gene). In a certain embodiments, the vectors are CMV vectors, T7 vectors, lac vectors, pCEP4 vectors, 5.0/F vectors, or vectors with a tetracycline-regulated promoter (e.g., pcDNATM5/FRT/TO from Invitrogen). In a specific embodiment, the vector is pET-27b.

Expression vectors containing the peptidyl tRNA hydrolase construct can be identified by three general approaches: (a) nucleic acid hybridization, (b) presence or absence of “marker” nucleic acid functions, (c) expression of inserted sequences, and (d) sequencing. In the first approach, the presence of the gene inserted in an expression vector can be detected by nucleic acid hybridization using probes comprising sequences that are homologous to the inserted gene. In the second approach, the recombinant vector/host system can be identified and selected based upon the presence or absence of certain “marker” nucleic acid functions (e.g., thymidine kinase activity, resistance to antibiotics, transformation phenotype, occlusion body formation in baculovirus, etc.) caused by the insertion of the nucleic acid of interest, i.e., the peptidyl tRNA hydrolase gene construct, in the vector. For example, if the nucleic acid of interest is inserted within the marker nucleic acid sequence of the vector, recombinants containing the insert can be identified by the absence of the marker nucleic acid function. In the third approach, recombinant expression vectors can be identified by assaying the gene product expressed by the recombinant. Such assays can be based, for example, on the physical or functional properties of the particular gene.

5.9.1.2 Expression Systems and Host Cells

Mammalian host cells include but are not limited to those derived from humans, monkeys and rodents, (see, for example, Kriegler M. in “Gene Transfer and Expression: A Laboratory Manual”, New York, Freeman & Co. 1990), such as monkey kidney cell line transformed by SV40 (COS-7, ATCC Accession No. CRL 1651); human embryonic kidney cell lines (293, 293-EBNA, or 293 cells subcloned for growth in suspension culture, Graham et al., J. Gen. Virol., 36:59, 1977; baby hamster kidney cells (BHK, ATCC Accession No. CCL 10); chinese hamster ovary-cells-DEFER(CHO, Umlaut and Chasing. Proc. Natl. Acad. Sci. 77; 4216, 1980); mouse sterol cells (Mother, Biol. Report. 23:243-251, 1980); mouse fibroblast cells (NIGH-3T3), monkey kidney cells (CIV ATCC Accession No. CCL 70); african green monkey kidney cells (VERO-76, ATCC Accession No. CRL-1587); human cervical carcinoma cells (HELA, ATCC Accession No. CCL 2); canine kidney cells (MDCK, ATCC Accession No. CCL 34); buffalo rat liver cells (BRL 3A, ATCC Accession No. CRL 1442); human lung cells (W138, ATCC Accession No. CCL 75); human liver cells (Hep G2, HB 8065); and mouse mammary tumor cells (MMT 060562, ATCC Accession No. CCL51).

A number of viral-based expression systems may also be utilized with mammalian cells to produce a peptidyl tRNA hydrolase enzyme. Vectors using DNA virus backbones have been derived from simian virus 40 (SV40) (Hamer et al., 1979, Cell 17:725), adenovirus (Van Doren et al., 1984, Mol Cell Biol 4:1653), adeno-associated virus (McLaughlin et al., 1988, J Virol 62:1963), and bovine papillomas virus (Zinn et al., 1982, Proc Natl Acad Sci 79:4897). In cases where an adenovirus is used as an expression vector, the donor DNA sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing heterologous products in infected hosts. (See e.g., Logan and Shenk, 1984, Proc. Natl. Acad. Sci. (USA) 81:3655-3659).

Other useful eukaryotic host-vector system may include yeast and insect systems. In yeast, a number of vectors containing constitutive or inducible promoters may be used with Saccharomyces cerevisiae (baker's yeast), Schizosaccharomyces pombe (fission yeast), Pichia pastoris, and Hansenula polymorpha (methylotropic yeasts). For a review see, Current Protocols in Molecular Biology, Vol. 2, 1988, Ed. Ausubel et al., Greene Publish. Assoc. & Wiley Interscience, Ch. 13; Grant et al., 1987, Expression and Secretion Vectors for Yeast, in Methods in Enzymology, Eds. Wu & Grossman, 1987, Acad. Press, N.Y., Vol. 153, pp. 516-544; Glover, 1986, DNA Cloning, Vol. II, IRL Press, Wash., D.C., Ch. 3; and Bitter, 1987, Heterologous Gene Expression in Yeast, Methods in Enzymology, Eds. Berger & Kimmel, Acad. Press, N.Y., Vol. 152, pp. 673-684; and The Molecular Biology of the Yeast Saccharomyces, 1982, Eds. Strathern et al., Cold Spring Harbor Press, Vols. I and II.

In an insect system, Autographa californica nuclear polyhidrosis virus (AcNPV) a baculovirus, can be used as a vector to express the peptidyl-tRNA hydrolase in Spodoptera frugiperda cells. The sequences encoding Pth may be cloned into non-essential regions (for example the polyhedrin gene) of the virus and placed under control of an AcNPV promoter (for example the polyhedrin promoter). These recombinant viruses are then used to infect host cells in which the inserted DNA is expressed. (See e.g., Smith et al., 1983, J Virol 46:584; Smith, U.S. Pat. No. 4,215,051.)

Any of the cloning and expression vectors described herein may be synthesized and assembled from known DNA sequences by well known techniques in the art. The regulatory regions and enhancer elements can be of a variety of origins, both natural and synthetic. Some vectors and host cells may be obtained commercially. Non-limiting examples of useful vectors are described in Appendix 5 of Current Protocols in Molecular Biology, 1988, ed. Ausubel et al., Greene Publish. Assoc. & Wiley Interscience, which is incorporated herein by reference; and the catalogs of commercial suppliers such as Clontech Laboratories, Stratagene Inc., and Invitrogen, Inc.

Expression constructs containing a cloned nucleotide sequence encoding a peptidyl tRNA hydrolase enzyme can be introduced into the host cell by a variety of techniques known in the art, including but not limited to, for prokaryotic cells, bacterial transformation (Hanahan, 1985, in DNA Cloning, A Practical Approach, 1:109-136), and for eukaryotic cells, calcium phosphate mediated transfection (Wigler et al., 1977, Cell 11:223-232), liposome-mediated transfection (Schaefer-Ridder et al., 1982, Science 215:166-168), electroporation (Wolff et al., 1987, Proc Natl Acad Sci 84:3344), and microinjection (Cappechi, 1980, Cell 22:479-488).

5.9.1.3 Purification of Recombinant Proteins

Generally, a recombinant peptidyl tRNA hydrolase enzyme can be recovered and purified from cell cultures by known methods, including ammonium sulfate precipitation, acid extraction, anion or cation exchange chromatography, phosphocellulose chromatography, immunoaffinity chromatography, hydroxyapatite chromatography, and lectin chromatography.

In certain embodiments, the expression vector is engineered so that the peptidyl tRNA hydrolase enzyme is produced with a molecular tag at one end in order to facilitate purification of the enzyme. For example, the enzyme produced as a fusion with an affinity tag can be purified by affinity chromatography. Examples of affinity tags include the constant regions of immunoglobulins (purified using protein A or protein G affinity), a polyhistidine tag (purified using metal chelate chromatography), glutathione-5-transferase (purified using glutathione affinity), the maltose binding protein (MBP) of E. coli (purified using an amylose resin), and peptide tags that contain an epitope for which polyclonal or monoclonal antibodies are available (purified by immunoaffinity chromatography or immunoprecipitation using the appropriate antibody).

Methods of affinity purification using these tags are well known and routinely practiced in the art. For example, Protein-A or -G sepharose (Pharmacia or Biorad) can used as the solid phase for affinity purification of a peptidyl tRNA hydrolase fused to an immunoglobulin constant region fragment (“Fc”). Bound enzyme-Fc fusion protein can be eluted by various buffer systems known in the art, including a succession of citrate, acetate and glycine-HCl buffers which gradually lowers the pH. See, for example, Langone, 1982, J. Immunol. meth. 51:3; Wilchek et al., 1982, Biochem. Intl. 4:629; Sjobring et al., 1991, J. Biol. Chem. 26:399; page 617-618, in Antibodies A Laboratory Manual, edited by Harlow and Lane, Cold Spring Harbor laboratory, 1988.

The polyhistidine tag, usually a sequence of six histidines, has a high affinity for divalent metal ions, such as nickel ions (Ni2+), which can be immobilized on a solid phase, such as nitrilotriacetic acid-matrices. Polyhistidine has a well characterized affinity for Ni2+-NTA-agarose, and can be eluted with either of two mild treatments: imidazole (0.1-0.2 M) will effectively compete with the resin for binding sites; or lowering the pH just below 6.0 will protonate the histidine sidechains and disrupt the binding. The purification method comprises loading the cell culture lysate onto the Ni2+-NTA-agarose column, washing the contaminants through, and eluting the peptidyl tRNA hydrolase subunit with imidazole or weak acid. Ni2+-NTA-agarose can be obtained from commercial suppliers such as Sigma (St. Louis) and Qiagen. Antibodies that recognize the polyhistidine tag are also available which can be used to detect and quantitate the peptidyl tRNA hydrolase.

A peptidyl tRNA hydrolase enzyme-GST fusion protein expressed in a prokaryotic host cell, such as E. coli, can be purified from the cell culture lysate by absorption with glutathione agarose beads, followed by elution in the presence of free reduced glutathione at neutral pH.

A peptidyl hydrolase enzyme fused to MBP binds to amylose resin while contaminants are washed away. The bound enzyme-MBP fusion is then eluted from the amylose resin by maltose. See, for example, Guan et al., 1987, Gene 67:21-30.

Examples of techniques for immunoaffinity purifications can be found, for example, in Chapter 13 of Antibodies A Laboratory Manual, edited by Harlow and Lane, Cold Spring Harbor laboratory, 1988; and Chapter 8, Sections I and II, in Current Protocols in Immunology, ed. by Coligan et al., John Wiley, 1991; the disclosure of which are both incorporated by reference herein.

In a specific embodiment, the peptidyl tRNA hydrolase is purified by chromatography over a metal affinity resin (Ni-NTA Superflow, Qiagen), followed by ion exchange chromatography. In one embodiment, the peptidyl tRNA hydrolase enzyme is greater than 95% pure and free of contaminating RNases. In certain embodiments, the peptidyl tRNA hydrolase enzyme is at least 80% pure, at least 85% pure, at least 90% pure, or at least 95% pure.

### 5.10 Characterization of Compounds

5.10.1 Characterization of Antibacterial Activity

The biological activity of the compounds provided herein is measured in various in vitro and in vivo assays as described herein. In one embodiment, the compounds provided herein exhibit an activity profile that is consistent with their ability to inhibit bacterial cell proliferation while maintaining low toxicity with respect to eukaryotic cells, such as mammalian cells. For example, compounds provided herein include those having the activity profiles described below. The biological activities referred to herein are determined by the methods described in Sections 5.8 and 5.12, and by methods known to those of skill in the art.

The compounds provided herein include those having a 50% inhibitory concentration (“IC₅₀”) of less than 1 micromolar against bacterial Pth. In certain embodiments, a compound provided herein has an IC₅₀ of less than 1.0, 0.50, 0.25 or 0.50 micromolar against bacterial Pth. In certain embodiments, a compound provided herein has an IC₅₀ of less than 0.10, 0.050, 0.025 or 0.05 micromolar against bacterial Pth.

The compounds provided herein include those having a minimal inhibitory concentration (“MIC”) of less than 1 micromolar, in one embodiment 0.50 or less micromolar, in assays of bacterial cell proliferation. In certain embodiments, the MIC of a compound provided herein is less than 0.10, 0.25, 0.50 or 0.75 micromolar. In certain embodiments, the MIC of a compound provided herein is 0.5 to 1 μM, 0.1 to 0.9 μM, 0.1 to 0.5 μM, or 0.05 to 0.1 μM.

The compounds provided herein include those having a fifty to one hundred fold therapeutic window between the in vitro MIC value and cytoxicity. In certain embodiments, the therapeutic window is at least 50-fold, 75-fold, 100-fold, 150-fold, or 200-fold. In certain embodiments, the therapeutic window is 50-75 fold, 50-100 fold, 75-150 fold, 100 to 175 fold, 150 to 200 fold.

In one embodiment, the compounds provided herein exhibit low binding to serum proteins, including but not limited to serum albumin. In certain embodiments, a compound provided herein exhibits binding to serum proteins of less than 65%, 70%, 80%, 90% or 95%. In certain embodiments, a compound provided herein exhibits binding to serum proteins of 80-95%, 85-95%, 90 to 99.5%.

In one embodiment, compounds provided herein can be maintained at a suitable serum level following administration to a subject. In certain embodiments, a compound provided herein is sustained at levels of at least 2-fold, 3-fold, 4-fold, 5-fold, or 10-fold above its MIC value. In a specific embodiment, the compound is sustained at levels of at least 4-fold above its MIC value. In a specific embodiment, the compound is sustained at levels of 2 to 15 fold, 2 to 10 fold, 4 to 10 fold, 8 to 10 fold, 10 to 15 fold above its MIC value.

5.10.2 Characterization of Structure

In certain embodiments, the compounds provided herein are from a library, such as a library of small molecules. If the library comprises arrays or microarrays of compounds, wherein each compound has an address or identifier, the compound can be deconvoluted, e.g., by cross-referencing the positive sample to original compound list that was applied to the individual test assays. If the library is a peptide or nucleic acid library, the sequence of the compound can be determined by direct sequencing of the peptide or nucleic acid. Such methods are well known to one of skill in the art.

In other embodiments, the compounds provided herein are synthesized de novo. A number of physio-chemical techniques can be used for the de novo characterization of compounds bound to the peptidyl tRNA hydrolase. Examples of such techniques include, but are not limited to, mass spectrometry, NMR spectroscopy, X-ray crystallography and vibrational spectroscopy.

### 5.11 Design of Congeners or Analogs

Compounds provided herein can be used for the synthesis of novel chemical entities having increased potency and enhanced pharmacokinetic properties compared to the original compounds. Synthesized compounds are subjected to a series of secondary tests, including IC₅₀ determinations, ability to inhibit bacterial cell proliferation (quantitated by the MIC value), cytotoxicity assays, and target specificity testing.

In specific embodiments, the novel compounds are selected for a combination of one or more of the following properties: low IC₅₀ value (<10 micromolar) against bacterial peptidyl tRNA hydrolase, antibacterial activity (MIC<5 ug/ml), and low cytotoxicity with respect to eukaryotic, such as mammalian cells (>50 fold difference over the MIC).

Once a compound is identified as an inhibitor of bacterial peptidyl tRNA hydrolase, schemes for synthesizing families of molecules around the structure of interest (“SOI”) are used to initiate structure activity relationship (“SAR”) studies. Derivatives or structural variants of identified compounds are prepared based on the results of the SAR studies. Computational approaches driven by pharmacophore models are also utilized to identify compounds for synthesis and testing. The SAR studies characterize the SOI and determine the regions of the molecule critical for activity. Specifically, the SAR studies are useful for the identification of the minimum pharmacophore in each scaffold, enhanced potency, reduced toxicity, improved selectivity, and maximum oral bioavailability. Regions of the molecule that are not critical for activity are then modified to improve the cell permeability and metabolic characteristics of the compound. For example, intestinal permeability can be estimated by measuring in cultured Caco-2 cells and metabolic stability can be modeled by incubation of the drug with microsomes followed by quantification of the remaining parent compound by HPLC.

5.11.1 Molecular Modeling Approaches

In certain embodiments, the compounds provided herein are designed and selected for their ability to block the approach of the peptidyl fragment to the catalytic site, or reduce the association of the substrate to the enzyme using molecular modeling techniques commonly known to those skilled in the art. Thus, according to certain embodiments, a compound provided herein binds to the tRNA recognition site of a peptidyl tRNA hydrolase enzyme. In other embodiments, a compound provided herein binds to the catalytic site of a peptidyl tRNA hydrolase enzyme.

In one embodiment, a compound provided herein is designed based in part on the steric, electronic, and hydrogen-bonding potentials (via molecular field analysis) of the tRNA recognition site or the catalytic site of a peptidyl tRNA hydrolase enzyme so that the compound is able to bind to either site.

In certain embodiments, the compounds provided herein are produced in silico, as “protomolecules.” Such compounds are useful as a basis for developing a pseudo-binding energy, including desolvation terms, which is used as the target function in a genetic algorithm for the construction of molecules with improved binding to the peptidyl tRNA hydrolase enzyme active sites. A stochastic selection process assembles these protomolecules from a collection of small molecular fragments. The fragments represent functional groups, rings, and other moieties commonly found in therapeutic agents. Elaboration of these protomolecules is performed by additional stochastic selection from a matrix of refinement rules, and a collection of optimization choices including geometric manipulation of translation, rotation, dihedral scanning, joining and/or trimming of fragments, fragment mutations, analysis of complementarity to the protein H-bonding environment, etc. From a collection of runs, the process can generate approximately 1000 protomolecules per site studied. Further prioritization of such protomolecules involves consideration of additional scoring functions, evaluation of synthetic suitability by medicinal chemistry, and structural clustering/maximal common substructure analysis to elucidate potential pharmacophores for further molecular design.

In certain embodiments, the compounds provided herein are designed based on the application of Quantitative Structure Activity Relationship (QSAR) techniques such as, but not limited to, Linear Free Energy Relationships, CoMFA, Pharmacophore identification and mapping, Maximal common Substructure deconvolution, database, mining, similarity or diversity metric analysis, or other computational techniques known to and practiced by those skilled in the art on single molecules or ensembles thereof.

In certain embodiments, the compounds provided herein are designed to bind to regions on the surface of bacterial and putative human peptidyl tRNA hydrolase enzymes that control selectivity or recognition events. In order to address potential selectivity issues, a full atomistic model of peptidyl tRNA hydrolase, e.g., from M. tuberculosis, can be generated using standard energy minimization protocols, proper orientation of side chains, and consideration of intra-peptide interactions (i.e., salt bridges, disulfide binding, etc.). A comparison of active sites, allosteric regions, and protein-tRNA interfaces between the known E. coli and predicted M. tuberculosis structures will isolate differences that may control selectivity or substrate recognition between these enzymes. In addition, a threading/minimization paradigm can be applied to a putative human homolog of the bacterial peptidyl tRNA hydrolase to elucidate similar information about the human enzyme.

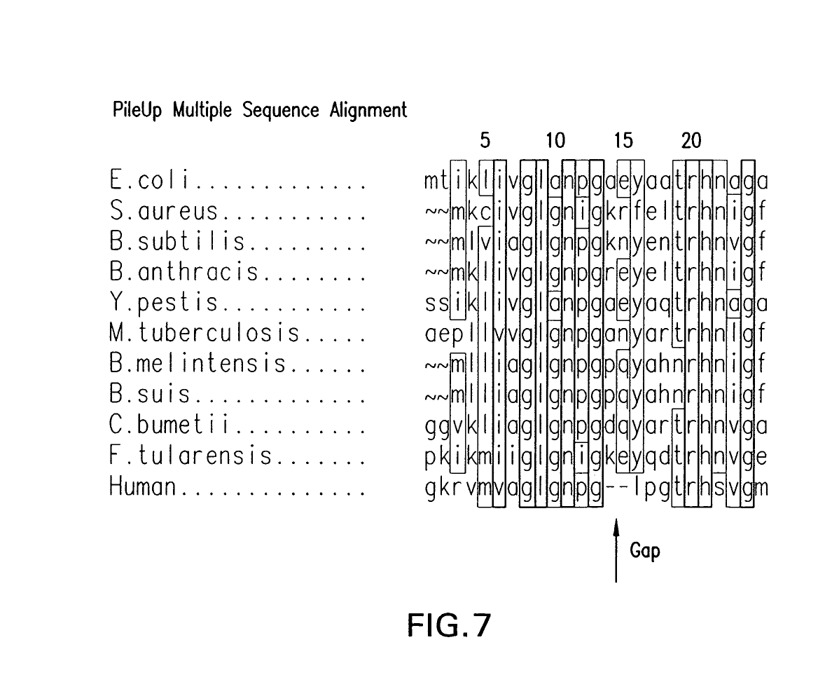

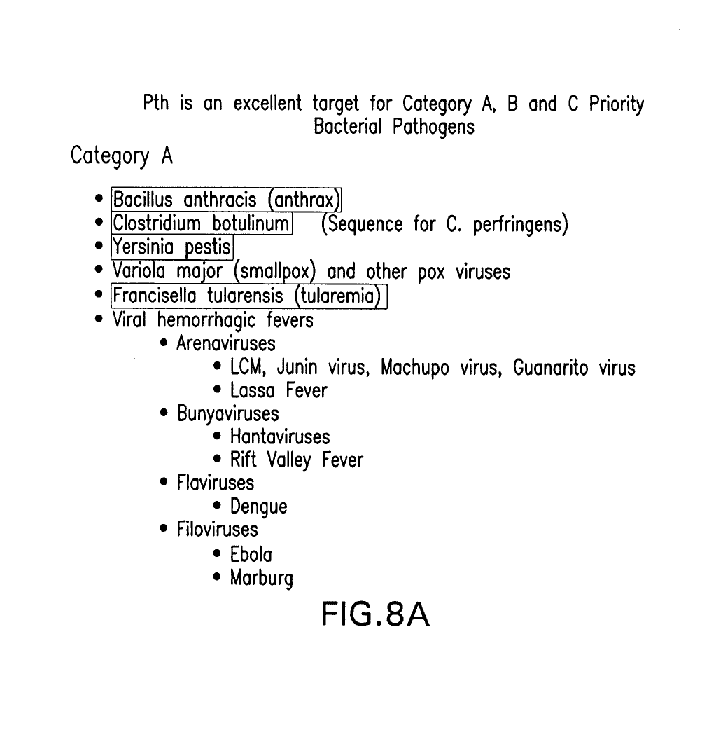

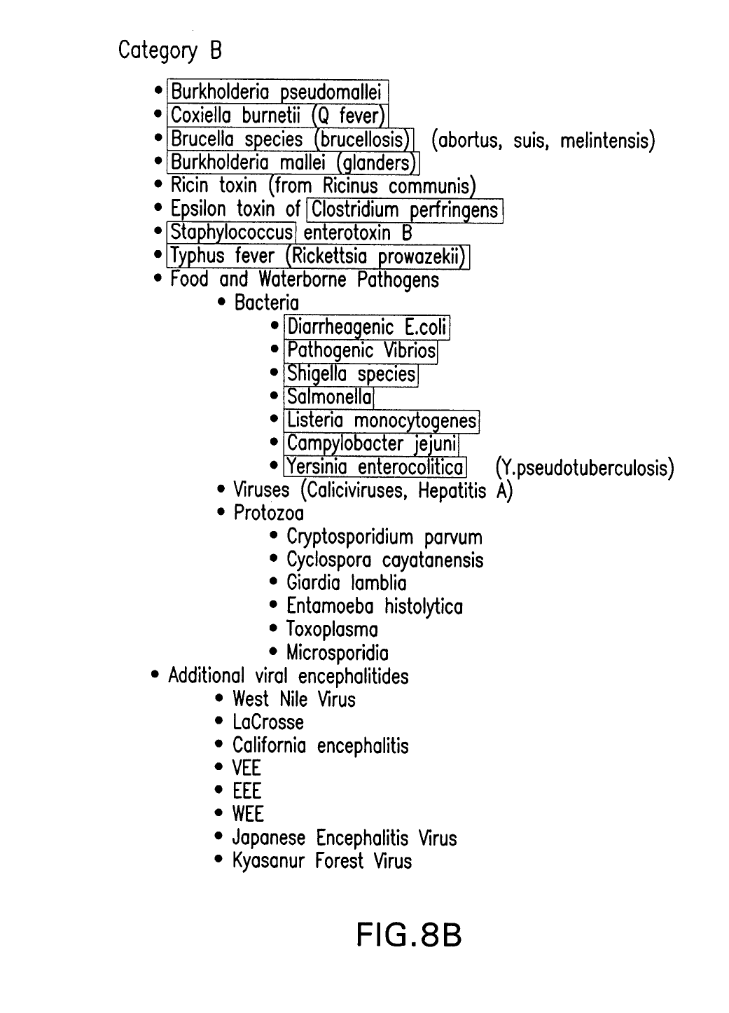

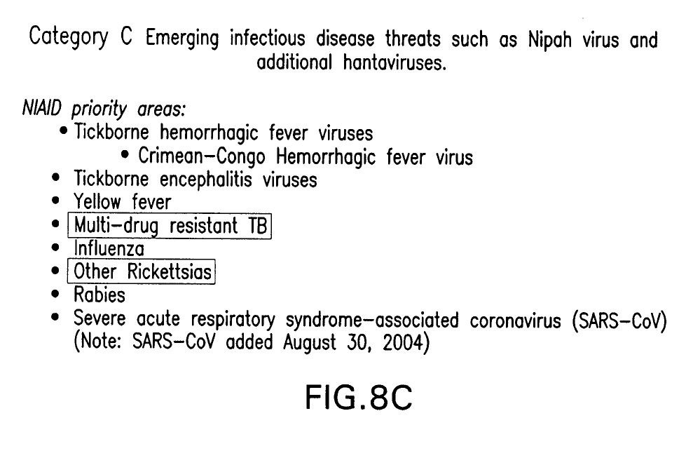

In certain embodiments, a compound provided herein is a highly selective inhibitor which binds preferentially to the loop region of the bacterial peptidyl tRNA hydrolase active site represented by the consensus sequence in FIG. 7. This active site sequence is conserved among various bacterial species. However, sequence alignment of the bacterial sequences with the human homolog introduces a two amino acid gap within this site (FIG. 7). Thus, this gap may introduce differences in the catalytic site between the human and bacterial peptidyl tRNA hydrolases.

In certain embodiments, a compound provided herein is a highly selective inhibitor which binds preferentially to the loop region of the bacterial peptidyl tRNA hydrolase active site represented by the consensus sequence in FIG. 7, and specifically interacts with the aromatic residue (tyrosine or phenylalanine) represented by amino acid number 15 in FIG. 7. While all bacterial species demonstrate variability in residues 13-17 of the loop region as represented by FIG. 7, the aromatic residue at position 15 is one residue that is highly conserved among bacteria. In the putative human enzyme, this residue is a leucine. Thus, this residue may identify a separate, targetable change in tRNA recognition elements.

In certain embodiments, the compounds provided herein bind to potential allosteric binding sites on the peptidyl tRNA hydrolase enzyme. Allosteric binding sites are identified by, but not limited to, surface analysis of three-dimensional structures for charge, hydrogen bonding patterns, lipophilicity, cavity size and depth, or by discrete sequential or structural modeling of related enzymes of families/ensembles of such enzymes.

In certain embodiments, the compounds provided herein bind to potential allosteric binding sites on the peptidyl tRNA hydrolase enzyme. Allosteric binding sites are identified by, e.g., surface analysis of three-dimensional structures for charge, hydrogen bonding patterns, lipophilicity, cavity size and depth, or by discrete sequential or structural modeling of related enzymes or families/ensembles of such enzymes.

### 5.12 Secondary Biological Assays

The compounds identified as inhibitors of bacterial peptidyl tRNA hydrolase by the methods provided herein are further optimized with respect to, and/or selected for, one or more of the following characteristics: the ability to preferentially inhibit the bacterial enzyme versus its eukaryotic homologs, the ability to inhibit bacterial cell growth and/or promote bacterial cell cytotoxicity, low cytotoxicity with respect to eukaryotic, such as mammalian cells, the ability to minimize the emergence of bacterial resistance, and improved pharmacokinetic properties. Assays which can be used to evaluate these characteristics are exemplified in the following sections. In a specific embodiment, the assays are conducted in a high throughput format.

5.12.1 Selectivity for Microbial Peptidyl tRNA Hydrolase

The human peptidyl tRNA hydrolase proteins (Pth and Pth2), which may be cloned and expressed using art-recognized techniques, are used for direct comparisons of inhibitor effects between prokaryotic and eukaryotic enzymes to identify inhibitors that are specific for the prokaryotic enzyme. In one embodiment, the same cloning and expression systems are used to produce both the prokaryotic and eukaryotic enzymes for use in this assay. In one embodiment, the assays are performed using a fluorescent substrate. In a specific embodiment, compounds are selected which inhibit the bacterial Pth enzyme at least 100-fold greater than they inhibit the eukaryotic Pth enzyme. Some other embodiments, compounds are selected which inhibit the bacterial Pth enzyme at least 10-fold greater, at least 20-fold greater, at least 30-fold greater, at least 40-fold greater, at least 50-fold greater, at least 60-fold greater, at least 70-fold greater, at least 80-fold greater, or at least 90-fold more than they inhibit the eukaryotic Pth enzyme in an assay described herein, e.g., the fluorescence polarization assay, or an assay known to one skill in the art. In other embodiments, the compounds are selected which inhibit the bacterial Pth enzyme 10 to 150 fold, 10-50 fold, 25 to 100 fold, 50 to 100 fold, 75 to 150 fold more than they inhibit the eukaryotic Pth enzyme in an assay described herein, e.g., the fluorescence polarization assay, or an assay known to one of skill in the art.

5.12.2 Antibacterial Activity

In certain embodiments, the compounds provided herein are tested in a preliminary antibacterial assay against a permeable E. coli imp mutant. This strain will not select against compounds with the inability to penetrate the wild-type cell wall or membrane. In other embodiments, the compounds are further tested against a panel of bacteria to determine their minimum inhibitory concentrations (“MICs”). In one embodiment, the assays are conducted according to the guidelines from the National Committee for Clinical Laboratory Standards (NCCLS) for antibacterial susceptibility testing and determining the MIC of a compound. The MIC may be determined, for example, using a reporter gene assay. Preferred reporter gene systems utilize a firefly or bacterial luciferase, or a beta-galactosidase reporter gene. The reporter gene will comprise the cDNA and/or regulatory sequences necessary for the expression of a gene whose expression is correlated with bacterial cell proliferation and/or viability.

In certain specific embodiments, the compounds provided herein are tested for their ability to inhibit the proliferation of one or more bacteria selected from among M. tuberculosis, E. coli, S. aureus, S. epidermidis, P. aeruginosa, E. faecalis, E. faecium, H. influenzae, N. meningitides, Streptococcus pneumoniae, and Mycobacterium bovis (BCG). In some embodiments, the compounds provided herein are tested against bacteria grown under conditions of nutrient and/or oxygen depravation.

In certain embodiments, the compounds are also assessed for activity in a macrophage-based assay. The ability of an intracellular bacteria, such as mycobacteria, to survive within the intracellular environment of the macrophage reduces the efficacy of many antibacterial agents. To be effective, the antibiotic must penetrate the cell membrane, remain stable in the macrophage cellular environment, and reach efficacious concentrations where the pathogen is located. In a specific embodiment, a Mycobacterium bovis BCG-reporter gene system is used to assess the efficacy of test compounds against infection of resting and LPS stimulated THP-1 monocytic cells. For these studies, the cells are infected with the BCG construct and incubated for a period of time up to 7 days in the presence of various concentrations of the test compounds.

In certain embodiments, the bactericidal activity of the compounds provided herein will be confirmed by use of the compounds at 2×, 4×, and 10× the MIC concentration on organisms for which inhibition of proliferation is observed.

In certain embodiments, the compounds provided herein are evaluated for synergistic activity with other protein synthesis inhibitors. In particular embodiments, a compound provided herein is combined with one or more protein synthesis inhibitors selected from the group consisting of streptomycin, neomycin, erythromycin, carbomycin, and spiramycin. In accordance with this embodiment, combination indices are generated for several molecules from each structural class of inhibitors by performing a checkerboard analysis. The combination indices are used to determine synergy, additivity, or antagonism of the drug combinations.

5.12.3 Mammalian Cytotoxicity

The compounds provided herein can be tested for cytotoxicity in mammalian, such as human, cell lines. In certain specific embodiments, cytotoxicity is assessed in one or more of the following cell lines: U937, a human monocyte cell line; primary peripheral blood mononuclear cells (PBMC); Huh7, a human hepatoblastoma cell line; 293T, a human embryonic kidney cell line; and THP-1, monocytic cells in which intracellular killing of Mycobacterium is tested.

Many assays well-known in the art can be used to assess viability of a cell or cell line following exposure to a compound provided herein and, thus, determine the cytotoxicity of the compound. For example, cell proliferation can be assayed by measuring Bromodeoxyuridine (BrdU) incorporation (see, e.g., Hoshino et al., 1986, Int. J. Cancer 38, 369; Campana et al., 1988, J. Immunol. Meth. 107:79) or (³H)-thymidine incorporation (see, e.g., Chen, J., 1996, Oncogene 13:1395-403; Jeoung, J., 1995, J. Biol. Chem. 270:18367-73), by direct cell count, by detecting changes in transcription, translation or activity of known genes such as proto-oncogenes (e.g., fos, myc) or cell cycle markers (Rb, cdc2, cyclin A, D1, D2, D3, E, etc). The levels of such protein and mRNA and activity can be determined by any method well known in the art. For example, protein can be quantitated by known immunodiagnostic methods such as Western blotting or immunoprecipitation using commercially available antibodies. mRNA can be quantitated using methods that are well known and routine in the art, for example, using northern analysis, RNase protection, the polymerase chain reaction in connection with the reverse transcription. Cell viability can be assessed by using trypan-blue staining or other cell death or viability markers known in the art. In a specific embodiment, the level of cellular ATP is measured to determined cell viability.

In specific embodiments, cell viability is measured in three-day and seven-day periods using an assay standard in the art, such as the CellTiter-Glo Assay Kit (Promega) which measures levels of intracellular ATP. A reduction in cellular ATP is indicative of a cytotoxic effect.

In a specific embodiment, the compounds provided herein demonstrate a therapeutic index of 50-fold or greater between the cytotoxicity CC50 and the MIC value. In some embodiments, the compounds provided herein demonstrate a therapeutic index between the cytotoxicity CC50 and MIC value is 10 to 100 fold, 25 to 75 fold, 50 to 100 fold, 50 to 150 fols, of 75 to 150 fold.

5.12.4 Ability of Microorganisms to Develop Resistance to Compounds

In certain embodiments, the compounds provided herein are selected in part using frequency of resistance information. To test an organism's ability to develop resistance to a compound provided herein, bacteria are incubated with inhibitors at increasing concentrations above the MIC value for the compound, using both liquid and solid phase growth conditions. In a specific embodiment, a compound provided herein is one against which bacteria are less able or even unable to mount resistance.

In certain embodiments, the compounds provided herein are further screened against peptidyl tRNA hydrolase enzyme isolated from strains of bacteria that were able to develop resistance in these assays. The peptidyl tRNA hydrolase enzyme genes from resistant strains can be PCR-amplified, cloned, and sequenced to determine the specific location of the mutation(s) leading to the observed phenotypes. This information can be used to synthesize compounds that the bacteria are unable to develop resistance to.

### 6. EXAMPLES

### 6.1 Initial Screen for Inhibitory Activity Against RNA Hydrolase

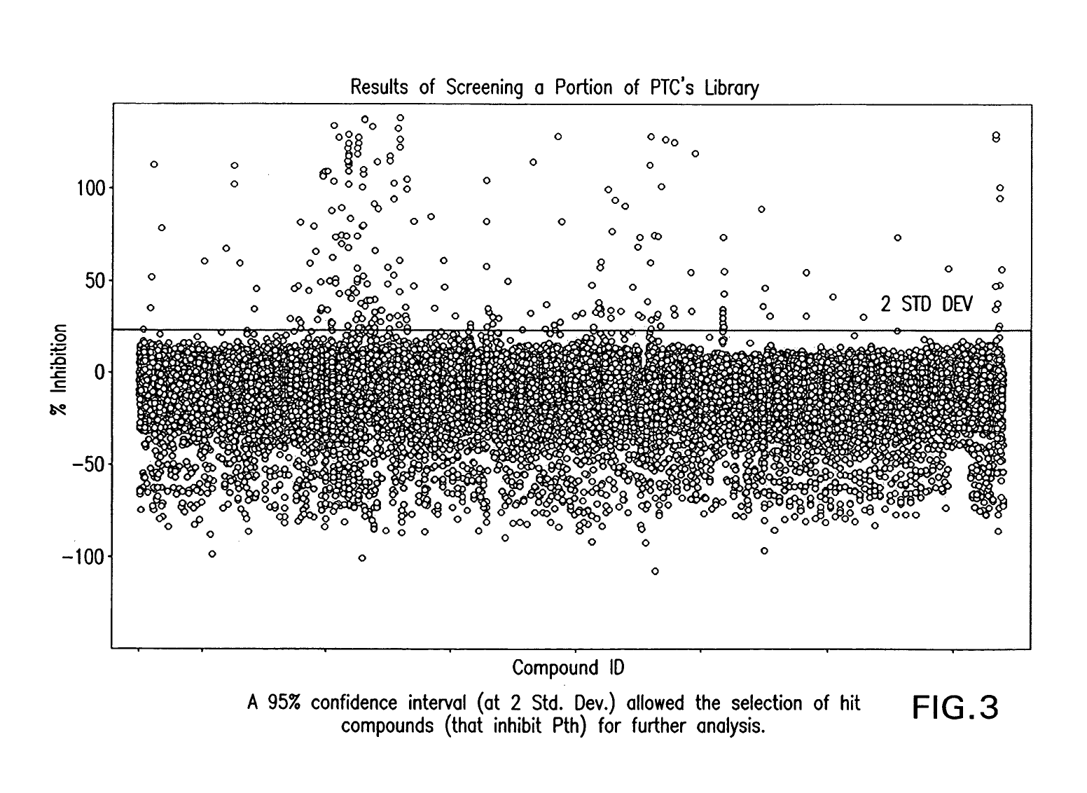

Using the E. coli peptidyl tRNA hydrolase as the prototype enzyme target and a 3,400-compound library subset, the fluorescence polarization assay for inhibition of peptidyl tRNA hydrolase activity was determined to be robust and sensitive, with Z′-values up to 0.65. FIG. 3 shows the analysis of percent inhibition profiles for 30,000 compounds screened against the E. coli peptidyl tRNA hydrolase enzyme.

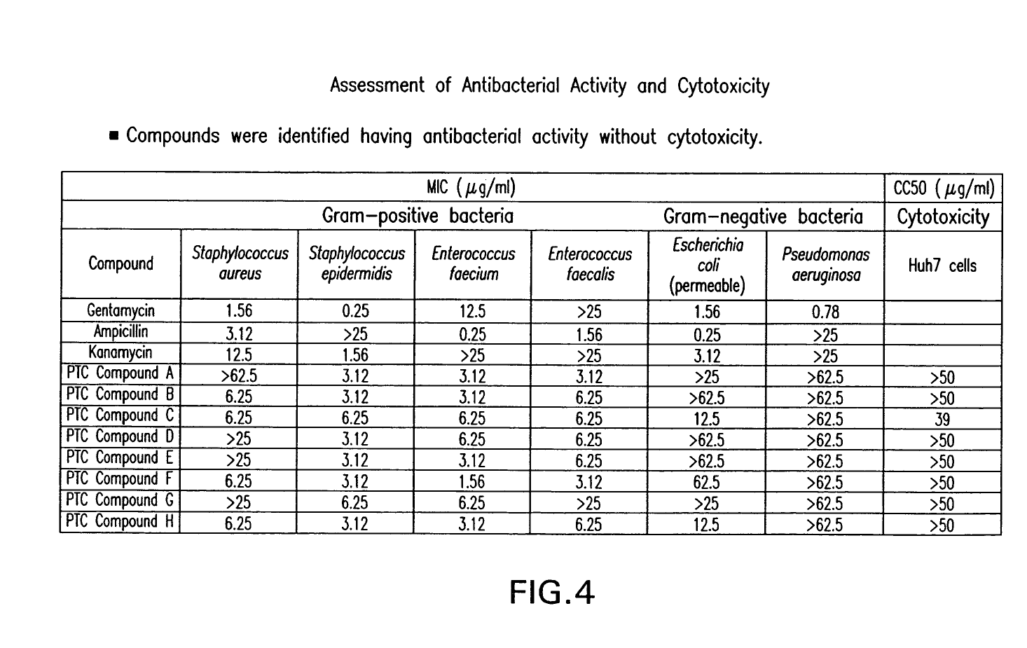

Compounds having inhibition greater than 25% were further evaluated for antibacterial activity (MIC), enzyme inhibition (IC₅₀) and cytotoxicity. The minimum inhibitory concentrations (MIC) of test compounds were determined using bacteria grown in brain heart infusion media (BHI). Logarithmically growing cells were diluted to approximately 5×105 CFU/ml and subjected to test compounds solubilized and serially diluted in DMSO. A 5% final DMSO concentration had no affect on cell viability or killing (2.5% final DMSO concentration routinely performed). After 18 hours at 37° C., the OD600 was determined by reading the ninety-six well microtiter plates on a microplate reader. For a given concentration, an MIC determination was made if: [OD600 Control−OD600 Test Conc.]/[OD600 Control−OD600 Media]×100≧90%. All organisms are grown in a universal rich media to minimize media effects on the inhibition assay. All bacteria utilized in the MIC assay have been demonstrated to grow in Brain Heart Infusion (BHI) media (Difco, Detroit, Mich.). Library compounds are at a concentration of 2.5 to 10 mg/ml. The MICs for the antibiotics Ampicillin, Kanamycin, and Gentamicin are also shown in FIG. 4. Antibiotic concentrations varied from 25 ug/ml (1 mM stock) to 0.39 ug/ml. The bacteria tested were Enterococcus faecium (ATCC 49624), Enterococcus faecalis (ATCC 29212), Staphylococcus aureus (ATCC 29213), Staphylococcus epidermidis (ATCC 12228), Escherichia coli (BAS849—permeable) and Pseudomonas aeruginosa (ATCC 27853).

Novel antibacterial peptidyl tRNA hydrolase inhibitors were identified using the MIC assay (inhibition ranged from 32-100%). The inhibitors were also bacteria specific, as evidenced by the low cytotoxicity observed for the human Huh7 cells (FIG. 4). Cytotoxicity was determined according to manufacturer's directions (CellTiter 96 Aqueous Non-Radioactive Cell Proliferation Assay, Promega).

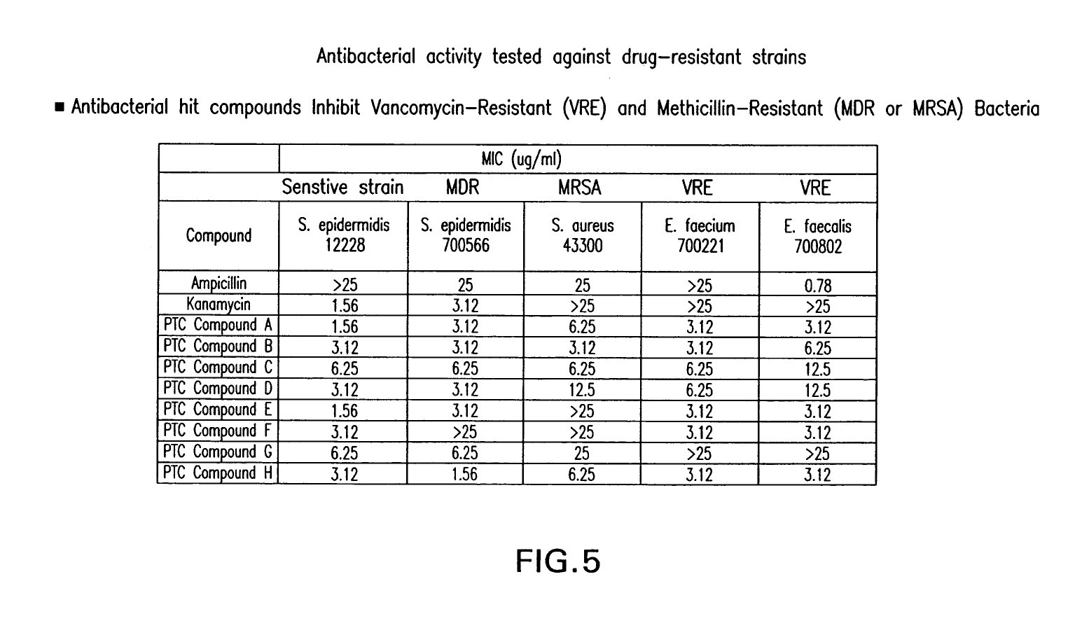

The inhibitors were also effective against antibiotic resistant strains of Staphylococcus epidermidis, Staphylococcus aureus, Enterococcus faecium, and Enterococcus faecalis (FIG. 5). Two unique classes of molecules have emerged from this initial screen. The significance of these compounds is two-fold. First, they can be used to standardize the assay for screening the full library. Second, these compounds serve as a starting point to evaluate analogs in a traditional drug discovery process. Analogs that exhibit activity will be analyzed in order to identify chemotypes that display the ideal behaviors of peptidyl tRNA hydrolase inhibition: antibacterial activity, limited cytotoxicity and good pharmacokinetic profiles. While the inhibitory concentrations are not sub-micromolar, it should be noted that the goal of the initial high throughput library screen is to identify compounds having the desired properties, which are then further optimized.

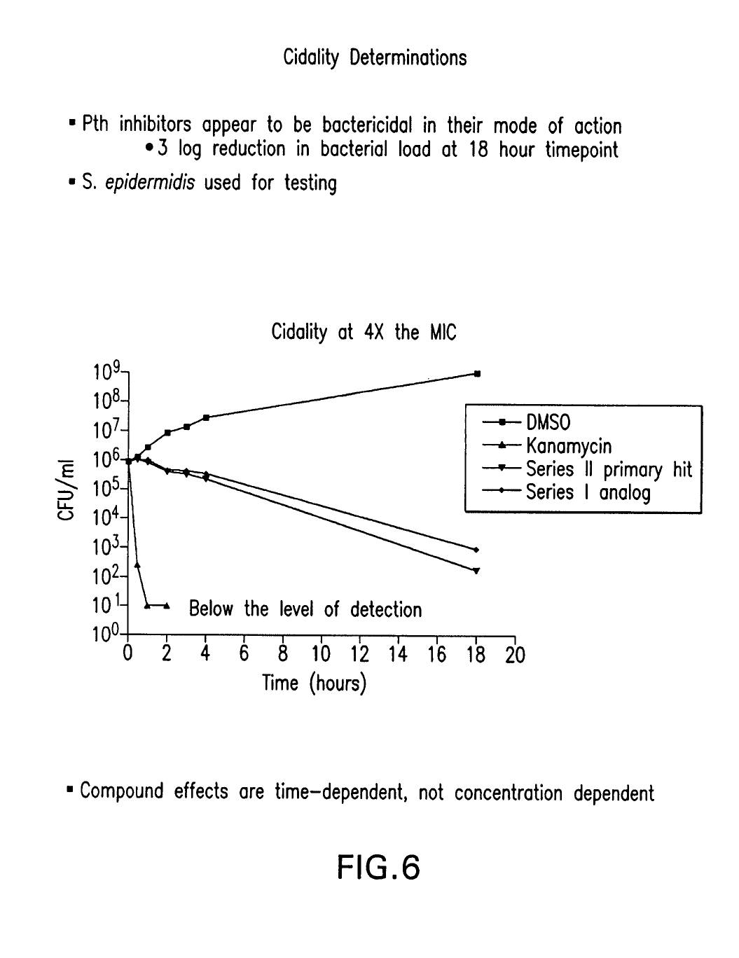

Preliminary testing of cidality on a representative compound against S. epidermidis suggests that the inhibitors identified were bactericidal (FIG. 6).

Further for representative compounds is set forth in Table 1 (MIC and cytotoxicity). Compounds were tested against S. aureus (ATCC 29213) and E. coli (BAS849-permeable) in the MIC assays. Certain compounds were also tested against other bacteria, including S. epidermis (ATCC 12228), E. faecium (ATCC 49624), and E. faecalis (ATCC 29212). Cytotoxicity was determined using Hu7 cells and Hep G2 cells. The MIC and cytotoxicity assays were performed as described above.

The MIC results are presented according to the following scheme:
0.1-1 μg/ml=5 stars
1.1-10 μg/ml=4 stars
10.1-20 μg/ml=3 stars
20.1-50 μg/ml=2 stars
>50 μg/ml=1 star

### 6.2 Synthetic Examples

### 6.2.1 Synthesis of 6-Benzyl-3-(4-chloro-phenylthio)-4-hydroxy-6H-pyrano[3,2-c]quinoline-2,5-dione (Compound 18)

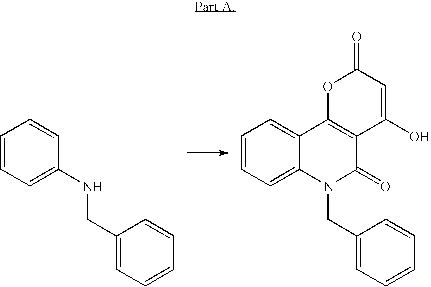

A solution of N-benzyl aniline (7.34 g, 40.0 mmol) and diethyl malonate (14.0 mL, 92.3 mmol) in diphenyl ether (50 mL) was heater to >200° C. in a round bottom flask fitted with a short-path stillhead equipped with a thermometer. As the reaction proceeded, the distillate (ethanol) was collected and the temperature of the vapor was monitored. When the reaction neared completion (and the theoretical volume of ethanol was mostly collected), the temperature of the vapor rose from about 80° C. to about 120° C., then dropped as the ethanol distillation was complete. The heat was turned off when the temperature began rising again (probably from the distillation of excess diethyl malonate). The mixture was allowed to cool and was poured into diethyl ether. The resulting solid was collected by filtration, washed with more diethyl ether, and dried under vacuum to afford the product, 6-benzyl-4-hydroxy-6H-pyrano[3,2-c]quinoline-2,5-dione, as a tan solid (5.07 g, 15.9 mmol, 40%).

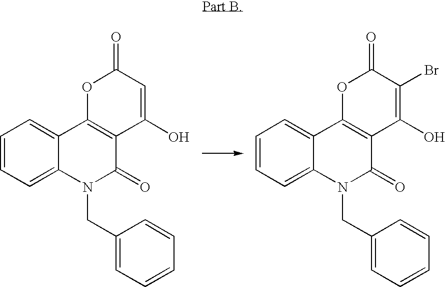

A suspension of 6-benzyl-4-hydroxy-6H-pyrano[3,2-c]quinoline-2,5-dione (240 mg, 0.752 mmol) and N-bromosuccinimide (134 mg, 0.753 mmol) in 5 mL acetonitrile was heated to reflux overnight. The mixture was cooled and filtered, and the resulting precipitate was washed with a small volume of acetonitrile, then diethyl ether. The solid product was dried under vacuum to give 6-benzyl-3-bromo-4-hydroxy-6H-pyrano[3,2-c]quinoline-2,5-dione (250 mg, 0.628 mmol, 83%) as a yellowish powder, m.p. 226-229° C. TLC R_{F} 0.42 (50:50 ethyl acetate-hexane). ¹H NMR (300 MHz, DMSO-d₆): δ 8.20 (1H, dd, J=7.8, 1.4 Hz), 7.84 (1H, t, J=6.7 Hz), 7.80 (1H, dd, J=7.2, 1.6 Hz), 7.52 (1H, t, J=7.2 Hz), 7.39-7.14 (5H, m), 5.67 (2H, s). MS (ES+): m/e 400 (95), 398 (100).

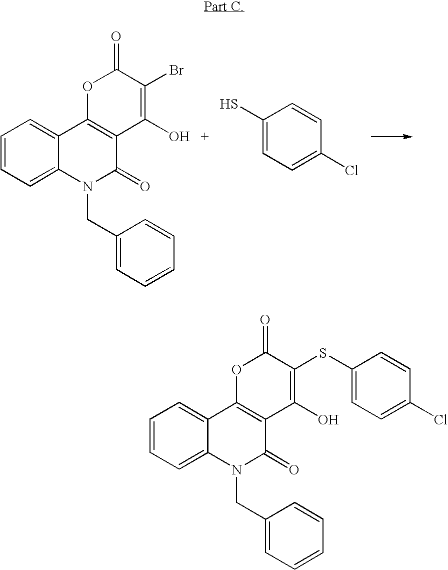

A suspension of 6-benzyl-3-bromo-4-hydroxy-6H-pyrano[3,2-c]quinoline-2,5-dione (133 mg, 0.333 mmol), 4-chlorothiophenol (50 mg, 0.346 mmol) and anhydrous potassium carbonate (50 mg, 0.362 mmol) in 2 mL dimethylformamide was heated to 70° C. overnight. The mixture was cooled and partitioned between 4 volumes each of 0.25 N aq. HCl and diethyl ether. The mixture was filtered, and the collected solid was washed with additional diethyl ether and dried under high vacuum to afford the title product (95 mg, 0.206 mmol, 62%) as a pale yellow powder, m.p. 273-275° C. TLC R_{F} 0.26 (50:50 ethyl acetate-hexane). ¹H NMR (300 MHz, DMSO-d₆): δ 8.23 (1H, dd, J=8.0, 1.4 Hz), 7.82 (1H, td, J=8.5, 1.5 Hz), 7.70 (1H, d, J=8.4 Hz), 7.53 (1H, t, J=7.6 Hz), 7.35-7.23 (9H, m), 5.67 (2H, s). MS (ES+): m/e 464 (35), 462 (100). MS (ES−): m/e 460 (100).

The following compounds were prepared in an analogous manner to that of Example 6.2.1.

Compound 20: m.p. 313-318° C. ¹H NMR (300 MHz, DMSO-d₆): δ 8.21 (1H, d, J=7.0 Hz), 7.70-7.56 (4H, m), 7.52-7.40 (3H, m), 7.29 (2H, d, J=8.8 Hz), 7.19 (2H, d, J=8.8 Hz), 6.68 (1H, d, J=8.8 Hz). MS (ES+): m/e 450 (50), 448 (100).

Compound 21: m.p. 225-227° C. ¹H NMR (300 MHz, DMSO-d₆): δ 8.27 (1H, dd, J=8.0, 1.3 Hz), 7.79-7.18 (1H, m), 6.78 (1H, d, J=8.5 Hz). MS (ES+): m/e 450 (45), 448 (100).

Compound 22: m.p. 213-214° C. ¹H NMR (300 MHz, DMSO-d₆): δ 8.26 (1H, dd, J=8.2, 1.5 Hz), 7.78-7.45 (8H, m), 7.18 (1H, t, J=7.9 Hz), 6.80-6.70 (4H, m), 3.70 (3H, s). MS (ES+): m/e 445 (20), 444 (100).

Compound 23: m.p. 264-266° C. ¹H NMR (300 MHz, DMSO-d₆): δ 8.22 (1H, dd, J=8.2, 1.4 Hz), 7.73-7.59 (4H, m), 7.53-7.36 (4H, m), 7.11 (2H, d, J=8.2 Hz), 7.06 (2H, d, J=8.2 Hz), 6.71 (1H, d, J=8.4 Hz), 2.22 (3H, s). MS (ES+): m/e 429 (20), 428 (100).

Compound 24: m.p. 211-212° C. ¹H NMR (300 MHz, DMSO-d₆): δ 8.22 (1H, dd, J=7.9, 1.2 Hz), 7.76-7.36 (7H, m), 7.27 (2H, d, J=8.8 Hz), 6.86 (2H, d, J=8.8 Hz), 6.74 (1H, d, J=8.5 Hz), 3.70 (3H, s). MS (ES+): m/e 445 (20), 444 (100).

Compound 25: m.p. 262-263° C. ¹H NMR (300 MHz, DMSO-d₆): δ 8.26 (1H, dd, J=8.2, 1.2 Hz), 7.78-7.44 (7H, m), 6.84 (2H, s), 6.77 (1H, s), 6.76 (1H, d, J=7.8 Hz), 2.19 (6H, s). MS (ES+): m/e 443 (20), 442 (100).

Compound 26: m.p. 269-271° C. ¹H NMR (300 MHz, DMSO-d₆): δ 7.55 (1H, d, J=2.9 Hz), 7.40 (1H, dd, J=9.3, 2.9 Hz), 7.36 (2H, d, J=9.0 Hz), 7.33 (2H, d, J=9.0 Hz), 7.25 (2H, d, J=9.0 Hz), 7.20 (2H, d, J=9.0 Hz), 6.79 (1H, d, J=9.3 Hz), 3.90 (3H, s), 3.85 (3H, s). MS (ES−): m/e 508 (100).

Compound 27: m.p. 197-198° C. ¹H NMR (300 MHz, DMSO-d₆): δ 8.24 (1H, dd, J=8.2, 1.5 Hz), 7.87-7.68 (4H, m), 7.58-7.51 (2H, m), 7.42 (1H, t, J=7.9 Hz), 7.35-7.24 (5H, m), 5.67 (2H, s), 3.82 (3H, s). MS (ES+): m/e 487 (25), 486 (100).

Compound 28: m.p. 244-245° C. ¹H NMR (300 MHz, DMSO-d₆): δ 8.25 (1H, dd, J=8.0, 1.5 Hz), 7.84 (1H, ddd, J=8.5, 7.0, 1.5 Hz), 7.71 (1H, d, J=8.5 Hz), 7.60-7.48 (5H, m), 7.36-7.26 (5H, m), 5.68 (2H, s). ¹⁹F NMR (300 MHz, DMSO-d₆): δ −61.53 (3F, s). MS (ES+): m/e 497 (100).

Compound 29: m.p. 214-216° C. ¹H NMR (300 MHz, DMSO-d₆): δ 8.24 (1H, dd, J=8.0, 1.3 Hz), 7.84 (1H, ddd, J=8.5, 7.3, 1.4 Hz), 7.71 (1H, d, J=8.5 Hz), 7.54 (1H, td, J=8.1, 0.9 Hz), 7.34-7.18 (9H, m), 5.68 (2H, s). MS (ES+): m/e 464(35), 462 (100). MS (ES−): m/e 462 (35), 460 (100).

Compound 30: m.p. 207-209° C. ¹H NMR (300 MHz, DMSO-d₆): δ 8.23 (1H, dd, J=8.2, 1.4 Hz), 7.86-7.80 (1H, m), 7.69 (1H, d, J=8.8 Hz), 7.53 (1H, t, J=7.6 Hz), 7.35-7.22 (5H, m), 7.18 (1H, t, J=7.9 Hz), 6.83-6.70 (3H, m), 5.67 (2H, s), 3.70 (3H, s). MS (ES+): m/e 458 (100). MS (ES−): m/e 457 (80), 238 (100).

Compound 31: m.p. 238-239° C. ¹H NMR (300 MHz, DMSO-d₆): δ 8.25 (1H, dd, J=8.2, 1.5 Hz), 7.85 (1H, ddd, J=8.5, 7.3, 1.5 Hz), 7.81 (2H, d, J=8.8 Hz), 7.72 (1H, d, J=8.5 Hz), 7.55 (1H, td, J=8.0, 0.9 Hz), 7.36 (2H, d, J=8.8 Hz), 7.35-7.25 (5H, m), 5.68 (2H, s), 3.80 (3H, s). MS (ES+): m/e 487 (25), 486 (100).

Compound 34: m.p. 207-209° C. ¹H NMR (300 MHz, DMSO-d₆): δ 8.23 (1H, dd, J=8.0, 1.3 Hz), 7.83 (1H, ddd, J=8.5, 7.0, 1.3 Hz), 7.69 (1H, d, J=8.5 Hz), 7.53 (1H, t, J=7.6 Hz), 7.33-7.23 (5H, m), 7.18 (1H, t, J=8.0 Hz), 6.83-6.70 (3H, m), 5.67 (2H, s), 3.70 (3H, s). MS (ES+): m/e 459 (25), 458 (100). MS (ES−): m/e 457 (25), 456 (100).

Compound 35: m.p. 298-300° C. ¹H NMR (300 MHz, DMSO-d₆): δ 12.87 (1H, br s), 8.25 (1H, dd, J=8.0, 1.3 Hz), 7.85 (1H, ddd, J=8.7, 7.0, 1.3 Hz), 7.80 (2H, d, J=8.5 Hz), 7.71 (1H, d, J=8.7 Hz), 7.54 (1H, t, J=7.6 Hz), 7.36 (2H, d, J=8.5 Hz), 7.35-7.25 (5H, m), 5.68 (2H, s). MS (ES+): m/e 472 (100). MS (ES−): m/e 470 (100).

Compound 36: m.p. 212-214° C. ¹H NMR (300 MHz, DMSO-d₆): δ 8.37-8.34 (1H, m), 8.26 (1H, dd, J=7.9, 1.5 Hz), 7.87 (1H, ddd, J=8.5, 7.0, 1.5 Hz), 7.74 (1H, d, J=8.5 Hz), 7.56 (1H, t, J=7.3 Hz), 7.36-7.22 (8H, m), 5.69 (2H, s). MS (ES+): m/e 445 (100). MS (ES−): m/e 443 (100).

Compound 37: m.p. 188-189° C. ¹H NMR (300 MHz, DMSO-d₆): δ 8.23 (1H, dd, J=7.9, 1.5 Hz), 7.83 (1H, ddd, J=8.5, 7.3, 1.5 Hz), 7.69 (1H, d, J=8.5 Hz), 7.53 (1H, t, J=7.6 Hz), 7.34-7.24 (9H, m), 7.17-7.11 1H (m). MS (ES+): m/e 428 (100). MS (ES−): m/e 426 (100).

Compound 38: m.p. 268-269° C. ¹H NMR (300 MHz, DMSO-d₆): δ 13.09 (1H, br), 8.24 (1H, dd, J=8.2, 1.2 Hz), 7.83 (1H, ddd, J=8.3, 7.0, 1.2 Hz), 7.78 (1H, t, J=1.8 Hz), 7.74-7.68 (2H, m), 7.56-7.49 (2H, m), 7.40 (1H, t, J=7.6 Hz), 7.36-7.24 (5H, m), 5.67 (2H, s). MS (ES+): m/e 473 (20), 472 (100).

Compound 86: m.p. 203-204° C. ¹H NMR (300 MHz, DMSO-d₆): δ 7.66 (1H, d, J=9.6 Hz), 7.55 (1H, d, J=2.9 Hz), 7.47 (1H, dd, J=9.6, 2.9 Hz), 7.35-7.17 (9H, m), 5.67 (2H, s), 3.89 (3H, s). MS (ES+): m/e 494 (45), 492 (100). MS (ES−): m/e 492 (25), 490 (100).

Compound 88: m.p. 173-174° C. ¹H NMR (300 MHz, DMSO-d₆): δ 7.65 (1H, d, J=9.4 Hz), 7.54 (1H, d, J=2.9 Hz), 7.47 (1H, dd, J=9.4, 2.9 Hz), 7.34-7.24 (5H, m), 7.18 (1H, t, J=7.9 Hz), 6.82-6.76 (2H, m), 6.72 (1H, dd, J=7.9, 2.1 Hz), 5.66 (2H, s), 3.88 (3H, s), 3.70 (3H, s). MS (ES+): m/e 489 (25), 488 (100). MS (ES−): m/e 486 (100).

Compound 113: m.p.>300° C. ¹H NMR (300 MHz, DMSO-d₆): δ 8.11 (1H, d, J=7.0 Hz), 7.64-7.45 (4H, m), 7.37-7.27 (3H, m), 6.83 (2H, d, J=8.5 Hz), 6.48-6.42 (1H, m), 6.39 (2H, d, J=8.5 Hz). MS (ES+): m/e 430.50 (25), 429.17 (100). MS (ES−): m/e 428.40 (25), 427.16 (100).

Compound 116: m.p. 226-228° C. ¹H NMR (300 MHz, DMSO-d₆): δ 13.11 (1H, s), 9.50 (1H, s), 8.21 (1H, dd, J=8.2, 1.2 Hz), 7.74-7.62 (4H, m), 7.52 (1H, t, J=7.9 Hz), 7.47-7.43 (3H, m), 7.18 (2H, d, J=8.8 Hz), 6.68 (2H, d, J=8.8 Hz). MS (ES+): m/e 430.16 (100). MS (ES−): m/e 429.47 (20), 428.18 (100).

Compound 117: m.p. 230-233° C. ¹H NMR (300 MHz, DMSO-d₆): δ 9.48 (1H, s), 8.26 (1H, dd, J=8.0, 1.3 Hz), 7.77-7.63 (4H, m), 7.55 (1H, t, J=7.3 Hz), 7.50-7.44 (2H, m), 7.06 (1H, t, J=7.9 Hz), 6.76 (1H, d, J=8.5 Hz), 6.66 (1H, dm, J=8 Hz), 6.60 (1H, t, J=2.0 Hz), 6.54 (1H, ddd, J=8, 2, 1 Hz). MS (ES+): m/e 431.44 (20), 430.20 (100). MS (ES−): m/e 429.40 (20), 428.20 (100).

Compound 136: m.p. 277-279° C. ¹H NMR (300 MHz, DMSO-d₆): δ 8.27 (1H, dd, J=7.9, 1.5 Hz), 7.80-7.44 (7H, m), 7.13-6.76 (5H, m), 3.85 (3H, s). MS (ES−): m/e 441.99 (100).

Compound 137: m.p. 233-234° C. ¹H NMR (300 MHz, DMSO-d₆): δ 8.27 (1H, dd, J=8.2, 1.5 Hz), 7.82 (2H, d, J=8.5 Hz), 7.78-7.63 (4H, m), 7.56 (1H, t, J=7.7 Hz), 7.49-7.46 (2H, m), 7.34 (2H, d, J=8.5 Hz), 6.79 (1H, d, J=8.4 Hz), 3.80 (3H, s). MS (ES+): m/e 471.96 (100). MS (ES−): m/e 471.00 (25), 469.93 (100).

Compound 138: m.p. 179-180° C. ¹H NMR (300 MHz, DMSO-d₆): δ 13.20 (1H, s), 8.26 (1H, dd, J=8.0, 1.3 Hz), 7.80-7.42 (11H, m), 6.77 (1H, d, J=8.5 Hz), 3.82 (3H, s). MS (ES+): m/e 471.92 (100). MS (ES−): m/e 470.96 (30), 469.96 (100).

Compound 139: m.p. 254-255° C. ¹H NMR (300 MHz, DMSO-d₆): δ 8.28 (1H, dd, J=8.0, 1.3 Hz), 7.80-7.41 (11H, m), 6.79 (1H, d, J=8.5 Hz). ¹⁹F NMR (300 MHz, DMSO-d₆): δ −61.11 (3F, s). MS (ES+): m/e 481.89 (100). MS (ES−): m/e 480.95 (25), 479.93 (100).

Compound 140: m.p. 226-227° C. ¹H NMR (300 MHz, DMSO-d₆): δ 8.27 (1H, dd, J=7.9, 1.5 Hz), 7.79-7.44 (11H, m), 6.78 (1H, d, J=8.5 Hz). ¹⁹F NMR (300 MHz, DMSO-d₆): δ −61.56 (3F, s). MS (ES+): m/e 481.90 (100). MS (ES−): m/e 480.96 (25), 479.94 (100).

Compound 141: m.p. 225-226° C. ¹H NMR (300 MHz, DMSO-d₆): δ 8.36 (2H, br), 7.73-7.58 (5H, m), 7.51 (1H, t, J=7.3 Hz), 7.47-7.42 (2H, m), 7.37 (1H, dd, J=7.9, 1.5 Hz), 7.00 (1H, td, J=7.6, 1.5 Hz), 6.71 (1H, d, J=8.5 Hz), 6.66 (1H, dd, J=8.0, 1.3 Hz), 6.47 (1H, td, J=7.3, 1.2 Hz). MS (ES+): m/e 430.12 (20), 428.95 (100). MS (ES−): m/e 427.99 (25), 427.00 (100).

Compound 172: m.p. 223-225° C. ¹H NMR (300 MHz, DMSO-d₆): δ 8.26 (1H, dd, J=8.2, 1.5 Hz), 7.79-7.45 (8H, m), 7.24-7.17 (2H, m), 7.12-7.06 (1H, m), 6.77 (1H, d, J=8.2 Hz). ¹⁹F NMR (282 MHz, DMSO-d₆): δ −114.20 (1F, q, J=8.0 Hz). MS (ES+): m/e 433.46 (20), 432.08 (100). MS (ES−): m/e 431.45 (20), 430.10 (100).

Compound 173: m.p. 197-199° C. ¹H NMR (300 MHz, DMSO-d₆): δ 8.27 (1H, dd, J=7.9, 1.4 Hz), 7.79-6.90 (11H, m), 6.78 (1H, d, J=8.5 Hz). ¹⁹F NMR (282 MHz, DMSO-d₆): δ −112.70 (1F, q, J=8.0 Hz). MS (ES+): m/e 433.45 (20), 432.07 (100). MS (ES−): m/e 431.42 (20), 430.08 (100).

Compound 174: m.p. 247-249° C. ¹H NMR (300 MHz, DMSO-d₆): δ 8.20 (1H, dd, J=8.2, 1.5 Hz), 7.72-7.59 (4H, m), 7.57-7.42 (3H, m), 7.27-7.20 (2H, m), 7.17-7.05 (2H, m), 6.68 (1H, d, J=8.8 Hz). ¹⁹F NMR (282 MHz, DMSO-d₆): δ −118 (1F). MS (ES+): m/e 433.46 (20), 432.07 (100). MS (ES−): m/e 431.38 (20), 430.07 (100).

Compound 175: m.p. 220-221° C. ¹H NMR (300 MHz, DMSO-d₆): δ 8.25 (1H, dd, J=7.9, 1.5 Hz), 7.78-7.61 (4H, m), 7.55 (1H, td, J=7.3, 0.9 Hz), 7.48-7.44 (2H, m), 7.35-7.28 (2H, m), 7.02 (1H, tdd, J=8.2 2.5, 1.0 Hz), 6.77 (1H, d, J=8.5 Hz). ¹⁹F NMR (282 MHz, DMSO-d₆): δ −108.77 (1F, s), −133.36 (1F, s). MS (ES+): m/e 451.43 (20), 450.08 (100). MS (ES−): m/e 449.35 (20), 448.06 (100).

Compound 176: m.p. 238-240° C. ¹H NMR (300 MHz, DMSO-d₆): δ 8.20 (1H, dd, J=8.2, 1.5 Hz), 7.96-7.86 (1H, m), 7.76-7.60 (4H, m), 7.53 (1H, td, J=7.3, 0.9 Hz), 7.48-7.44 (2H, m), 6.73 (1H, d, J=8.5 Hz). ¹⁹F NMR (282 MHz, DMSO-d₆): δ −135.51-−135.66 (2F, m), −139.36 (2F, dt, J=23.2, 11.9 Hz). MS (ES+): m/e 487.46 (20), 486.06 (100). MS (ES−): m/e 485.39 (20), 484.04 (100).

Compound 177: m.p. 158-160° C. ¹H NMR (300 MHz, DMSO-d₆): δ 8.20 (1H, dd, J=8.0, 1.5 Hz), 7.93-7.44 (7H, m), 6.74 (1H, d, J=8.5 Hz). ¹⁹F NMR (282 MHz, DMSO-d₆): δ −132.8-−133.4 (4F, m), −133.9-−134.3 (2F, m), −135.1-−135.4 (2F, m), −161.16 (1F, t, J=20.8 Hz). MS (ES+): m/e 685.46 (25), 684.07 (100). MS (ES−): m/e 683.34 (30), 682.06 (100).

Compound 178: m.p. 259-260° C. ¹H NMR (300 MHz, DMSO-d₆): δ 9.91 (1H, s), 8.26 (1H, dd, J=7.9, 1.5 Hz), 7.77-7.45 (7H, m), 6.97-6.64 (5H, m). MS (ES+): m/e 430.09 (100). MS (ES−): m/e 429.36 (20), 428.08 (100).

Compound 220: m.p. 215-218° C. ¹H NMR (300 MHz, DMSO-d₆): δ 8.21 (1H, dd, J=8.1, 1.3 Hz), 7.99 (1H, d, J=8.8 Hz), 7.92 (1H, td, J=7.0, 1.4 Hz), 7.56 (1H, t, J=7.5 Hz), 7.29-7.20 (4H, m), 7.17-7.10 (1H, m), 4.96 (1H, br), 4.49 (2H, t, J=6.0 Hz), 3.75 (2H, br t, J=6 Hz). MS (ES+): m/e 383.0 (20), 382.0 (100). MS (ES−): m/e 380.9 (20), 379.9 (100).

Compound 221: m.p. 172-174° C. ¹H NMR (300 MHz, DMSO-d₆): δ 8.22 (1H, dd, J=7.9, 1.2 Hz), 8.00 (1H, d, J=8.5 Hz), 7.93 (1H, td, J=7.0, 1.5 Hz), 7.57 (1H, t, J=7.3 Hz), 7.30-7.16 (4H, m), 4.98 (1H, br), 4.49 (2H, t, J=6.0 Hz), 3.75 (2H, t, J=6.0 Hz). MS (ES+): m/e 417.9 (30), 415.9 (100). MS (ES−): m/e 415.9 (40), 413.8 (100).

Compound 222: m.p. 169-170° C. ¹H NMR (300 MHz, DMSO-d₆): δ 8.21 (1H, d, J=7.9 Hz), 7.98 (1H, d, J=8.5 Hz), 7.92 (1H, td, J=7.0, 1.5 Hz), 7.56 (1H, t, J=7.5 Hz), 7.17 (1H, t, J=7.9 Hz), 6.79-6.69 (3H, m), 4.96 (1H, br), 4.49 (2H, t, J=5.9 Hz), 3.75 (2H, t, J=5.9 Hz), 3.69 (3H, s). MS (ES+): m/e 413.0 (20), 412.0 (100). MS (ES−): m/e 410.9 (25), 409.9 (100).

Compound 223: m.p. 208-210° C. ¹H NMR (300 MHz, DMSO-d₆): δ 8.21 (1H, dd, J=8.0, 1.3 Hz), 7.97-7.88 (2H, m), 7.56 (1H, ddd, J=7.3, 6.6, 1.4 Hz), 7.28-7.19 (4H, m), 7.16-7.10 (1H, m), 4.37 (2H, t, J=7.6 Hz), 1.72-1.61 (2H, m), 1.49-1.36 (2H, m), 0.93 (3H, t, J=7.4 Hz). MS (ES−): m/e 392.9 (25), 391.9 (100).

Compound 224: m.p. 186-187° C. ¹H NMR (300 MHz, DMSO-d₆): δ 8.23 (1H, dd, J=7.9, 1.5 Hz), 7.99-7.90 (2H, m), 7.61-7.54 (1H, m), 7.30-7.16 (4H, m), 4.38 (2H, t, J=7.6 Hz), 1.72-1.61 (2H, m), 1.49-1.36 (2H, m), 0.93 (3H, t, J=7.3 Hz). MS (ES+): m/e 430.0 (35), 428.0 (100). MS (ES−): m/e 427.9 (35), 425.9 (100).

Compound 225: m.p. 159-160° C. ¹H NMR (300 MHz, DMSO-d₆): δ 8.21 (1H, dd, J=7.9, 1.2 Hz), 7.97-7.88 (2H, m), 7.59-7.53 (1H, m), 7.16 (1H, t, J=7.9 Hz), 6.79-6.67 (3H, m), 4.37 (2H, t, J=7.6 Hz), 3.69 (3H, s), 1.69-1.61 (2H, m), 1.46-1.38 (2H, m), 0.93 (3H, t, J=7.3 Hz). MS (ES+): m/e 425.0 (25), 424.0 (100). MS (ES−): m/e 422.9 (25), 421.9 (100).

Compound 226: m.p. 151-152° C. ¹H NMR (300 MHz, DMSO-d₆): δ 8.23 (1H, dd, J=8.2, 1.4 Hz), 8.05 (1H, d, J=8.8 Hz), 7.95 (1H, td, J=7.3, 1.5 Hz), 7.59 (1H, t, J=7.9 Hz), 7.17 (1H, t, J=7.9 Hz), 6.79-6.69 (3H, m), 4.70 (2H, t, J=6.6 Hz), 3.69 (3H, s), 3.03 (2H, t, J=6.6 Hz). MS (ES+): m/e 422.0 (20), 421.0 (100). MS (ES−): m/e 419.9 (20), 418.9 (100).

Compound 344: m.p. 182-184° C. ¹H NMR (300 MHz, DMSO-d₆): δ 8.15 (1H, d, J=9.6 Hz), 7.43 (2H, t, J=7.7 Hz), 7.30-6.98 (15H, m), 5.46 (2H, s). MS (ES+): m/e 521.64 (25), 520.51 (100). MS (ES−): m/e 519.64 (30), 518.55 (100).

Compound 345: m.p. 198-199° C. ¹H NMR (300 MHz, DMSO-d₆): δ 8.18 (1H, d, J=8.8 Hz), 7.44 (2H, t, J=7.8 Hz), 7.31-7.02 (14H, m), 5.49 (2H, s). MS (ES+): m/e 556.45 (40), 554.48 (100). MS (ES−): m/e 554.45 (40), 552.55 (100).

Compound 346: m.p. 155-156° C. ¹H NMR (300 MHz, DMSO-d₆): δ 8.21 (1H, d, J=8.8 Hz), 7.44 (2H, t, J=7.9 Hz), 7.33-7.24 (3H, m), 7.20-7.03 (9H, m), 6.82-6.69 (2H, m), 5.53 (2H, s), 3.70 (3H, s). MS (ES+): m/e 551.71 (25), 550.56 (100). MS (ES−): m/e 549.57 (30), 548.57 (100).

Compound 347: m.p. 177-178° C. ¹H NMR (300 MHz, DMSO-d₆): δ 8.22 (1H, d, J=8.8 Hz), 7.45 (2H, t, J=7.8 Hz), 7.34-7.26 (4H, m), 7.16-7.03 (9H, m), 6.95 (1H, td, J=8.8, 1.8 Hz). ¹⁹F NMR (282 MHz, DMSO-d₆): δ −112.72 (1F, m). MS (ES+): m/e 539.67 (25), 538.49 (100). MS (ES−): m/e 537.59 (25), 536.51 (100).

Compound 348: m.p. 203-205° C. ¹H NMR (300 MHz, DMSO-d₆): δ 8.19 (1H, d, J=9.0 Hz), 7.44 (2H, t, J=7.9 Hz), 7.34-7.27 (6H, m), 7.15-7.03 (8H, m), 5.52 (2H, s). ¹⁹F NMR (282 MHz, DMSO-d₆): δ −117.53 (1F, m). MS (ES+): m/e 539.60 (25), 538.52 (100). MS (ES−): m/e 536.54 (100).

Compound 349: m.p. 172-174° C. ¹H NMR (300 MHz, DMSO-d₆): δ 8.12 (1H, d, J=8.2 Hz), 7.57 (1H, s), 7.39-7.22 (10H, m), 7.18-7.11 (1H, m), 5.65 (2H, s), 2.43 (3H, s). MS (ES+): m/e 443.61 (25), 442.42 (100).

Compound 350: m.p. 200-201° C. ¹H NMR (300 MHz, DMSO-d₆): δ 8.13 (1H, d, J=8.2 Hz), 7.58 (1H, s), 7.40-7.17 (10H, m), 5.66 (2H, s), 2.43 (3H, s). MS (ES+): m/e 478.33 (40), 476.40 (100). MS (ES−): m/e 476.41 (40), 474.42 (100).

Compound 549: m.p. 166-167° C. ¹H NMR (300 MHz, DMSO-d₆): δ 8.12 (1H, d, J=8.4 Hz), 7.57 (1H, s), 7.38-7.23 (6H, m), 7.17 (1H, t, J=7.9 Hz), 6.82-6.69 (3H, m), 5.65 (2H, s), 3.70 (3H, s), 2.43 (3H, s). MS (ES+): m/e 473.64 (20), 472.46 (100). MS (ES−): m/e 471.63 (25), 470.50 (100).

Compound 550: m.p. 194-195° C. ¹H NMR (300 MHz, DMSO-d₆): δ 8.13 (1H, d, J=8.2 Hz), 7.58 (1H, s), 7.40-7.24 (7H, m), 7.11-7.06 (2H, m), 6.99-6.92 (1H, m), 5.66 (2H, s), 2.43 (3H, s). ¹⁹F NMR (282 MHz, DMSO-d₆): δ −112.74 (1F, m). MS (ES+): m/e 461.62 (20), 460.39 (100). MS (ES−): m/e 459.52 (25), 458.43 (100).

Compound 551: m.p. 192-194° C. ¹H NMR (300 MHz, DMSO-d₆): δ 8.09 (1H, d, J=8.2 Hz), 7.55 (1H, s), 7.37-7.23 (8H, m), 7.13-7.07 (2H, m), 5.63 (2H, s), 2.41 (3H, s). ¹⁹F NMR (282 MHz, DMSO-d₆): δ −117.61 (1F, m). MS (ES+): m/e 460.40 (100). MS (ES−): m/e 459.51 (25), 458.41 (100).

Compound 365: m.p. 196-197° C. ¹H NMR (300 MHz, DMSO-d₆): δ 8.27 (1H, d, J=8.8 Hz), 7.61 (1H, s), 7.49 (1H, d, J=8.8 Hz), 7.38-7.13 (8H, m), 5.69 (2H, s), 3.65 (2H, q, J=7.3 Hz), 1.66 (3H, s), 0.85 (3H, t, J=7.3 Hz). ¹⁹F NMR (282 MHz, DMSO-d₆): δ −115.29 (1F, m). MS (ES−): m/e 565.49 (40), 563.54 (100).

Compound 366: m.p. 102-103° C. ¹H NMR (300 MHz, DMSO-d₆): δ 8.26 (1H, d, J=8.5 Hz), 7.60 (1H, s), 7.48 (1H, d, J=8.5 Hz), 7.40-7.32 (2H, m), 7.21-7.11 (3H, m), 6.80-6.70 (3H, m), 5.68 (2H, s), 3.70 (3H, s), 3.64 (2H, q, J=7.0 Hz), 1.66 (3H, s), 0.85 (3H, t, J=7.0 Hz). ¹⁹F NMR (282 MHz, DMSO-d₆): δ −115.34 (1F, m). MS (ES+): m/e 562.79 (25), 561.58 (100). MS (ES−): m/e 560.67 (30), 559.56 (100).

Compound 367: m.p. 152-153° C. ¹H NMR (300 MHz, DMSO-d₆): δ 8.25 (1H, d, J=8.5 Hz), 7.64 (1H, s), 7.51 (1H, d, J=8.5 Hz), 7.38-7.13 (8H, m), 5.66 (2H, s), 3.77 (2H, t, J=5.6 Hz), 3.25 (2H, t, J=5.6 Hz), 3.01 (3H, s), 1.71 (3H, s). ¹⁹F NMR (282 MHz, DMSO-d₆): δ −115.39 (1F, m). MS (ES−): m/e 595.58 (40), 593.53 (100).

Compound 1088: m.p. 284-286° C. ¹H NMR (300 MHz, DMSO-d₆): δ 11.17 (1H, s), 8.09 (1H, d, J=8.7 Hz), 7.38-7.15 (8H, m), 6.99 (1H, dd, J=9.0, 2.0 Hz), 6.92 (1H, d, J=2.0 Hz), 5.54 (2H, s). MS (ES+): m/e 496.1 (100). MS (ES−): m/e 495.9 (35), 493.8 (100).

### 6.2.2 Synthesis of 3-(Benzoxazol-2-ylthio)-6-benzyl-4-hydroxy-6H-pyrano[3,2-c]quinoline-2,5-dione (Compound 33)

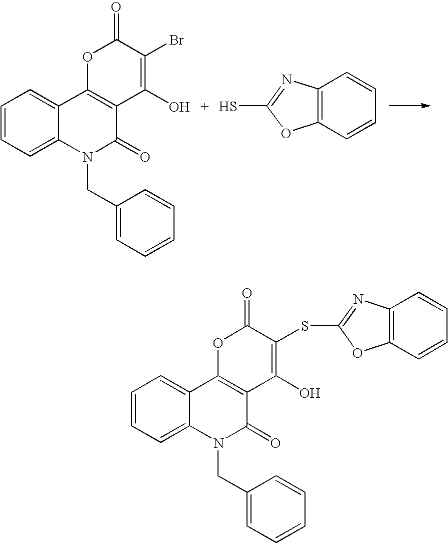

A suspension of sodium hydride in mineral oil (24 mg, 60% w/w) was washed with hexane, and the hexane was decanted off. Dimethylformamide (2 mL) was added, followed by 2-mercaptobenzoxazole (91 mg, 0.60 mmol). After stirring for 30 minutes, the mixture was treated with a solution of 6-benzyl-3-bromo-4-hydroxy-6H-pyrano[3,2-c]quinoline-2,5-dione (200 mg, 0.50 mmol), and the resulting solution was heated to 70° C. overnight. The mixture was cooled and diluted with 4 volumes ethyl acetate and 4 volumes water. The resulting precipitate was collected by filtration and dried under high vacuum to afford the title product (183 mg, 0.39 mmol, 78%) as a pale yellow powder, m.p. 196-198° C. ¹H NMR (300 MHz, DMSO-d₆): δ 8.26 (1H, dd, J=8.2, 1.5 Hz), 7.86 (1H, ddd, J=8.5, 7.0, 1.5 Hz), 7.71 (1H, d, J=8.5 Hz), 7.66-7.52 (3H, m), 7.35-7.24 (7H, m), 5.68 (2H, s). MS (ES+): m/e 470 (25), 469 (100).

The following compounds were prepared in an analogous manner to that of Example 6.2.2.

Compound 50: m.p. 191-192° C. ¹H NMR (300 MHz, DMSO-d₆): δ 8.25 (1H, dd, J=7.9, 1.5 Hz), 7.96-7.91 (2H, m), 7.86 (1H, ddd, J=8.5, 7.0, 1.5 Hz), 7.71 (1H, d, J=8.5 Hz), 7.63-7.52 (4H, m), 7.35-7.23 (5H, m), 5.68 (2H, s). MS (ES+): m/e 497 (25), 496 (100).

Compound 1025: m.p. 225-226° C. ¹H NMR (300 MHz, DMSO-d₆): δ 8.29 (1H, d, J=7.6 Hz), 7.87 (1H, d, J=1.8 Hz), 7.82-7.45 (8H, m), 7.39 (1H, dd, J=8.5, 1.8 Hz), 6.79 (1H, d, J=8.5 Hz). MS (ES+): m/e 490.90 (55), 489.05 (100). MS (ES−): m/e 489.02 (45), 487.09 (100).

Compound 1026: m.p. 240-241° C. ¹H NMR (300 MHz, DMSO-d₆): δ 7.87 (1H, d, J=1.8 Hz), 7.63-7.58 (2H, m), 7.45-7.37 (4H, m), 7.21 (2H, d, J=8.8 Hz), 6.81 (1H, d, J=8.8 Hz), 3.92 (3H, s), 3.86 (3H, s). MS (ES+): m/e 550.81 (55), 549.10 (100). MS (ES−): m/e 549.27 (30), 547.15 (100).

Compound 1027: m.p. 227-228° C. ¹H NMR (300 MHz, DMSO-d₆): δ 8.16 (1H, d, J=8.2 Hz), 7.87 (1H, d, J=2.0 Hz), 7.64-7.60 (2H, m), 7.42-7.26 (7H, m), 5.67 (2H, s), 2.45 (3H, s). MS (ES+): m/e 518.87 (50), 517.12 (100). MS (ES−): m/e 516.56 (35), 515.14 (100).

Compound 1028: m.p. 250-252° C. ¹H NMR (300 MHz, DMSO-d₆): δ 7.87 (1H, d, J=2.0 Hz), 7.61 (1H, d, J=8.4 Hz), 7.40-7.30 (4H, m), 7.25 (1H, t, J=7.6 Hz), 7.02 (2H, d, J=9.0 Hz), 6.95 (1H, t, J=7.7 Hz), 6.81 (1H, d, J=7.6 Hz), 3.78 (3H, s), 2.98-2.88 (2H, m), 2.86-2.76 (2H, m). MS (ES+): m/e 572.76 (45), 571.13 (100). MS (ES−): m/e 571.33 (35), 569.15 (100).

Compound 1029: m.p. 235-236° C. ¹H NMR (300 MHz, DMSO-d₆): δ 7.87 (1H, d, J=1.8 Hz), 7.61 (1H, d, J=8.4 Hz), 7.54-7.46 (2H, m), 7.40-7.25 (5H, m), 6.96 (1H, t, J=7.4 Hz), 6.74 (1H, d, J=7.9 Hz), 2.92-2.72 (2H, m), 2.55-2.48 (2H, m). MS (ES+): m/e 560.73 (45), 559.12 (100). MS (ES−): m/e 559.29 (30), 557.10 (100).

Compound 1030: m.p. 216-218° C. ¹H NMR (300 MHz, DMSO-d₆): δ 8.18 (1H, d, J=9.0 Hz), 7.86 (1H, d, J=1.8 Hz), 7.61 (1H, d, J=8.5 Hz), 7.46-7.36 (3H, m), 7.21-7.11 (4H, m), 5.66 (2H, s), 3.87 (3H, s). MS (ES+): m/e 552.92 (55), 551.06 (100). MS (ES−): m/e 550.95 (50), 549.03 (100).

Compound 1025: m.p. 225-226° C. ¹H NMR (300 MHz, DMSO-d₆): δ 8.29 (1H, d, J=7.6 Hz), 7.87 (1H, d, J=1.8 Hz), 7.82-7.45 (8H, m), 7.39 (1H, dd, J=8.5, 1.8 Hz), 6.79 (1H, d, J=8.5 Hz). MS (ES+): m/e 490.90 (55), 489.05 (100). MS (ES−): m/e 489.02 (45), 487.09 (100).

Compound 1026: m.p. 240-241° C. ¹H NMR (300 MHz, DMSO-d₆): δ 7.87 (1H, d, J=1.8 Hz), 7.63-7.58 (2H, m), 7.45-7.37 (4H, m), 7.21 (2H, d, J=8.8 Hz), 6.81 (1H, d, J=8.8 Hz), 3.92 (3H, s), 3.86 (3H, s). MS (ES+): m/e 550.81 (55), 549.10 (100). MS (ES−): m/e 549.27 (30), 547.15 (100).

Compound 1027: m.p. 227-228° C. ¹H NMR (300 MHz, DMSO-d₆): δ 8.16 (1H, d, J=8.2 Hz), 7.87 (1H, d, J=2.0 Hz), 7.64-7.60 (2H, m), 7.42-7.26 (7H, m), 5.67 (2H, s), 2.45 (3H, s). MS (ES+): m/e 518.87 (50), 517.12 (100). MS (ES−): m/e 516.56 (35), 515.14 (100).

Compound 1028: m.p. 250-252° C. ¹H NMR (300 MHz, DMSO-d₆): δ 7.87 (1H, d, J=2.0 Hz), 7.61 (1H, d, J=8.4 Hz), 7.40-7.30 (4H, m), 7.25 (1H, t, J=7.6 Hz), 7.02 (2H, d, J=9.0 Hz), 6.95 (1H, t, J=7.7 Hz), 6.81 (1H, d, J=7.6 Hz), 3.78 (3H, s), 2.98-2.88 (2H, m), 2.86-2.76 (2H, m). MS (ES+): m/e 572.76 (45), 571.13 (100). MS (ES−): m/e 571.33 (35), 569.15 (100).

Compound 1029: m.p. 235-236° C. ¹H NMR (300 MHz, DMSO-d₆): δ 7.87 (1H, d, J=1.8 Hz), 7.61 (1H, d, J=8.4 Hz), 7.54-7.46 (2H, m), 7.40-7.25 (5H, m), 6.96 (1H, t, J=7.4 Hz), 6.74 (1H, d, J=7.9 Hz), 2.92-2.72 (2H, m), 2.55-2.48 (2H, m). MS (ES+): m/e 560.73 (45), 559.12 (100). MS (ES−): m/e 559.29 (30), 557.10 (100).

Compound 1030: m.p. 216-218° C. ¹H NMR (300 MHz, DMSO-d₆): δ 8.18 (1H, d, J=9.0 Hz), 7.86 (1H, d, J=1.8 Hz), 7.61 (1H, d, J=8.5 Hz), 7.46-7.36 (3H, m), 7.21-7.11 (4H, m), 5.66 (2H, s), 3.87 (3H, s). MS (ES+): m/e 552.92 (55), 551.06 (100). MS (ES−): m/e 550.95 (50), 549.03 (100).

### 6.2.3 Synthesis of Propane-1-sulfonic acid [4-(4-hydroxy-2,5-dioxo-6-phenyl-5,6-dihydro-2H-pyrano[3,2-c]quinolin-3-ylthio)-phenyl]-amide) (Compound 113)

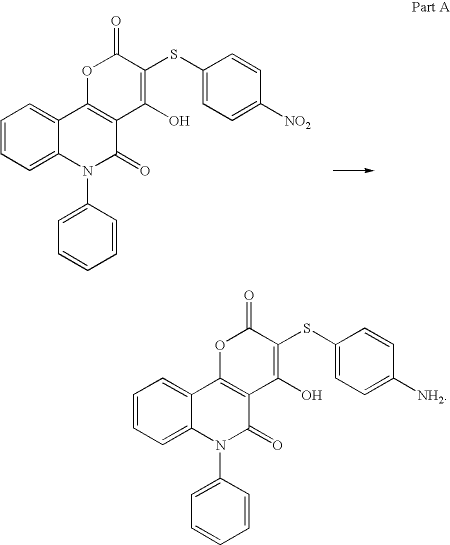

A solution of 4-hydroxy-3-(4-nitro-phenylthio)-6-phenyl-6H-pyrano[3,2-c]quinoline-2,5-dione (prepared using a modification of the procedure of Example 2) (176 mg, 0.383 mmol) and tin (II) chloride dihydrate (826 mg, 3.66 mmol) in 10 mL ethanol was heated to reflux for 14 hours. The reaction mixture was poured into water and extracted with ethyl acetate (2×). The extracts were washed with brine, combined, dried over anhydrous magnesium sulfate, filtered and evaporated. The residue was triturated with diethyl ether, collected by filtration and dried under high vacuum to afford the product, 3-(4-amino-phenylthio)-4-hydroxy-6-phenyl-6H-pyrano[3,2-c]quinoline-2,5-dione, as an off-white powder (140 mg, 0.327 mmol, 85%), m.p.>300° C. ¹H NMR (300 MHz, DMSO-d₆): δ 8.11 (1H, d, J=7.0 Hz), 7.64-7.45 (4H, m), 7.37-7.27 (3H, m), 6.83 (2H, d, J=8.5 Hz), 6.48-6.42 (1H, m), 6.39 (2H, d, J=8.5 Hz). MS (ES+): m/e 430.50 (25), 429.17 (100). MS (ES−): m/e 428.40 (25), 427.16 (100).

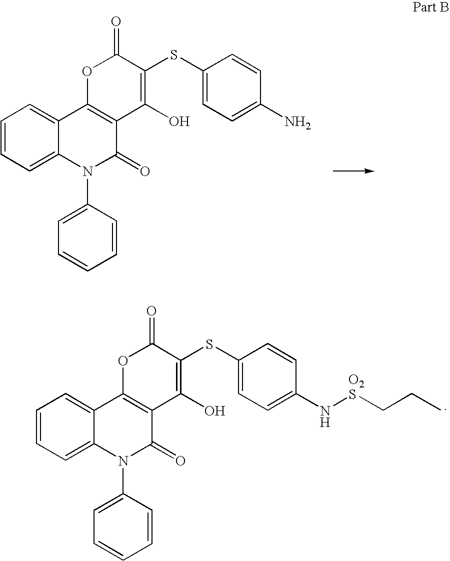

A suspension of 3-(4-amino-phenylthio)-4-hydroxy-6-phenyl-6H-pyrano[3,2-c]quinoline-2,5-dione (60 mg, 0.140 mmol) in anhydrous pyridine (1 mL) was treated with 1-propanesulfonyl chloride (20 μL, 0.172 mmol). The resulting mixture was stirred at ambient temperature overnight, then poured into 4 volumes of 0.5 N HCl and extracted with ethyl acetate (2×). The extracts were washed with more dilute HCl and brine, combined, dried over anhydrous magnesium sulfate, filtered and evaporated. The residual material was purified by trituration with diethyl ether, collected by filtration and dried under high vacuum to afford the title product (41 mg, 0.077 mmol, 55%) as a tan solid, m.p. 249-251° C. ¹H NMR (300 MHz, DMSO-d₆): δ 9.74 (1H, s), 8.24 (1H, dd, J=8.0, 1.3 Hz), 7.77-7.61 (4H, m), 7.55 (1H, t, J=7.8 Hz), 7.48-7.43 (2H, m), 7.23 (2H, d, J=8.5 Hz), 7.11 (2H, d, J=8.5 Hz), 6.76 (1H, d, J=8.7 Hz), 3.04-2.98 (2H, m), 1.70-1.57 (2H, m), 0.90 (3H, t, J=7.4 Hz). MS (ES+): m/e 535.25 (100). MS (ES−): m/e 533.27 (100).

Also prepared in this manner was:

Compound 115: m.p. 260-261° C. ¹H NMR (300 MHz, DMSO-d₆): δ 9.60 (1H, s), 8.23 (1H, dd, J=7.9, 1.5 Hz), 7.76-7.62 (4H, m), 7.54 (1H, t, J=7.6 Hz), 7.48-7.43 (2H, m), 7.36 (2H, d, J=8.5 Hz), 7.20 (2H, d, J=8.5 Hz), 6.75 (1H, d, J=8.8 Hz), 4.08 (2H, q, J=7.0 Hz), 1.20 (3H, t, J=7.0 Hz). MS (ES+): m/e 502(20), 501.25 (100). MS (ES−): m/e 500.49 (20), 499.29 (100).

### 6.2.4 Synthesis of 4-Hydroxy-3-[4-(2-methoxy-ethoxy)-phenylthio]-6-phenyl-6H-pyrano[3,2-c]quinoline-2,5-dione (Compound 122)

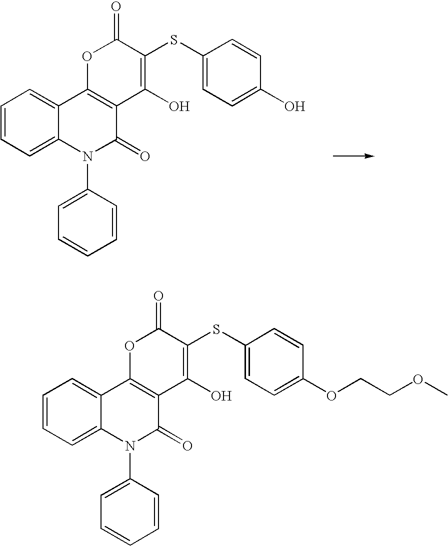

A mixture of 4-hydroxy-3-(4-hydroxy-phenylthio)-6-phenyl-6H-pyrano[3,2-c]quinoline-2,5-dione (prepared in a manner analogous to that of Example 2, above) (102 mg, 0.238 mmol), 1-bromo-2-methoxyethane (50 μL, 0.532 mmol) and potassium carbonate (70 mg, 0.506 mmol) in dimethylformamide (2 mL) was stirred at 60° C. for 14 hours, then was cooled and poured into ethyl acetate. The resulting mixture was washed with water (3) and brine, and the aqueous layers were back-extracted in sequence with ethyl acetate. The extracts were combined, dried over anhydrous magnesium sulfate, filtered and evaporated, and the residual material was separated by silica gel chromatography (eluting with 5:95 methanol-dichloromethane) to afford the pure title product (79 mg, 0.162 mmol, 68%) as an off-white powder, m.p. 144-146° C. TLC R_{F} 0.30 (5:95 methanol-dichloromethane). ¹H NMR (300 MHz, CDCl₃): δ 8.36 (1H, d, J=7.9 Hz), 7.70-7.55 (4H, m), 7.52-7.42 (3H, m), 7.34-7.27 (3H, m), 6.86-6.80 (2H, m), 4.09-4.06 (2H, m), 3.74-3.70 (2H, m), 3.43 (3H, s). MS (ES+): m/e 488.20 (100). MS (ES−): m/e 487.42 (20), 486.23 (100).

Prepared in a similar manner were the following compounds:

Compound 123: m.p. 164-165° C. ¹H NMR (300 MHz, CDCl₃): δ 8.39 (1H, dd, J=8, 2 Hz), 7.73-7.58 (3H, m), 7.46 (1H, t, J=7.5 Hz), 7.36-7.31 (2H, m), 7.16 (1H, t, J=8.4 Hz), 6.98 (1H, d, J=7.9 Hz), 6.93 (1H, t, J=2.0 Hz), 6.85 (1H, d, J=8.8 Hz), 6.72 (1H, dd, J=8.3, 2.6 Hz), 6.60-6.53 (1H, m), 4.10-4.06 (2H, m), 3.71-3.67 (2H, m), 3.41 (3H, s). MS (ES+): m/e 489.54 (20), 488.20 (100). MS (ES−): m/e 486.21 (100).

Compound 124: m.p. 187-188° C. TLC R_{F} 0.45 (5:95 methanol-dichloromethane). ¹H NMR (300 MHz, CDCl₃): δ 8.36 (1H, dd, J=7.9, 1.3 Hz), 7.71-7.55 (3H, m), 7.50-7.43 (3H, m), 7.48 (2H, d, J=8 Hz), 7.33-7.29 (2H, m), 6.85-6.80 (2H, m), 4.59 (2H, s), 3.79 (3H, s). MS (ES+): m/e 502.18 (100). MS (ES−): m/e 500.22 (100).

Compound 125: m.p. 126-128° C. ¹H NMR (300 MHz, CDCl₃): δ 8.39 (1H, dd, J=7.9, 1.3 Hz), 7.72-7.59 (4H, m), 7.47 (1H, td, J=7.1, 0.9 Hz), 7.36-7.32 (2H, m), 7.18 (1H, t, J=7.9 Hz), 7.02 (1H, ddd, J=7.9, 1.8, 0.9 Hz), 6.90 (1H, t, J=2.2 Hz), 6.85 (1H, d, J=8.3 Hz), 6.69 (1H, ddd, J=8.3, 2.6, 0.9 Hz), 4.59 (2H, s), 3.78 (3H, s). MS (ES+): m/e 501.94 (100). MS (ES−): m/e 500.03 (100).

Compound 126: m.p. 222-224° C. ¹H NMR (300 MHz, CDCl₃): δ 8.35 (1H, dd, J=8, 2 Hz), 7.70-7.27 (14H, m), 6.88 (2H, d, J=9.2 Hz), 6.82 (1H, d, J=8.8 Hz), 5.02 (2H, s). MS (ES+): m/e 520.24 (100). MS (ES−): m/e 519.28 (25), 518.16 (100).

Compound 127: m.p. 169-170° C. ¹H NMR (300 MHz, CDCl₃): δ 8.39 (1H, dd, J=7.9, 1.3 Hz), 7.72-7.58 (4H, m), 7.49-7.24 (8H, m), 7.17 (1H, t, J=8.1 Hz), 6.99 (1H, ddd, J=8.8, 1.8, 0.9 Hz), 6.95 (1H, t, J=2.0 Hz), 6.85 (1H, d, J=8.4 Hz), 6.77 (1H, ddd, J=8.3, 2.6, 0.9 Hz), 5.02 (2H, s). MS (ES+): m/e 520.23 (100). MS (ES−): m/e 519.47 (20), 518.23 (100).

### 6.2.5 Synthesis of 4,5-Dihydro-9-(3-Fluorophenylthio)-8-hydroxy-5-methyl-7H,10H-Pyrano[3,2-c]pyrrolo[3,2,1-ij]quinoline-7,10-dione (Compound 206)

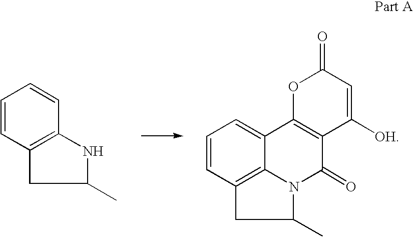

A mixture of 2-methyl-2,3-dihydro-1H-indole (3.50 mL, 26.8 mmol) and diethyl malonate (9.00 mL, 59.3 mmol) in diphenyl ether (30 mL) was heated to 240° C. in a manner similar to that described in Example 2. Cooling, filtering and washing with diethyl ether afforded a dark yellow solid. Analysis by LC/MS showed the material to be a mixture of the desired product and a compound that had incorporated only one mole of malonate. This solid was re-dissolved in diphenyl ether, and diisopropyl malonate (6.0 mL, 31.6 mmol) was added. Distillation was performed at 250-280° C., and cooling again gave a solid, which after filtering, washing and drying under high vacuum, proved to be pure product (4,5-dihydro-8-hydroxy-5-methyl-7H,10H-pyrano[3,2-c]pyrrolo[3,2,1-ij]quinoline-7,10-dione) by LC/MS (2.64 g, 9.80 mmol, 37%), m.p. 199-200° C. ¹H NMR (300 MHz, DMSO-d₆): δ 7.81 (1H, d, J=8.2 Hz), 7.68 (1H, d, J=7.3 Hz), 7.42 (1H, dd, J=8.2, 7.3 Hz), 5.60 (1H, d, J=0.9 Hz), 5.11-5.02 (1H, m), 3.71 (1H, dd, J=17.5, 9.6 Hz), 3.07 (1H, dd, J=17.5, 2.4 Hz), 1.53 (3H, d, J=6.4 Hz). MS (ES+): m/e 271.6 (15), 270.5 (100). MS (ES−): m/e 269.3 (15), 268.4 (100).

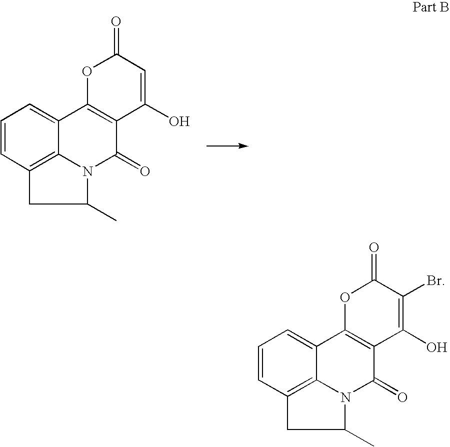

A suspension of 4,5-dihydro-8-hydroxy-5-methyl-7H,10H-pyrano[3,2-c]pyrrolo[3,2,1-ij]quinoline-7,10-dione (2.62 g, 9.73 mmol) and N-bromosuccinimide (1.90 g, 10.7 mmol) in 30 mL acetonitrile was heated to reflux overnight, then was cooled and filtered. The filter cake was washed with acetonitrile and diethyl ether, and the solid was dried under high vacuum to afford the product, 4,5-dihydro-9-bromo-8-hydroxy-5-methyl-7H,10H-pyrano[3,2-c]pyrrolo[3,2,1-ij]quinoline-7,10-dione, as a yellow powder (2.83 g, 8.13 mmol, 84%), m.p. 221-222° C. ¹H NMR (300 MHz, DMSO-d₆): δ 7.82 (1H, d, J=8.2 Hz), 7.70 (1H, d, J=7.3 Hz), 7.44 (1H, t, J=7.8 Hz), 5.14-5.05 (1H, m), 3.72 (1H, dd, J=17.2, 9.3 Hz), 3.09 (1H, dd, J=17.2, 3.2 Hz), 1.54 (3H, d, J=6.4 Hz). MS (ES+): m/e 351.5 (15), 350.5 (100), 348.5 (90). MS (ES−): m/e 349.3 (15), 348.4 (100), 346.4 (98).

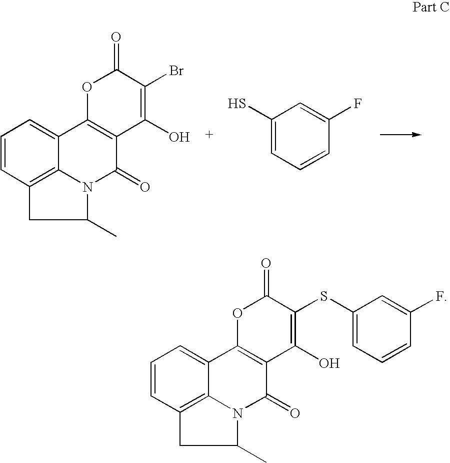

A suspension of 4,5-dihydro-9-bromo-8-hydroxy-5-methyl-7H,10H-pyrano[3,2-c]pyrrolo[3,2,1-ij]quinoline-7,10-dione (150 mg, 0.431 mmol), 3-fluorothiophenol (141 μL, 1.30 mmol) and cesium carbonate (140 mg, 0.43 mmol) in 2 mL dimethylformamide was heated with stirring to 70° C. overnight. The mixture was cooled and partitioned between 10 mL each of 0.25 N HCl and diethyl ether. The mixture was filtered, and the collected precipitate was washed with more diethyl ether and dried under high vacuum to give the title product (113 mg, 0.286 mmol, 66%) as a yellow powder, m.p. 202-203° C. ¹H NMR (300 MHz, DMSO-d₆): δ 7.88 (1H, d, J=8.2 Hz), 7.74 (1H, d, J=7.3 Hz), 7.47 (1H, dd, J=8.2, 7.3 Hz), 7.33-7.25 (1H, m), 7.08-7.03 (2H, m), 6.98-6.91 (1H, m), 5.16-5.09 (1H, m), 3.74 (1H, dd, J=17.2, 9.1 Hz), 3.11 (1H, dd, J=17.2, 3.8 Hz), 1.55 (3H, d, J=6.7 Hz). ¹⁹F NMR (282 MHz, DMSO-d₆): δ −112.78 (1F, q, J=7.9 Hz). MS (ES+): m/e 397.52 (20), 396.20 (100). MS (ES−): m/e 395.42 (20), 394.21 (100).

In a similar manner, the following compounds were prepared:

Compound 205: m.p. 182-183° C. ¹H NMR (300 MHz, DMSO-d₆): δ 7.88 (1H, d, J=7.6 Hz), 7.74 (1H, d, J=6.4 Hz), 7.47 (1H, t, J=7.0 Hz), 7.25-7.02 (4H, m), 5.16-5.09 (1H, m), 3.74 (1H, dd, J=17.2, 9.1 Hz), 3.11 (1H, dd, J=17.2, 3.6 Hz), 1.55 (3H, d, J=6.5 Hz). ¹⁹F NMR (282 MHz, DMSO-d₆): δ −114.44 (1F, dd, J=15.9, 6.0 Hz). MS (ES+): m/e 397.52 (20), 396.20 (100). MS (ES−): m/e 395.49 (20), 394.24 (100).

Compound 207: m.p. 201-202° C. ¹H NMR (300 MHz, DMSO-d₆): δ 7.86 (1H, d, J=8.2 Hz), 7.72 (1H, d, J=7.0 Hz), 7.46 (1H, t, J=7.6 Hz), 7.29 (2H, dd, J=8.8, 5.2 Hz), 7.10 (2H, t, J=8.8 Hz), 5.15-5.08 (1H, m), 3.73 (1H, dd, J=17.3, 9.4 Hz), 3.10 (1H, dd, J=17.3, 3.5 Hz), 1.55 (3H, d, J=6.4 Hz). ¹⁹F NMR (282 MHz, DMSO-d₆): δ −117.56 (1F, dt, J=9.9, 6.9 Hz). MS (ES+): m/e 397.57 (20), 396.22 (100). MS (ES−): m/e 395.51 (10), 394.22 (100).

Compound 208: m.p. 190-191° C. ¹H NMR (300 MHz, DMSO-d₆): δ 7.87 (1H, d, J=8.2 Hz), 7.73 (1H, d, J=7.0 hz), 7.47 (1H, t, J=7.6 Hz), 7.30-7.16 (4H, m), 5.15-5.08 (1H, m), 3.74 (1H, dd, J=17.2, 9.0 Hz), 3.11 (1H, dd, J=17.2, 3.3 Hz), 1.55 (3H, d, J=6.4 Hz). MS (ES+): m/e 413.97 (40), 412.18 (100). MS (ES−): m/e 412.13 (45), 410.16 (100).

Compound 209: m.p. 162-163° C. ¹H NMR (300 MHz, DMSO-d₆): δ 7.86 (1H, d, J=8.2 Hz), 7.72 (1H, d, J=6.8 Hz), 7.46 (1H, t, J=7.5 Hz), 7.16 (1H, t, J=7.9 Hz), 6.78-6.68 (3H, m), 5.15-5.08 (1H, m), 3.74 (1H, dd, J=17.2, 8.8 Hz), 3.69 (3H, s), 3.10 (1H, dd, J=17.2, 3.5 Hz), 1.55 (3H, d, J=6.4 Hz). MS (ES+): m/e 409.57 (20), 408.22 (100). MS (ES−): m/e 407.43 (20), 406.29 (100).

Compound 210: m.p. 159-160° C. ¹H NMR (300 MHz, DMSO-d₆): δ 7.88 (1H, d, J=8.2 Hz), 7.74 (1H, d, J=6.4 Hz), 7.55-7.43 (5H, m), 5.16-5.09 (1H, m), 3.75 (1H, dd, J=17.2, 9.3 Hz), 3.11 (1H, dd, J=17.2, 3.8 Hz), 1.56 (3H, d, J=6.4 Hz). ¹⁹F NMR (282 MHz, DMSO-d₆): δ −61.56 (3F, s). MS (ES+): m/e 447.53 (20), 446.17 (100). MS (ES−): m/e 445.53 (20), 444.18 (100).

### 6.2.6 Synthesis of 6-(4-Fluorobenzyl)-4-hydroxy-7,9-dimethyl-3-(phenylthio)pyrano[2,3-d]pyrazolo[3,4-b]pyridine-2,5(6H,7H)-dione (Compound 552)

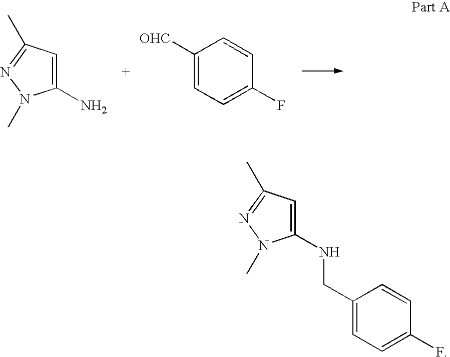

A solution of 5-amino-1,3-dimethylpyrazole (2.22 g, 20.0 mmol) and 4-fluorobenzaldehyde (2.20 mL, 20.4 mmol) in 20 mL methanol was cooled to 0° C., and 0.5 mL acetyl chloride was slowly added. After stirring for 20 minutes, sodium cyanoborohydride (2.50 g, 39.8 mmol) was added in portions over 30 minutes. The resulting solution was stirred for 14 hours, then poured into satd. aq. sodium bicarbonate and stirred for 1 hour. The mixture was extracted 2× with ethyl acetate, and the extracts were washed with brine, combined, dried over anhydrous sodium sulfate, filtered and evaporated to afford the product, 1,3-dimethyl-5-[N-(4-fluorobenzyl)amino]pyrazole (1.75 g, 7.98 mmol, 40%), as an oil. TLC R_{F} 0.22 (ethyl acetate). ¹H NMR (300 MHz, CDCl₃): δ 7.32 (2H, dd, J=9.0, 5.5 Hz), 7.03 (2H, t, J=8.8 Hz), 5.27 (1H, s), 4.20 (2H, s), 3.77 (1H, br s), 3.58 (3H, s), 2.16 (3H, s). ¹⁹F NMR (282 MHz, CDCl₃): δ −115.29 (1F, m). MS (ES+): m/e 221.3 (20), 220.3 (100).

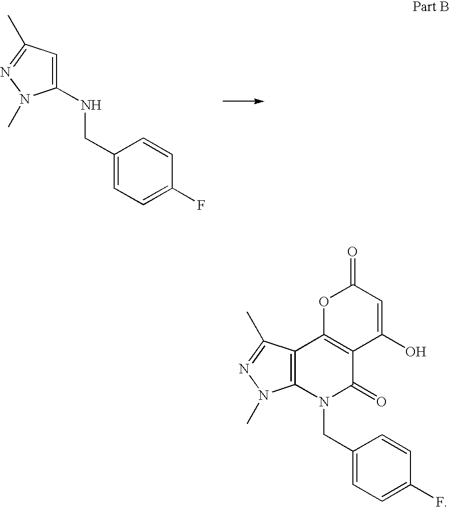

The usual high-temperature method was used to convert 1,3-dimethyl-5-[N-(4-fluorobenzyl)amino]pyrazole (1.73 g, 7.89 mmol) to 6-(4-fluorobenzyl)-4-hydroxy-7,9-dimethylpyrano[2,3-d]pyrazolo[3,4-b]pyridine-2,5(6H,7H)-dione (first crop 1.58 g, 4.44 mmol, 56%), m.p. 238-240° C. ¹H NMR (300 MHz, DMSO-d₆): δ 12.91 (1H, s), 7.26-7.15 (4H, m), 5.61 (2H, s), 5.43 (1H, s), 3.82 (3H, s), 2.45 (3H, s). ¹⁹F NMR (282 MHz, DMSO-d₆): δ −115.44 (1F, m). MS (ES+): m/e 357.40 (15), 356.25 (100). MS (ES−): m/e 355.43 (15), 354.27 (100).

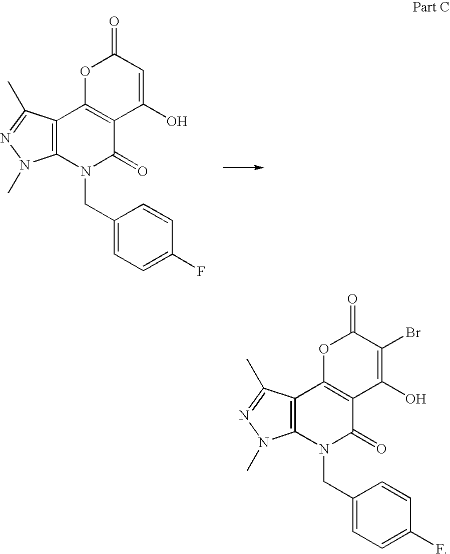

The usual bromination procedure was employed using 6-(4-fluorobenzyl)-4-hydroxy-7,9-dimethylpyrano[2,3-d]pyrazolo[3,4-b]pyridine-2,5(6H,7H)-dione (531 mg, 1.49 mmol) which gave the product, 3-bromo-6-(4-fluorobenzyl)-4-hydroxy-7,9-dimethylpyrano[2,3-d]pyrazolo[3,4-b]pyridine-2,5(6H,7H)-dione, as a yellow powder (425 mg, 0.979 mmol, 66%), m.p. 262-264° C. ¹H NMR (300 MHz, DMSO-d₆): δ 7.24-7.15 (4H, m), 5.65 (2H, s), 3.84 (3H, s), 2.46 (3H, s). ¹⁹F NMR (282 MHz, DMSO-d₆): δ −115.32 (1F, m). MS (ES+): m/e 436 (95), 434 (100). MS (ES−): m/e 434 (100), 432 (95).

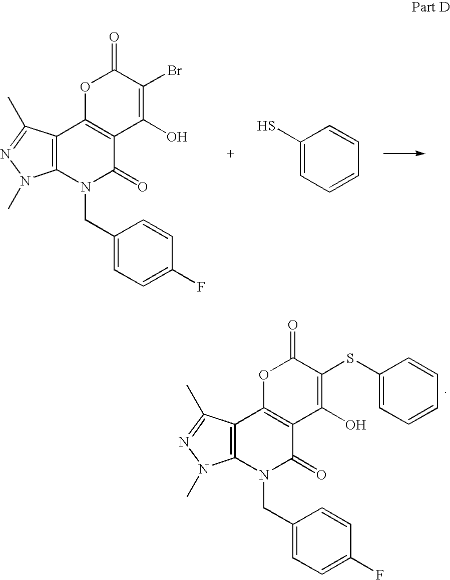

A solution of 3-bromo-6-(4-fluorobenzyl)-4-hydroxy-7,9-dimethylpyrano[2,3-d]pyrazolo[3,4-b]pyridine-2,5(6H,7H)-dione (68 mg, 0.157 mmol), thiophenol (66 mL, 0.624 mmol) and potassium carbonate (44 mg, 0.318 mmol) in dimethylformamide (2 mL) was heated to 60° C. overnight. The reaction mixture was cooled and partitioned between 10 mL each of dilute HCl and diethyl ether. The mixture was filtered, and the collected precipitate was washed with more diethyl ether and dried under high vacuum to give the title product as a light yellow powder (57 mg, 0.123 mmol, 78%), m.p. 159-160° C. ¹H NMR (300 MHz, DMSO-d₆): δ 7.37-7.09 (9H, m), 5.65 (2H, s), 3.85 (3H, s), 2.48 (3H, s). ¹⁹F NMR (282 MHz, DMSO-d₆): δ −115.32 (1F, s). MS (ES+): m/e 465.60 (20), 464.42 (100). MS (ES−): m/e 463.56 (25), 462.46 (100).

Also prepared using variations of this procedure were the following compounds:

Compound 553: m.p. 189-190° C. ¹H NMR (300 MHz, DMSO-d₆): δ 7.30-7.16 (8H, m), 5.66 (2H, s), 3.85 (3H, s), 2.48 (3H, s). ¹⁹F NMR (282 MHz, DMSO-d₆): δ −115.30 (1F, m). MS (ES+): m/e 500.32 (40), 498.39 (100).

Compound 554: m.p. 173-174° C. ¹H NMR (300 MHz, DMSO-d₆): δ 7.30-7.14 (5H, m), 6.76-6.68 (3H, m), 5.65 (2H, s), 3.84 (3H, s), 3.70 (3H, s), 2.48 (3H, s). ¹⁹F NMR (282 MHz, DMSO-d₆): δ −115.34 (1F, m). MS (ES+): m/e 495.65 (25), 494.44 (100). MS (ES−): m/e 493.55 (25), 492.46 (100).

Compound 555: m.p. 199-201° C. ¹H NMR (300 MHz, DMSO-d₆): δ 7.34-7.17 (5H, m), 7.06-6.91 (3H, m), 5.66 (2H, s), 3.85 (3H, s), 2.47 (3H, s). ¹⁹F NMR (282 MHz, DMSO-d₆): δ −112.77 (1F, m), −115.30 (1F, m). MS (ES+): m/e 483.60 (20), 482.40 (100). MS (ES−): m/e 481.55 (25), 480.46 (100).

Compound 556: m.p. 221-222° C. ¹H NMR (300 MHz, DMSO-d₆): δ 7.30-7.08 (8H, m), 5.65 (2H, s), 3.85 (3H, s), 2.48 (3H, s). ¹⁹F NMR (282 MHz, DMSO-d₆): δ −115.32 (1F, m), −117.71 (1F, m). MS (ES+): m/e 483.61 (20), 482.41 (100). MS (ES−): m/e 481.52 (25), 480.42 (100).

Compound 680: m.p. 254-256° C. ¹H NMR (300 MHz, DMSO-d₆): δ 7.30-7.10 (4H, m), 4.22 (2H, t, J=5.7 Hz), 4.06 (2H, t, J=5.8 Hz), 2.50 (3H, s), 2.32 (2H, p, J=5.3 Hz). MS (ES+): m/e 417.8 (35), 415.8 (100). MS (ES−): m/e 415.7 (35), 413.7 (100).

Compound 681: m.p. 202-203° C. ¹H NMR (300 MHz, DMSO-d₆): δ 7.15 (1H, t, J=8.4 Hz), 6.72-6.66 (3H, m), 4.21 (2H, t, J=5.5 Hz), 4.05 (2H, t, J=5.5 Hz), 3.68 (3H, s), 2.49 (3H, s), 2.32 (2H, m). MS (ES+): m/e 412.9 (20), 411.9 (100). MS (ES−): m/e 410.8 (15), 409.8 (100).

### 6.2.7 Synthesis of 4-Hydroxy-7,9-dimethyl-3-(phenylthio)-6-(4-(trifluoromethoxy)phenyl)pyrano[2,3-d]pyrazolo[3,4-b]pyridine-2,5(6H,7H)-dione (Compound 791)

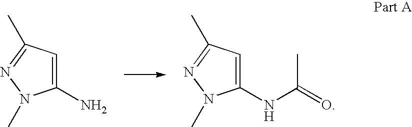

A mixture of 5-amino-1,3-dimethylpyrazole (2.33 g, 21.0 mmol) and acetic anhydride (2.00 mL, 21.2 mmol) in acetic acid (50 mL) was heated to 50° C. for 12 hours, then cooled and poured into water. This mixture was neutralized with solid sodium bicarbonate addition, and then was extracted twice with ethyl acetate. The extracts were washed with brine, combined, dried over magnesium sulfate, filtered and evaporated to afford the product, 5-acetylamino-1,3-dimethylpyrazole, as an oil, which solidified upon standing to a waxy solid (3.09 g, 20.2 mmol, 96%), m.p. 45-46° C. ¹H NMR (300 MHz, CDCl₃): δ 8.26 (1H, br s), 5.96 (1H, s), 3.58 (3H, s), 2.17 (3H, s), 2.12 (3H, s). MS (ES+): m/e 153.9 (100).

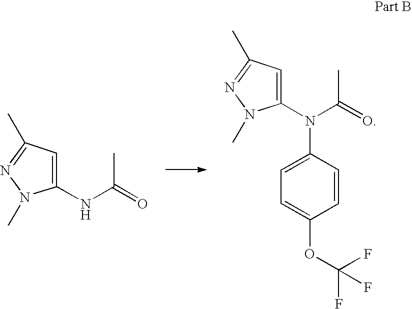

A mixture of 5-acetylamino-1,3-dimethylpyrazole (1.61 g, 10.5 mmol), 4-iodo-1-trifluoromethoxybenzene (2.20 mL, 13.8 mmol), copper (I) iodide (50 mg, 0.263 mmol) and potassium triphosphate (4.68 g, 22.0 mmol) in 30 mL dioxane was degassed by three successive cycles of vacuum pumping with dry nitrogen purging. Trans 1,2-diaminocyclohexane (0.127 mL, 1.05 mmol) was introduced by syringe, and the resulting mixture stirred and heated to reflux for 12 hours. The mixture was cooled and filtered through celite. The filtrate was partially evaporated, and the residual material was separated by column chromatography (silica gel, eluting with 50:50 ethyl acetate-hexane) to afford the pure product, N-(1,3-dimethyl-2H-pyrazol-5-yl)-N-(4-trifluoromethoxy-phenyl)-acetamide, as an oil (2.72 g, 8.68 mmol, 83%). TLC R_{F} 0.30 (50:50 ethyl acetate-hexane). ¹H NMR (300 MHz, CDCl₃): δ 7.30 (2H, d, J=9.0 Hz), 7.22 (1H, s), 7.21 (2H, d, J=9.0 Hz), 3.64 (3H, s), 2.25 (3H, s), 2.07 (3H, s). MS (ES+): m/e 315.0 (15), 314.0 (100).

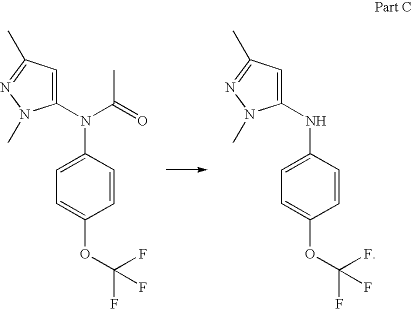

A mixture of N-(1,3-dimethyl-2H-pyrazol-5-yl)-N-(4-trifluoromethoxy-phenyl)-acetamide (1.78 g, 5.68 mmol) and HCl (4 N in dioxane, 10 mL, 40 mmol) in 10 mL ethanol was heated to reflux overnight. The mixture was cooled and poured into water, which was then neutralized with solid sodium bicarbonate. This mixture was then extracted twice with ethyl acetate, and the extracts were washed with brine, combined, dried over sodium sulfate, filtered and evaporated to afford sufficiently pure product, (1,3-dimethyl-2H-pyrazol-5-yl)-(4-trifluoromethoxy-phenyl)-amine, as a waxy solid (1.50 g, 5.53 mmol, 97%), m.p. 80-82° C. TLC R_{F} 0.17 (50:50 ethyl acetate-hexane). ¹H NMR (300 MHz, CDCl₃): δ 8.13 (1H, s), 7.16 (2H, d, J=8.8 Hz), 6.83 (2H, d, J=8.8 Hz), 5.79 (1H, s), 3.53 (3H, s), 2.08 (3H, s). MS (ES+): m/e 273.34 (15), 272.38 (100). MS (ES−): m/e 271.37 (15), 270.18 (100).

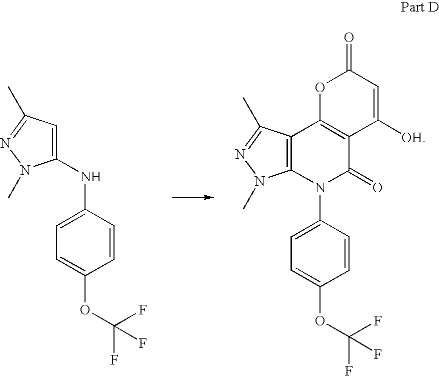

The standard high-temperature condensation reaction described in Example 2 was used to convert (1,3-dimethyl-2H-pyrazol-5-yl)-(4-trifluoromethoxy-phenyl)-amine (1.50 g, 5.53 mmol) to 4-hydroxy-7,9-dimethyl-6-(4-(trifluoromethoxy)phenyl)pyrano[2,3-d]pyrazolo[3,4-b]pyridine-2,5(6H,7H)-dione (1.23 g, 3.01 mmol, 54%), m.p. 266-267° C. ¹H NMR (300 MHz, DMSO-d₆): δ 12.68 (1H, s), 7.79 (2H, d, J=8.8 Hz), 7.66 (2H, d, J=8.8 Hz), 5.44 (1H, s), 3.05 (3H, s), 2.48 (3H, s). MS (ES+): m/e 408.17 (100). MS (ES−): m/e 406.16 (100).

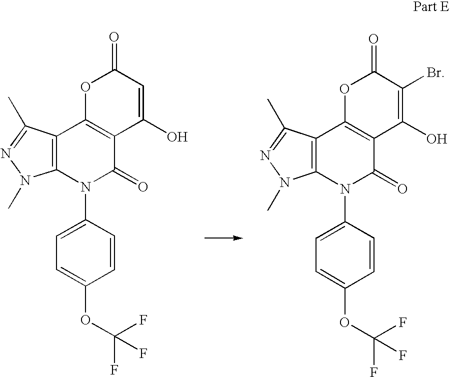

The standard bromination procedure described in Example 2 was used to convert 4-hydroxy-7,9-dimethyl-6-(4-(trifluoromethoxy)phenyl)pyrano[2,3-d]pyrazolo[3,4-b]pyridine-2,5(6H,7H)-dione (1.18 g, 2.90 mmol) to 3-bromo-4-hydroxy-7,9-dimethyl-6-(4-(trifluoromethoxy)phenyl)pyrano[2,3-d]pyrazolo[3,4-b]pyridine-2,5(6H,7H)-dione (1.26 g, 2.59 mmol, 89%), m.p. 308-309° C. ¹H NMR (300 MHz, DMSO-d₆): δ 13.56 (1H, s), 7.80 (2H, d, J=9.0 Hz), 7.67 (2H, d, J=9.0 Hz), 3.07 (3H, s), 2.48 (3H, s). MS (ES+): m/e 488 (100), 486 (95). MS (ES−): m/e 486 (100), 484 (95).

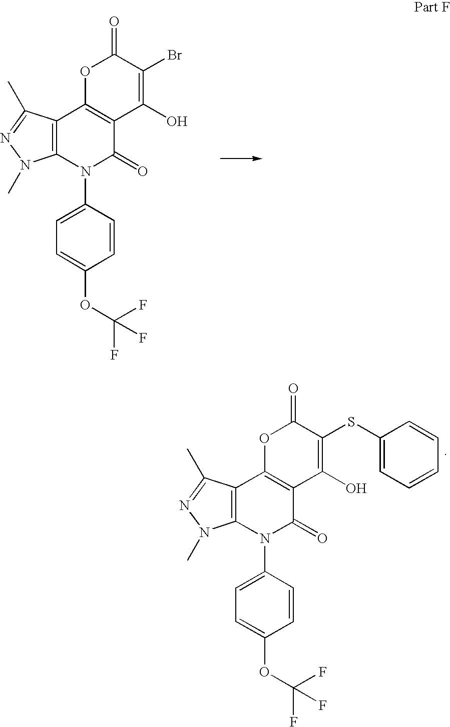

The standard thiol alkylation procedure described in Example 2 was used to convert 3-bromo-4-hydroxy-7,9-dimethyl-6-(4-(trifluoromethoxy)phenyl)pyrano[2,3-d]pyrazolo[3,4-b]pyridine-2,5(6H,7H)-dione to the title compound, m.p. 136-139° C. ¹H NMR (300 MHz, DMSO-d₆): δ 7.80 (2H, d, J=9 Hz), 7.66 (2H, d, J=9 Hz), 7.40-7.10 (5H, m), 3.08 (3H, s), 2.52 (3H, s). ¹⁹F NMR (282 MHz, DMSO-d₆): δ −57.26 (3F, s). MS (ES+): m/e 516.8 (25), 515.8 (100). MS (ES−): m/e 514.7 (25), 513.7 (100).

The following compounds were also prepared in a manner similar to that of Example 6.2.7.

Compound 792: m.p. 217-219° C. ¹H NMR (300 MHz, DMSO-d₆): δ 7.81 (2H, d, J=9.0 Hz), 7.67 (2H, d, J=9.0 Hz), 7.29 (1H, t, J=8.0 Hz), 7.21-7.14 (3H, m), 3.09 (3H, s), 2.52 (3H, s). ¹⁹F NMR (282 MHz, DMSO-d₆): δ −57.26 (3F, s). MS (ES+): m/e 551.7 (50), 549.7 (100). MS (ES−): m/e 549.6 (30), 547.5 (100).

Compound 793: m.p. 230-231° C. ¹H NMR (300 MHz, DMSO-d₆): δ 7.81 (2H, d, J=9.0 Hz), 7.67 (2H, d, J=9.0 Hz), 7.32 (2H, d, J=8.8 Hz), 7.20 (2H, d, J=8.8 Hz), 3.08 (3H, s), 2.51 (3H, s). ¹⁹F NMR (282 MHz, DMSO-d₆): δ −57.26 (3F, s). MS (ES+): m/e 551.7 (45), 549.8 (100). MS (ES−): m/e 549.7 (40), 547.5 (100).

Compound 794: m.p. 154-156° C. ¹H NMR (300 MHz, DMSO-d₆): δ 7.81 (2H, d, J=9.0 Hz), 7.67 (2H, d, J=9.0 Hz), 7.18 (1H, t, J=8.5 Hz), 6.75-6.69 (3H, m), 3.70 (3H, s), 3.08 (3H, s), 2.52 (3H, s). ¹⁹F NMR (282 MHz, DMSO-d₆): δ −57.26 (3F, s). MS (ES+): m/e 546.8 (25), 545.9 (100). MS (ES−): m/e 544.7 (20), 543.6 (100).

Compound 795: MS (ES+): m/e 558.27 (100). MS (ES−): m/e 556.23 (100).

Compound 796: m.p. 221-222° C. ¹H NMR (300 MHz, DMSO-d₆): δ 7.81 (2H, d, J=9.0 Hz), 7.67 (2H, d, J=9.0 Hz), 7.36-7.25 (1H, m), 7.05-6.90 (3H, m), 3.09 (3H, s), 2.52 (3H, s). ¹⁹F NMR (282 MHz, DMSO-d₆): δ −57.26 (3F, s), −112.76 (1F, m). MS (ES+): m/e 534.9 (25), 533.9 (100). MS (ES−): m/e 532.8 (25), 531.7 (100).

Compound 797: m.p. 219-220° C. ¹H NMR (300 MHz, DMSO-d₆): δ 7.80 (2H, d, J=8.8 Hz), 7.67 (2H, d, J=8.8 Hz), 7.30-7.10 (4H, m), 3.08 (3H, s), 2.51 (3H, s). ¹⁹F NMR (282 MHz, DMSO-d₆): δ −57.26 (3F, s), −117.56 (1F, s). MS (ES+): m/e 534.9 (25), 533.9 (100). MS (ES−): m/e 532.7 (25), 531.7 (100).

Compound 798: m.p. 229-231° C. ¹H NMR (300 MHz, DMSO-d₆): δ 7.81 (2H, d, J=8.8 Hz), 7.67 (2H, d, J=8.8 Hz), 7.51-7.48 (4H, m), 3.09 (3H, s), 2.52 (3H, s). ¹⁹F NMR (282 MHz, DMSO-d₆): δ −57.27 (3F, s), −61.60 (3F, s). MS (ES+): m/e 584.8 (25), 583.8 (100). MS (ES−): m/e 582.7 (25), 581.7 (100).

Compound 832: m.p. 187-189° C. ¹H NMR (300 MHz, DMSO-d₆): δ 7.53 (2H, d, J=8.8 Hz), 7.32-7.26 (1H, m), 7.17 (2H, d, J=8.8 Hz), 7.04-6.94 (3H, m), 3.84 (3H, s), 3.09 (3H, s), 2.51 (3H, s). ¹⁹F NMR (282 MHz, DMSO-d₆): δ −112.78 (1F, m). MS (ES+): m/e 481.53 (20), 480.17 (100). MS (ES−): m/e 479.41 (20), 478.20 (100).

Compound 833: m.p. 183-184° C. ¹H NMR (300 MHz, DMSO-d₆): δ 7.53 (2H, d, J=8.8 Hz), 7.29-7.14 (6H, m), 3.84 (3H, s), 3.09 (3H, s), 2.51 (3H, s). MS (ES+): m/e 497.90 (50), 496.12 (100). MS (ES−): m/e 496.01 (50), 494.17 (100).

Compound 834: m.p. 198-199° C. ¹H NMR (300 MHz, DMSO-d₆): δ 7.52 (2H, d, J=8.8 Hz), 7.28-7.09 (6H, m), 3.84 (3H, s), 3.08 (3H, s), 2.50 (3H, s). ¹⁹F NMR (282 MHz, DMSO-d₆): δ −117.65 (1F, m). MS (ES+): m/e 481.53 (20), 480.14 (100). MS (ES−): m/e 479.48 (20), 478.19 (100).

Compound 835: m.p. 203-204° C. ¹H NMR (300 MHz, DMSO-d₆): δ 7.55-7.48 (6H, m), 7.17 (2H, d, J=9.0 Hz), 3.84 (3H, s), 3.09 (3H, s), 2.51 (3H, s). ¹⁹F NMR (282 MHz, DMSO-d₆): δ −61.58 (3F, s). MS (ES+): m/e 531.56 (20), 530.19 (100). MS (ES−): m/e 529.41 (20), 528.19 (100).

Compound 836: m.p. 157-158° C. ¹H NMR (300 MHz, DMSO-d₆): δ 7.53 (2H, d, J=8.8 Hz), 7.21-7.14 (3H, m), 6.74-6.69 (3H, m), 3.84 (3H, s), 3.70 (3H, s), 3.08 (3H, s), 2.51 (3H, s). MS (ES+): m/e 493.58 (20), 492.18 (100). MS (ES−): m/e 491.43 (20), 490.24 (100).

Compound 837: m.p. 154-155° C. ¹H NMR (300 MHz, DMSO-d₆): δ 12.84 (1H, s), 7.58-7.50 (1H, m), 7.34-7.16 (4H, m), 7.05-6.91 (3H, m), 3.80 (3H, s), 3.11 (3H, s), 2.51 (3H, s). ¹⁹F NMR (282 MHz, DMSO-d₆): δ −112.77 (1F, m). MS (ES+): m/e 481.55 (20), 480.15 (100). MS (ES−): m/e 479.40 (20), 478.18 (100).

Compound 838: m.p. 144-146° C. ¹H NMR (300 MHz, DMSO-d₆): δ 7.55 (1H, t, J=8.2 Hz), 7.32-7.14 (7H, m), 3.80 (3H, s), 3.11 (3H, s), 2.52 (3H, s). MS (ES+): m/e 497.93 (45), 496.12 (100). MS (ES−): m/e 496.15 (45), 494.14 (100).

Compound 839: m.p. 259-261° C. ¹H NMR (300 MHz, DMSO-d₆): δ 7.55 (1H, t, J=8.0 Hz), 7.26-7.08 (7H, m), 3.79 (3H, s), 3.10 (3H, s), 2.50 (3H, s). ¹⁹F NMR (282 MHz, DMSO-d₆): δ −117.61 (1F, m). MS (ES+): m/e 481.54 (20), 480.18 (100), 479.41 (20), 478.21 (100).

Compound 840: m.p. 191-192° C. ¹H NMR (300 MHz, DMSO-d₆): δ 7.58-7.47 (5H, m), 7.28-7.17 (3H, m), 3.80 (3H, s), 3.11 (3H, s), 2.52 (3H, s). ¹⁹F NMR (282 MHz, DMSO-d₆): δ −61.58 (3F, s). MS (ES+): m/e 531.53 (20), 530.19 (100). MS (ES−): m/e 529.48 (20), 528.21 (100).

Compound 841: m.p. 194-197° C. ¹H NMR (300 MHz, DMSO-d₆): δ 7.60-7.50 (2H, m), 7.30-7.15 (4H, m), 6.79-6.69 (2H, m), 3.79 (3H, s), 3.70 (3H, s), 3.07 (3H, s), 2.48 (3H, s). MS (ES+): m/e 493.54 (20), 492.17 (100). MS (ES−): m/e 491.41 (20), 490.20 (100).

Compound 933: m.p. 213-216° C. ¹H NMR (300 MHz, DMSO-d₆): δ 10.12 (1H, s), 7.43 (1H, t, J=7.9 Hz), 7.34-7.27 (1H, m), 7.05-6.91 (6H, m), 5.74 (1H, s), 3.12 (3H, s), 2.51 (3H, s). ¹⁹F NMR (282 MHz, DMSO-d₆): δ −112.77 (1F, m). MS (ES+): m/e 467.54 (20), 466.13 (100). MS (ES−): m/e 465.41 (20), 464.18 (100).

Compound 934: m.p. 151-153° C. ¹H NMR (300 MHz, DMSO-d₆): δ 10.14 (1H, s), 7.37 (2H, d, J=8.8 Hz), 7.34-7.26 (1H, m), 7.04-6.90 (5H, m), 3.10 (3H, s), 2.50 (3H, s). ¹⁹F NMR (282 MHz, DMSO-d₆): δ −112.77 (1F, m). MS (ES+): m/e 467.55 (20), 466.16 (100). MS (ES−): m/e 465.45 (20), 464.21 (100).

### 6.2.8 Synthesis of 4-Hydroxy-6-phenyl-3-(phenylthio)indeno[1,2-b]pyrano[2,3-d]pyridine-2,5(6H,11H)-dione (Compound 354)

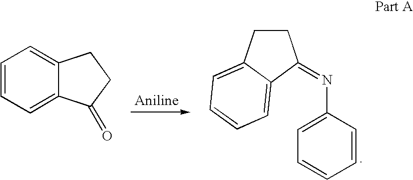

To a solution of 1-indanone (2.64 g, 20.0 mmol) and aniline (1.90 mL, 20.8 mmol) in 20 mL dichloroethane was added 20 g 4 Å molecular sieves, and the mixture was stirred at ambient temperature for 42 hours. The sieves were removes by filtration through celite, and the filtrate was evaporated to afford the imine product, indan-1-ylidene-phenyl-amine, as a solid (1.19 g, 5.72 mmol), m.p. 109-112° C. TLC R_{F} 0.37 (20:80 ethyl acetate-hexane). ¹H NMR (300 MHz, CDCl₃): δ 8.07 (1H, br d, J=7.0 Hz), 7.50-7.34 (5H, m), 7.16-7.10 (1H, m), 6.98 (2H, d, J=7.3 Hz), 3.10-3.06 (2H, m), 2.74-2.69 (2H, m). MS (ES+): m/e 209.31 (25), 208.27 (100).

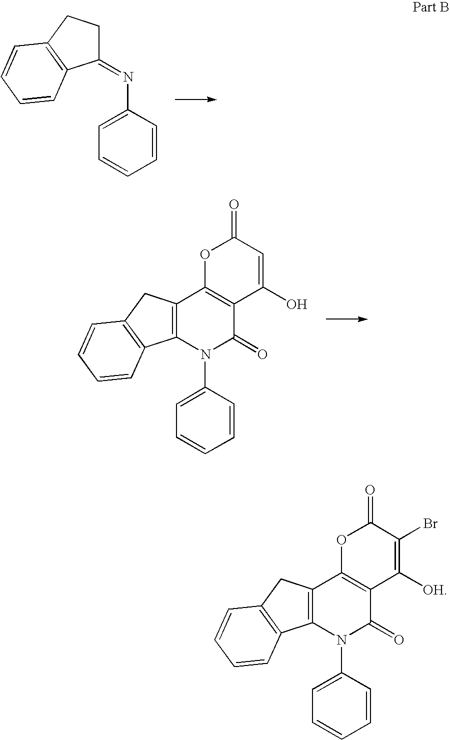

The standard high-temperature condensation reaction was used to convert indan-1-ylidene-phenyl-amine (1.19 g, 5.72 mmol) to 4-hydroxy-6-phenylindeno[1,2-b]pyrano[2,3-d]pyridine-2,5(6H,11H)-dione (1.51 g, 4.40 mmol, 77%). A portion of this sample (834 mg, 2.43 mmol) and N-bromosuccinimide (432 mg, 2.43 mmol) were suspended in 5 mL acetonitrile, and heated to reflux overnight. The mixture was cooled, and the solid product (3-bromo-4-hydroxy-6-phenylindeno[1,2-b]pyrano[2,3-d]pyridine-2,5(6H, 1H)-dione) was obtained by filtration, washing and vacuum drying (820 mg, 1.94 mmol, 80%), m.p.>300° C. ¹H NMR (300 MHz, DMSO-d₆): δ 7.74-7.69 (4H, m), 7.62-7.57 (2H, m), 7.45 (1H, t, J=7.2 Hz), 7.12 (1H, t, J=7.6 Hz), 5.74 (1H, d, J=8.2 Hz), 4.04 (2H, s). MS (ES+): m/e 424 (100), 422 (90). MS (ES−): m/e 422 (100), 420 (95).

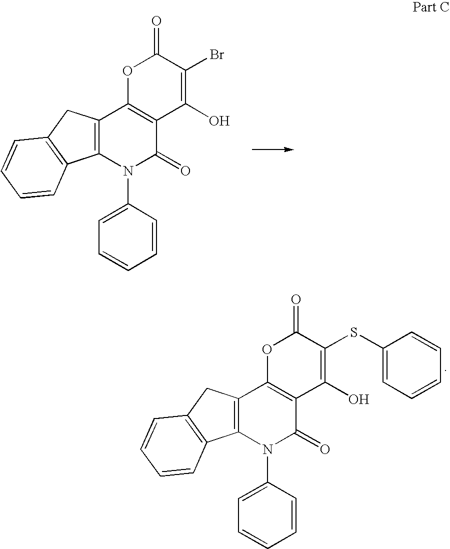

A mixture of 3-bromo-4-hydroxy-6-phenylindeno[1,2-b]pyrano[2,3-d]pyridine-2,5(6H,11H)-dione (147 mg, 0.349 mmol), thiophenol (148 μL), and potassium carbonate (96 mg) in 3 mL dimethylformamide was heated to 70° C. overnight. The mixture was cooled and partitioned between dilute HCl and diethyl ether. This two-phase mixture containing a precipitate was filtered, and the solid was washed with additional ether and dried under high vacuum to afford the title product as a yellow powder (90 mg, 0.199 mmol, 57%), m.p. 274-275° C. ¹H NMR (300 MHz, DMSO-d₆): δ 7.75-7.70 (4H, m), 7.63-7.58 (2H, m), 7.46 (1H, t, J=7.6 Hz), 7.30-7.10 (6H, m), 5.75 (1H, d, J=7.9 Hz), 4.05 (2H, s). MS (ES+): m/e 453.64 (20), 452.49 (100).

Using this procedure and appropriate substrates, the following compounds were prepared:

Compound 355: m.p. 235-236° C. ¹H NMR (300 MHz, DMSO-d₆): δ 7.76-7.69 (4H, m), 7.64-7.58 (2H, m), 7.47 (1H, t, J=7.5 Hz), 7.19 (1H, t, J=8.2 Hz), 7.13 (1H, t, J=7.6 Hz), 6.76-6.69 (3H, m), 5.75 (1H, d, J=8.2 Hz), 4.06 (2H, s), 3.70 (3H, s). MS (ES+): m/e 483.69 (20), 482.48 (100). MS (ES−): m/e 481.61 (20), 480.51 (100).

Compound 356: m.p. 244-245° C. ¹H NMR (300 MHz, DMSO-d₆): δ 7.78-7.70 (4H, m), 7.64-7.59 (2H, m), 7.47 (1H, t, J=7.6 Hz), 7.32 (1H, q, J=7.3 Hz), 7.14 (1H, t, J=7.9 Hz), 7.07-6.92 (3H, m), 5.76 (1H, d, J=8.2 Hz), 4.07 (2H, s). ¹⁹F NMR (282 MHz, DMSO-d₆): δ −112.76 (1F, m). MS (ES+): m/e 471.70 (25), 470.48 (100). MS (ES−): m/e 468.51 (100).

Compound 357: m.p. 212-213° C. ¹H NMR (300 MHz, DMSO-d₆): δ 7.50-7.34 (6H, m), 7.29-7.11 (6H, m), 6.86 (1H, t, J=7.8 Hz), 6.71 (1H, d, J=8.2 Hz), 2.95-2.85 (2H, m), 2.83-2.73 (2H, m). MS (ES+): m/e 467.70 (25), 466.49 (100). MS (ES−): m/e 465.68 (20), 464.54 (100).

Compound 358: m.p. 225-226° C. ¹H NMR (300 MHz, DMSO-d₆): δ 7.49-7.17 (11H, m), 6.87 (1H, t, J=7.6 Hz), 6.71 (1H, d, J=7.9 Hz), 2.96-2.83 (2H, m), 2.82-2.70 (2H, m). MS (ES+): m/e 502.38 (40), 500.47 (100). MS (ES−): m/e 500.49 (35), 498.47 (100).

Compound 359: m.p. 248-249° C. ¹H NMR (300 MHz, DMSO-d₆): δ 7.50-7.37 (6H, m), 7.25-7.13 (2H, m), 6.86 (1H, t, J=7.9 Hz), 6.79-6.69 (4H, m), 3.70 (3H, s), 2.94-2.84 (2H, m), 2.82-2.72 (2H, m). MS (ES+): m/e 497.71 (25), 496.54 (100). MS (ES−): m/e 495.68 (20), 494.50 (100).

Compound 360: m.p. 253-254° C. ¹H NMR (300 MHz, DMSO-d₆): δ 7.51-7.20 (8H, m), 7.06-6.84 (4H, m), 6.71 (1H, d, J=7.9 Hz), 2.95-2.85 (2H, m), 2.83-2.73 (2H, m). ¹⁹F NMR (282 MHz, DMSO-d₆): δ −112.75 (1F, m). MS (ES+): m/e 485.68 (25), 484.51 (100). MS (ES−): m/e 483.69 (25), 482.52 (100).

Compound 361: m.p. 163-165° C. ¹H NMR (300 MHz, DMSO-d₆): δ 7.50-7.18 (11H, m), 6.86 (1H, t, J=7.0 Hz), 6.71 (1H, d, J=7.6 Hz), 2.96-2.86 (2H, m), 2.84-2.74 (2H, m). MS (ES+): m/e 502.39 (45), 500.48 (100). MS (ES−): m/e 500.52 (45), 498.46 (100).

Compound 362: m.p. 220-221° C. ¹H NMR (300 MHz, DMSO-d₆): δ 7.49-7.07 (11H, m), 6.86 (1H, t, J=7.5 Hz), 6.70 (1H, d, J=7.9 Hz), 2.90-2.81 (2H, m), 2.80-2.70 (2H, m). ¹⁹F NMR (282 MHz, DMSO-d₆): δ −117.54 (1F, m). MS (ES+): m/e 485.67 (20), 484.51 (100). MS (ES−): m/e 483.59 (25), 482.49 (100).

Compound 363: m.p. 184-185° C. ¹H NMR (300 MHz, DMSO-d₆): δ 7.55-7.35 (10H, m), 7.22 (1H, t, J=7.2 Hz), 6.87 (1H, t, J=7.6 Hz), 6.71 (1H, d, J=7.9 Hz), 2.95-2.86 (2H, m), 2.84-2.75 (2H, m). ¹⁹F NMR (282 MHz, DMSO-d₆): δ −61.54 (3F, s). MS (ES+): m/e 535.71 (25), 534.54 (100). MS (ES−): m/e 533.65 (30), 532.50 (100).

Compound 364: m.p. 128-129° C. ¹H NMR (300 MHz, DMSO-d₆): δ 7.49-7.34 (7H, m), 7.22 (2H, d, J=8.8 Hz), 6.90-6.80 (3H, m), 6.69 (1H, d, J=7.9 Hz), 3.70 (3H, s), 2.94-2.83 (2H, m), 2.81-2.72 (2H, m). MS (ES−): m/e 495.68 (25), 494.49 (100).

Compound 505: m.p. 243-244° C. ¹H NMR (300 MHz, DMSO-d₆): δ 7.48-7.32 (5H, m), 7.30-7.10 (5H, m), 6.93 (1H, d, J=7.6 Hz), 6.83 (1H, t, J=8.2 Hz), 6.35 (1H, d, J=7.6 Hz), 3.79 (3H, s), 2.90-2.80 (2H, m), 2.77-2.67 (2H, m). MS (ES+): m/e 497.8 (40), 496.7 (100). MS (ES−): m/e 495.5 (35), 494.6 (100).

Compound 506: m.p. 249-251° C. ¹H NMR (300 MHz, DMSO-d₆): δ 7.48-7.15 (9H, m), 6.94 (1H, d, J=8.2 Hz), 6.83 (1H, t, J=8.2 Hz), 6.35 (1H, d, J=8.2 Hz), 3.80 (3H, s), 2.89-2.84 (2H, m), 2.74-2.69 (2H, m). MS (ES+): m/e 530.6 (100). MS (ES−): m/e 528.3 (100).

Compound 507: m.p. 216-217° C. ¹H NMR (300 MHz, DMSO-d₆): δ 7.46-7.35 (6H, m), 7.20-7.14 (1H, m), 6.93 (1H, d, J=7.9 Hz), 6.83 (1H, t, J=8.2 Hz), 6.76-6.69 (2H, m), 6.35 (1H, d, J=7.3 Hz), 3.79 (3H, s), 3.70 (3H, s), 2.86-2.80 (2H, m), 2.76-2.60 (2H, m). MS (ES+): m/e 526.7 (100). MS (ES−): m/e 524.4 (100).

Compound 508: m.p. 244-246° C. ¹H NMR (300 MHz, DMSO-d₆): δ 7.50-7.23 (6H, m), 7.07-6.80 (5H, m), 6.35 (1H, d, J=7.9 Hz), 3.80 (3H, s), 2.90-2.80 (2H, m), 2.77-2.67 (2H, m). ¹⁹F NMR (282 MHz, DMSO-d₆): δ −112.76 (1F, m). MS (ES+): m/e 514.6 (100). MS (ES−): m/e 512.4 (100).

Compound 509: m.p. 251-253° C. ¹H NMR (300 MHz, DMSO-d₆): δ 7.54-7.09 (10H, m), 6.95 (1H, d, J=2.6 Hz), 6.62 (1H, d, J=9.0 Hz), 6.42 (1H, dd, J=9.0, 2.6 Hz), 3.71 (3H, s), 2.91-2.81 (2H, m), 2.79-2.69 (2H, m). MS (ES+): m/e 496.7 (100). MS (ES−): m/e 494.5 (100).

Compound 510: m.p. 219-220° C. ¹H NMR (300 MHz, DMSO-d₆): δ 7.53-7.16 (9H, m), 6.95 (1H, d, J=2.6 Hz), 6.62 (1H, d, J=9.0 Hz), 6.43 (1H, dd, J=9.0, 2.6 Hz), 3.71 (3H, s), 2.92-2.82 (2H, m), 2.80-2.70 (2H, m). MS (ES+): m/e 530.7 (100). MS (ES−): m/e 528.5 (100).

Compound 511: m.p. 222-223° C. ¹H NMR (300 MHz, DMSO-d₆): δ 7.52-7.39 (5H, m), 7.17 (1H, dd, J=8.8, 7.6 Hz), 6.95 (1H, d, J=2.6 Hz), 6.77-6.65 (3H, m), 6.61 (1H, d, J=9.0 Hz), 6.42 (1H, dd, J=9.0, 2.6 Hz), 3.71 (3H, s), 3.70 (3H, s), 2.91-2.80 (2H, m), 2.79-2.69 (2H, m). MS (ES+): m/e 526.8 (100). MS (ES−): m/e 524.6 (100).

Compound 512: m.p. 231-233° C. ¹H NMR (300 MHz, DMSO-d₆): δ 7.54-7.25 (6H, m), 7.05-6.91 (4H, m), 6.62 (1H, d, J=9.0 Hz), 6.43 (1H, dd, J=9.0, 2.6 Hz), 3.71 (3H, s), 2.92-2.81 (2H, m), 2.79-2.69 (2H, m). ¹⁹F NMR (282 MHz, DMSO-d₆): δ −112.78 (1F, m). MS (ES+): m/e 514.7 (100). MS (ES−): m/e 512.7 (100).

Compound 513: m.p. 236-237° C. ¹H NMR (300 MHz, DMSO-d₆): δ 7.58-7.41 (5H, m), 7.31-7.18 (5H, m), 6.81 (1H, dd, J=8.5, 2.6 Hz), 6.30 (1H, d, J=2.4 Hz), 3.26 (3H, s), 2.85-2.71 (4H, m). MS (ES−): m/e 528.4 (100).

Compound 514: m.p. 185-187° C. ¹H NMR (300 MHz, DMSO-d₆): δ 7.57-7.40 (5H, m), 7.30-7.16 (2H, m), 6.82-6.69 (4H, m), 6.72 (1H, d, J=1.2 Hz), 3.70 (3H, s), 3.31 (3H, s), 2.86-2.68 (4H, m). MS (ES−): m/e 524.4 (100).

Compound 515: m.p. 216-218° C. ¹H NMR (300 MHz, DMSO-d₆): δ 7.54-7.38 (5H, m), 7.36-7.21 (2H, m), 7.02-6.90 (3H, m), 6.83-6.76 (1H, m), 6.30-6.25 (1H, m), 3.26 (3H, s), 2.83-2.65 (4H, m). ¹⁹F NMR (282 MHz, DMSO-d₆): δ −112.99 (1F, m). MS (ES−): m/e 512.4 (100).

Compound 516: m.p. 215-217° C. ¹H NMR (300 MHz, DMSO-d₆): δ 7.50-7.10 (10H, m), 6.93 (1H, s), 6.42 (1H, s), 2.81-2.62 (4H, m), 2.26 (3H, s), 1.81 (3H, s). MS (ES+): m/e 494.7 (100). MS (ES−): m/e 492.4 (100).

Compound 517: m.p. 255-257° C. ¹H NMR (300 MHz, DMSO-d₆): δ 7.50-7.19 (9H, m), 6.93 (1H, d, J=0.4 Hz), 6.42 (1H, d, J=0.4 Hz), 2.81-2.65 (4H, m), 2.26 (3H, s), 1.81 (3H, s). MS (ES+): m/e 530.7 (30), 528.7 (100). MS (ES−): m/e 526.5 (100).

Compound 518: m.p. 201-203° C. ¹H NMR (300 MHz, DMSO-d₆): δ 7.52-7.42 (3H, m), 7.40-7.32 (2H, m), 7.18 (1H, t, J=9.0 Hz), 6.93 (1H, s), 6.78-6.66 (3H, m), 6.42 (1H, s), 3.70 (3H, s), 2.80-2.63 (4H, m), 2.26 (3H, s), 1.81 (3H, s). MS (ES+): m/e 525.8 (30), 524.6 (100). MS (ES−): m/e 522.5 (100).

Compound 519: m.p. 257-259° C. ¹H NMR (300 MHz, DMSO-d₆): δ 7.50-7.40 (3H, m), 7.39-7.24 (3H, m), 7.06-6.97 (3H, m), 6.94 (1H, s), 6.42 (1H, s), 2.81-2.63 (4H, m), 2.26 (3H, s), 1.81 (3H, s). ¹⁹F NMR (282 MHz, DMSO-d₆): δ −112.76 (1F, m). MS (ES+): m/e 513.7 (35), 512.7 (100). MS (ES−): m/e 510.5 (100).

Compound 520: m.p. 155-158° C. ¹H NMR (300 MHz, DMSO-d₆): δ 13.40 (1H, s), 7.77 (1H, t, J=7 Hz), 7.48 (1H, q, J=7 Hz), 7.32-7.10 (9H, m), 6.98-6.65 (3H, m), 3.08-2.93 (1H, m), 2.90-2.63 (2H, m), 2.20-1.97 (2H, m), 1.70-1.54 (1H, m). MS (ES+): m/e 480.6 (100). MS (ES−): m/e 478.5 (100).

Compound 521: m.p. 183-184° C. ¹H NMR (300 MHz, DMSO-d₆): δ 13.40 (1H, s), 7.79 (1H, d, J=7.9 Hz), 7.50 (1H, t, J=7.3 Hz), 7.33-7.13 (7H, m), 6.95 (1H, t, J=7.6 Hz), 6.85 (1H, d, J=7.3 Hz), 6.73 (1H, d, J=7.9 Hz), 3.09-2.97 (1H, m), 2.85-2.63 (2H, m), 2.22-1.99 (2H, m), 1.70-1.55 (1H, m). MS (ES+): m/e 514.6 (100). MS (ES−): m/e 512.4 (100).

Compound 522: m.p. 216-219° C. ¹H NMR (300 MHz, DMSO-d₆): δ 13.40 (1H, s), 7.78 (1H, t, J=7.1 Hz), 7.53-7.45 (1H, m), 7.32-7.11 (5H, m), 6.97-6.70 (6H, m), 3.71 (3H, s), 3.08-2.97 (1H, m), 2.88-2.62 (2H, m), 2.20-1.98 (2H, m), 1.69-1.55 (1H, m). MS (ES+): m/e 511.6 (30), 510.6 (100). MS (ES−): m/e 508.5 (100).

Compound 523: m.p. 227-228° C. ¹H NMR (300 MHz, DMSO-d₆): δ 7.79 (1H, d, J=7.9 Hz), 7.50 (1H, t, J=7.2 Hz), 7.35-6.90 (9H, m), 6.85 (1H, d, J=7.6 Hz), 6.72 (1H, d, J=7.6 Hz), 3.09-2.97 (1H, m), 2.89-2.62 (2H, m), 2.23-1.98 (2H, m), 1.71-1.56 (1H, m). ¹⁹F NMR (282 MHz, DMSO-d₆): δ −112.73 (1F, m). MS (ES+): m/e 499.6 (25), 498.6 (100). MS (ES−): m/e 497.4 (30), 496.5 (100).

Compound 682: m.p. 201-202° C. ¹H NMR (300 MHz, DMSO-d₆): δ 7.40-7.06 (9H, m), 6.98 (2H, d, J=9.0 Hz), 6.91 (1H, t, J=8.5 Hz), 6.76 (1H, d, J=7.6 Hz), 3.76 (3H, s), 2.90-2.82 (2H, m), 2.79-2.71 (2H, m). MS (ES+): m/e 496.31 (100). MS (ES−): m/e 494.32 (100).

Compound 683: m.p. 199-200° C. ¹H NMR (300 MHz, DMSO-d₆): δ 7.40-7.16 (8H, m), 7.00 (2H, d, J=9.1 Hz), 6.93 (1H, td, J=8.2, 1.2 Hz), 6.78 (1H, d, J=7.6 Hz), 3.77 (3H, s), 2.93-2.83 (2H, m), 2.79-2.69 (2H, m). MS (ES+): m/e 532.13 (45), 530.29 (100). MS (ES−): m/e 530.16 (45), 528.24 (100).

Compound 684: m.p. 213-214° C. ¹H NMR (300 MHz, DMSO-d₆): δ 7.35 (1H, d, J=7 Hz), 7.30 (2H, d, J=8.8 Hz), 7.26-7.14 (2H, m), 7.00 (2H, d, J=8.8 Hz), 6.92 (1H, td, J=8.3, 1.5 Hz), 6.79-6.69 (4H, m), 3.77 (3H, s), 3.70 (3H, s), 2.92-2.82 (2H, m), 2.80-2.70 (2H, m). MS (ES+): m/e 527.65 (25), 526.36 (100). MS (ES−): m/e 525.44 (35), 524.25 (100).

Compound 685: m.p. 230-231° C. ¹H NMR (300 MHz, DMSO-d₆): δ 7.34-7.20 (5H, m), 7.05-6.90 (6H, m), 6.78 (1H, d, J=7.6 Hz), 3.77 (3H, s), 2.94-2.84 (2H, m), 2.80-2.70 (2H, m). ¹⁹F NMR (282 MHz, DMSO-d₆): δ −112.77 (1F, m). MS (ES+): m/e 515.60 (20), 514.32 (100). MS (ES−): m/e 513.45 (25), 512.35 (100).

Compound 686: m.p. 169-170° C. ¹H NMR (300 MHz, DMSO-d₆): δ 7.34 (1H, d, J=8 Hz), 7.29 (2H, d, J=8.8 Hz), 7.24-7.19 (1H, m), 7.21 (2H, d, J=9.0 Hz), 6.99 (2H, d, J=9.0 Hz), 6.92 (1H, td, J=8.2, 1.2 Hz), 6.85 (2H, d, J=9.0 Hz), 6.76 (1H, d, J=7.3 Hz), 3.77 (3H, s), 3.69 (3H, s), 2.90-2.80 (2H, m), 2.78-2.68 (2H, m). MS (ES−): m/e 524.32 (100).

Compound 687: m.p. 189-190° C. ¹H NMR (300 MHz, DMSO-d₆): δ 7.39-7.07 (8H, m), 7.00 (2H, d, J=9.0 Hz), 6.92 (1H, t, J=7.7 Hz), 6.77 (1H, d, J=8.2 Hz), 3.77 (3H, s), 2.92-2.82 (2H, m), 2.79-2.69 (2H, m). ¹⁹F NMR (282 MHz, DMSO-d₆): δ −117.58 (1F, m). MS (ES+): m/e 515.62 (20), 514.33 (100). MS (ES−): m/e 513.68 (30), 512.49 (100).

Compound 688: m.p. 186-187° C. ¹H NMR (300 MHz, DMSO-d₆): δ 7.53 (1H, br s), 7.50-7.47 (3H, m), 7.36 (1H, d, J=6.7 Hz), 7.30 (2H, d, J=9.0 Hz), 7.23 (1H, td, J=6.9, 0.9 Hz), 7.00 (2H, d, J=9.0 Hz), 6.93 (1H, t, J=7.9 Hz), 6.78 (1H, d, J=7.6 Hz), 3.77 (3H, s), 2.93-2.83 (2H, m), 2.80-2.70 (2H, m). ¹⁹F NMR (282 MHz, DMSO-d₆): δ −61.56 (3F, s). MS (ES+): m/e 565.63 (25), 564.33 (100). MS (ES−): m/e 562.36 (100).

Compound 689: m.p. 223-224° C. ¹H NMR (300 MHz, DMSO-d₆): δ 7.52-7.09 (11H, m), 6.94 (1H, td, J=7.8, 1.2 Hz), 6.71 (1H, d, J=7.9 Hz), 2.93-2.83 (2H, m), 2.81-2.71 (2H, m). ¹⁹F NMR (282 MHz, DMSO-d₆): δ −112.37 (1F, m). MS (ES+): m/e 485.56 (25), 484.28 (100). MS (ES−): m/e 483.54 (25), 482.26 (100).

Compound 690: m.p. 181-182° C. ¹H NMR (300 MHz, DMSO-d₆): δ 7.53-7.07 (10H, m), 6.94 (1H, t, J=7.2 Hz), 6.71 (1H, d, J=7.9 Hz), 2.96-2.86 (2H, m), 2.84-2.74 (2H, m). ¹⁹F NMR (282 MHz, DMSO-d₆): δ −112.33 (1F, m). MS (ES+): m/e 520.03 (50), 518.26 (100). MS (ES−): m/e 518.18 (45), 516.26 (100).

Compound 691: m.p. 166-167° C. ¹H NMR (300 MHz, DMSO-d₆): δ 7.50-7.45 (2H, m), 7.39-7.14 (5H, m), 6.94 (1H, td, J=7.0, 1.2 Hz), 6.76-6.69 (4H, m), 3.70 (3H, s), 2.92-2.82 (2H, m), 2.80-2.70 (2H, m). ¹⁹F NMR (282 MHz, DMSO-d₆): δ −112.37 (1F, m). MS (ES+): m/e 515.62 (25), 514.28 (100). MS (ES−): m/e 513.44 (25), 512.27 (100).

Compound 692: m.p. 198-199° C. ¹H NMR (300 MHz, DMSO-d₆): δ 7.50-7.21 (7H, m), 7.06-6.92 (4H, m), 6.72 (1H, d, J=8.2 Hz), 2.94-2.84 (2H, m), 2.81-2.71 (2H, m). ¹⁹F NMR (282 MHz, DMSO-d₆): δ −112.35 (1F, m), −112.75 (1F, m). MS (ES+): m/e 503.56 (25), 502.24 (100). MS (ES−): m/e 501.43 (25), 500.26 (100).

Compound 693: m.p. 175-176° C. ¹H NMR (300 MHz, DMSO-d₆): δ 7.46 (2H, dd, J=9.0, 5.0 Hz), 7.38-7.20 (6H, m), 6.93 (1H, t, J=7.9 Hz), 6.85 (2H, d, J=9.0 Hz), 6.69 (1H, d, J=8.2 Hz), 3.70 (3H, s), 2.92-2.82 (2H, m), 2.79-2.69 (2H, m). ¹⁹F NMR (282 MHz, DMSO-d₆): δ −112.43 (1F, m). MS (ES−): m/e 512.31 (100).

Compound 694: m.p. 234-236° C. ¹H NMR (300 MHz, DMSO-d₆): δ 7.47 (2H, dd, J=9.0, 5.0 Hz), 7.38-7.21 (6H, m), 7.12 (2H, t, J=9.0 Hz), 6.94 (1H, t, J=7.2 Hz), 6.70 (1H, d, J=7.6 Hz), 2.93-2.83 (2H, m), 2.80-2.70 (2H, m). ¹⁹F NMR (282 MHz, DMSO-d₆): δ −112.35 (1F, m), −117.50 (1F, m). MS (ES+): m/e 503.55 (25), 502.24 (100). MS (ES−): m/e 501.51 (25), 500.23 (100).

Compound 695: m.p. 197-198° C. ¹H NMR (300 MHz, DMSO-d₆): δ 7.53 (1H, br s), 7.50-7.45 (4H, m), 7.39-7.22 (5H, m), 6.95 (1H, t, J=7.3 Hz), 6.72 (1H, d, J=8.5 Hz), 2.95-2.85 (2H, m), 2.81-2.71 (2H, m). ¹⁹F NMR (282 MHz, DMSO-d₆): δ −61.56 (3F, s), −112.34 (1F, m). MS (ES+): m/e 553.60 (25), 552.31 (100). MS (ES−): m/e 551.46 (25), 550.25 (100).

Compound 799: m.p. 203-204° C. ¹H NMR (300 MHz, DMSO-d₆): δ 7.82 (1H, d, J=8.2 Hz), 7.54 (1H, t, J=7.6 Hz), 7.40-7.18 (7H, m), 7.08 (1H, td, J=8.5, 2.3 Hz), 6.75 (1H, d, J=6.1 Hz), 6.72 (1H, dd, J=9.9, 2.3 Hz), 3.08-3.00 (1H, m), 2.79-2.65 (2H, m), 2.20-2.00 (2H, m), 1.71-1.60 (1H, m). ¹⁹F NMR (282 MHz, DMSO-d₆): δ −117.16 (1F, m). MS (ES+): m/e 533.9 (30), 531.9 (100). MS (ES−): m/e 531.8 (30), 529.7 (100).

Compound 800: m.p. 231-232° C. ¹H NMR (300 MHz, DMSO-d₆): δ 7.82 (1H, d, J=7.6 Hz), 7.54 (1H, t, J=7.5 Hz), 7.40-7.03 (8H, m), 6.75 (1H, d, J=8.5 Hz), 6.71 (1H, dd, J=9.9, 2.5 Hz), 3.07-2.98 (1H, m), 2.80-2.65 (2H, m), 2.20-2.00 (2H, m), 1.75-1.59 (1H, m). ¹⁹F NMR (282 MHz, DMSO-d₆): δ −117.16 (1F, m). MS (ES+): m/e 531.8 (100). MS (ES−): m/e 531.7 (30), 529.6 (100).

Compound 801: m.p. 208-209° C. ¹H NMR (300 MHz, DMSO-d₆): δ 7.82 (1H, d, J=7.6 Hz), 7.53 (1H, t, J=7.6 Hz), 7.38-7.28 (2H, m), 7.24-7.17 (2H, m), 7.07 (1H, td, J=8.8, 2.0 Hz), 6.80-6.67 (5H, m), 3.71 (3H, s), 3.08-2.99 (1H, m), 2.78-2.68 (2H, m), 2.19-1.99 (2H, m), 1.71-1.60 (1H, m). ¹⁹F NMR (282 MHz, DMSO-d₆): δ −117.20 (1F, m). MS (ES+): m/e 528.9 (30), 527.8 (100). MS (ES−): m/e 526.8 (30), 525.8 (100).

Compound 802: m.p. 228-229° C. ¹H NMR (300 MHz, DMSO-d₆): δ 7.81 (1H, d, J=8 Hz), 7.53 (1H, t, J=7.8 Hz), 7.39-7.25 (2H, m), 7.23-7.02 (2H, m), 7.15 (4H, s), 6.79-6.67 (2H, m), 3.07-2.97 (1H, m), 2.82 (1H, hp, J=7.0 Hz), 2.79-2.69 (2H, m), 2.20-2.00 (2H, m), 1.70-1.60 (1H, m), 1.14 (6H, d, J=7.0 Hz). ¹⁹F NMR (282 MHz, DMSO-d₆): δ −117.21 (1F, m). MS (ES+): m/e 541.0 (35), 540.0 (100). MS (ES−): m/e 538.8 (35), 537.8 (100).

Compound 803: m.p. 191-192° C. ¹H NMR (300 MHz, DMSO-d₆): δ 7.82 (1H, d, J=7.9 Hz), 7.54 (1H, t, J=7.7 Hz), 7.40-6.94 (8H, m), 6.80-6.65 (2H, m), 3.10-3.00 (1H, m), 2.80-2.65 (2H, m), 2.20-2.00 (2H, m), 1.73-1.61 (1H, m). ¹⁹F NMR (282 MHz, DMSO-d₆): δ −112.70 (1F, m), −117.15 (1F, m). MS (ES+): m/e 516.9 (30), 515.9 (100). MS (ES−): m/e 514.7 (30), 513.7 (100).

Compound 804: m.p. 251-252° C. ¹H NMR (300 MHz, DMSO-d₆): δ 7.81 (1H, d, J=7.9 Hz), 7.53 (1H, t, J=8 Hz), 7.38-7.02 (8H, m), 6.79-6.67 (2H, m), 3.09-2.98 (1H, m), 2.77-2.66 (2H, m), 2.18-1.98 (2H, m), 1.75-1.62 (1H, m). ¹⁹F NMR (282 MHz, DMSO-d₆): δ −73.84 (1F, m), −117.19 (1F, m). MS (ES+): m/e 517.56 (25), 516.25 (100). MS (ES−): m/e 515.46 (25), 514.24 (100).

Compound 805: m.p. 184-185° C. ¹H NMR (300 MHz, DMSO-d₆): δ 7.82 (1H, d, J=8.5 Hz), 7.56-7.49 (5H, m), 7.39-7.27 (2H, m), 7.20 (1H, t, J=7.8 Hz), 7.08 (1H, td, J=8.8, 2.7 Hz), 6.76 (1H, d, J=7.6 Hz), 6.70 (1H, dd, J=10.0, 2.7 Hz), 3.09-3.00 (1H, m), 2.80-2.70 (2H, m), 2.20-2.00 (2H, m), 1.78-1.60 (1H, m). ¹⁹F NMR (282 MHz, DMSO-d₆): δ −61.55 (3F, s), −117.16 (1F, m). MS (ES+): m/e 566.8 (25), 565.8 (100). MS (ES−): m/e 564.7 (30), 563.7 (100).

Compound 807: m.p. 215-216° C. ¹H NMR (300 MHz, DMSO-d₆): δ 7.82 (1H, d, J=8.2 Hz), 7.55 (1H, t, J=7.6 Hz), 7.40-7.17 (8H, m), 6.93-6.81 (2H, m), 6.64 (1H, d, J=7.6 Hz), 4.62-4.51 (1H, m), 4.50-4.40 (1H, m), 3.23-3.14 (1H, m), 2.30-2.18 (1H, m). MS (ES+): m/e 518.13 (45), 516.32 (100). MS (ES−): m/e 516.28 (40), 514.32 (100).

Compound 808: m.p. 253-255° C. ¹H NMR (300 MHz, DMSO-d₆): δ 7.82 (1H, d, J=7.9 Hz), 7.55 (1H, t, J=7.9 Hz), 7.39-7.16 (8H, m), 6.93-6.81 (2H, m), 6.66-6.60 (1H, m), 4.61-4.49 (1H, m), 4.48-4.40 (1H, m), 3.24-3.16 (1H, m), 2.30-2.20 (1H, m). MS (ES+): m/e 518.10 (45), 516.31 (100). MS (ES−): m/e 516.29 (40), 514.31 (100).

Compound 809: m.p. 182-183° C. ¹H NMR (300 MHz, DMSO-d₆): δ 7.83 (1H, d, J=7.6 Hz), 7.55 (1H, t, J=7.6 Hz), 7.38-7.29 (2H, m), 7.25-7.14 (3H, m), 6.92-6.70 (5H, m), 6.64 (1H, d, J=8.2 Hz), 4.60-4.49 (1H, m), 4.47-4.37 (1H, m), 3.71 (3H, s), 3.23-3.14 (1H, m), 2.30-2.20 (1H, m). MS (ES+): m/e 513.61 (30), 512.37 (100). MS (ES−): m/e 511.62 (30), 510.40 (100).

Compound 810: m.p. 230-231° C. ¹H NMR (300 MHz, DMSO-d₆): δ 7.82 (1H, d, J=7.6 Hz), 7.55 (1H, t, J=7.6 Hz), 7.37-7.11 (9H, m), 6.93-6.81 (2H, m), 6.64 (1H, d, J=7.9 Hz), 4.62-4.50 (1H, m), 4.49-4.38 (1H, m), 3.22-3.12 (1H, m), 2.29-2.17 (1H, m). MS (ES+): m/e 483.59 (25), 482.32 (100). MS (ES−): m/e 481.60 (25), 480.34 (100).

Compound 811: m.p. 241-242° C. ¹H NMR (300 MHz, DMSO-d₆): δ 7.82 (1H, d, J=8.5 Hz), 7.55 (1H, t, J=7.6 Hz), 7.38-7.26 (3H, m), 7.24-7.15 (2H, m), 7.10-7.02 (2H, m), 6.99-6.82 (3H, m), 6.63 (1H, d, J=7.9 Hz), 4.61-4.50 (1H, m), 4.49-4.40 (1H, m), 3.23-3.15 (1H, m), 2.34-2.19 (1H, m). ¹⁹F NMR (282 MHz, DMSO-d₆): δ −112.72 (1F, m). MS (ES+): m/e 501.63 (25), 500.32 (100). MS (ES−): m/e 499.52 (25), 498.40 (100).

Compound 812: m.p. 261-263° C. ¹H NMR (300 MHz, DMSO-d₆): δ 7.82 (1H, d, J=8.2 Hz), 7.55 (1H, t, J=7.5 Hz), 7.38-7.09 (8H, m), 6.92-6.80 (2H, m), 6.63 (1H, d, J=7.6 Hz), 4.60-4.50 (1H, m), 4.49-4.39 (1H, m), 3.21-3.13 (1H, m), 2.30-2.19 (1H, m). ¹⁹F NMR (282 MHz, DMSO-d₆): δ −117.59 (1F, m). MS (ES+): m/e 501.60 (20), 500.34 (100). MS (ES−): m/e 499.46 (25), 498.33 (100).

Compound 813: m.p. 223-224° C. ¹H NMR (300 MHz, DMSO-d₆): δ 7.83 (1H, d, J=7.6 Hz), 7.58-7.48 (5H, m), 7.38-7.30 (2H, m), 7.24-7.16 (2H, m), 6.92-6.82 (2H, m), 6.64 (1H, d, J=7.6 Hz), 4.60-4.50 (1H, m), 4.48-4.40 (1H, m), 3.23-3.17 (1H, m), 2.31-2.20 (1H, m). ¹⁹F NMR (282 MHz, DMSO-d₆): δ −61.53 (3F, s). MS (ES+): m/e 551.62 (25), 550.37 (100). MS (ES−): m/e 549.59 (25), 548.35 (100).

Compound 936: m.p. 271-273° C. ¹H NMR (300 MHz, DMSO-d₆): δ 13.34 (1H, s), 9.89 (1H, s), 7.42 (1H, d, J=7.0 Hz), 7.38-7.11 (9H, m), 6.94 (1H, t, J=7.0 Hz), 6.79 (2H, d, J=8.2 Hz), 2.93-2.82 (2H, m), 2.80-2.69 (2H, m). MS (ES+): m/e 483.52 (25), 482.20 (100). MS (ES−): m/e 481.45 (30), 480.22 (100).

### 7. Equivalents

Those skilled in the art will recognize or be able to ascertain using no more than routine experimentation, many equivalents to the specific embodiments described herein. Such equivalents are intended to be encompassed by the following claims.

All references cited herein are incorporated herein by reference in their entirety and for all purposes to the same extent as if each individual publication or patent or patent application was specifically and individually indicated to be incorporated by reference in its entirety for all purposes.

The present disclosure not to be limited in scope by the specific embodiments described herein. Indeed, various modifications of the embodiments in addition to those described herein will become apparent to those skilled in the art from the foregoing description and accompanying figures. Such modifications are intended to fall within the scope of the appended claims.

LENGTHY TABLES
The patent application contains a lengthy table section. A copy of the table is available in electronic form from the USPTO web site (http://seqdata.uspto.gov/?pageRequest=docDetail&DocID=US20100069380A1). An electronic copy of the table will also be available from the USPTO upon request and payment of the fee set forth in 37 CFR 1.19(b)(3).

1. A compound having the formula: 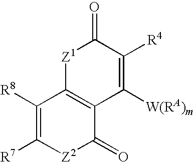
or a pharmaceutically acceptable salt thereof, wherein:
Z¹ and Z² are independently O or NR⁶;
W is O, S or N or a direct bond, wherein m is 1 when W is O, S or a direct bond and m is 2 when W is N;
R^{A} is H, (C₁₋₈)alkyl, substituted or unsubstituted aryl, substituted or unsubstituted C(O)—(C₁₋₈)alkyl, C(O)-amino, or substituted or unsubstituted C(O)-aryl;
R⁴ is H, halo, NO₂, CN, substituted or unsubstituted (C₁₋₈)alkyl, substituted or unsubstituted aryl, substituted or unsubstituted heteroaryl, substituted or unsubstituted heterocycloalkyl, substituted or unsubstituted aryl(C₁₋₈)alkyl, OR⁵, S—C(O)—R⁵ or S(O)ₙ—R⁵, wherein n is 0, 1 or 2;
R⁵ is (C₁₋₈)alkyl, amino, CN, substituted or unsubstituted aryl, substituted or unsubstituted heteroaryl or substituted or unsubstituted aryl(C₁₋₈)alkyl;
R⁶ is independently at each occurrence H, substituted or unsubstituted (C₁₋₈)alkyl, substituted or unsubstituted C₂₋₈alkenyl, substituted or unsubstituted cycloalkyl, substituted or unsubstituted aryl, substituted or unsubstituted heteroaryl, substituted or unsubstituted aryl(C₁₋₈)alkyl, or substituted or unsubstituted heteroaryl(C₁₋₈)alkyl;
R⁷ is H, halo, hydroxyl, (C₁₋₈)alkyl, (C₁₋₈)alkoxy, trihalomethyl, substituted or unsubstituted aryl, or substituted or unsubstituted heteroaryl;
or, when Z² is NR⁶, then R⁶ and R⁷ together with the atoms to which they are attached may form a moiety of the following formula: 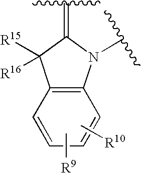
wherein R⁹ and R¹⁰ are independently H, halo, hydroxy, substituted or unsubstituted (C₁₋₈)alkyl, substituted or unsubstituted (C₁₋₈)alkoxy, or trihalomethyl;
wherein R¹⁵ and R¹⁶ are independently (C₁₋₄)alkyl or (C₁₋₂)perfluoroalkyl, or R¹⁵ and R¹⁶ together with the carbon atom to which they are attached form a 3-7 membered cycloalkyl ring, wherein one CH₂ ring member may be optionally replaced by O; and
R⁸ is H, (C₁₋₈)alkyl, halo, hydroxyl, (C₁₋₈)alkoxy, trihalomethyl, S-aryl, substituted or unsubstituted aryl or substituted or unsubstituted heteroaryl; or
R⁷ and R⁸ together with the carbons to which they are attached form a 5, 6 or 7-membered ring optionally containing 1-2 nitrogen atoms, 1-3 double bonds and 1-2 carbonyl groups; and said 5, 6 or 7-membered ring being optionally substituted by one to four substituents selected from halo, hydroxy, (C₁₋₈)alkyl, (C₁₋₈)alkoxy, piperonyl, (C₁₋₈)alkylsulfoxide, or trihalomethyl or said 5, 6 or 7-membered ring being optionally fused to a substituted or unsubstituted phenyl ring; or
R⁶, R⁷ and R⁸ together with the atoms to which they are attached form a 8, 9 or 10-membered bicyclic ring containing 1-3 nitrogen atoms and 1-3 double bonds; and said 8, 9 or 10-membered ring being optionally substituted by one to four substituents selected from halo, hydroxy, (C₁₋₈)alkyl, (C₁₋₈)alkoxy, piperonyl, (C₁₋₈)alkylsulfoxide, or trihalomethyl; or R⁵ and one of R^{A} together with the atoms to which they are attached form a substituted or unsubstituted heteroaryl ring; or
R⁵ and one of R^{A} together with the atoms to which they are attached form a substituted or unsubstituted heteroaryl ring; or
R⁴ and one of R^{A} together with the atoms to which they are attached form a substituted or unsubstituted heteroaryl ring or a substituted or unsubstituted heterocycloalkyl ring;
with the proviso that the compound is not 6-benzyl-4-hydroxy-3-(2,4,5-trichlorophenylsulfonyl)-2H-pyrano[3,2-c]quinoline-2,5(6H)-dione.

2. The compound of claim 1, wherein Z¹ is O and Z² is NR⁶.

3. The compound of claim 1, wherein R⁴ is S—R⁵.

4. The compound of claim 1, wherein W(R_{A})ₘ is OH.

5. The compound of claim 1, wherein R⁷ and R⁸ together with the carbons to which they are attached form a substituted or unsubstituted phenyl ring.

6. The compound of claim 1, wherein R⁶ is unsubstituted aryl(C₁₋₈)alkyl or aryl(C₁₋₈)alkyl substituted with one or more of halo, hydroxy, (C₁₋₈)alkyl, (C₁₋₈)alkoxy, (C₁₋₈)alkylsulfoxide, or trihalomethyl.

7. The compound of claim 6, wherein the substituted or unsubstituted aryl(C₁₋₈)alkyl is substituted or unsubstituted benzyl.

8. The compound of claim 1 wherein R⁵ is unsubstituted aryl or aryl substituted with one or more of halo, hydroxy, (C₁₋₈)alkyl, (C₁₋₈)alkoxy, (C₁₋₈)alkylsulfoxide, or trihalomethyl.

9. The compound of claim 8, wherein the substituted or unsubstituted aryl is substituted or unsubstituted phenyl.

10. The compound of claim 1, wherein:
R⁶ is unsubstituted aryl(C₁₋₈)alkyl or aryl(C₁₋₈)alkyl substituted with one or more of halo, hydroxy, (C₁₋₈)alkyl, (C₁₋₈)alkoxy, (C₁₋₈)alkylsulfoxide, or trihalomethyl; and
R⁵ is unsubstituted aryl or aryl substituted with one or more of halo, hydroxy, (C₁₋₈)alkyl, (C₁₋₈)alkoxy, (C₁₋₈)alkylsulfoxide, or trihalomethyl.

11. A pharmaceutical composition comprising a compound of claim 1 and a pharmaceutically acceptable excipient, carrier or diluent.

12. The pharmaceutical composition of claim 10 suitable for oral, parenteral, mucosal, transdermal or topical administration.

13. The pharmaceutical composition of claim 12, wherein the pharmaceutical composition is suitable for oral administration.

14. A method of preventing or inhibiting replication of a bacterial organism, comprising contacting the microorganism with an effective amount of a compound of claim 1.

15. A method of preventing, treating or managing a bacterial infection, comprising administering to a subject in need thereof an effective amount of a compound of claim 1.

16. A method for identifying a compound that inhibits the activity of a peptidyl tRNA hydrolase enzyme, said method comprising:
(a) contacting one or more compounds with a peptidyl tRNA hydrolase enzyme and a substrate for the enzyme under conditions permitting the cleavage of the substrate by the enzyme; and
(b) measuring the amount of substrate cleaved by the enzyme,
wherein a compound that inhibits peptidyl tRNA hydrolase enzyme activity is identified if the amount of substrate cleaved by the enzyme in the presence of the compound is reduced compared to the amount of substrate cleaved in the absence of the compound.

17. A method for identifying a compound having antibacterial activity, said method comprising:
(a) contacting one or more compounds with a peptidyl tRNA hydrolase enzyme and a substrate for the enzyme under conditions permitting the cleavage of the substrate by the enzyme; and
(b) measuring the amount of substrate cleaved by the enzyme,
wherein a compound that has antibacterial activity is identified if the amount of substrate cleaved by the enzyme in the presence of the compound is reduced compared to the amount of substrate cleaved in the absence of the compound.

18. A method for preventing or inhibiting protein synthesis in a bacterial cell, the method comprising contacting the bacterial cell with the compound of claim 1.

19. A method for preventing or inhibiting bacterial cell proliferation, the method comprising contacting the bacterial cell with the compound of claim 1.

20. A compound, or a pharmaceutically acceptable salt thereof, wherein the compound is: 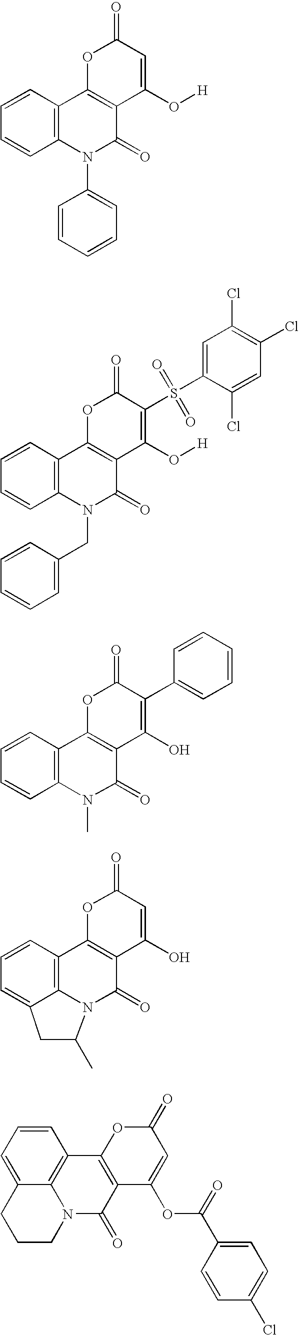 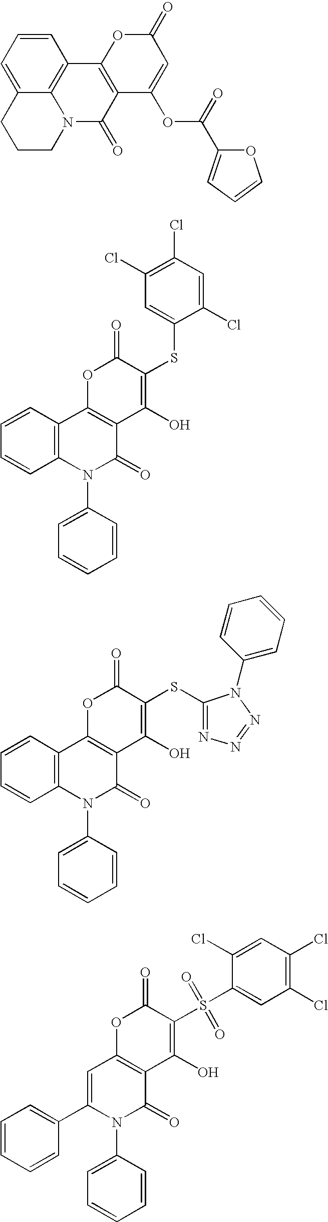 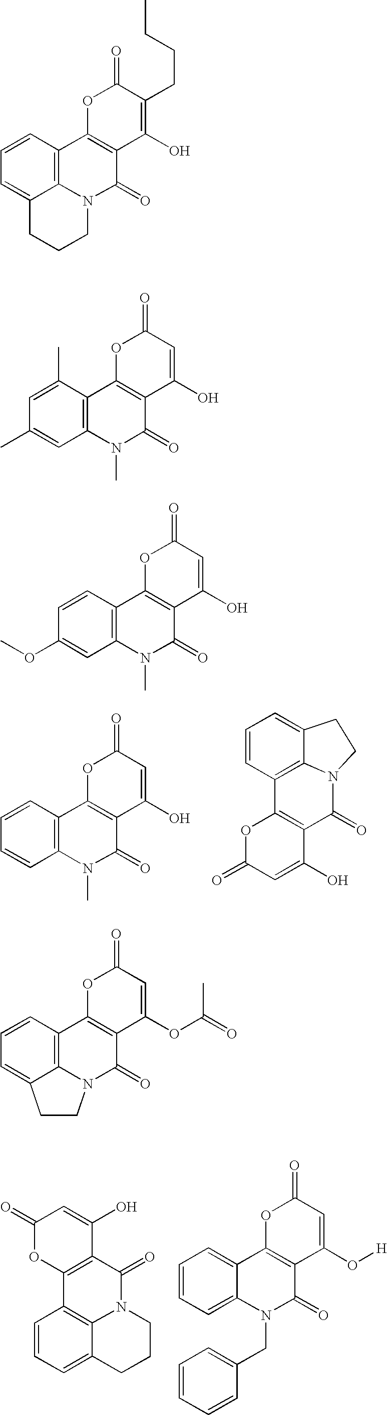 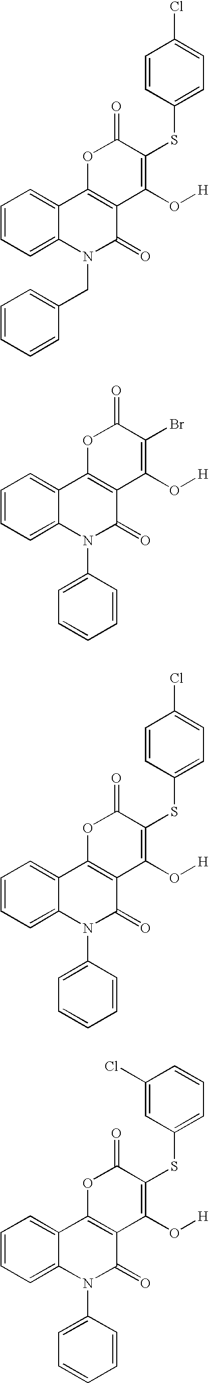 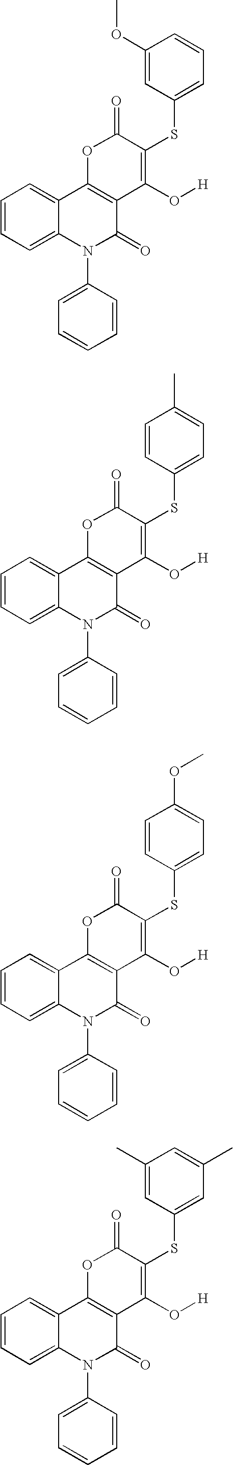 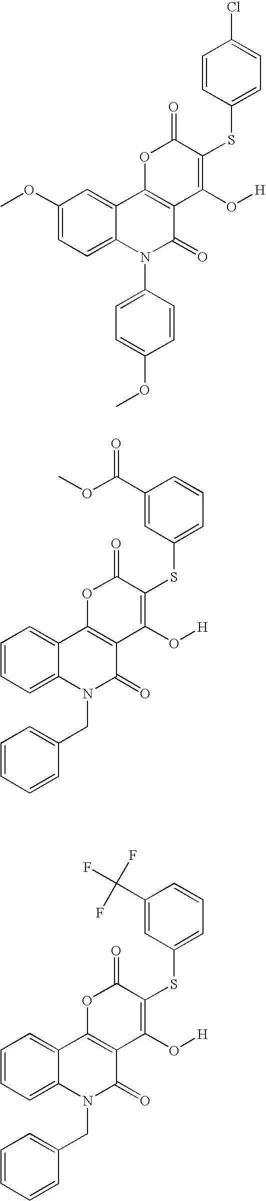 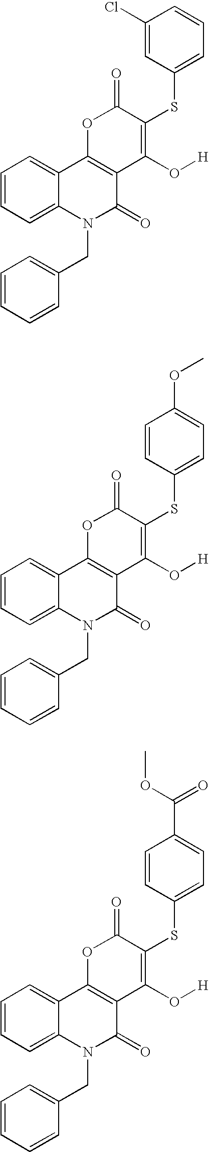 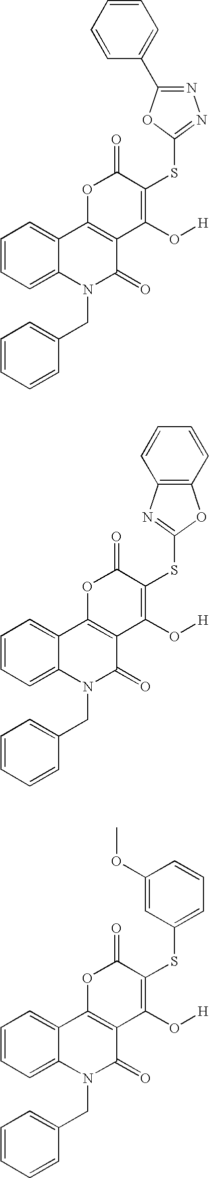 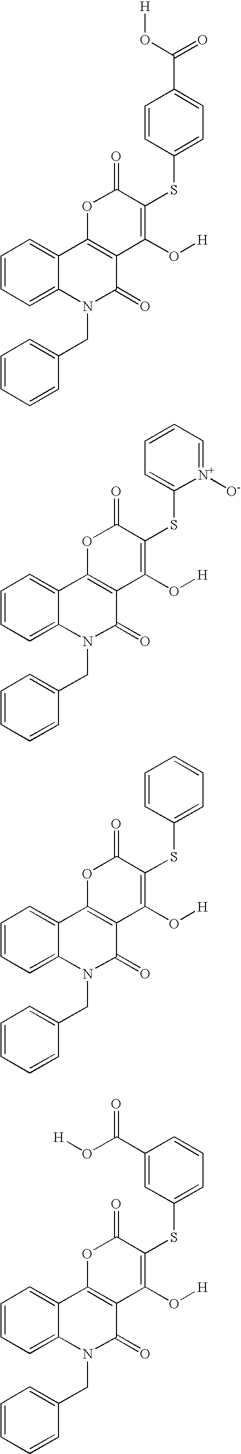 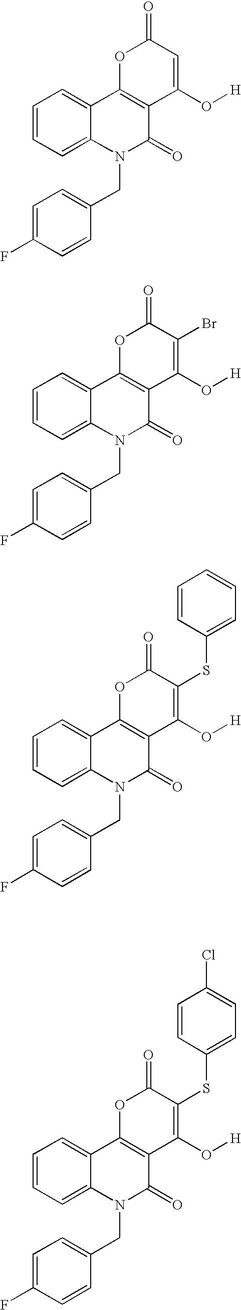 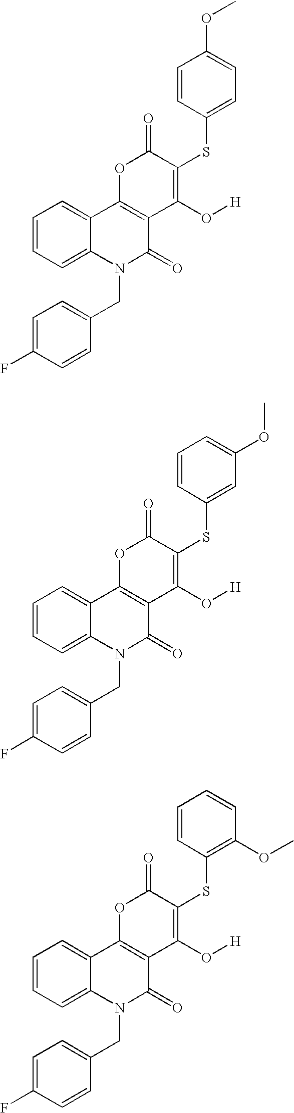 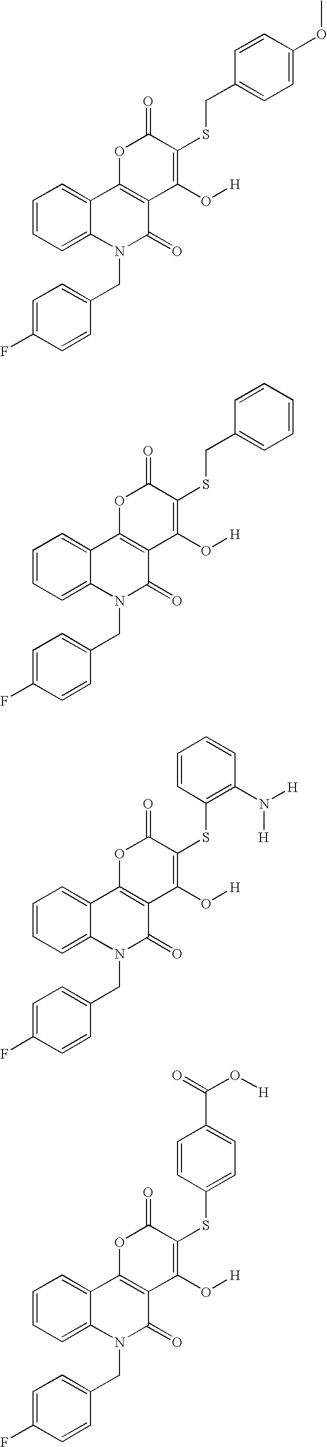 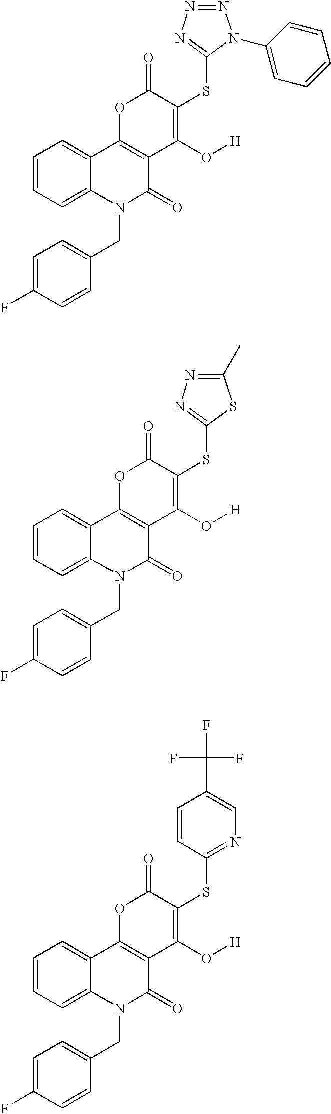 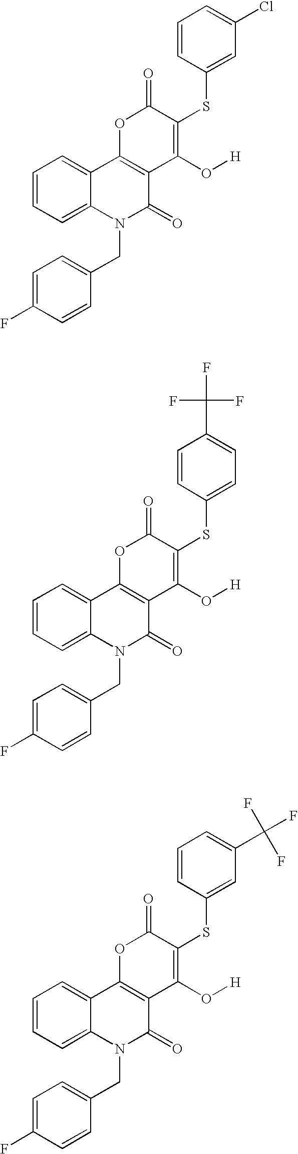 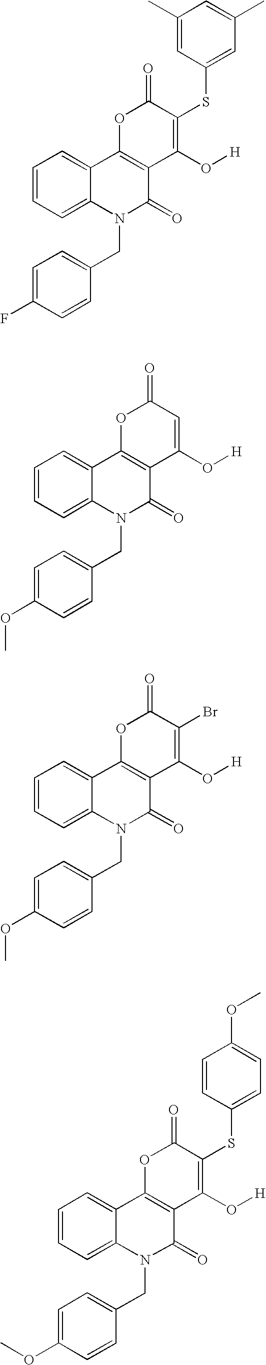 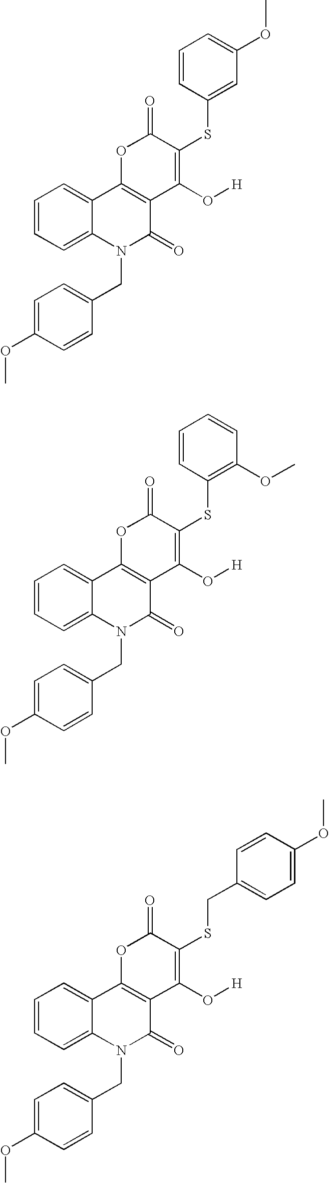 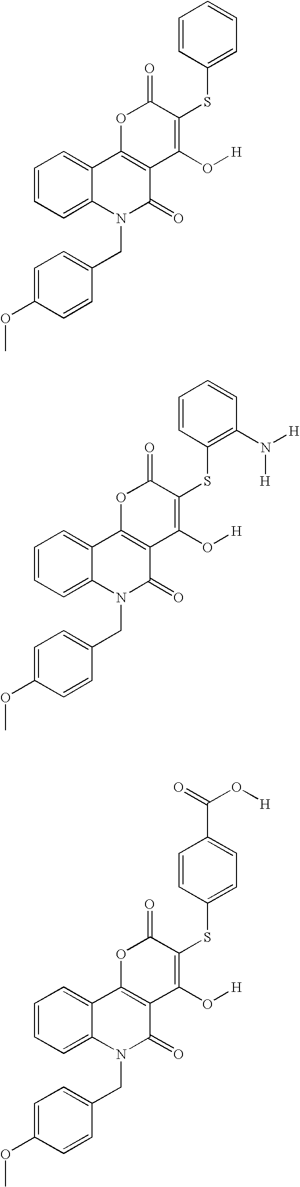 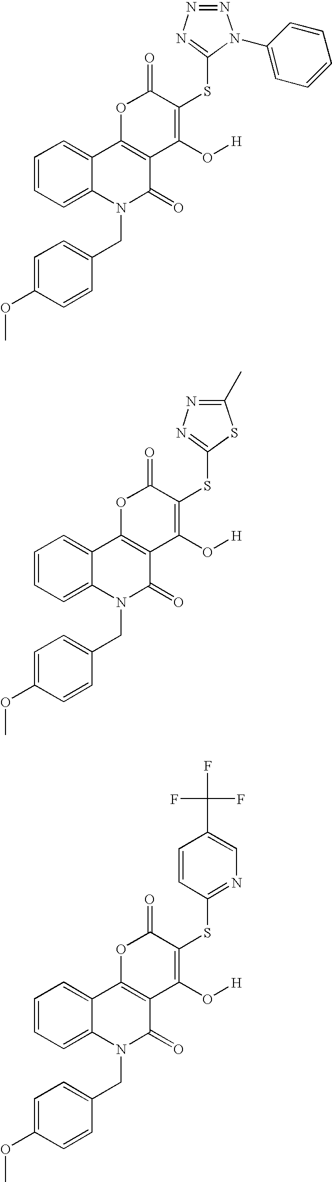 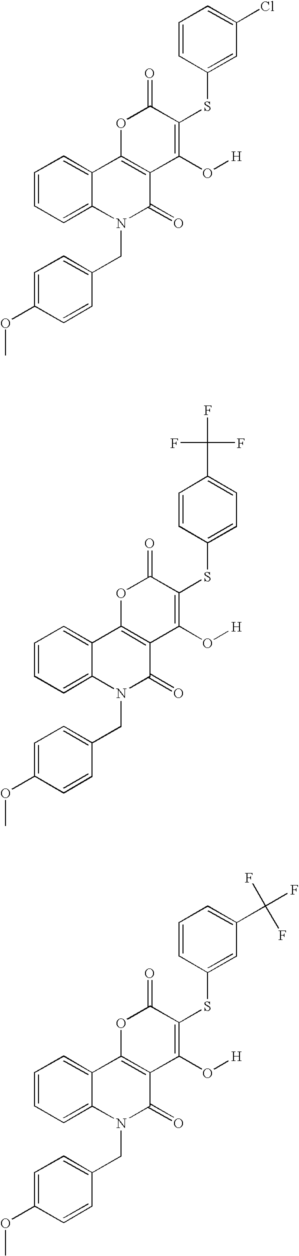 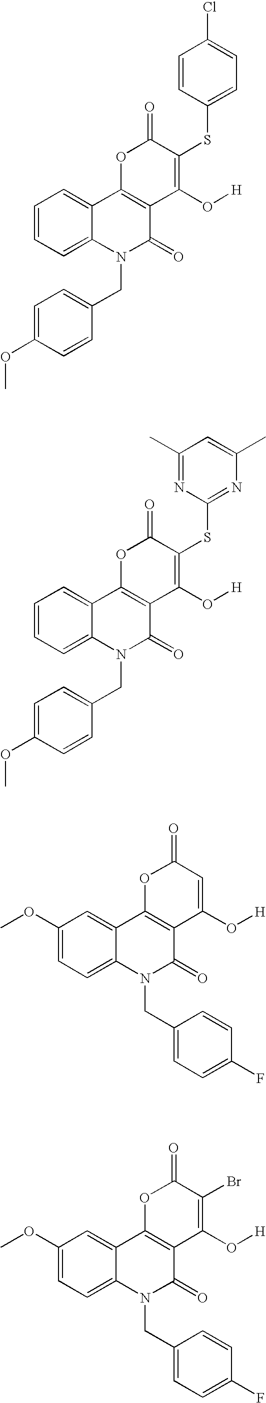 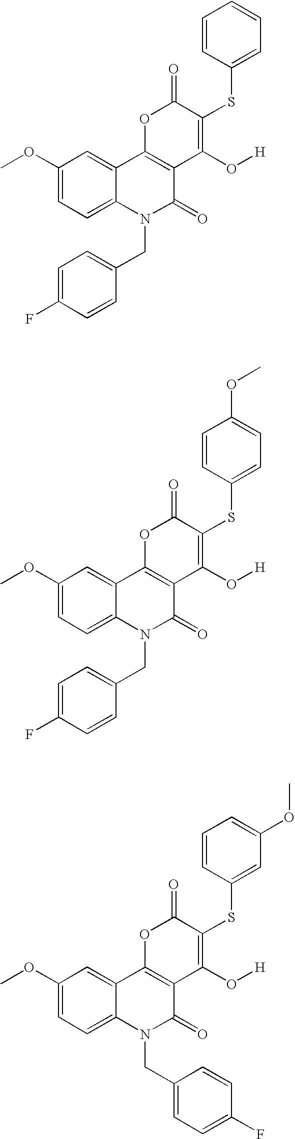 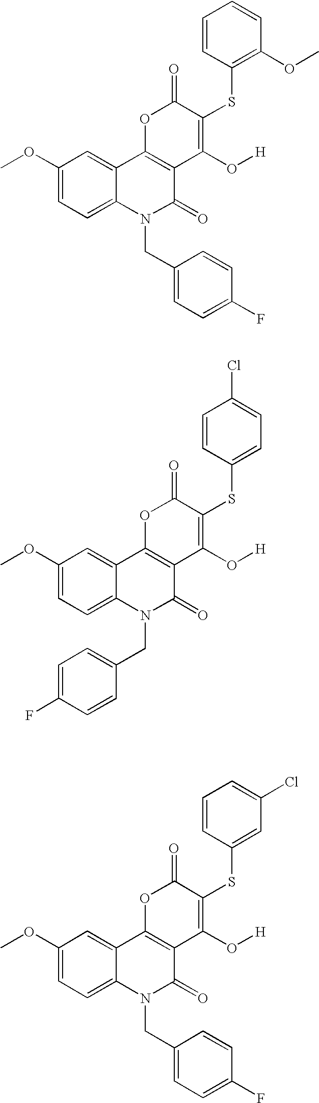 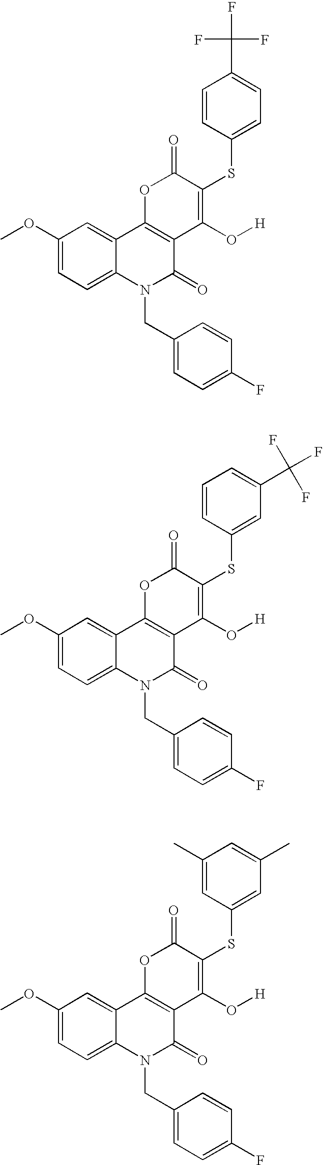 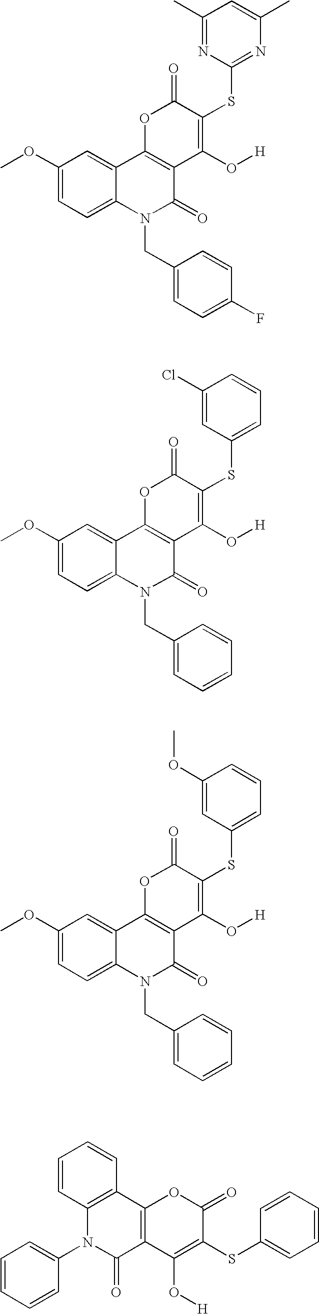 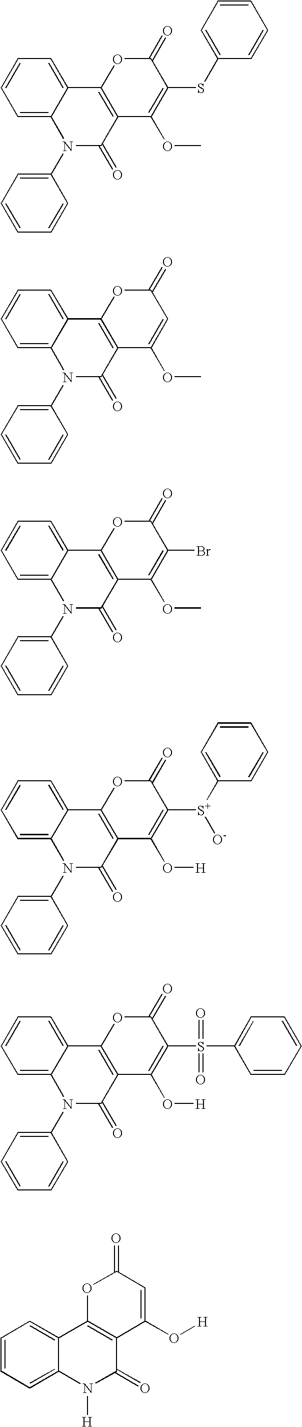 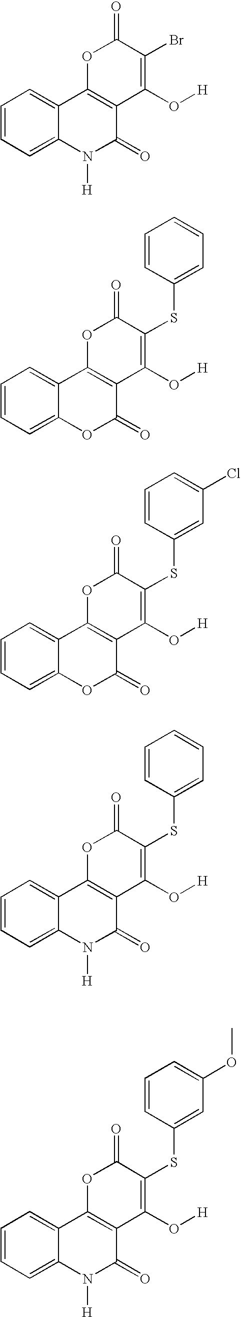 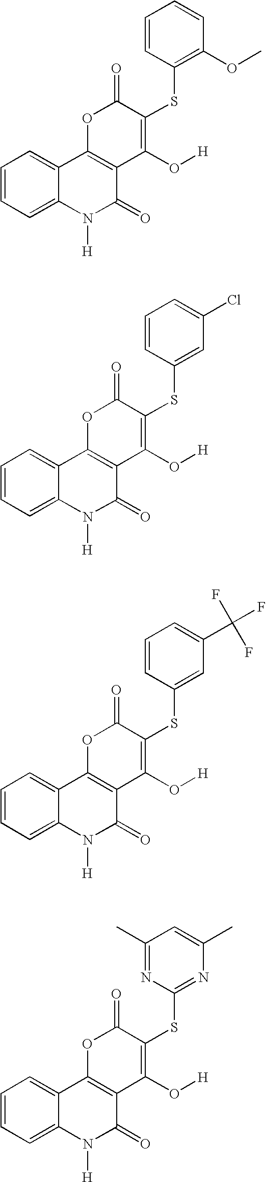 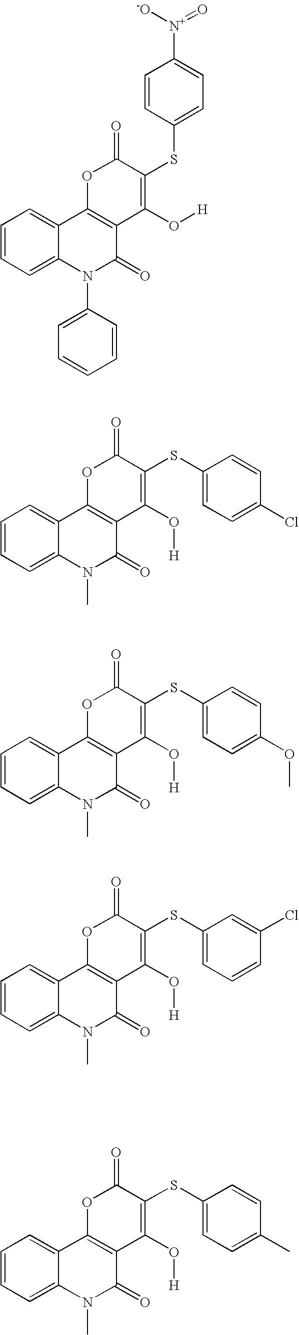 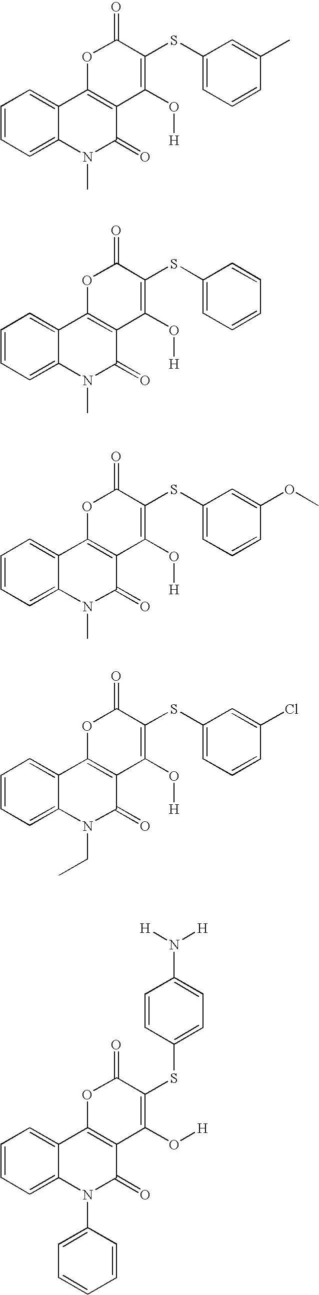 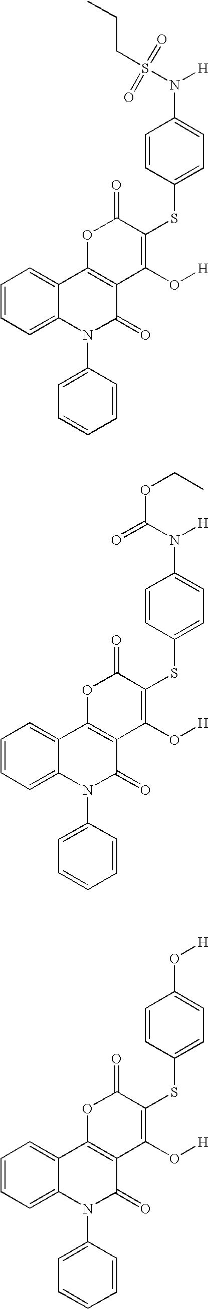 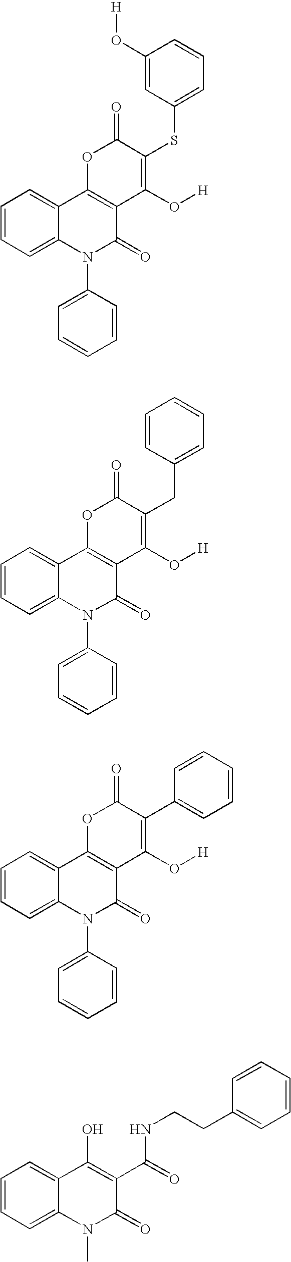 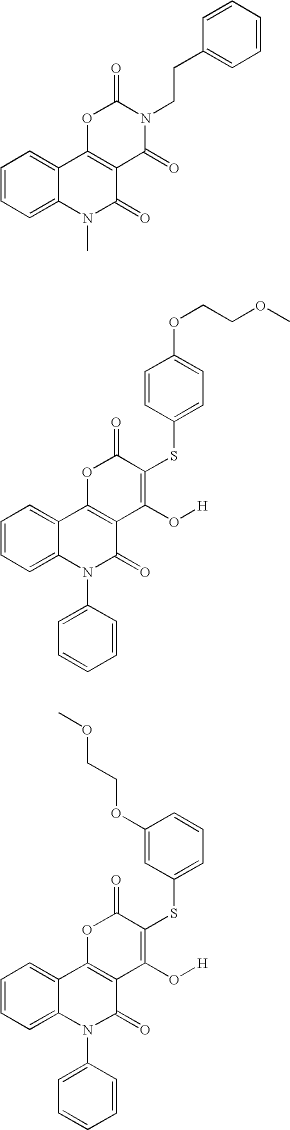 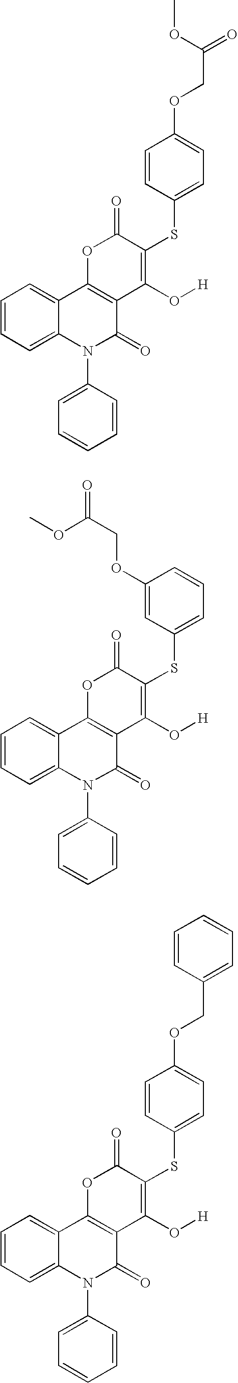 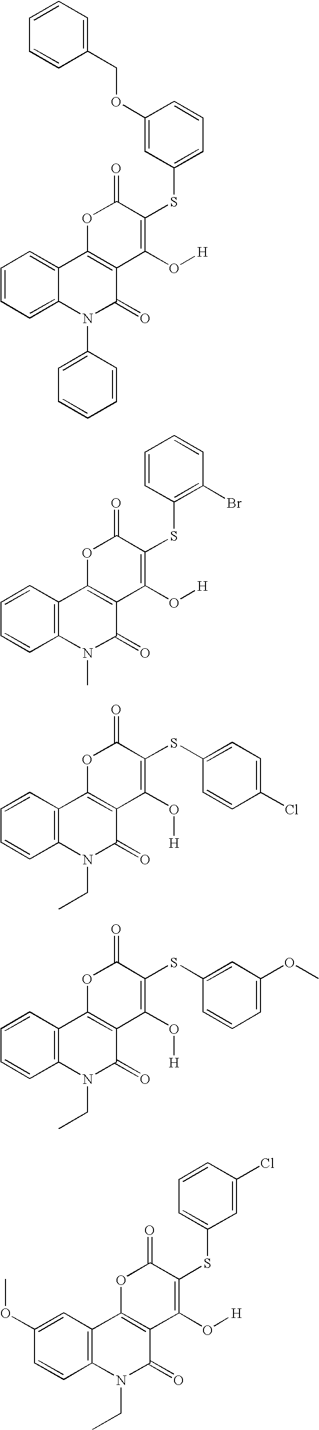 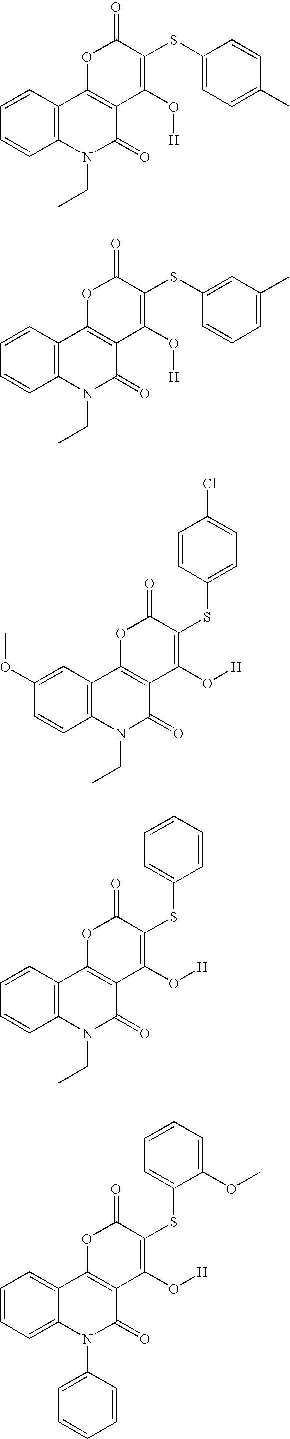 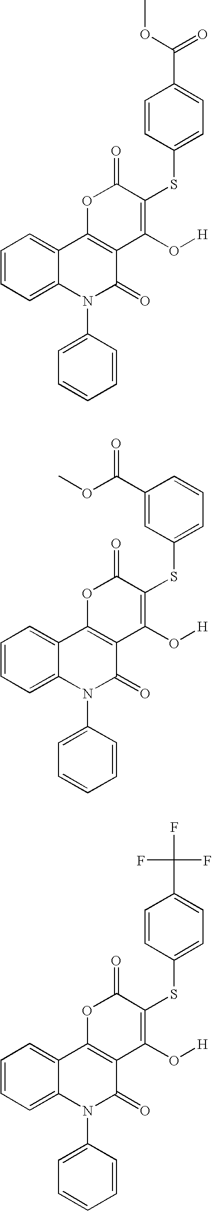 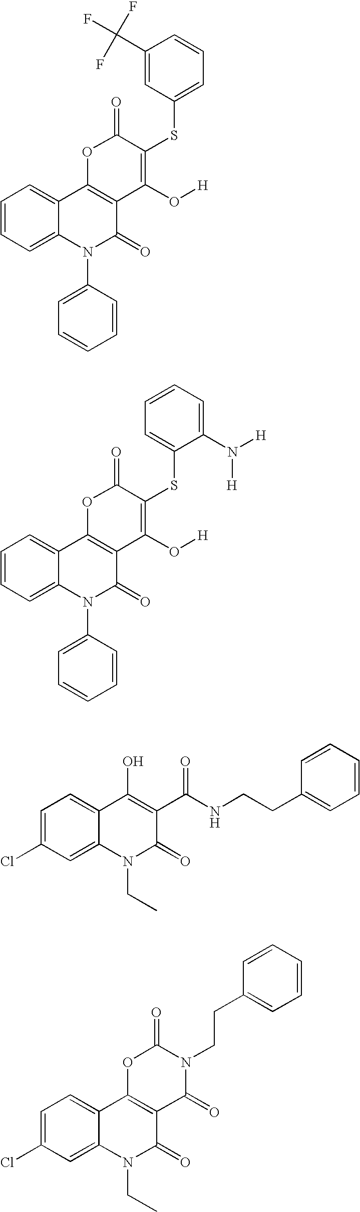 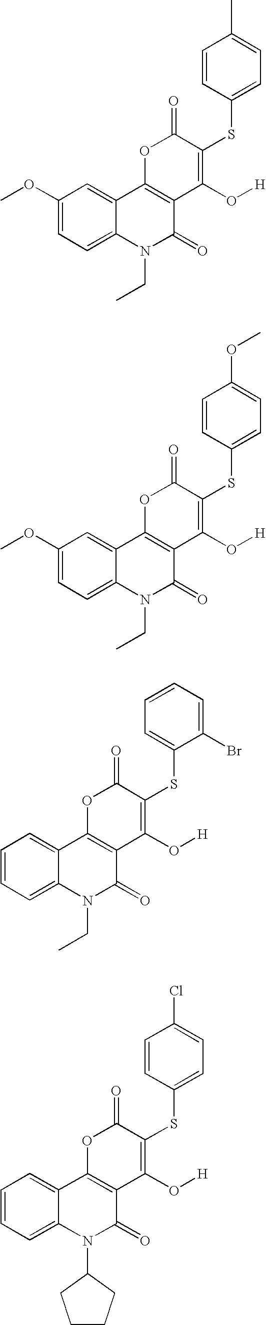 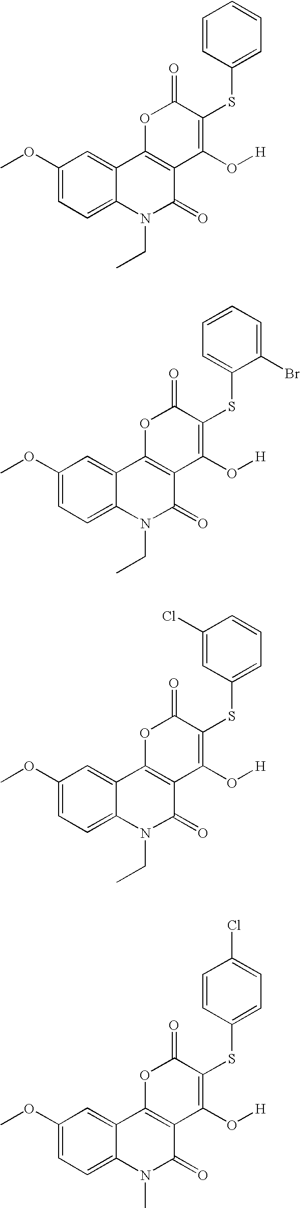 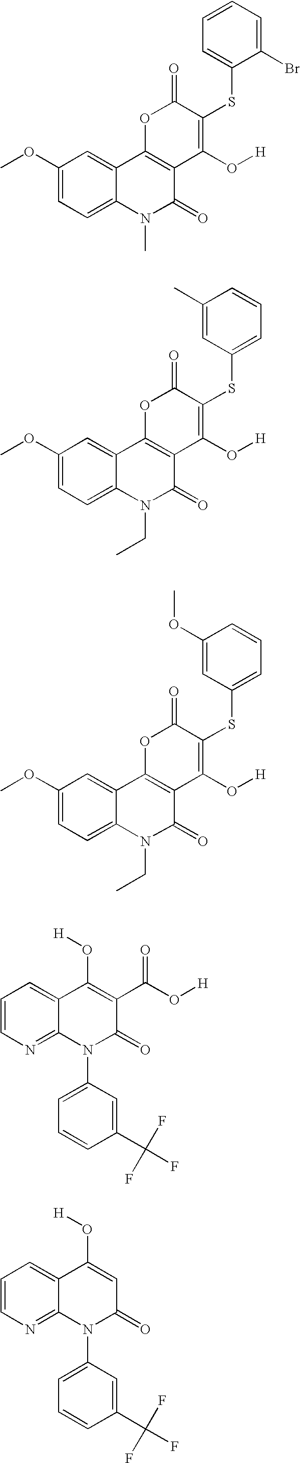 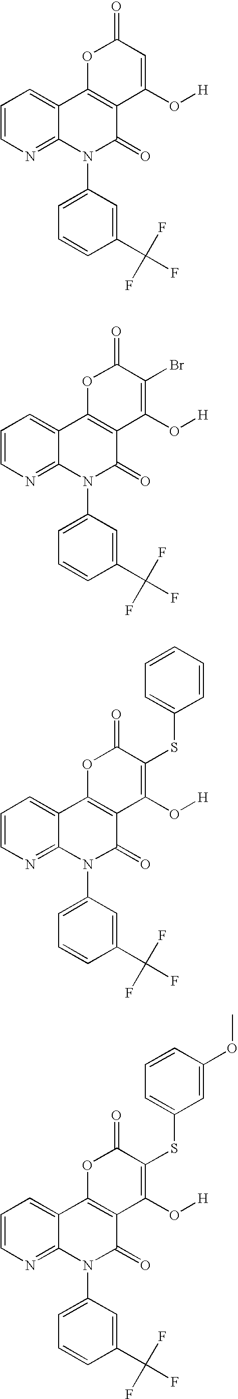 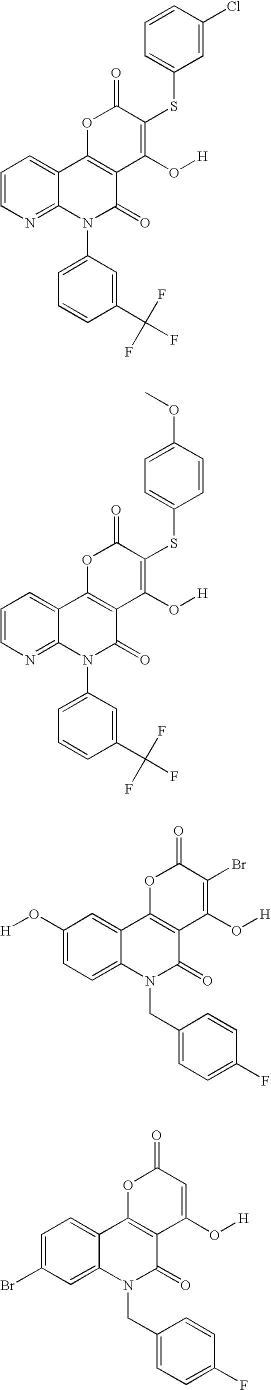 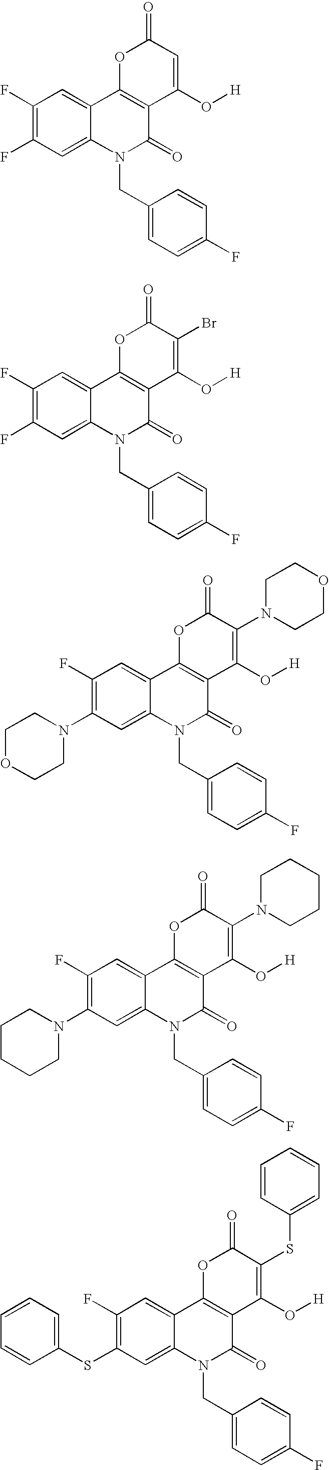 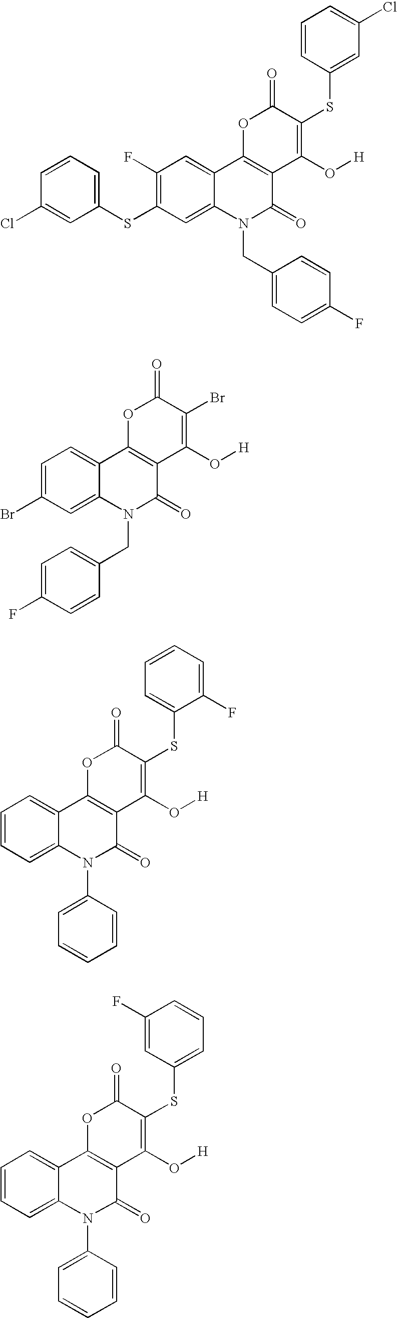 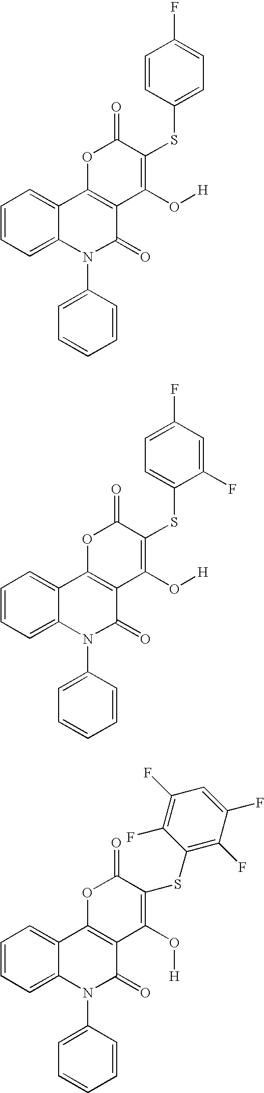 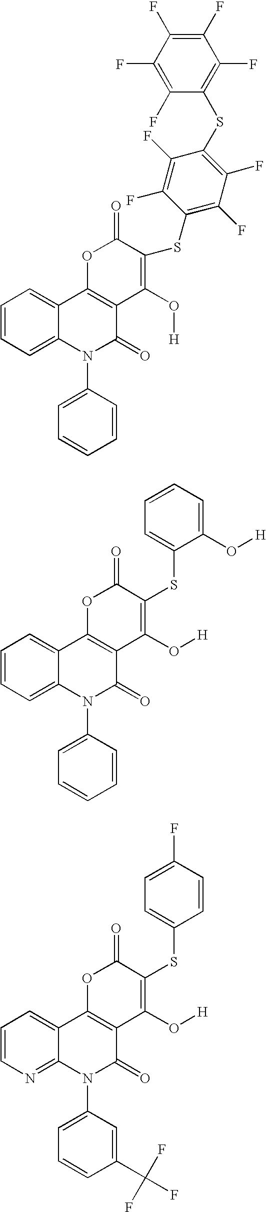 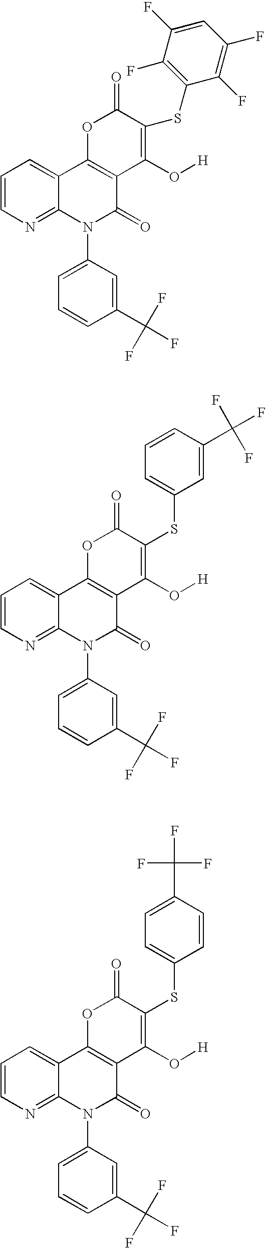 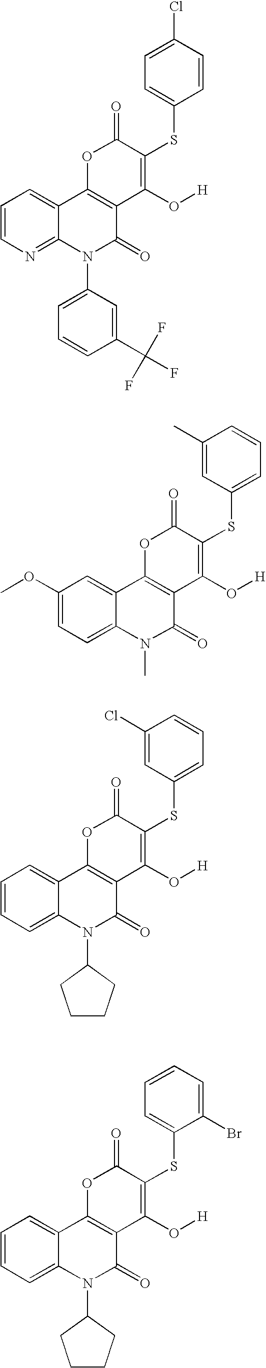 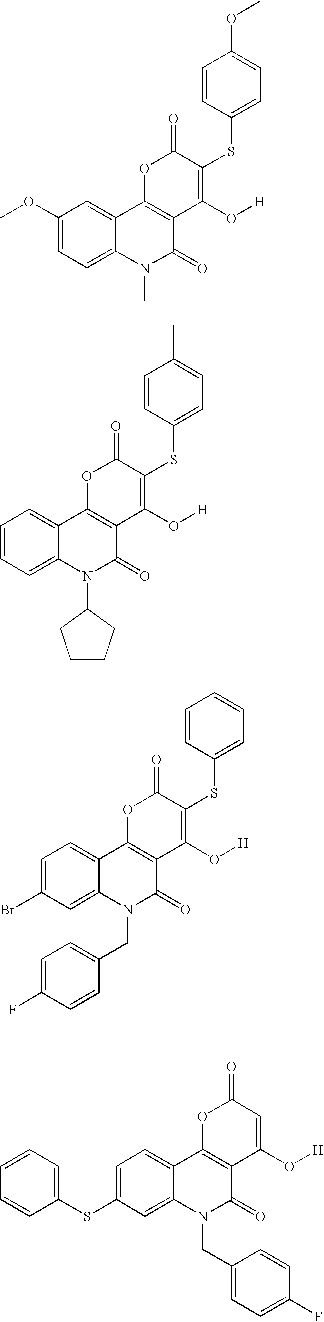 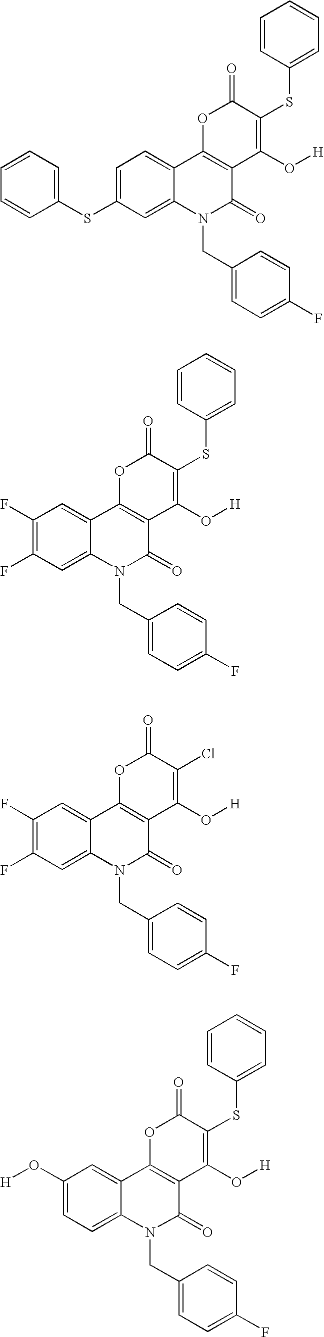 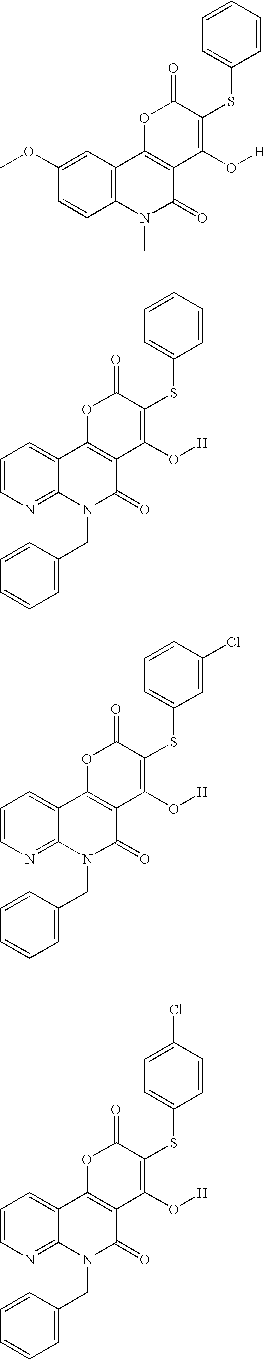 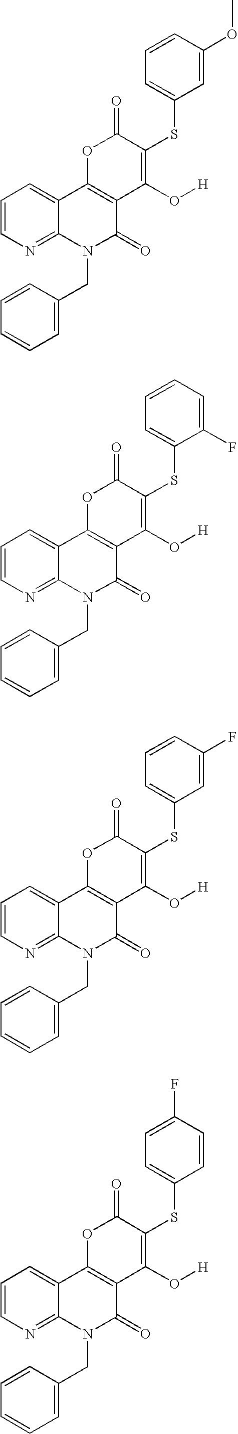 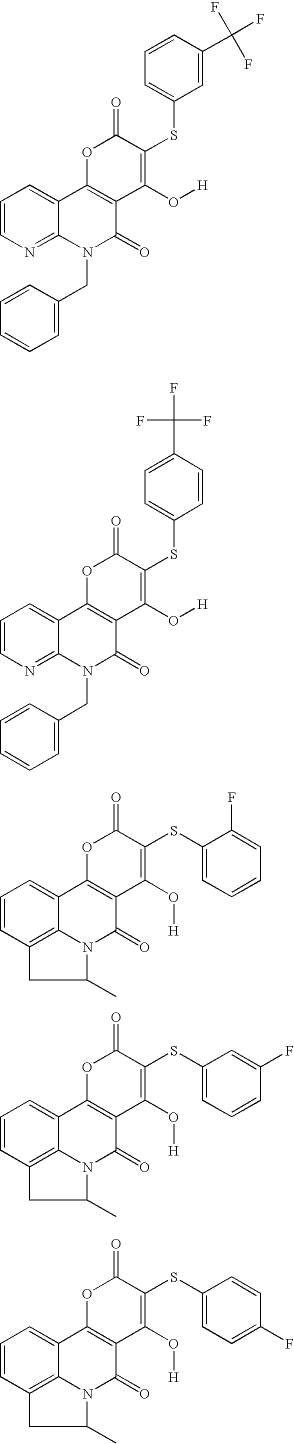 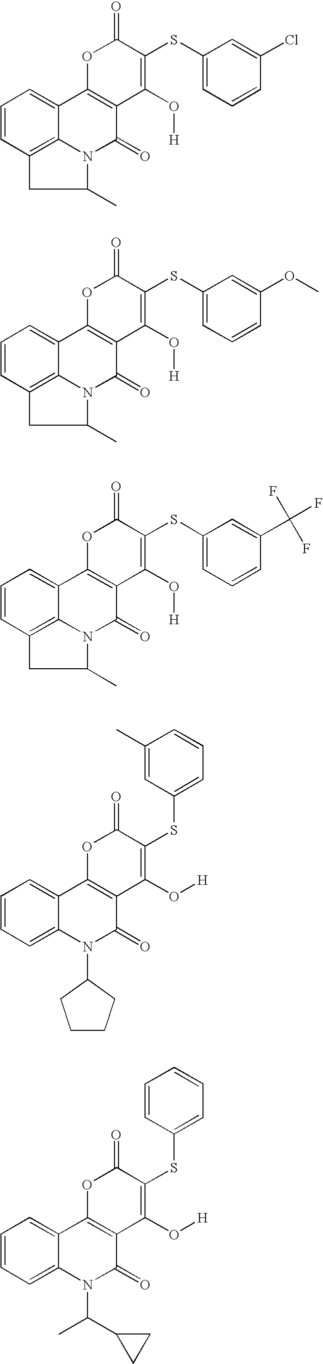 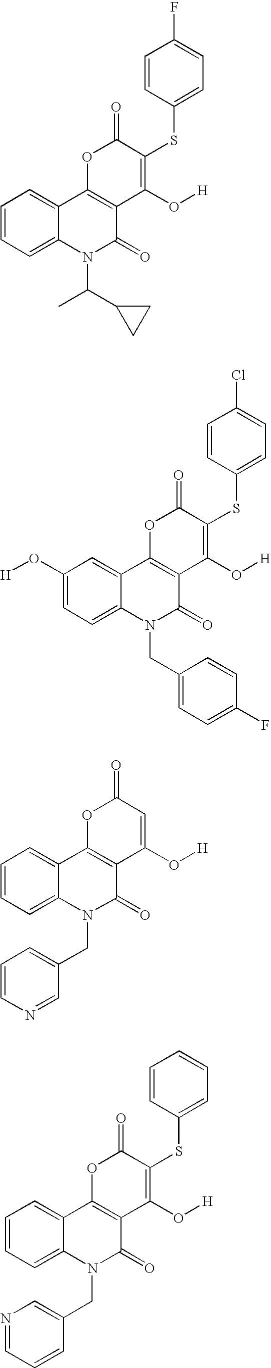 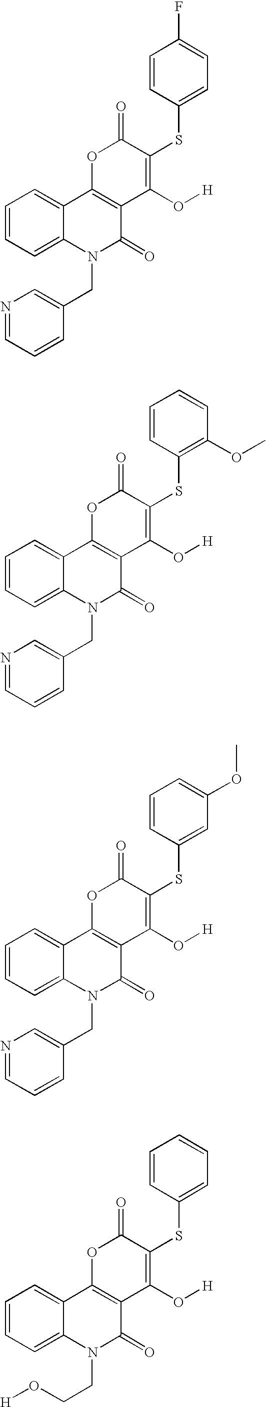 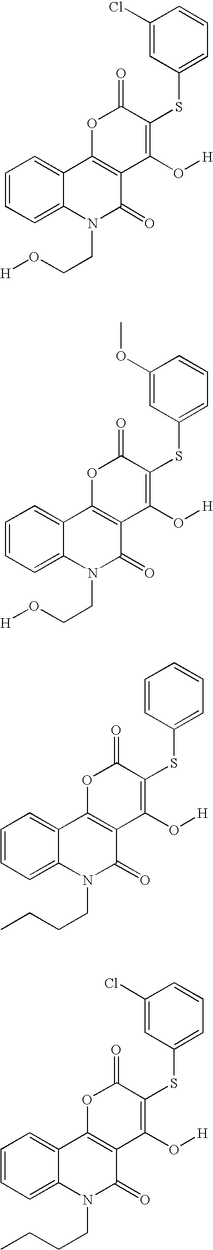 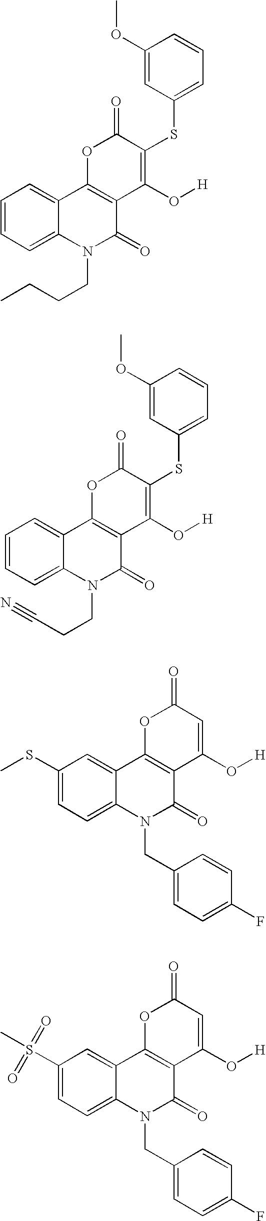 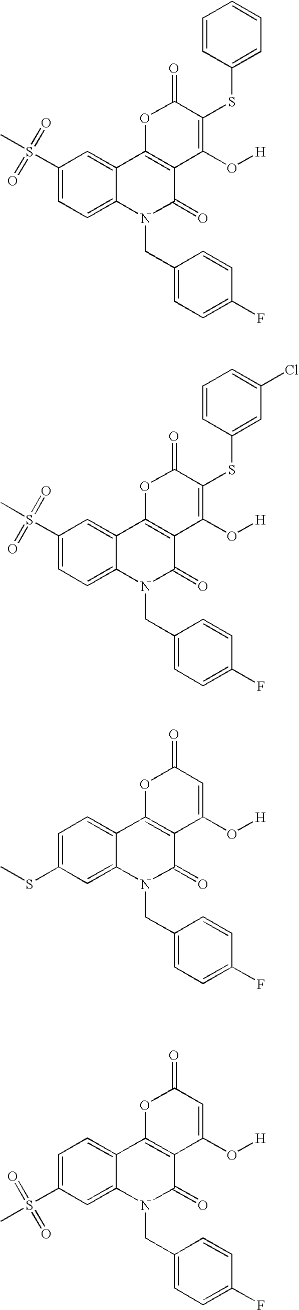 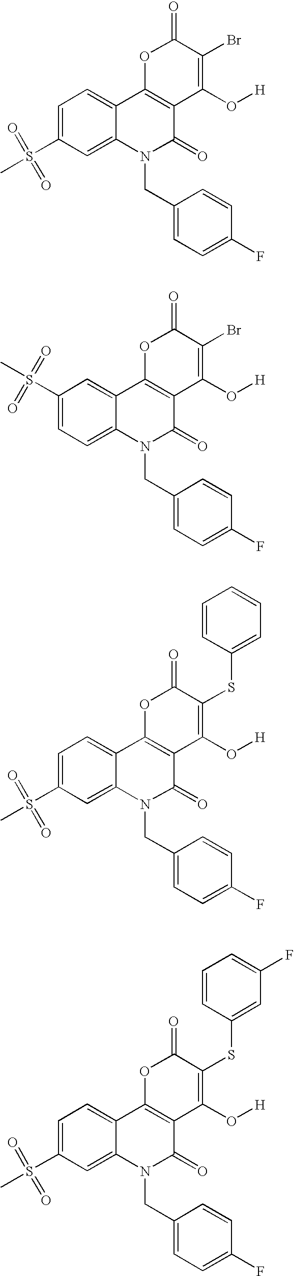 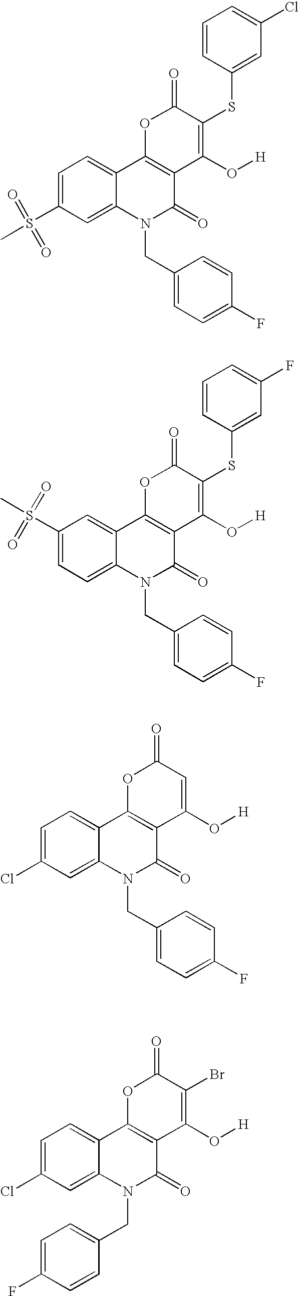 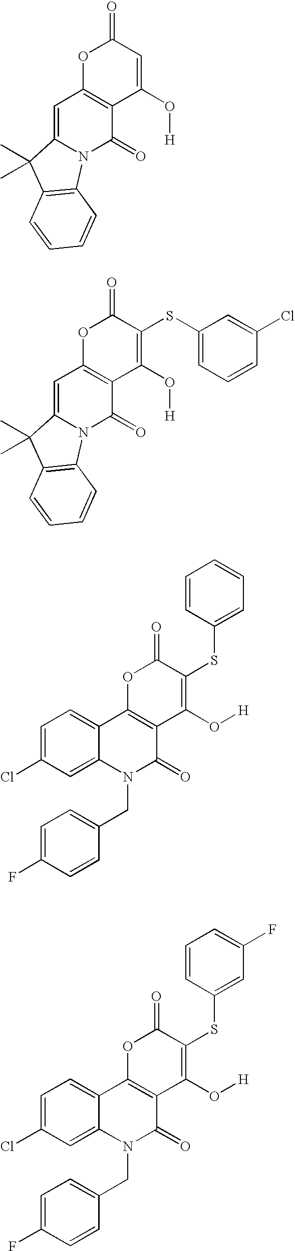 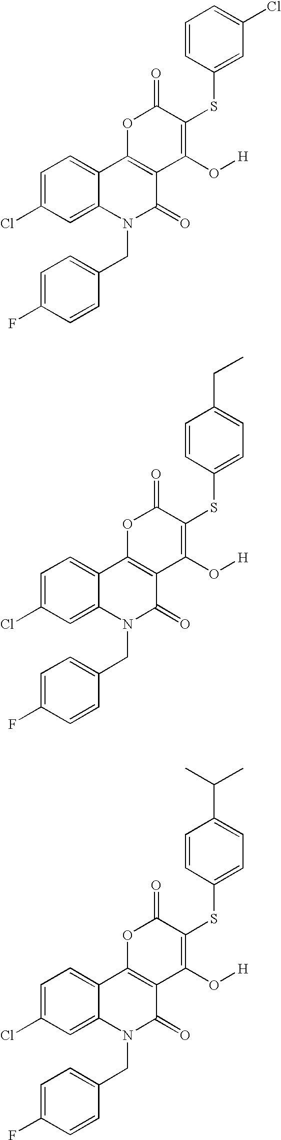 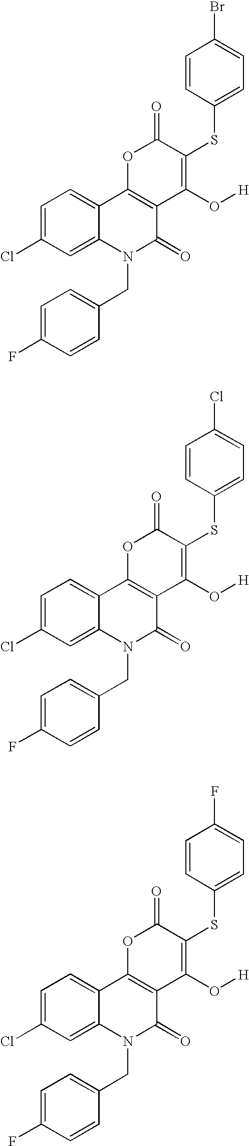 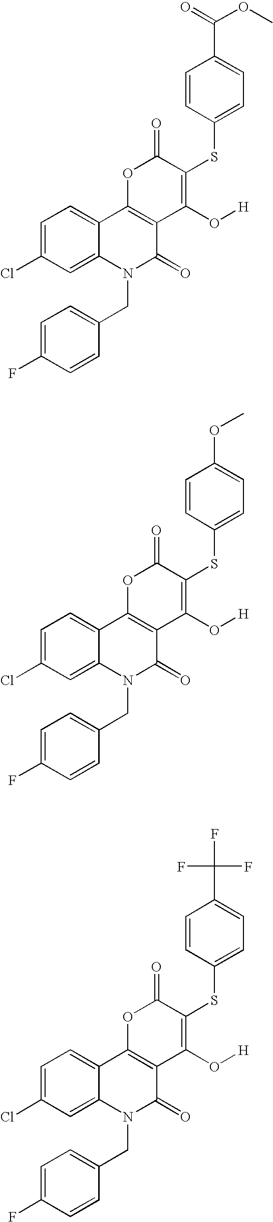 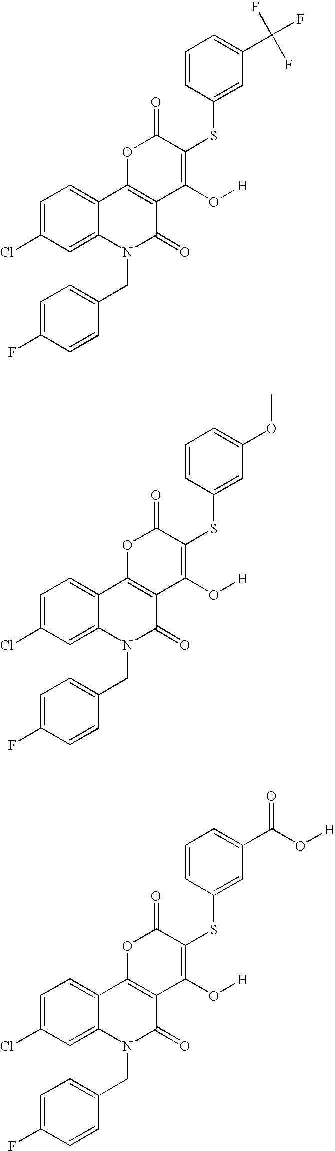 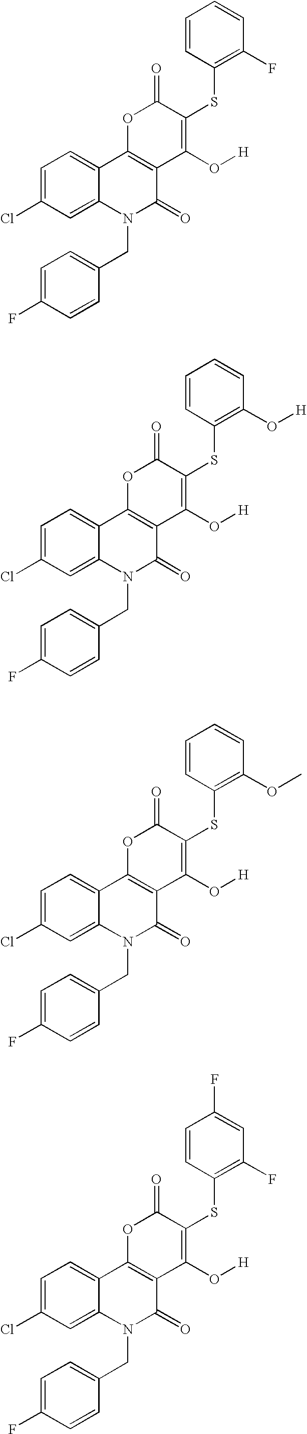 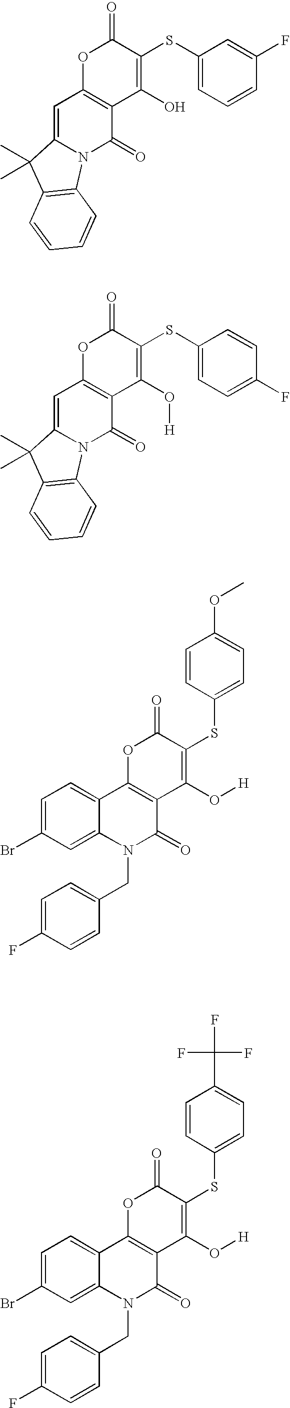 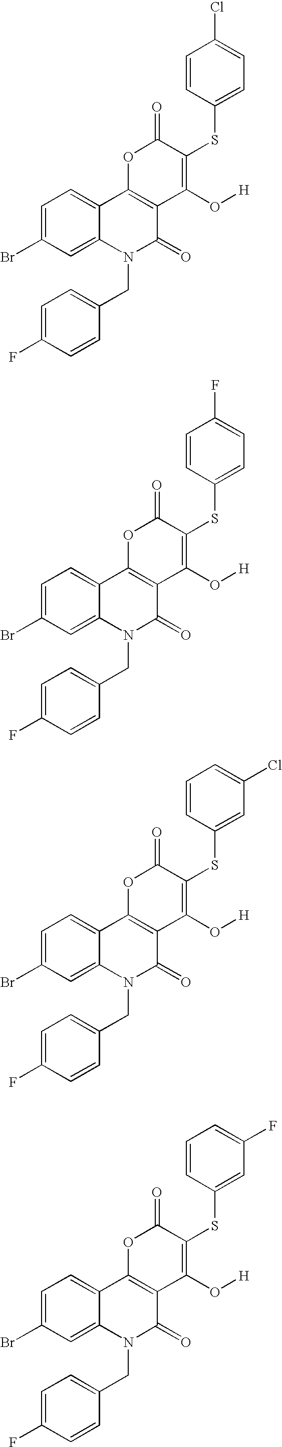 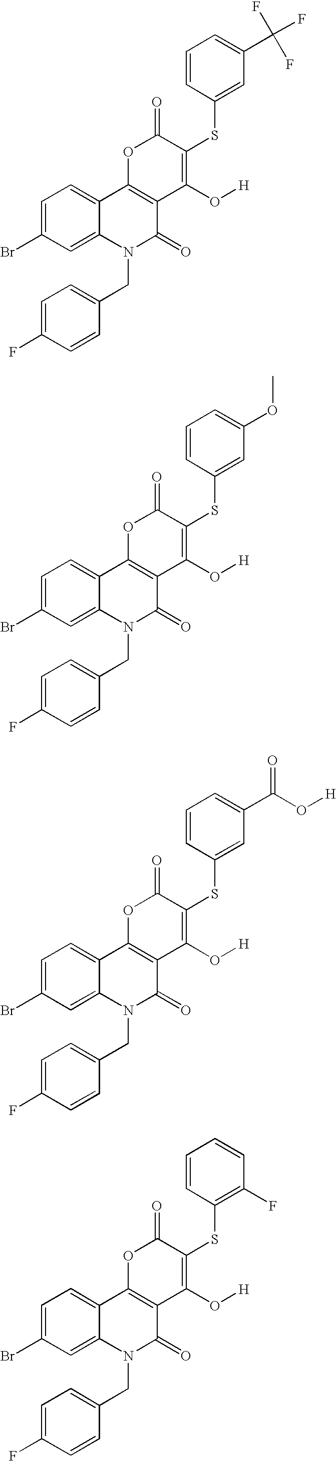 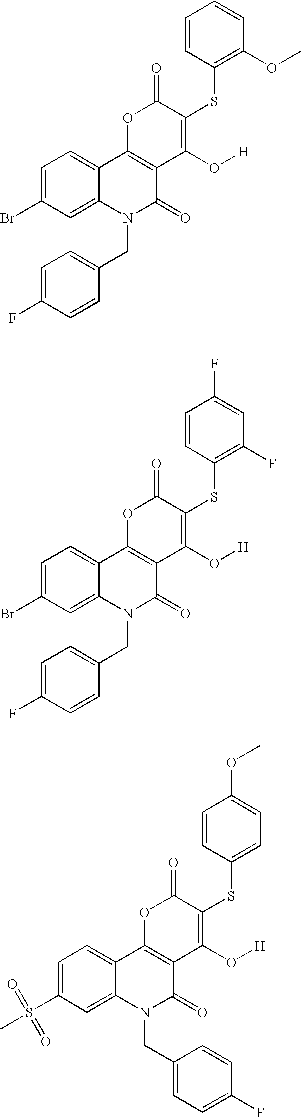 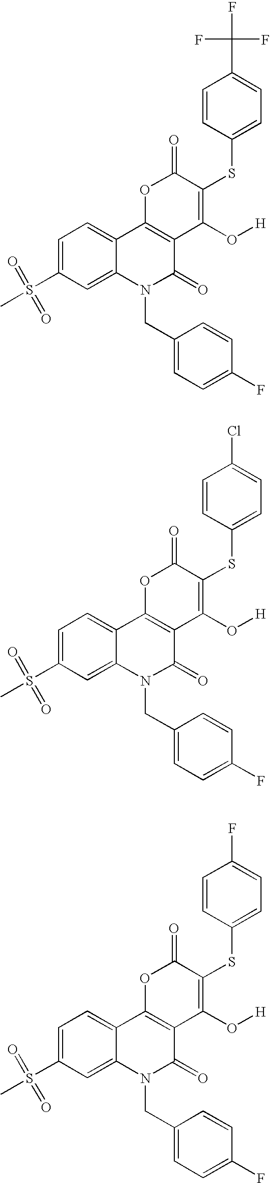 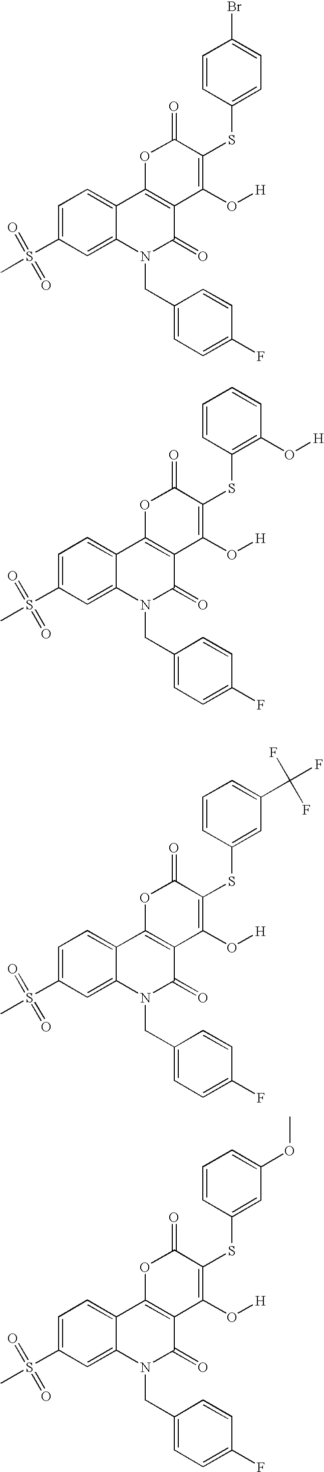 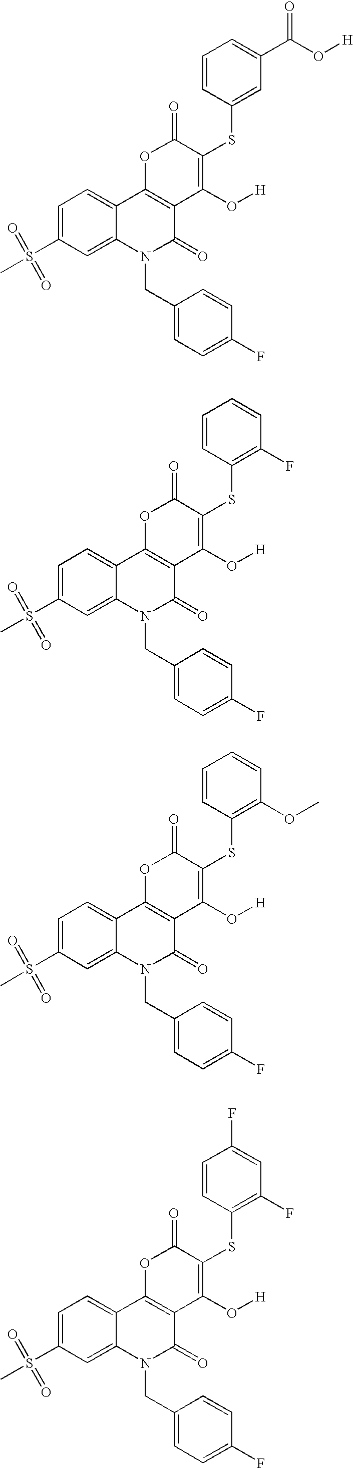 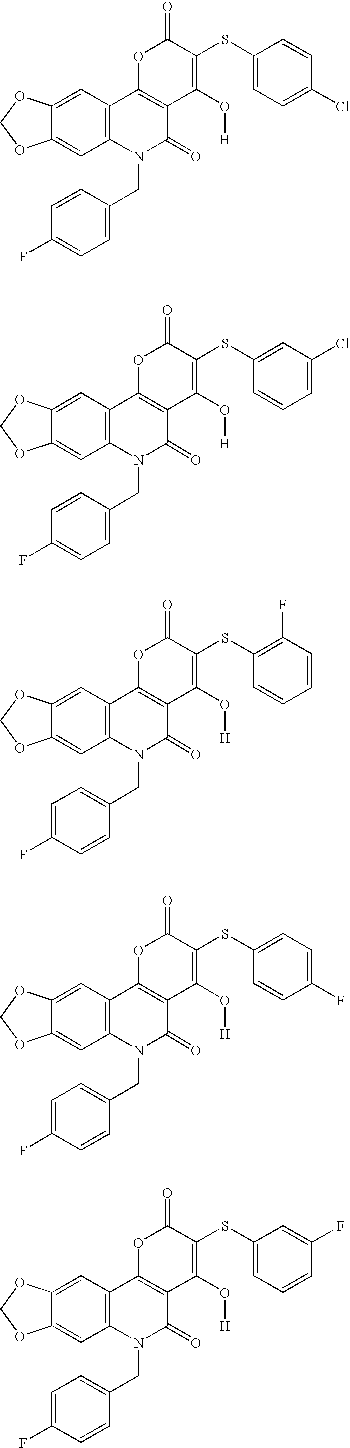 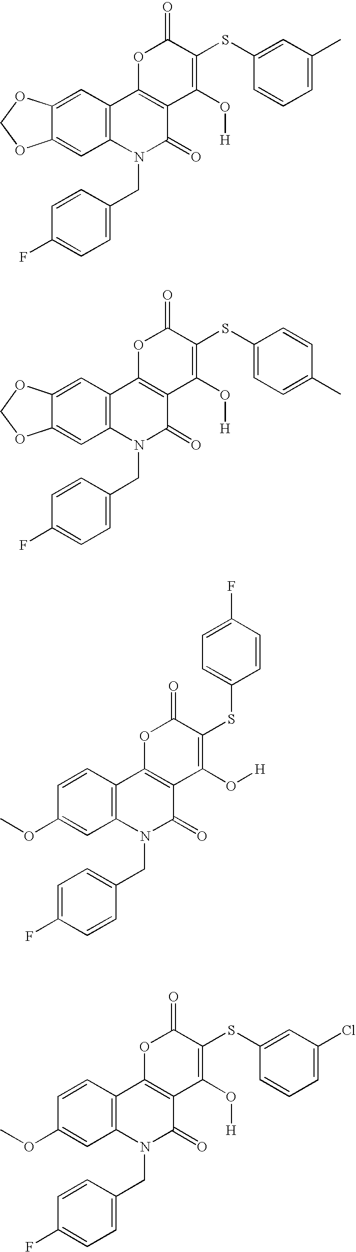 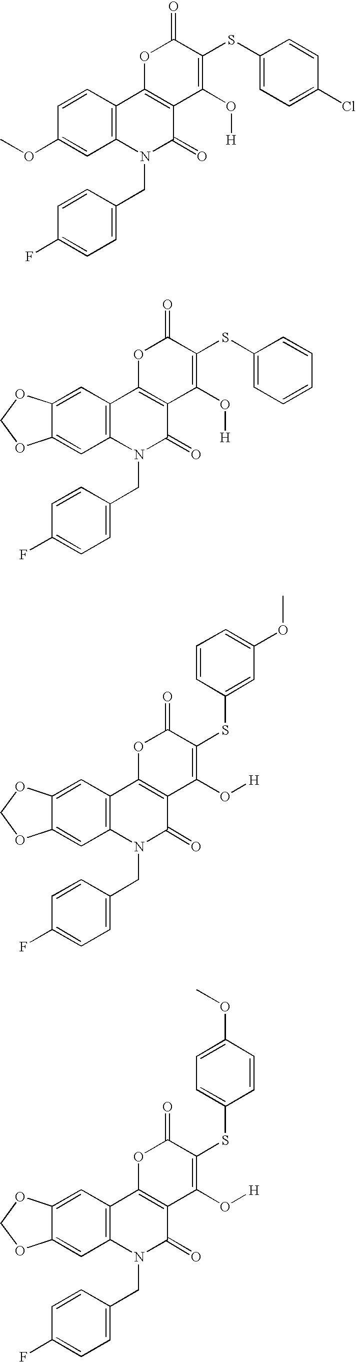 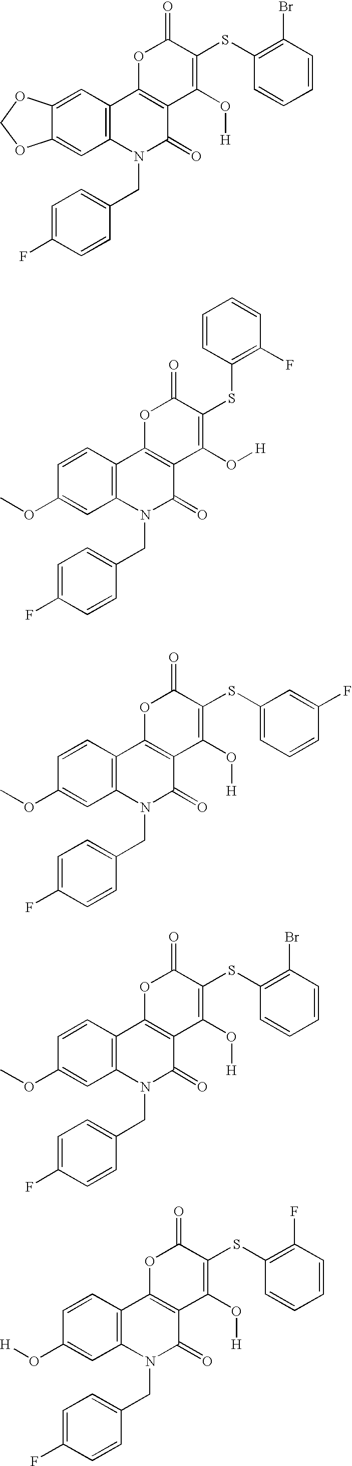 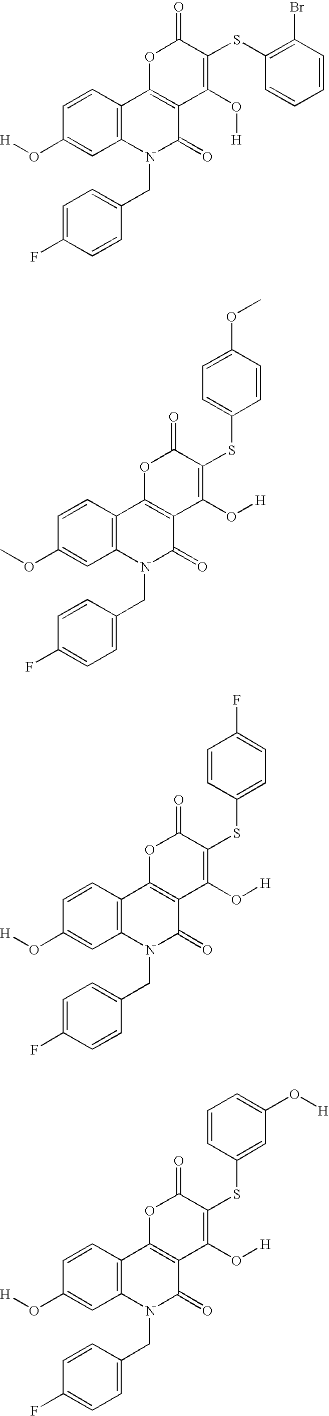 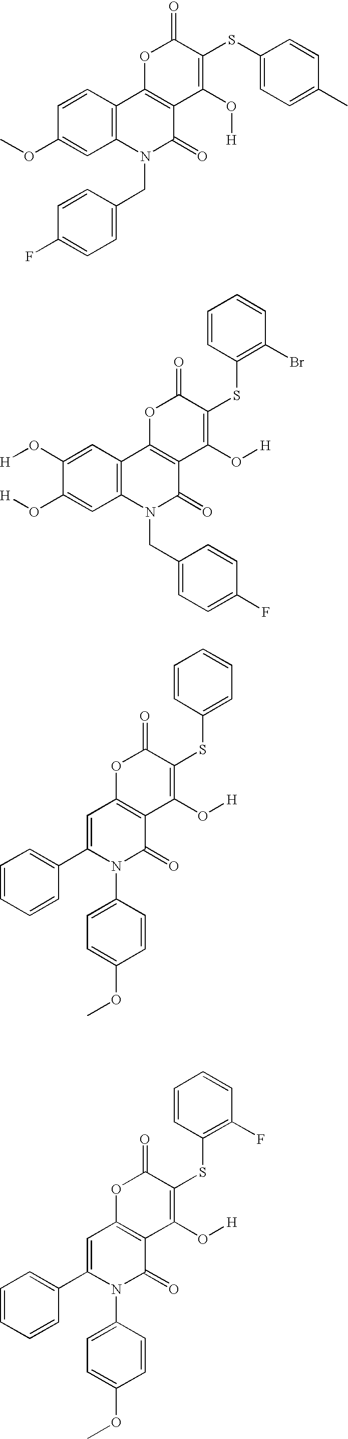 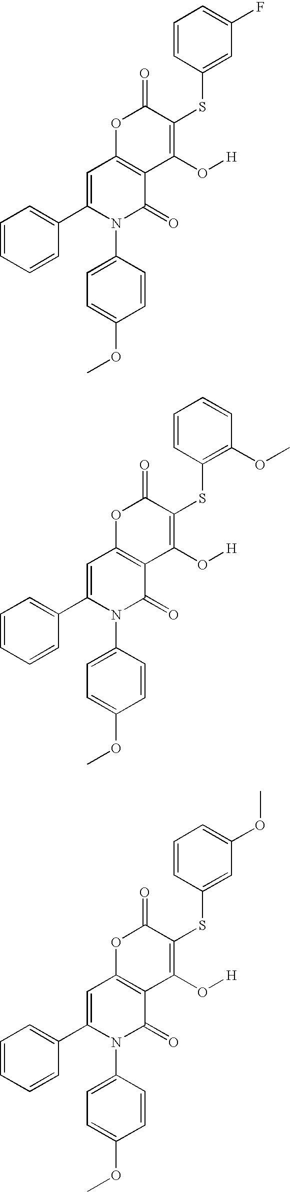 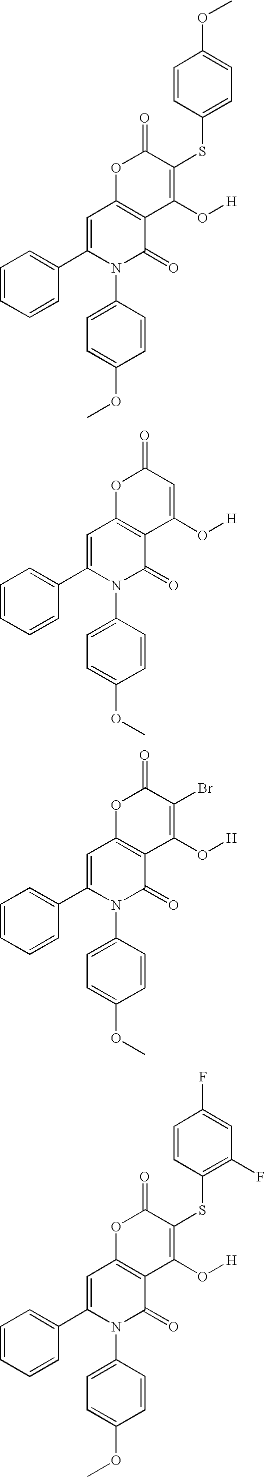 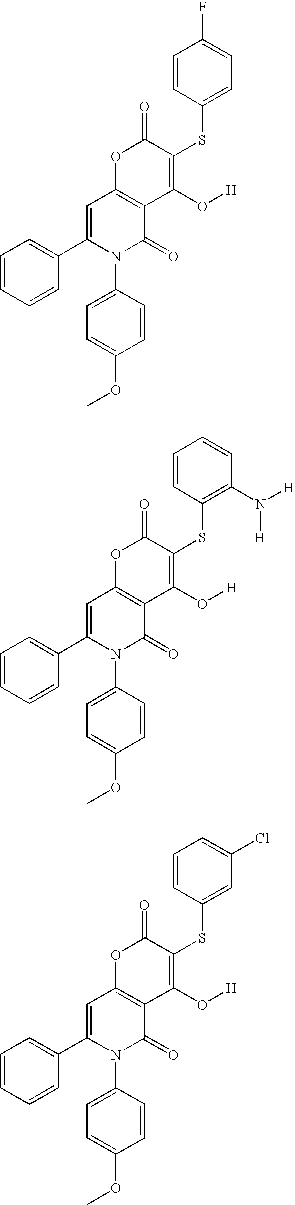 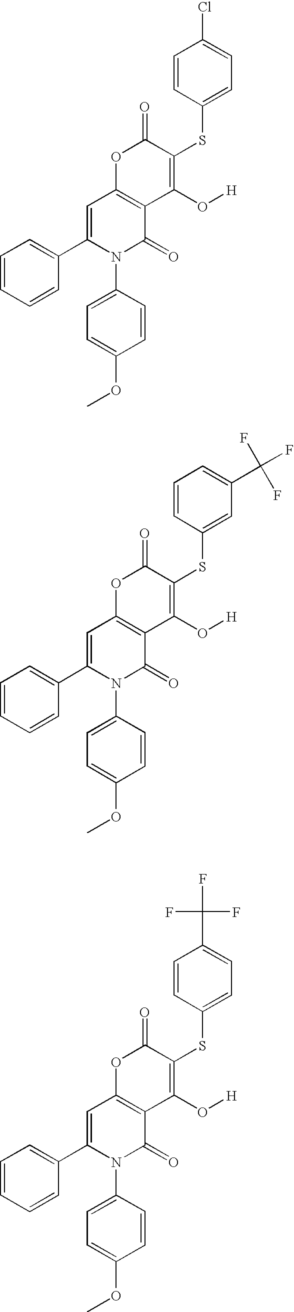 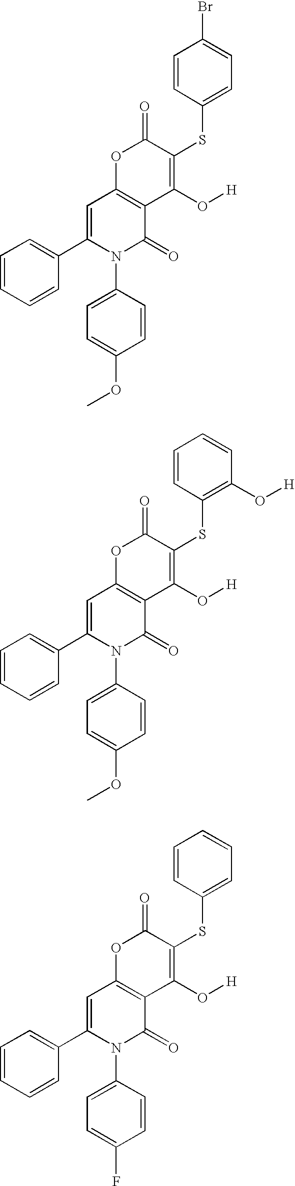 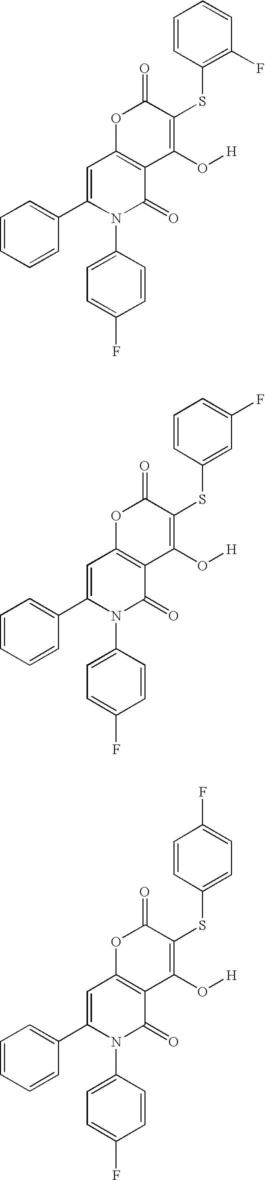 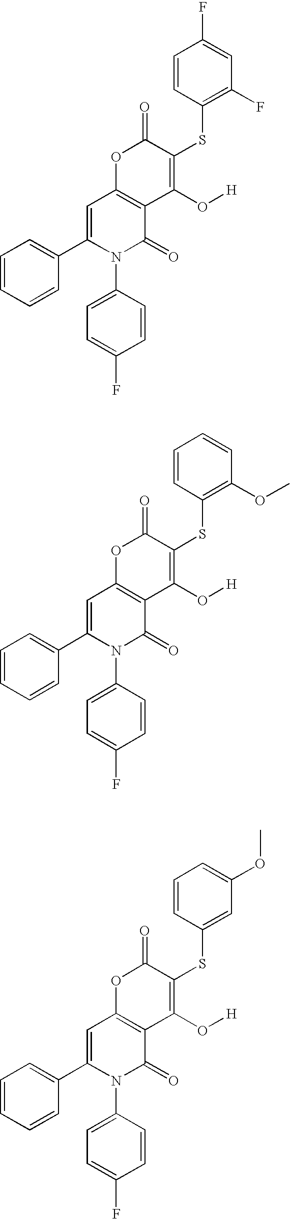 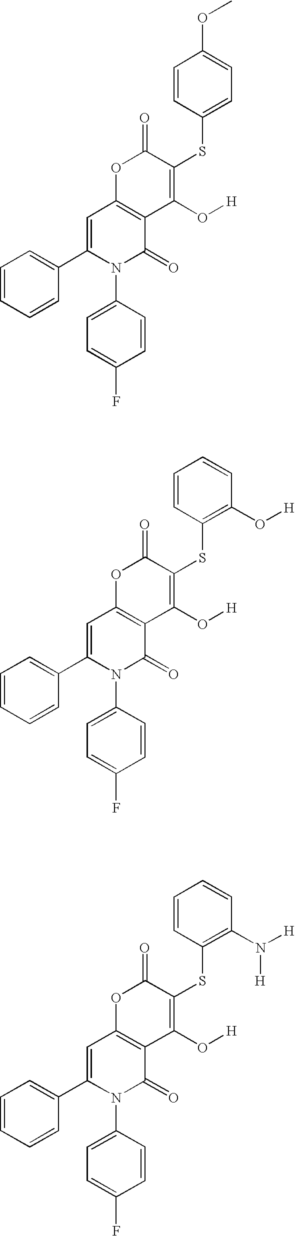 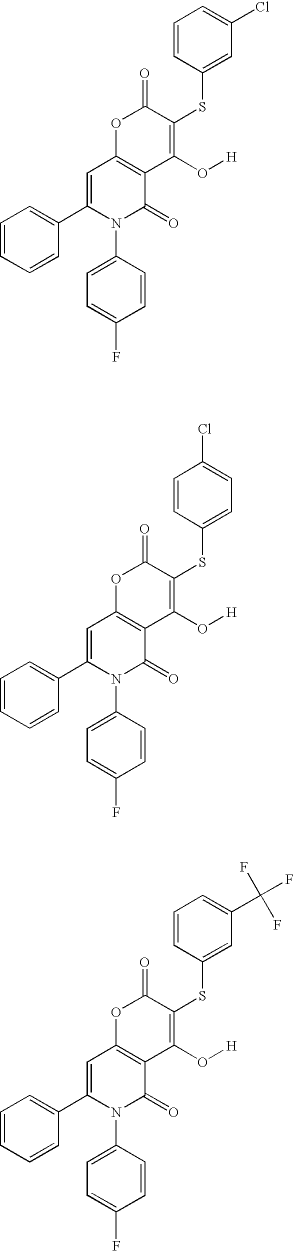 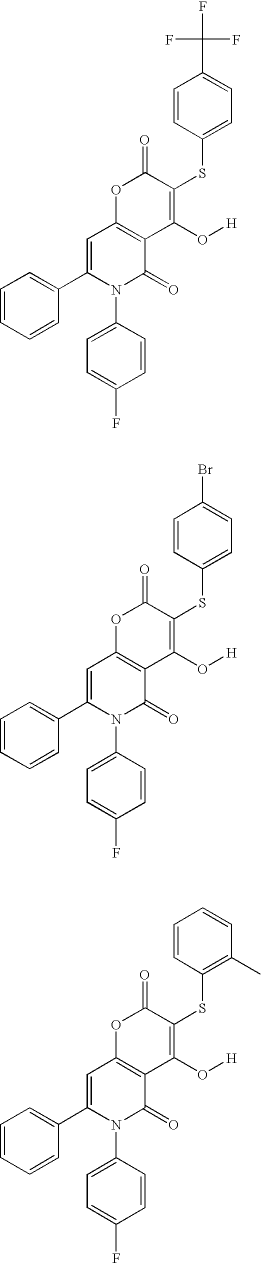 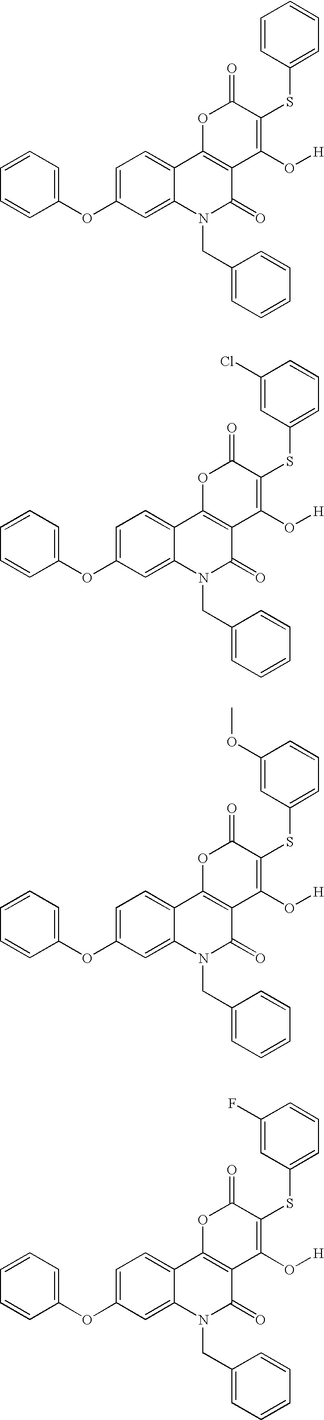 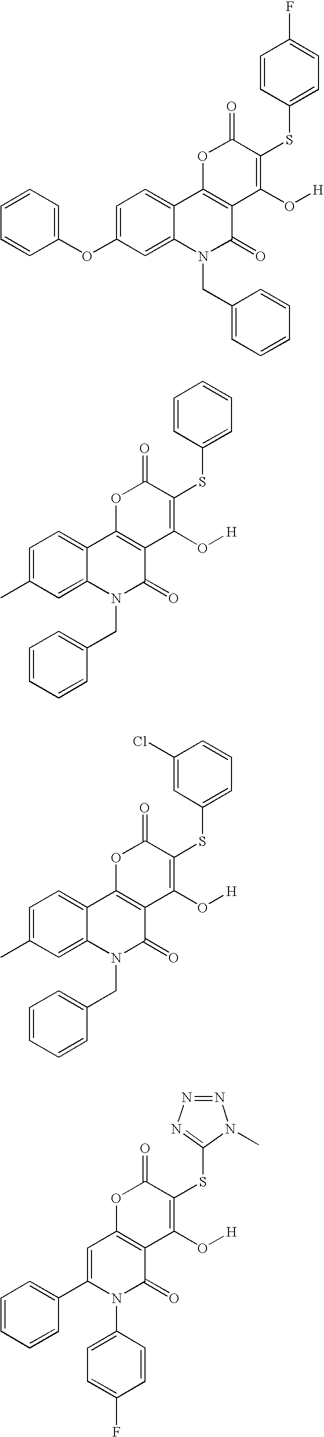 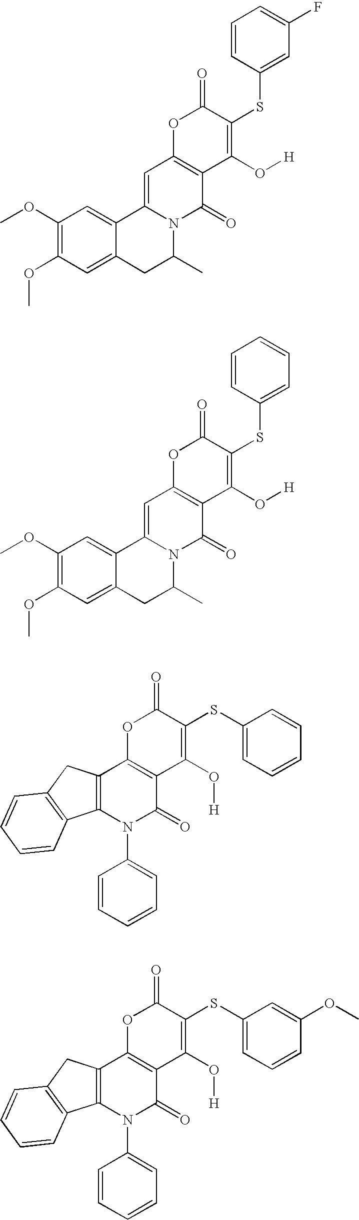 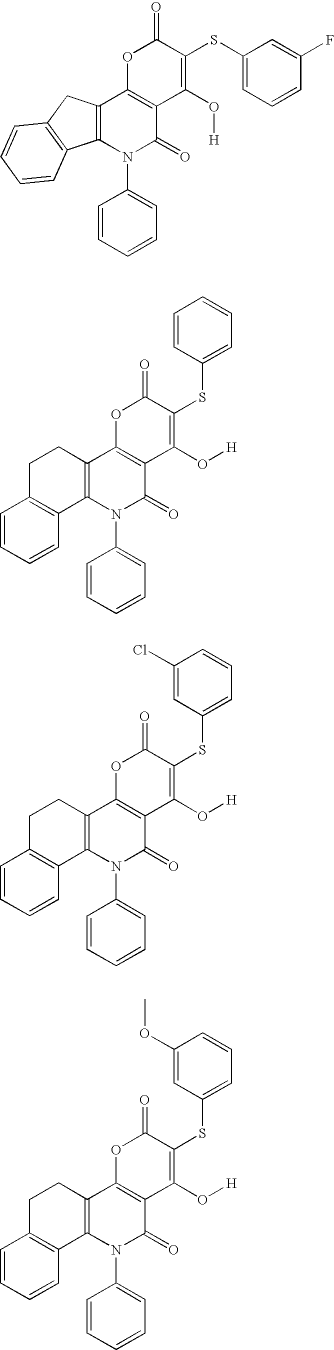 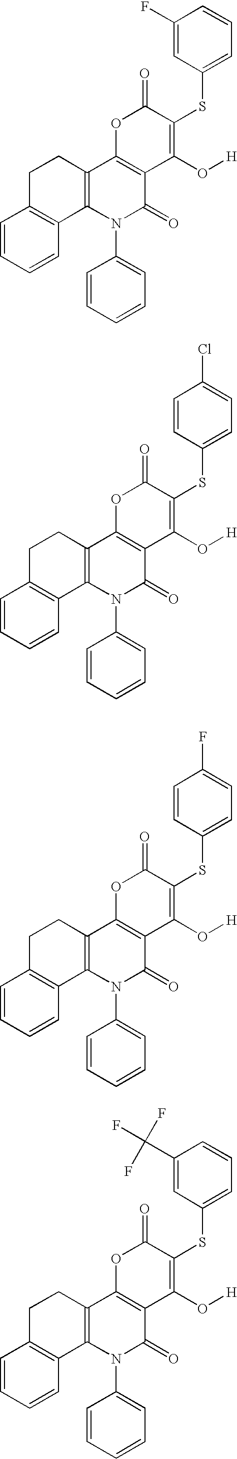 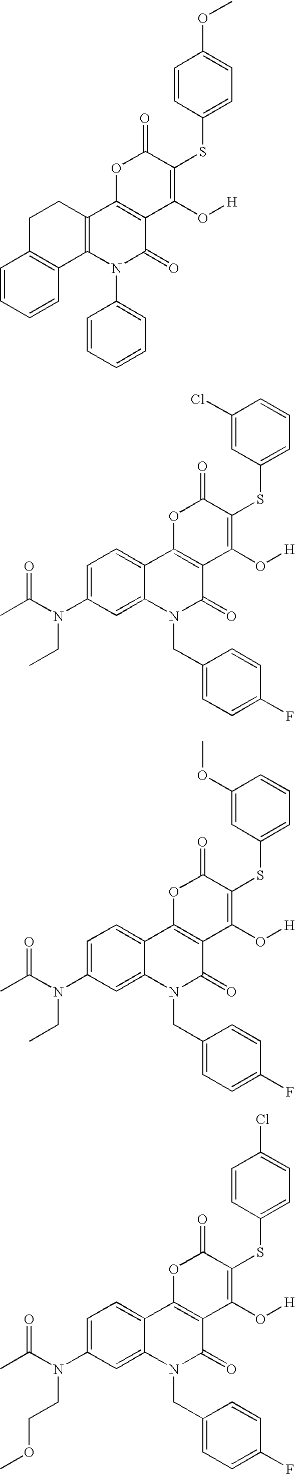 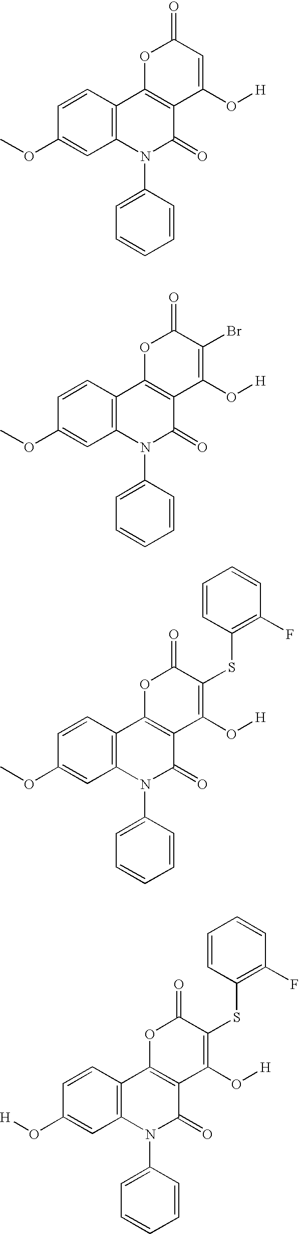 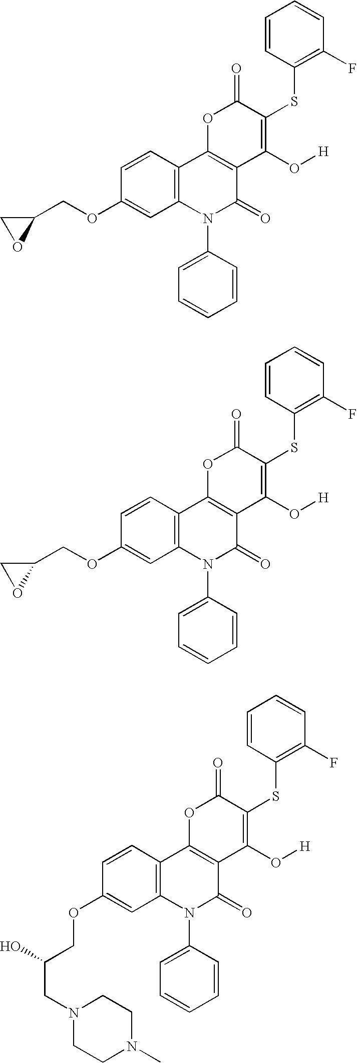 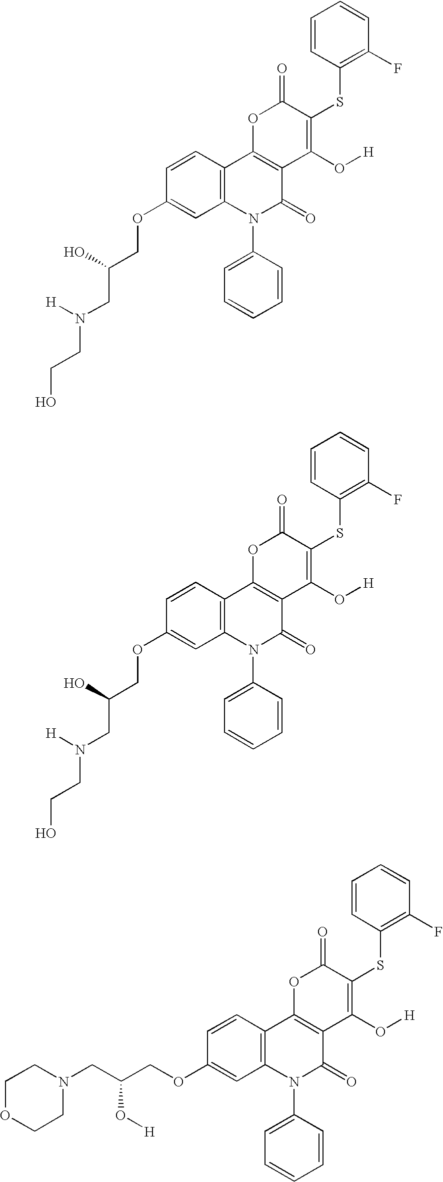 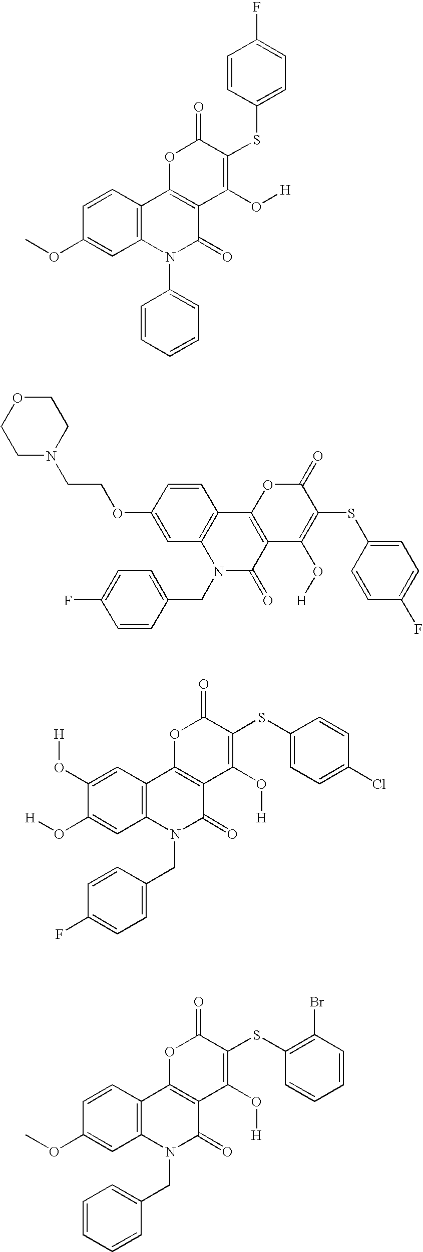 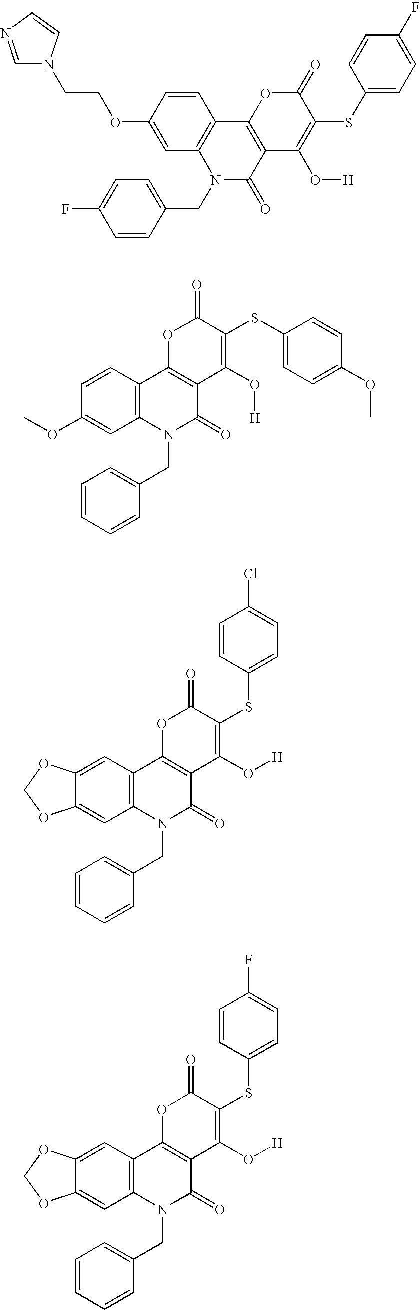 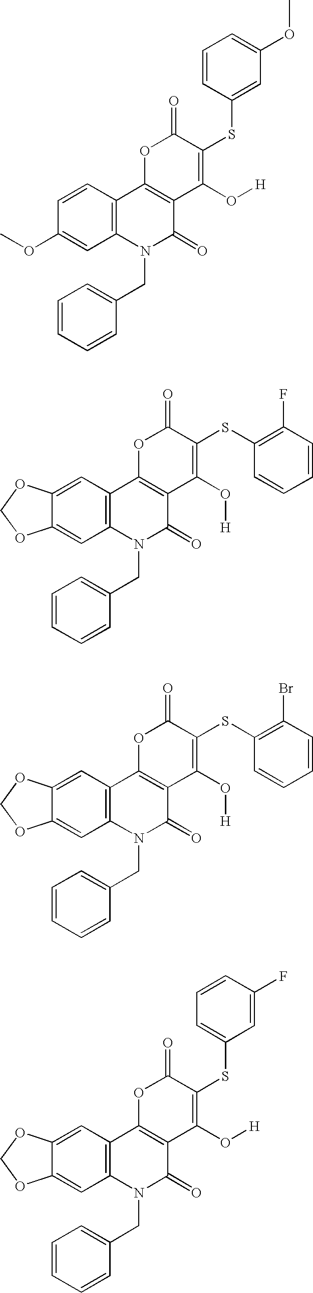 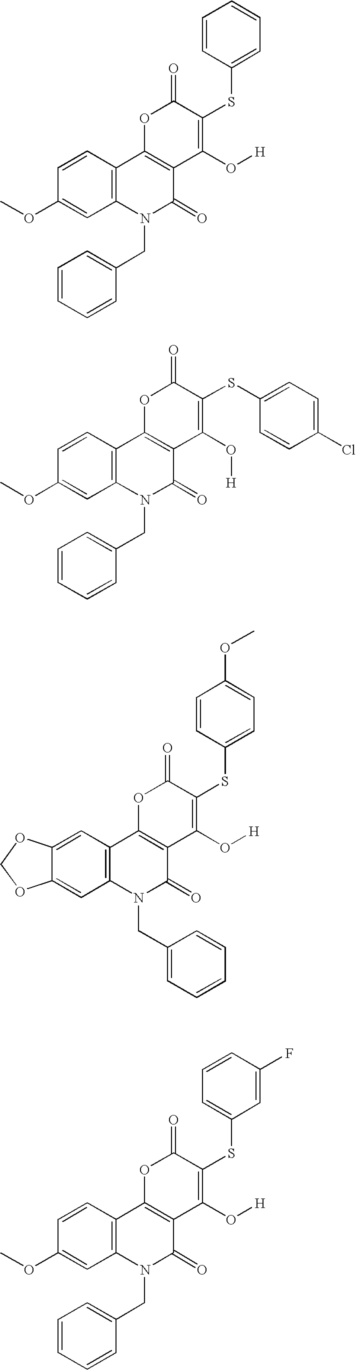 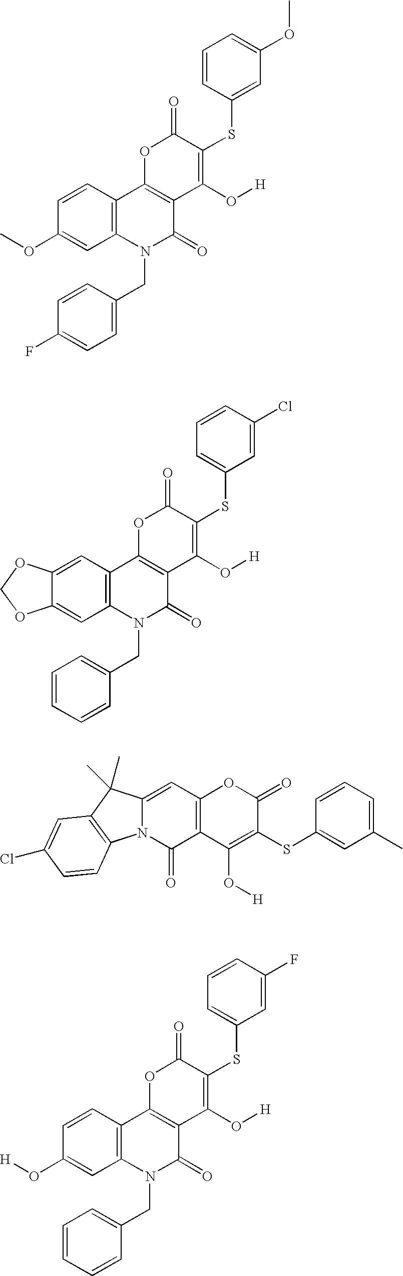 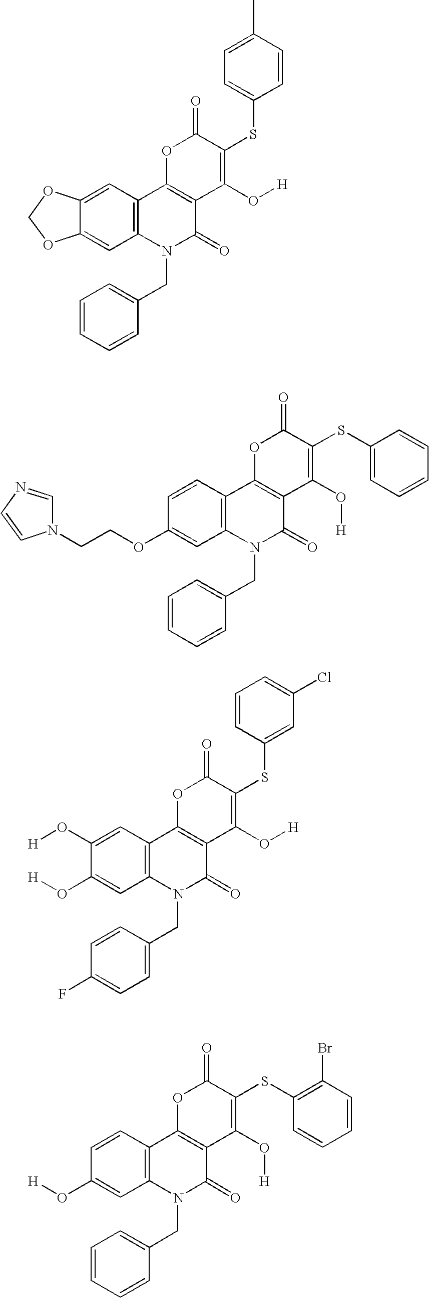 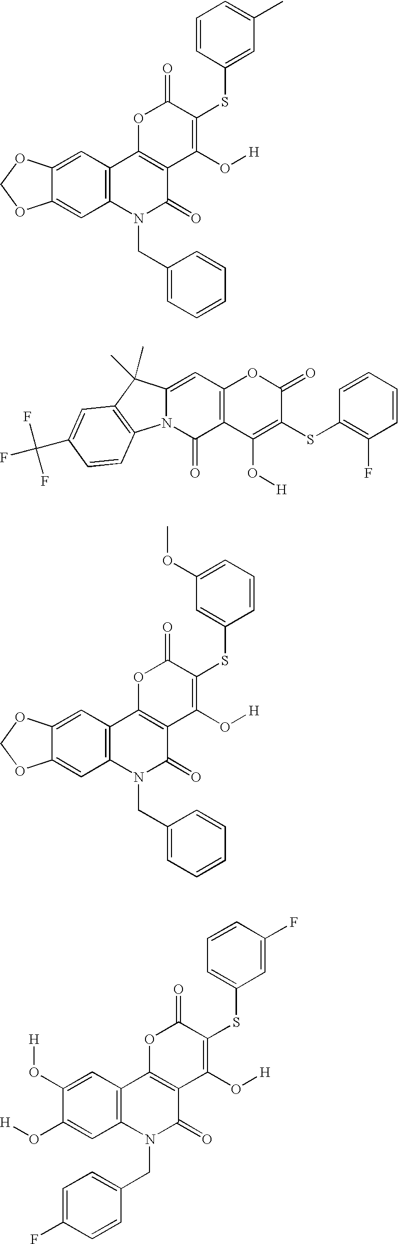 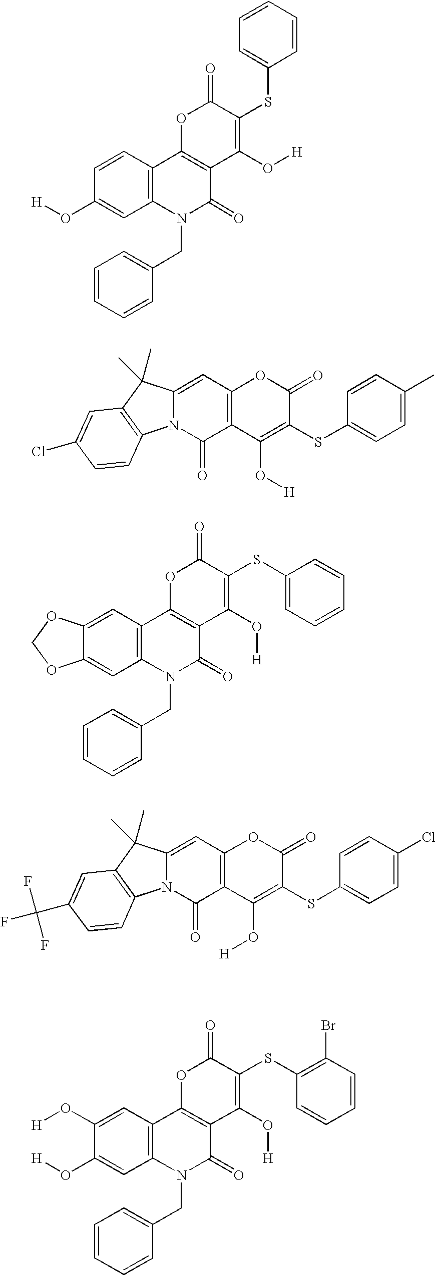 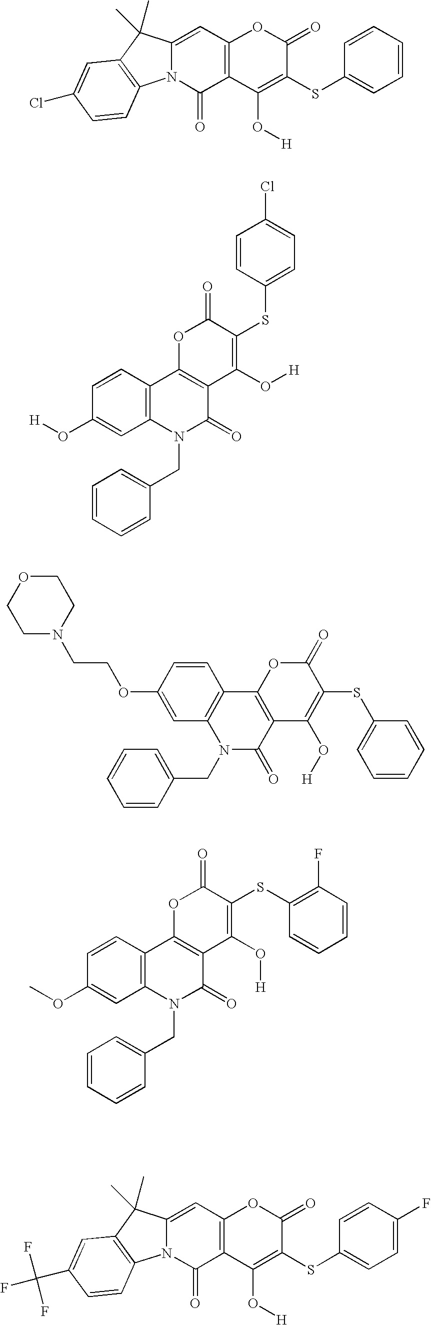 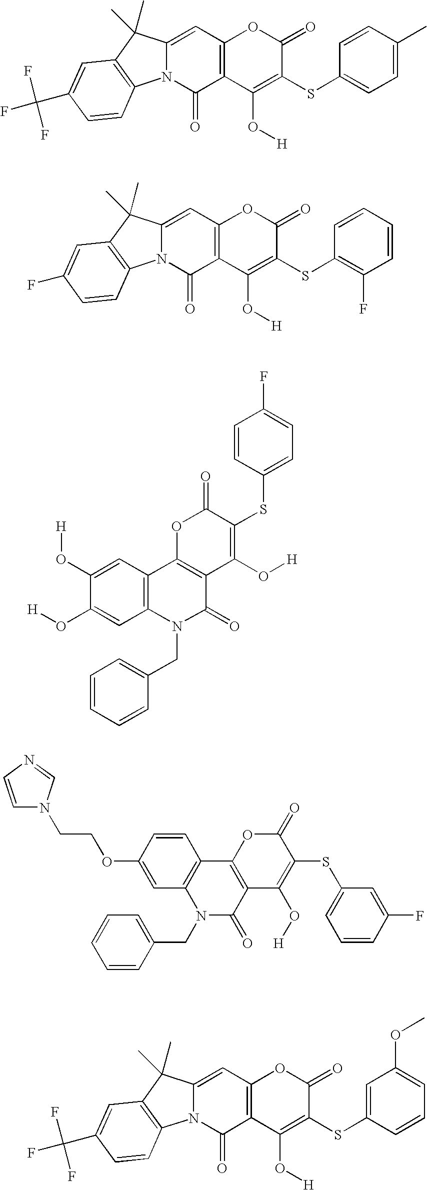 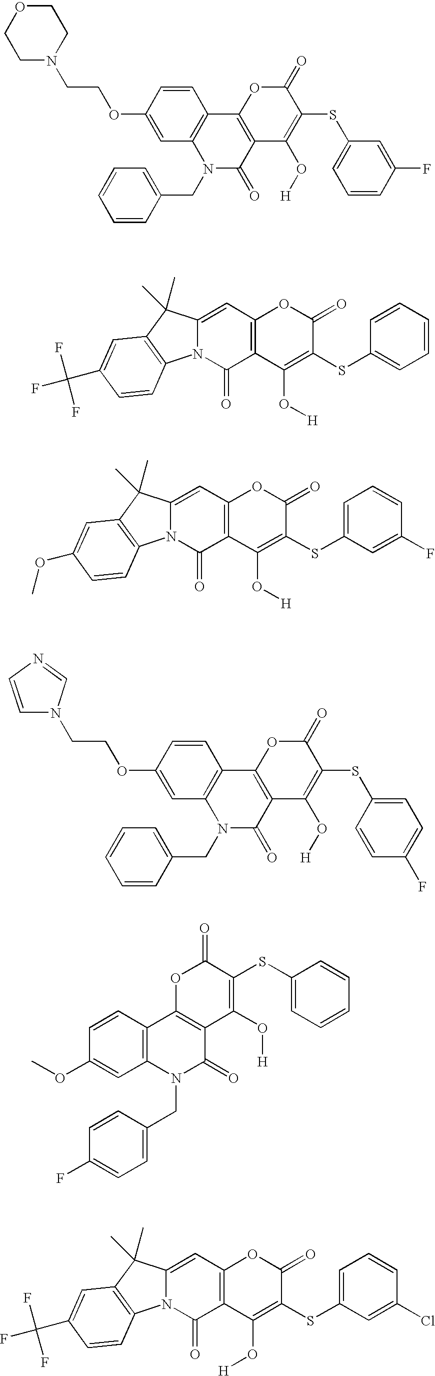 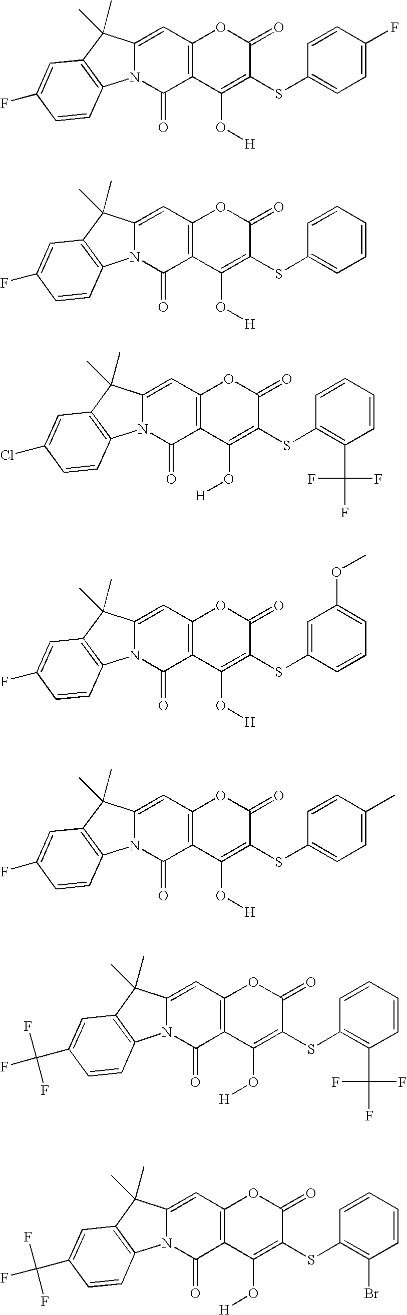 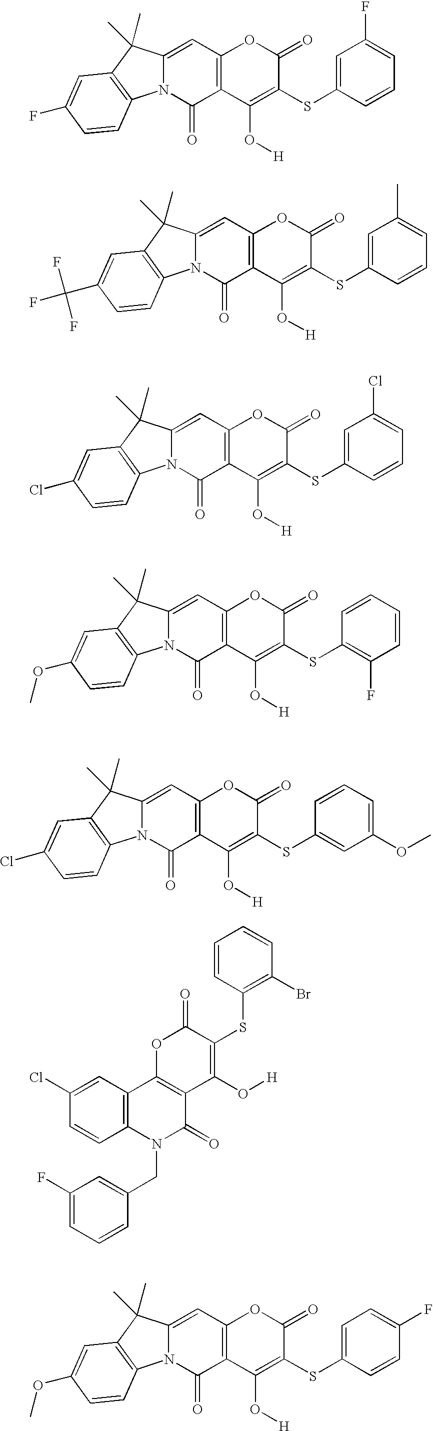 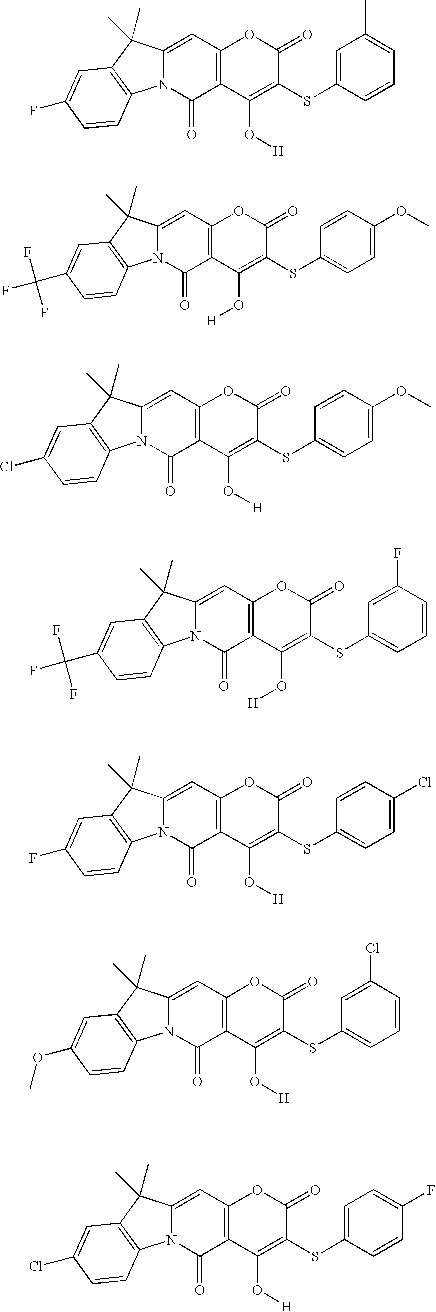 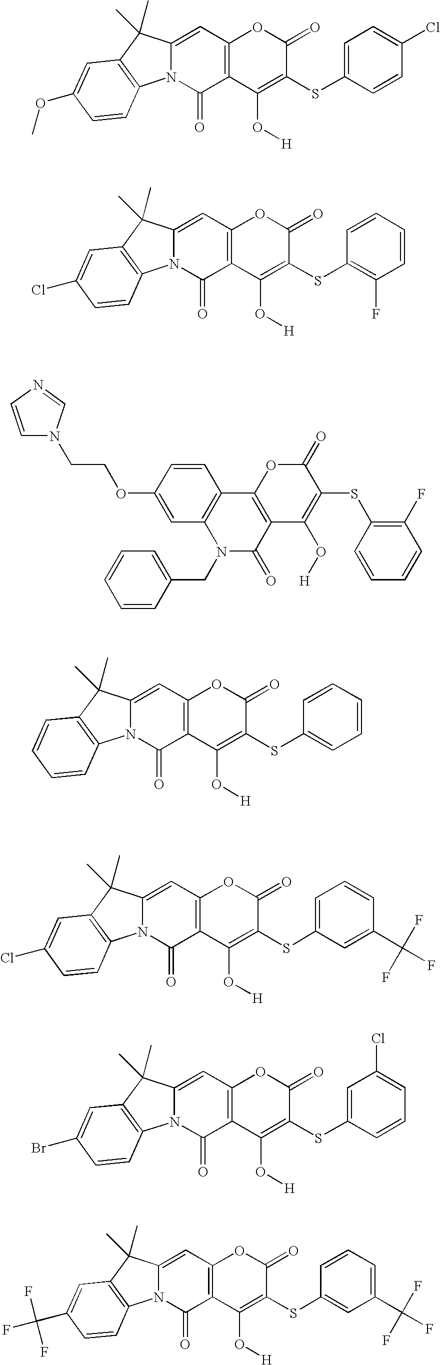 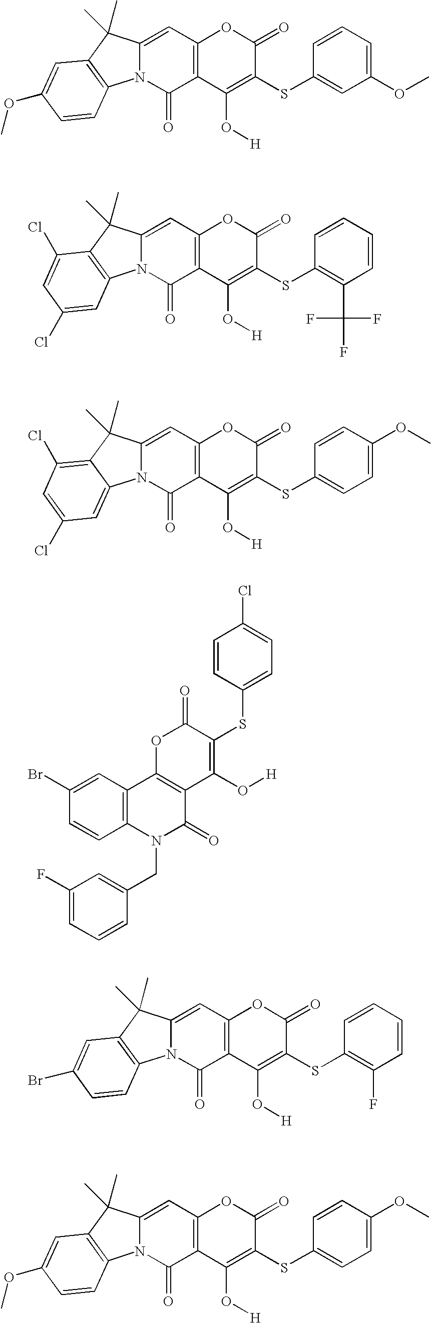 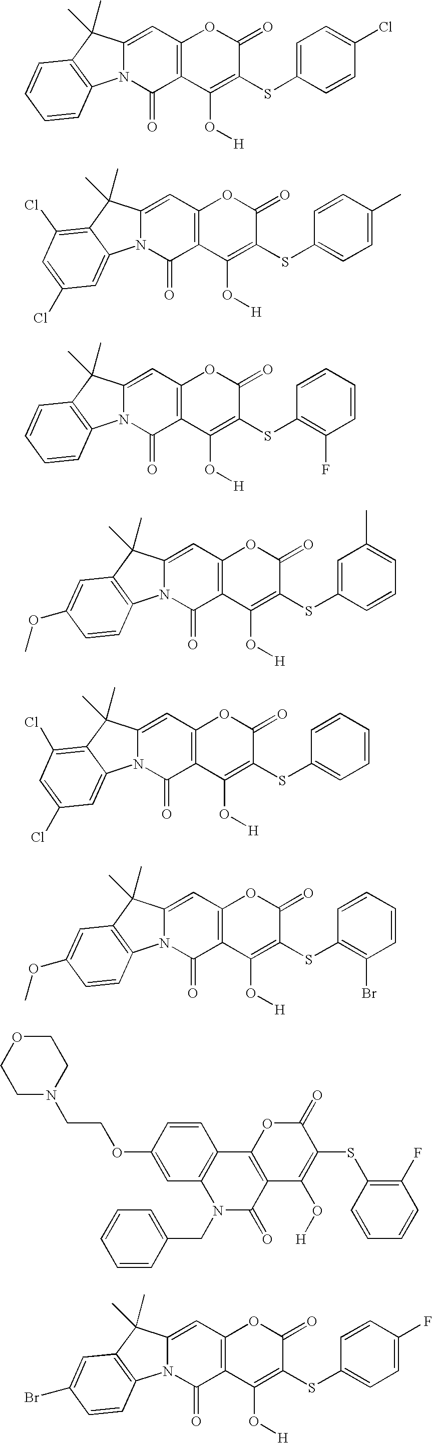 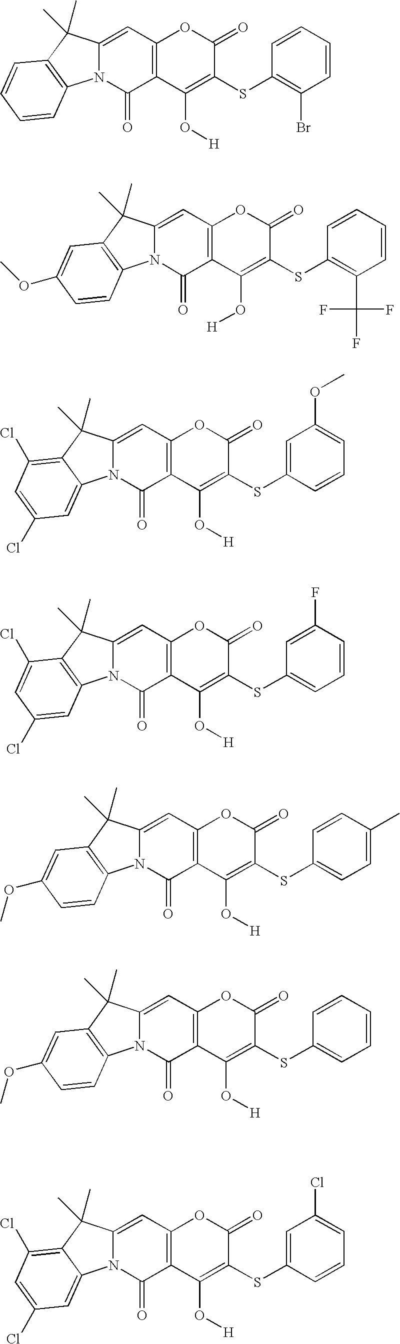 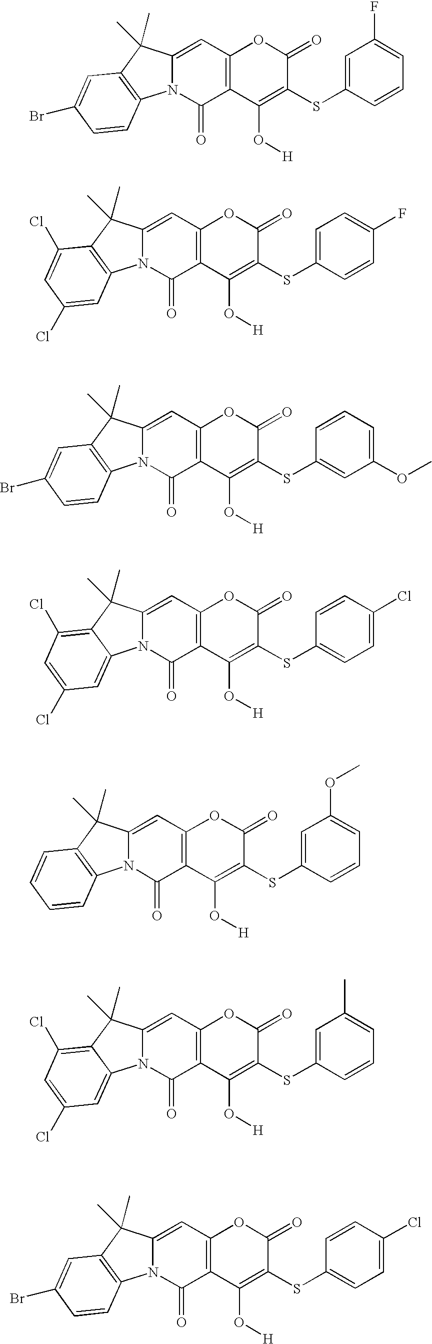 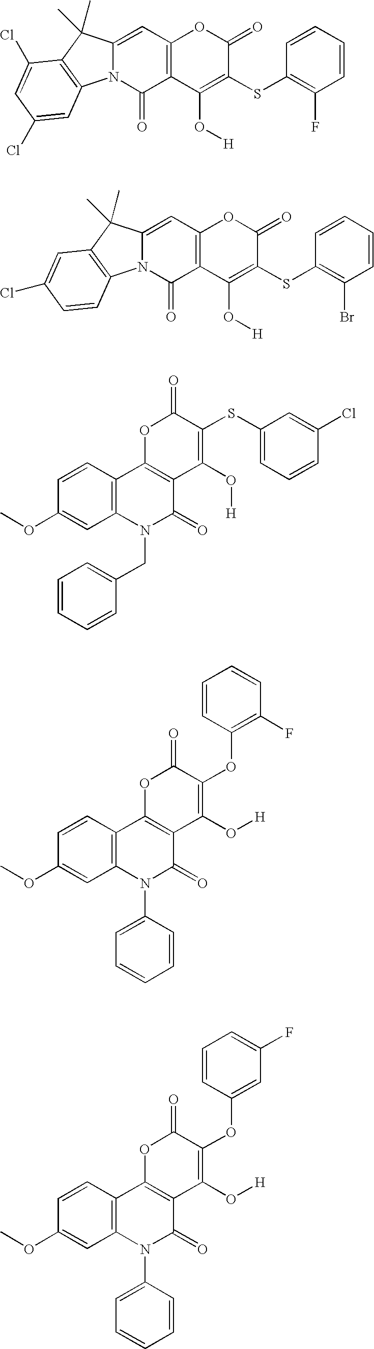 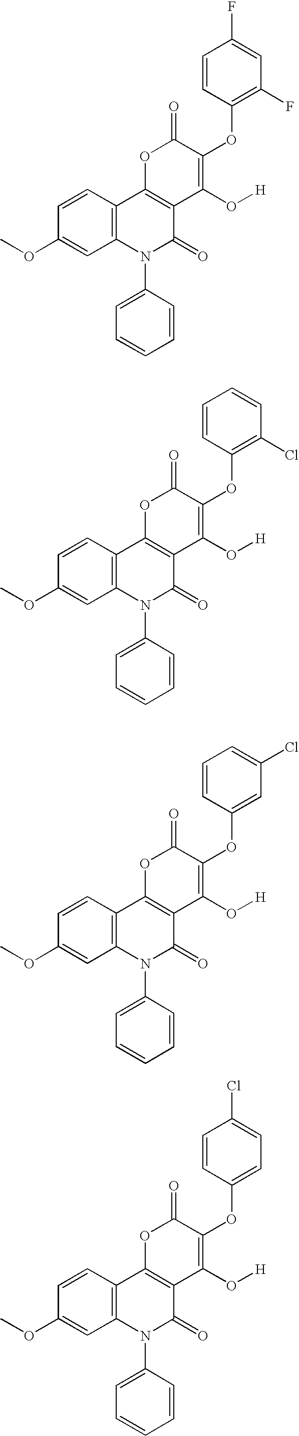 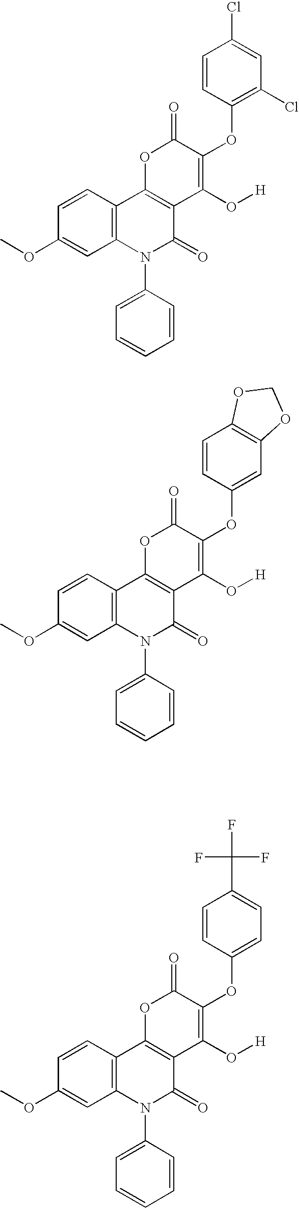 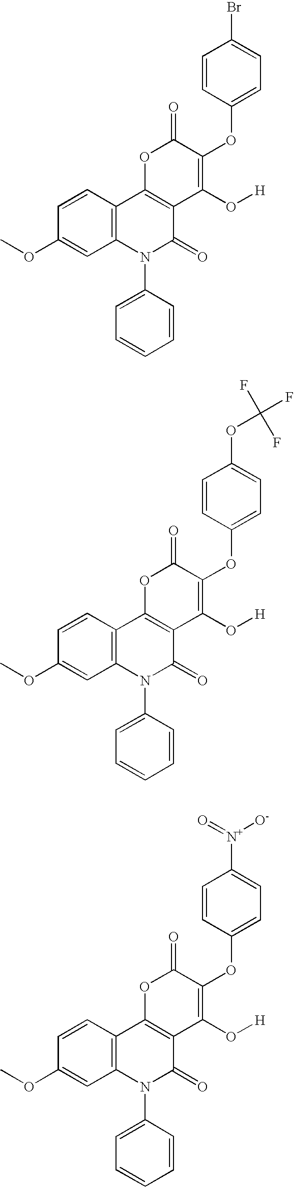 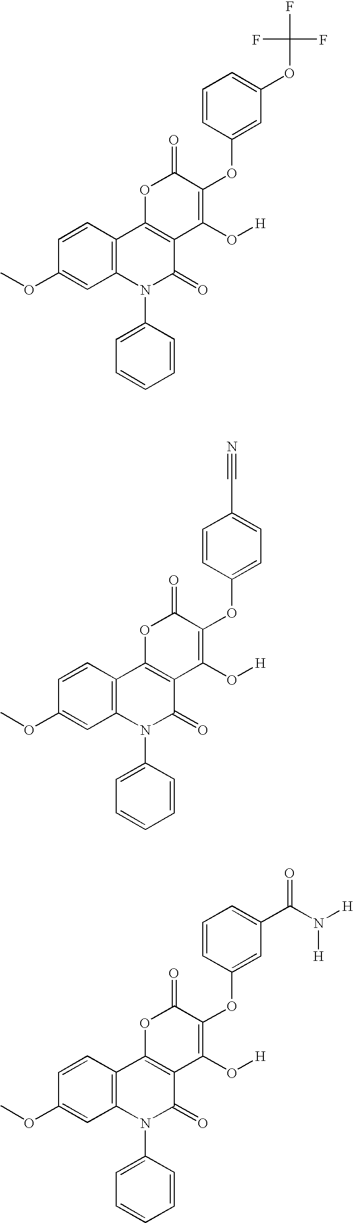 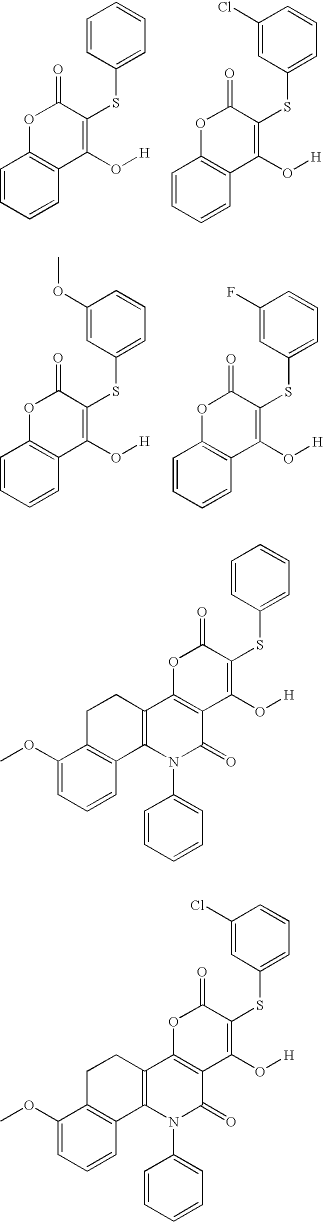 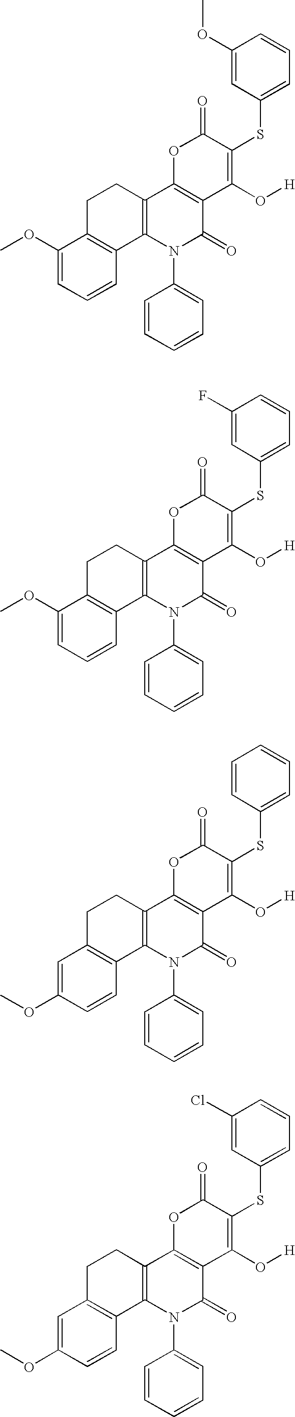 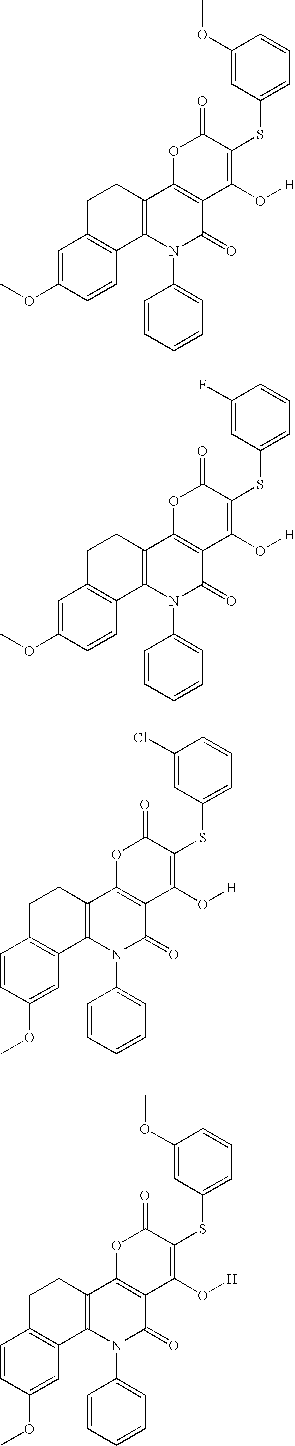 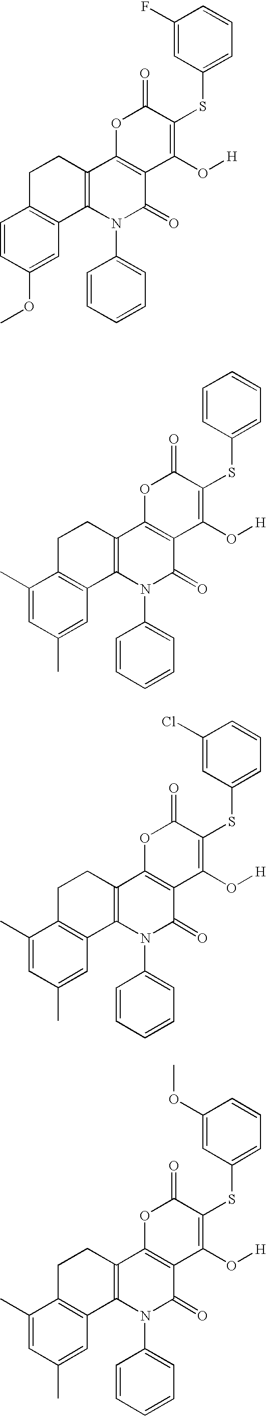 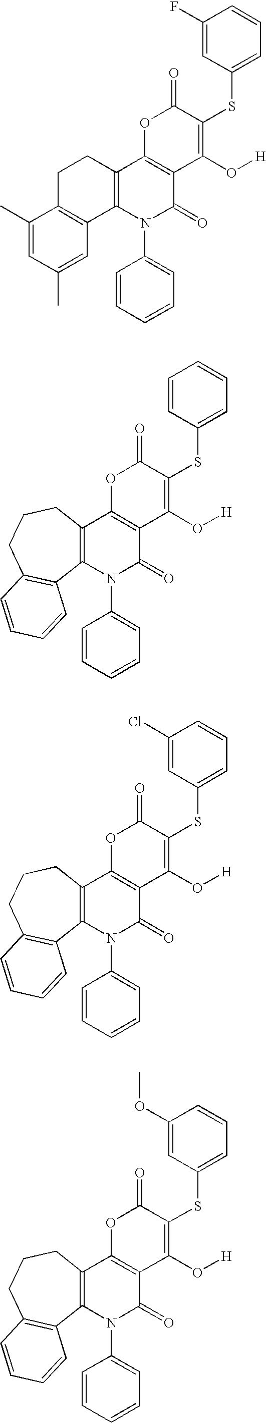 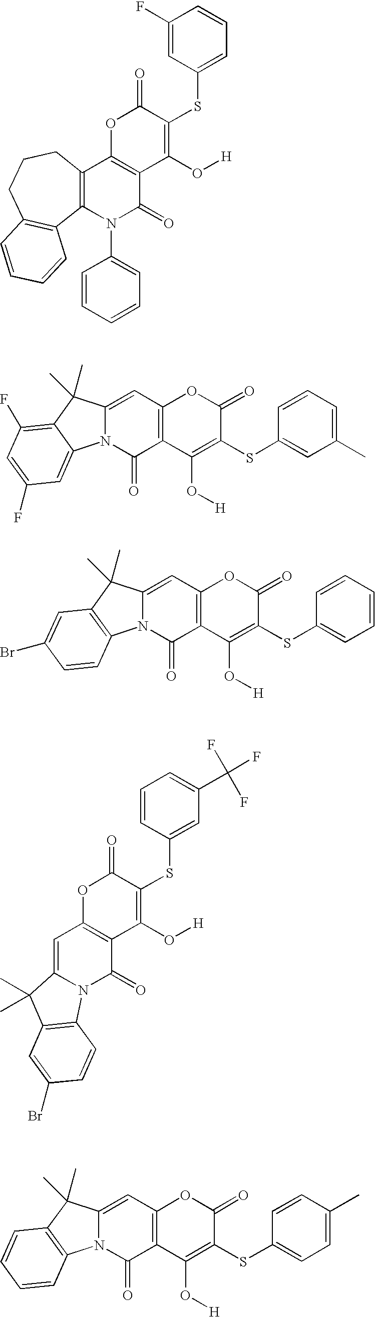 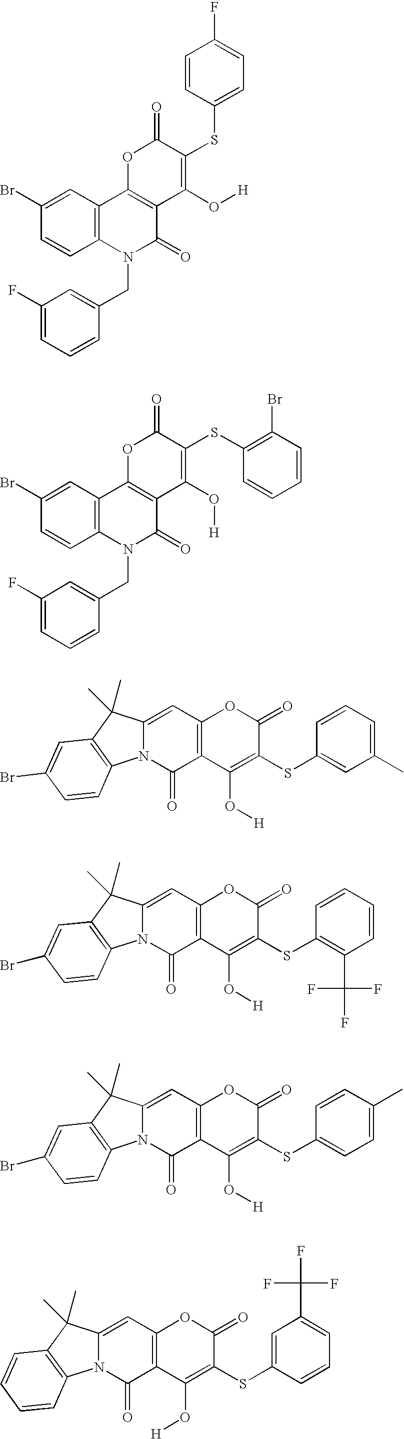 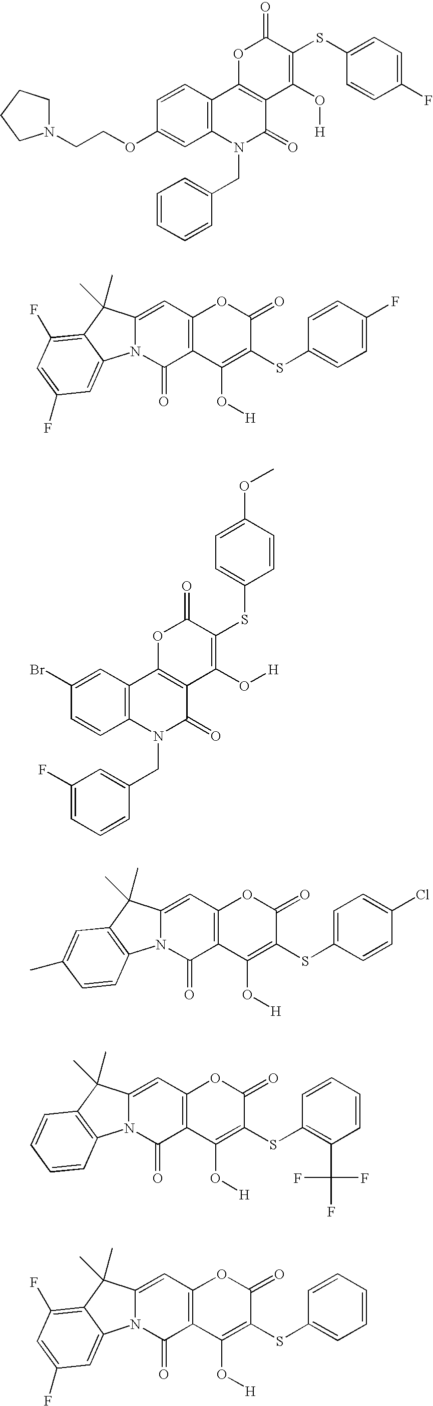 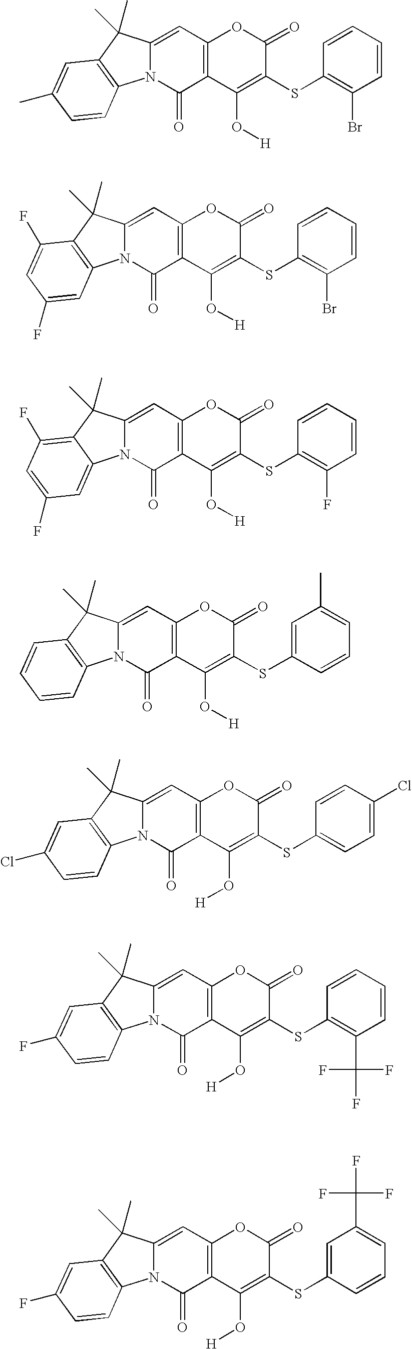 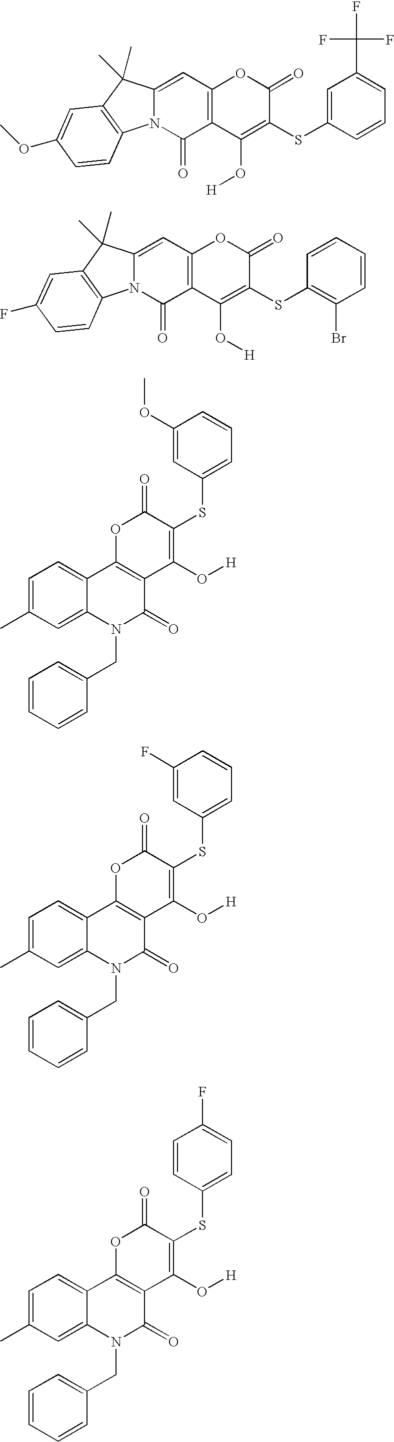 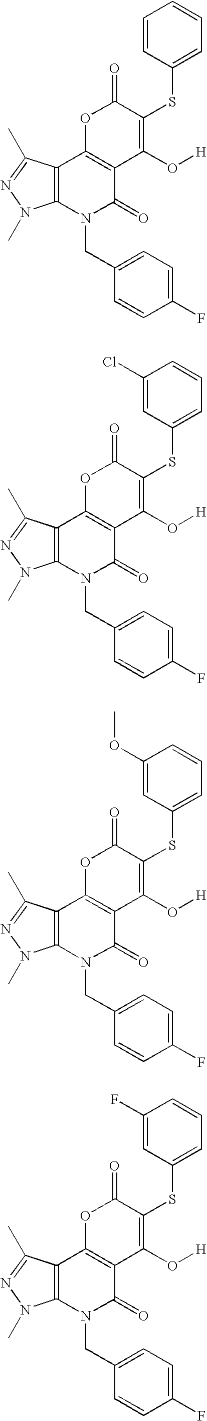 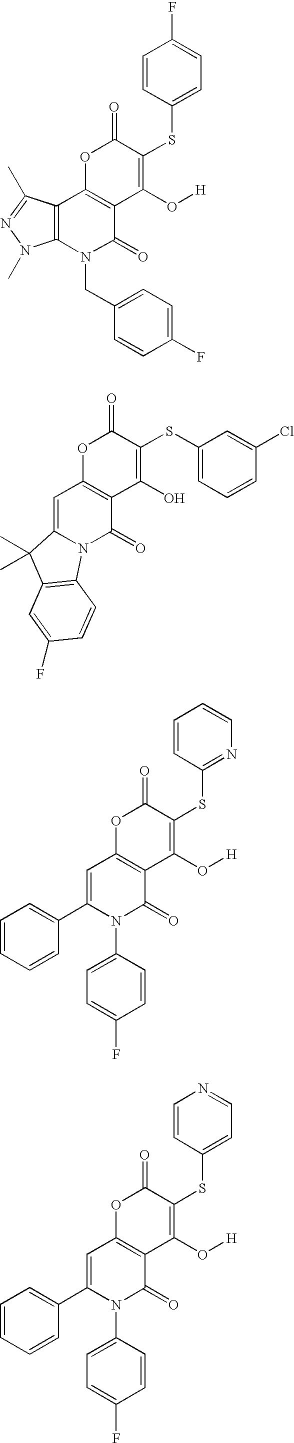 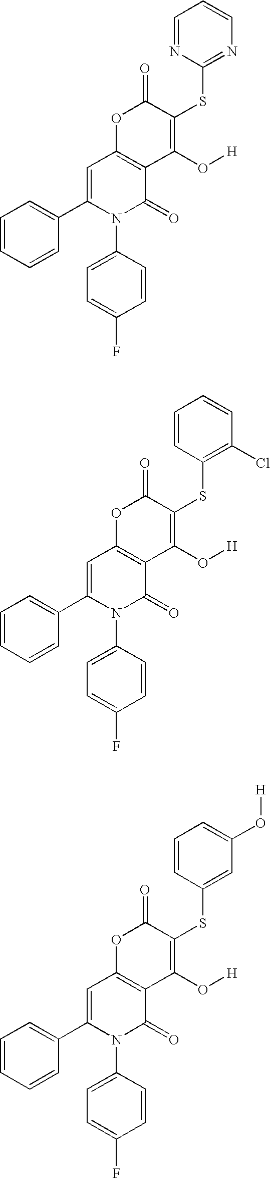 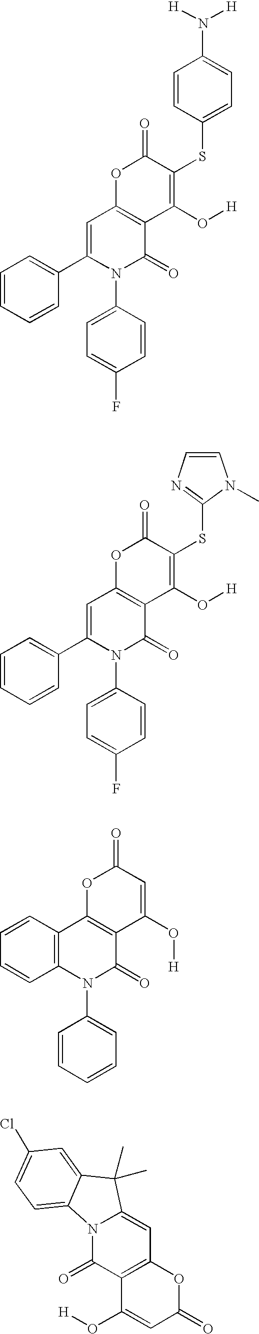 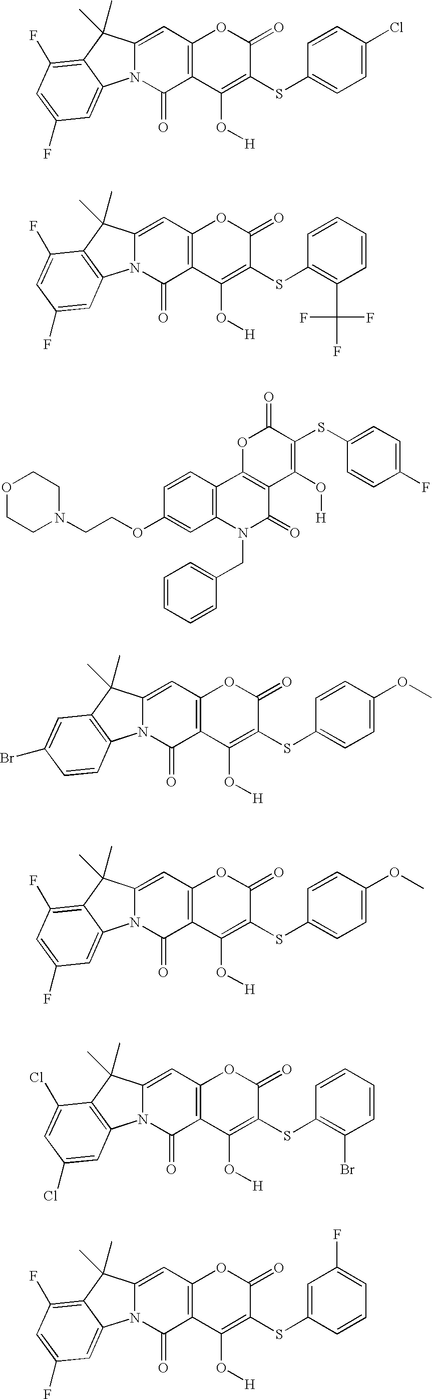 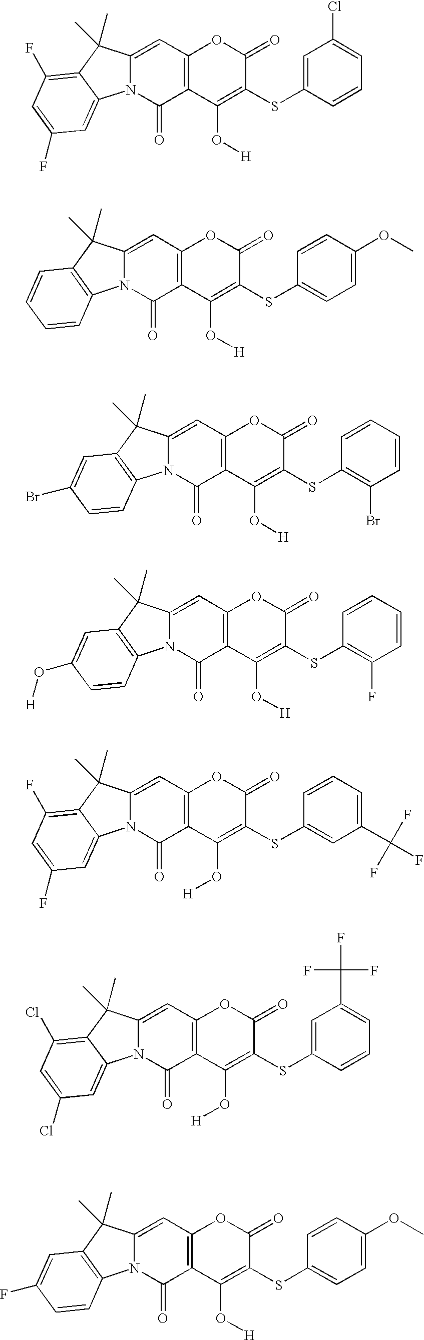 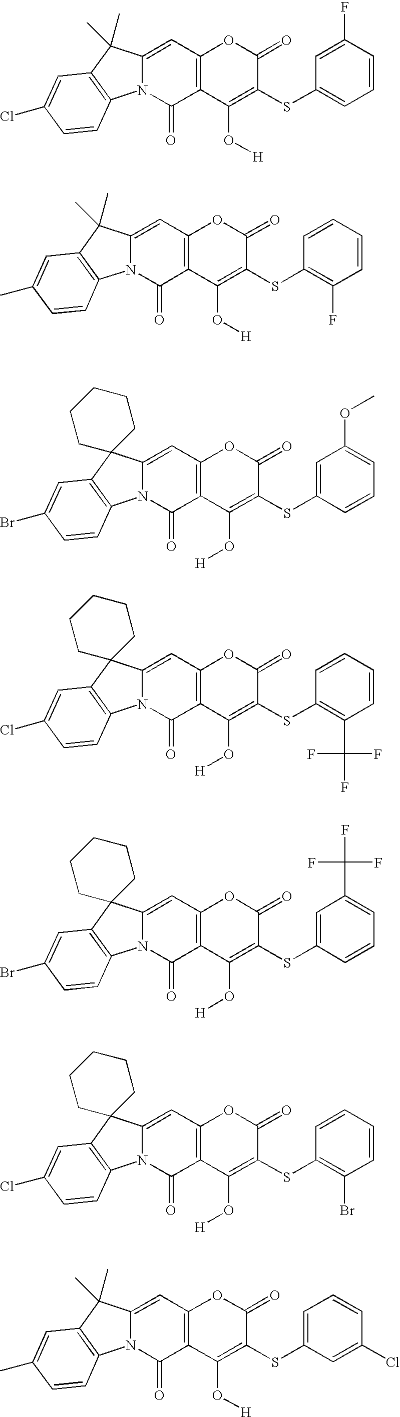 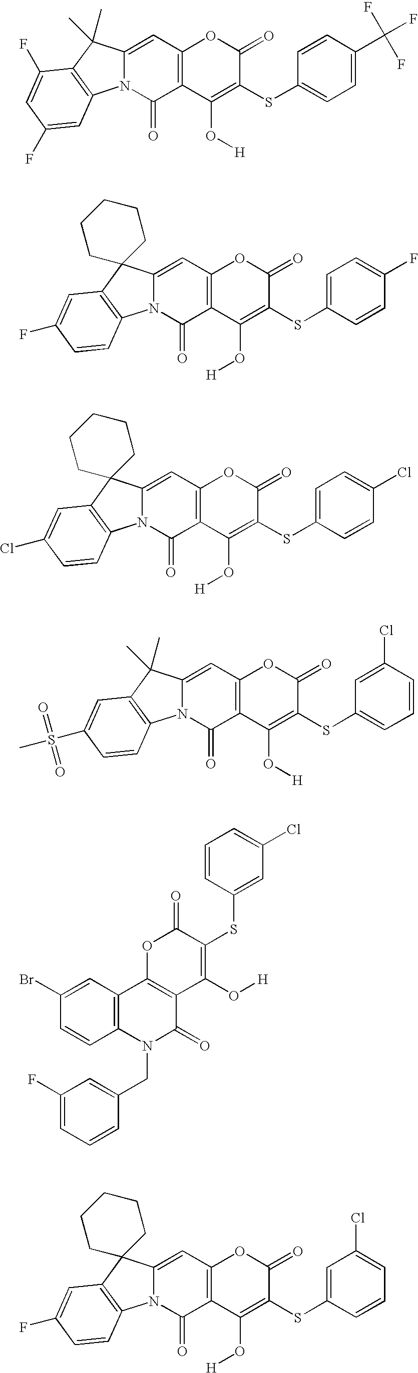 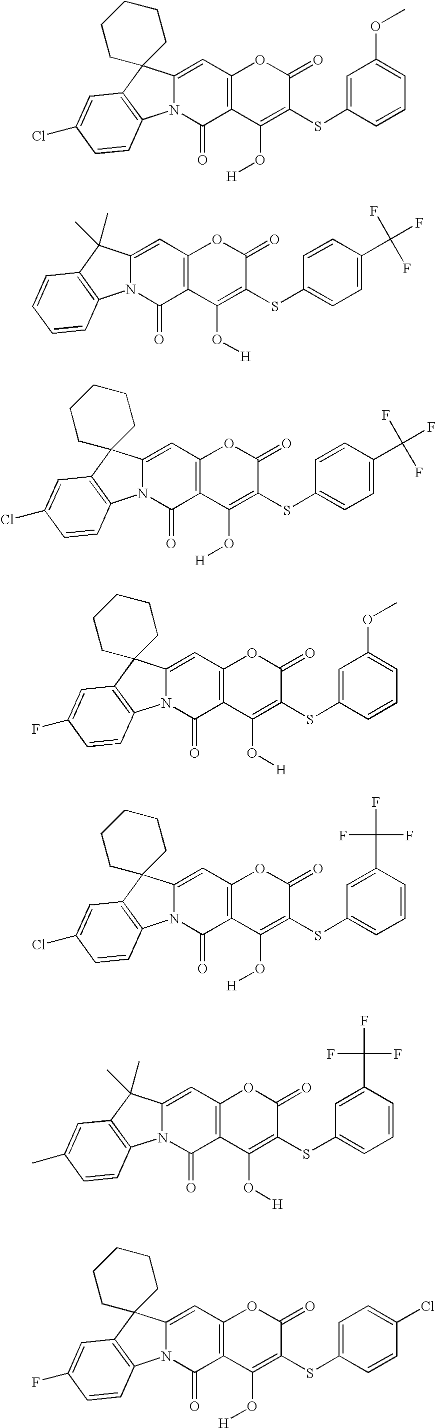 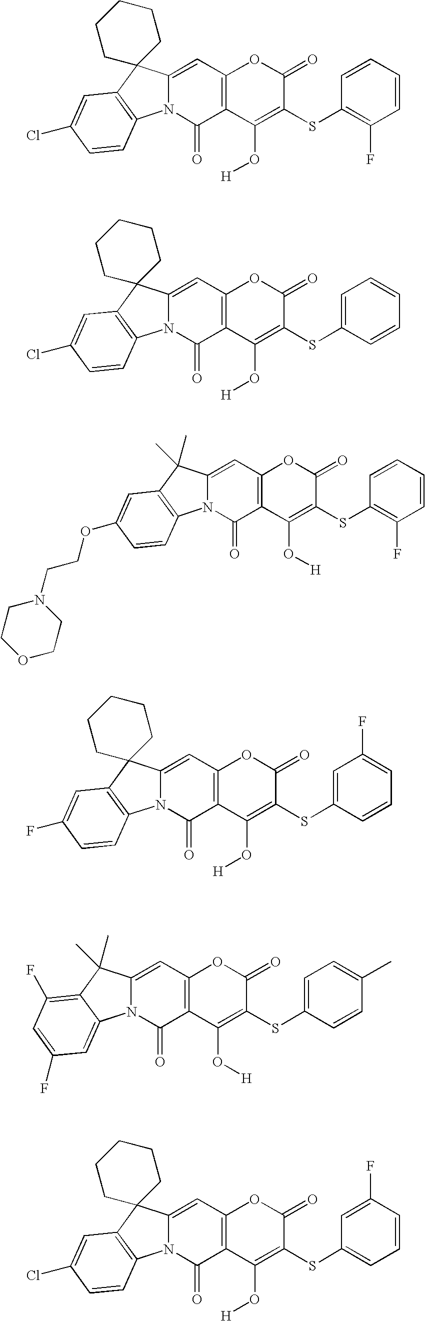 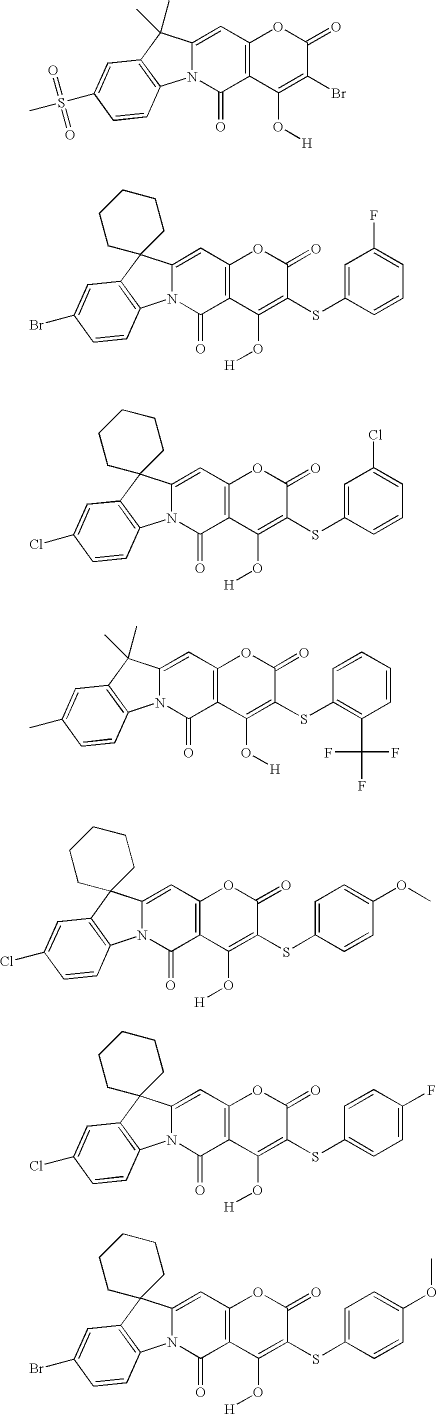 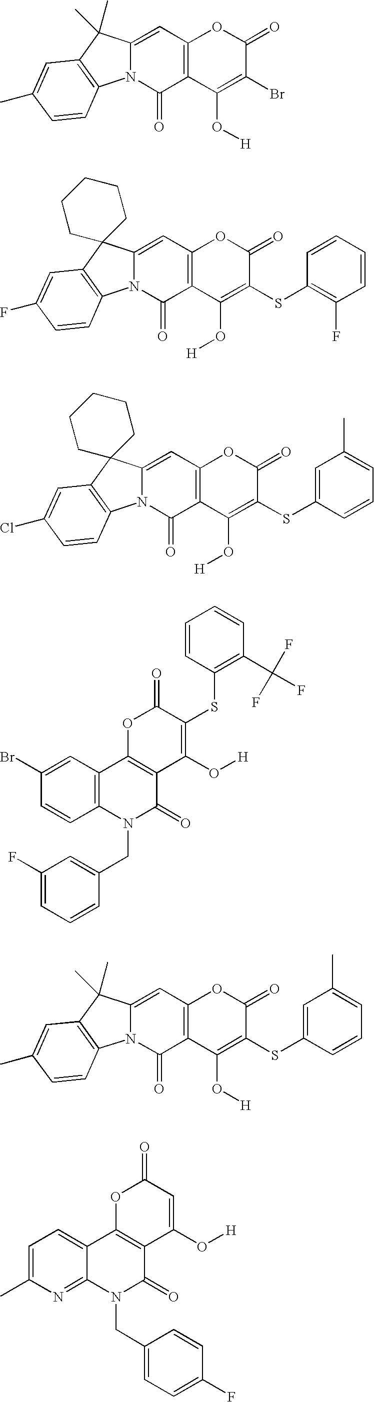 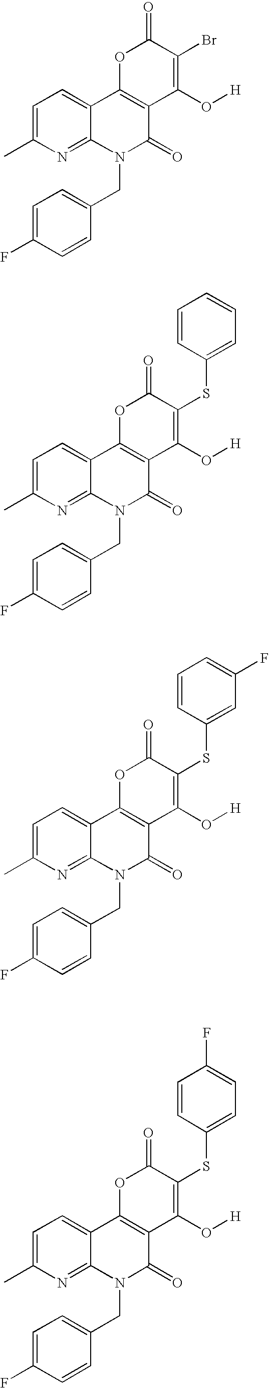 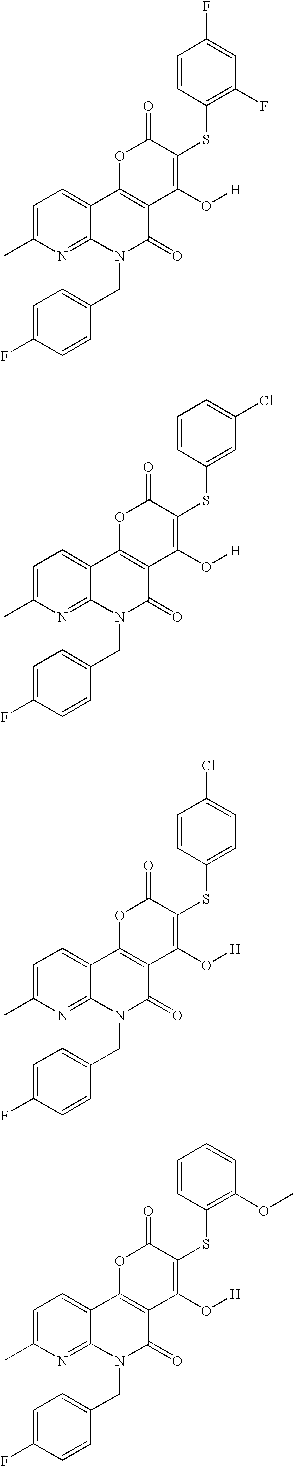 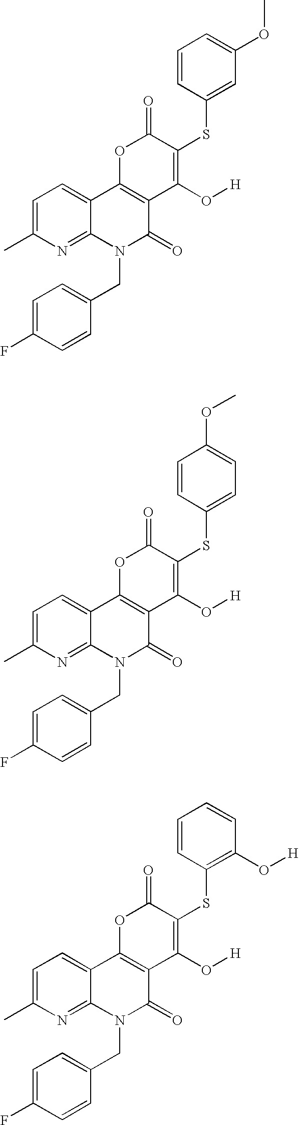 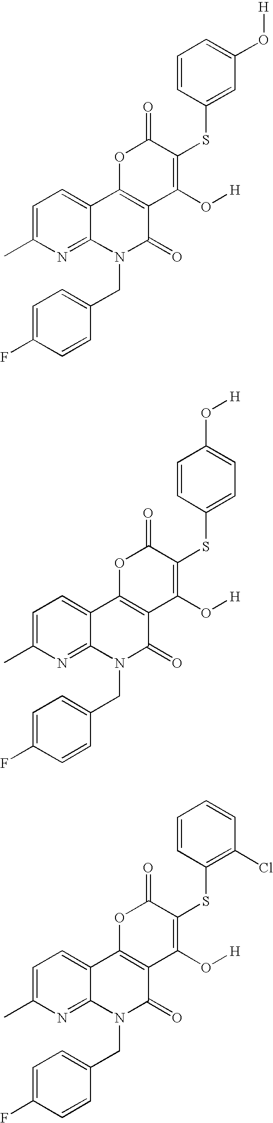 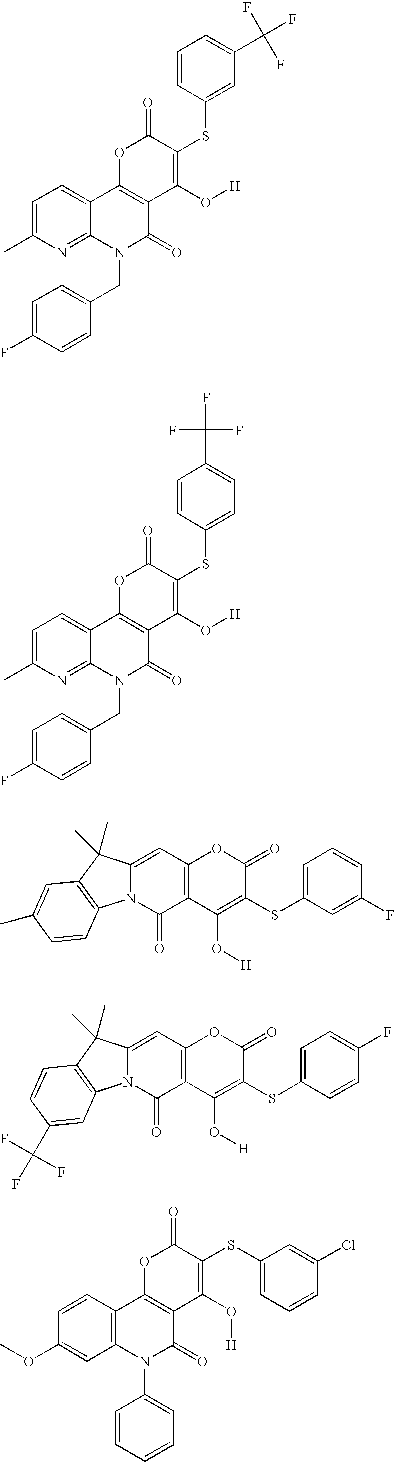 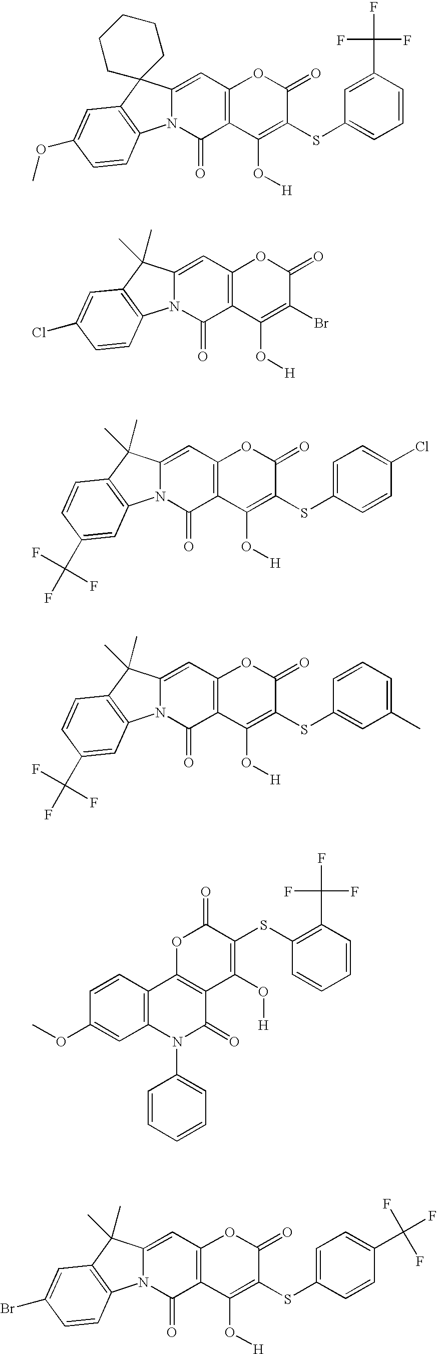 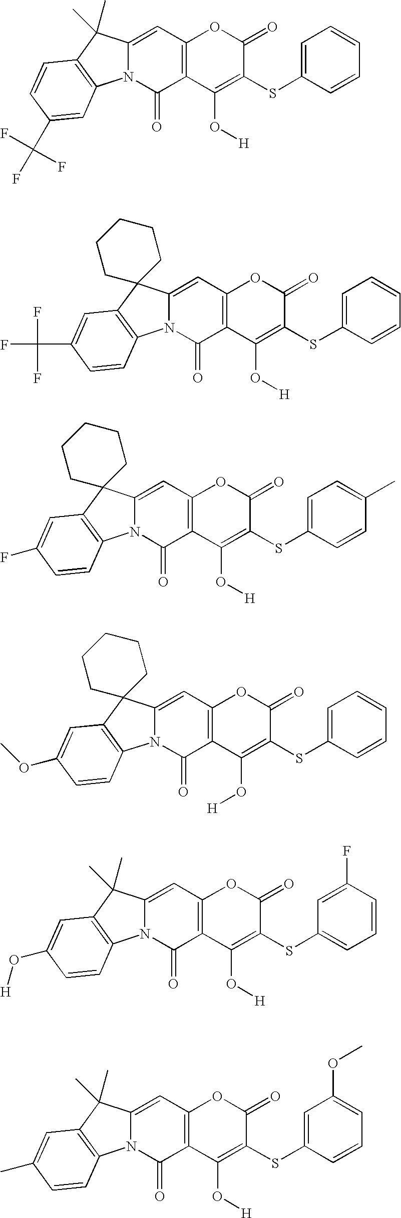 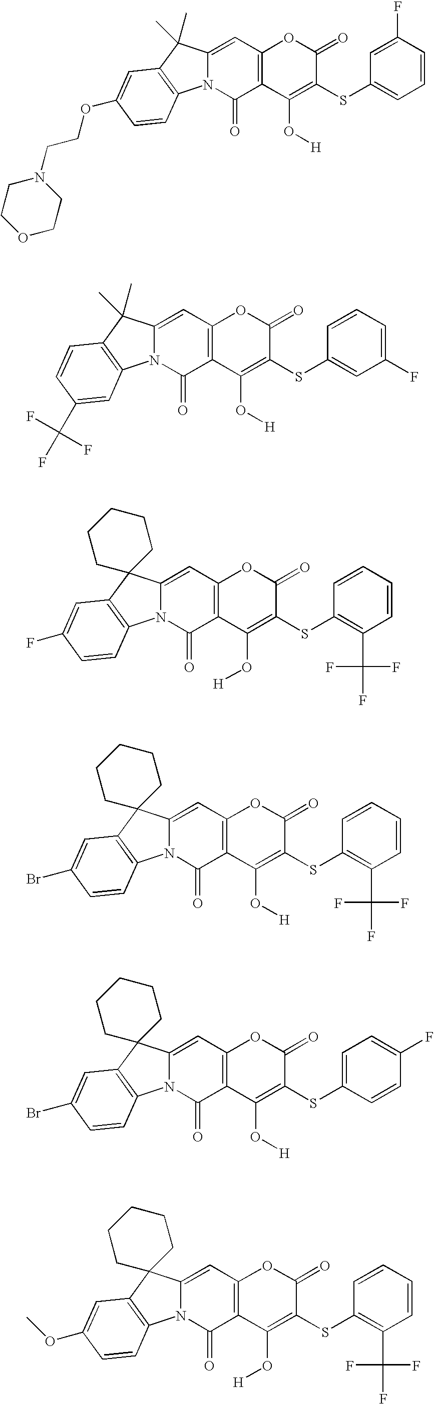 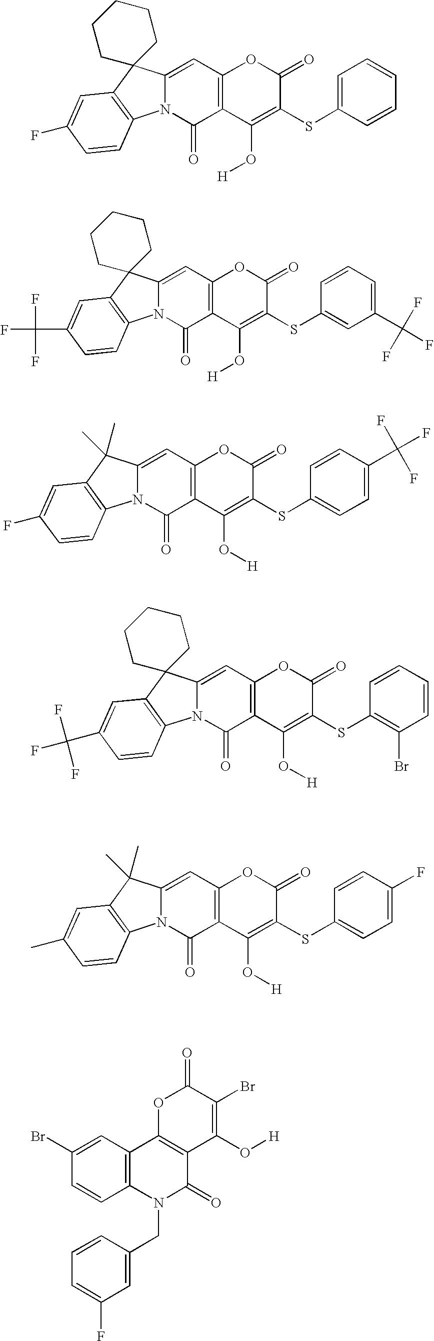 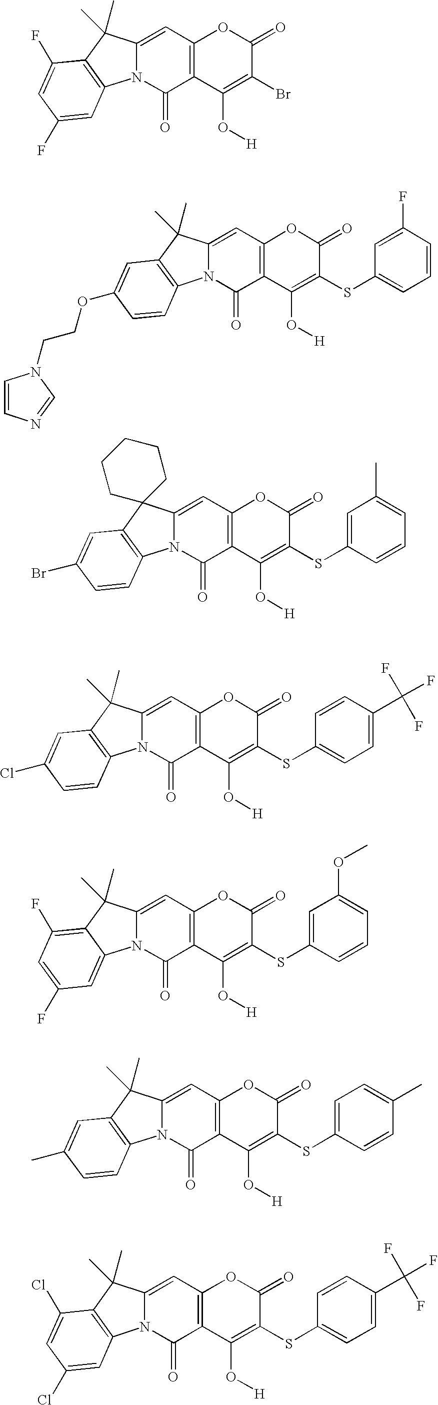 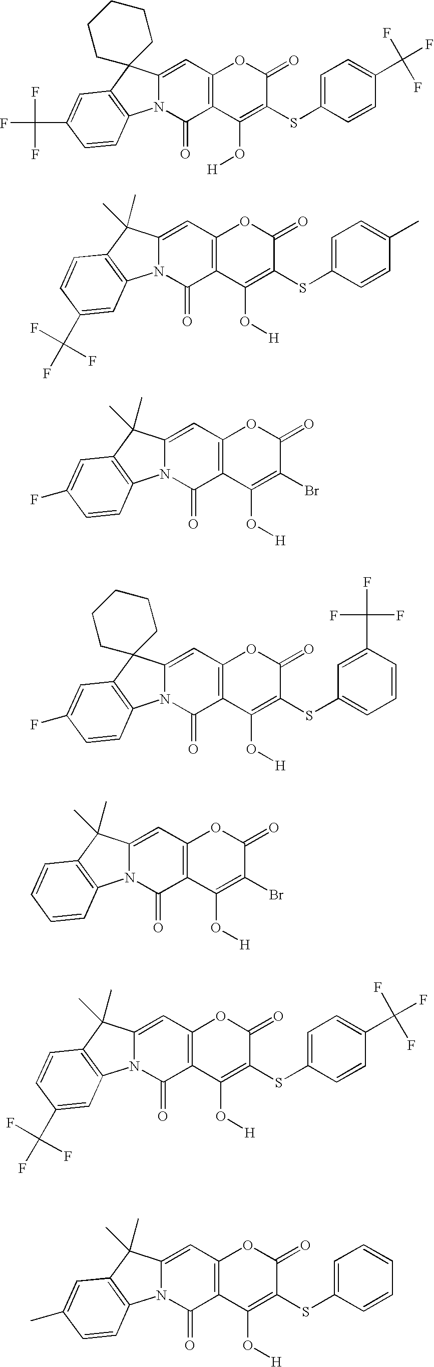 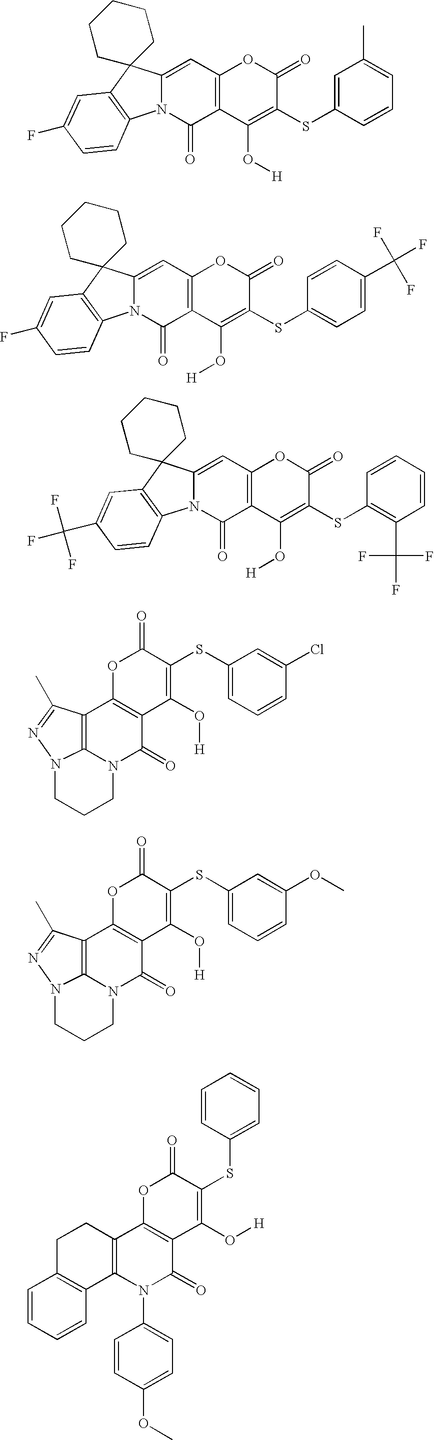 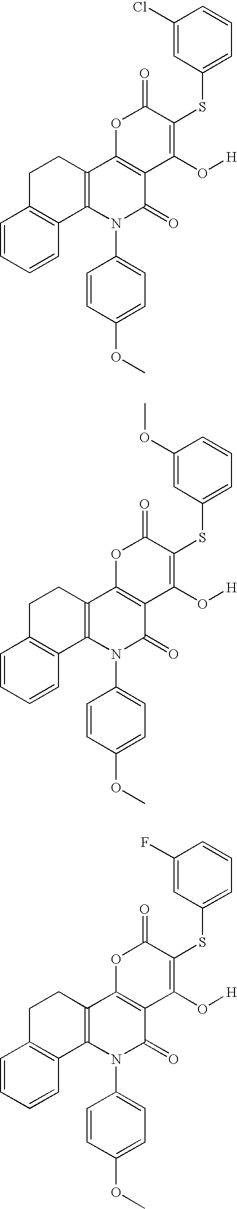 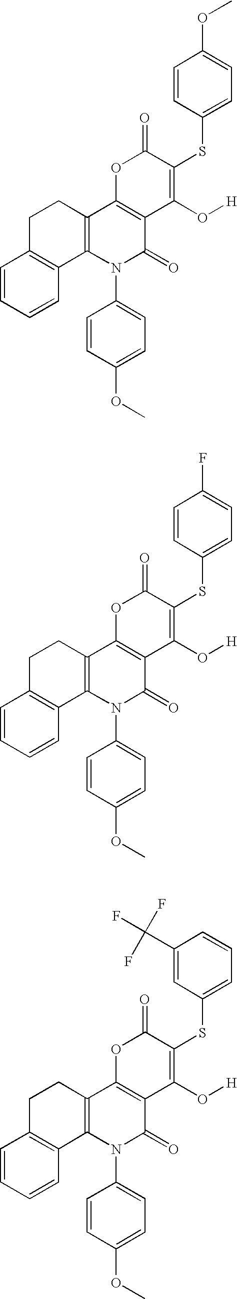 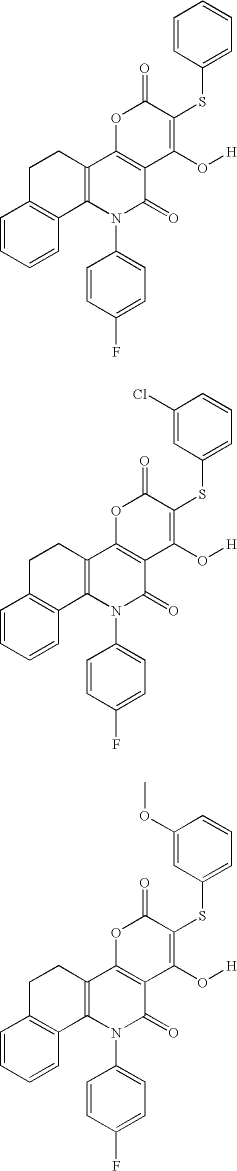 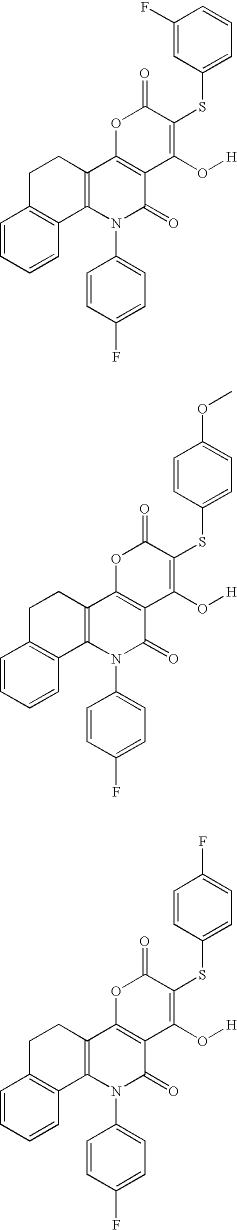 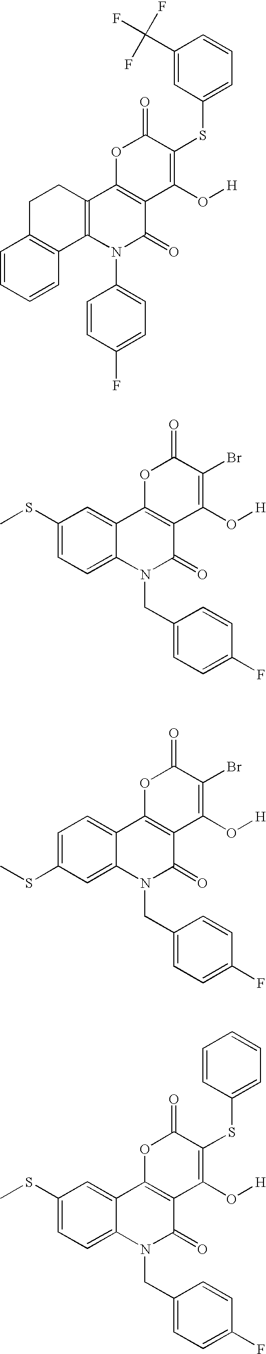 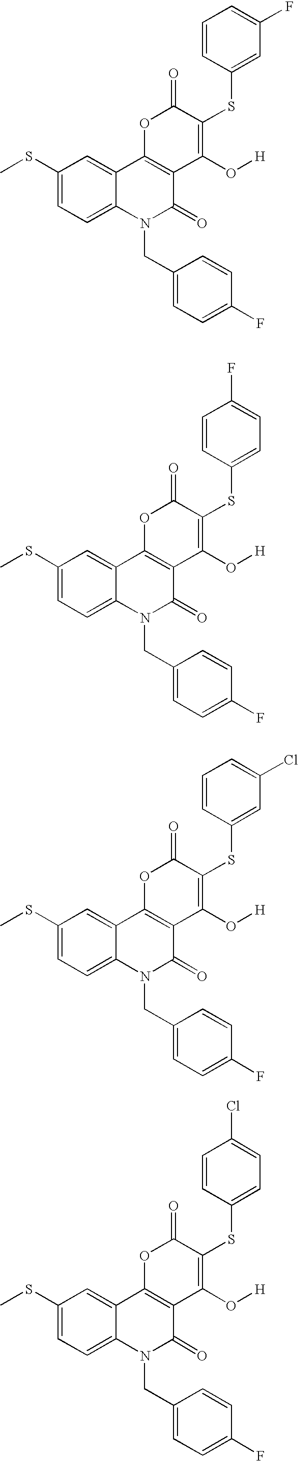 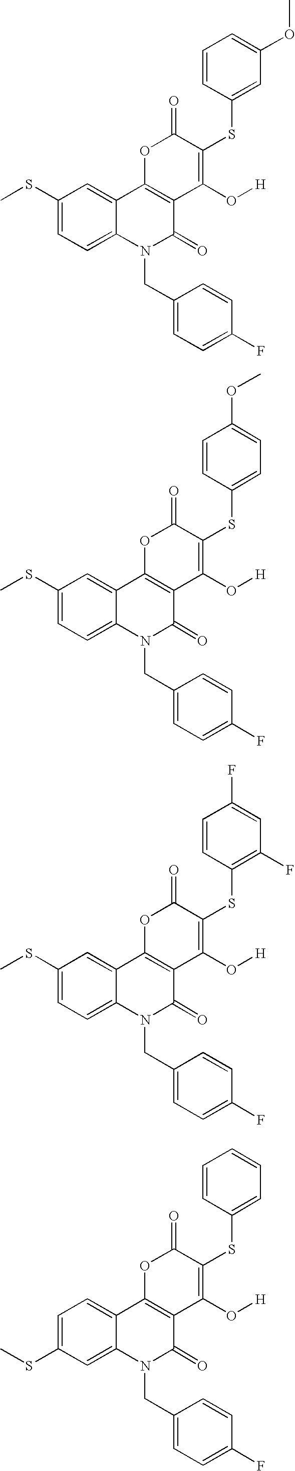 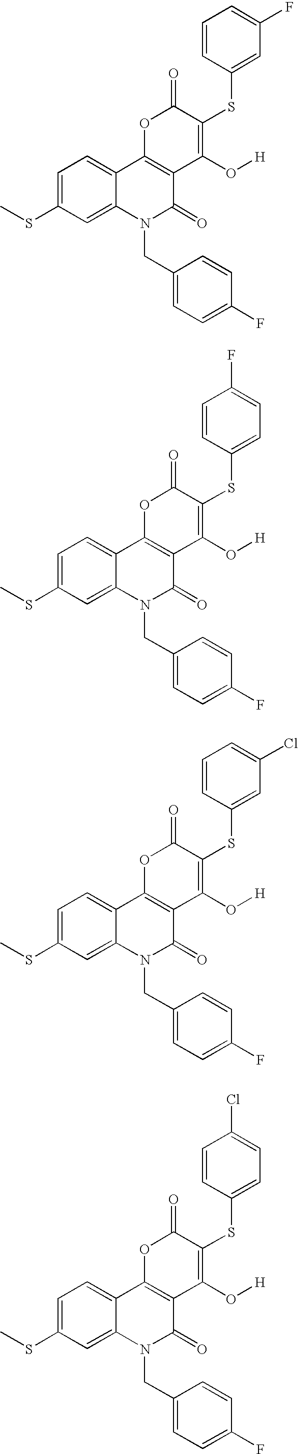 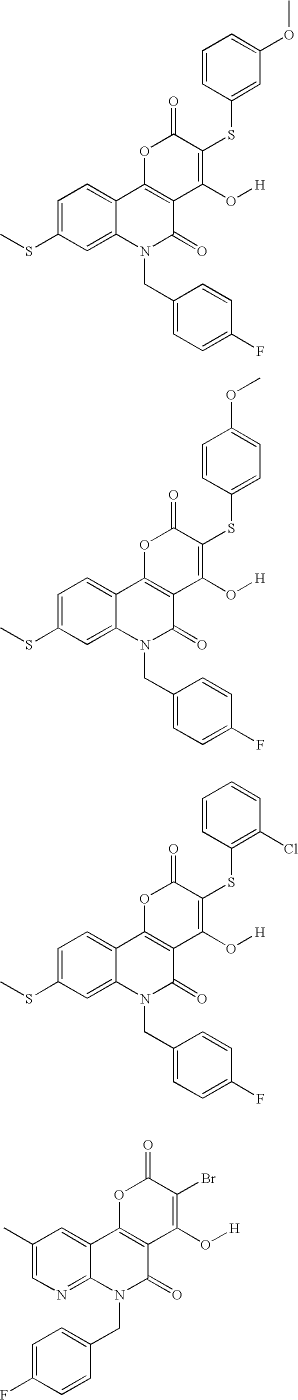 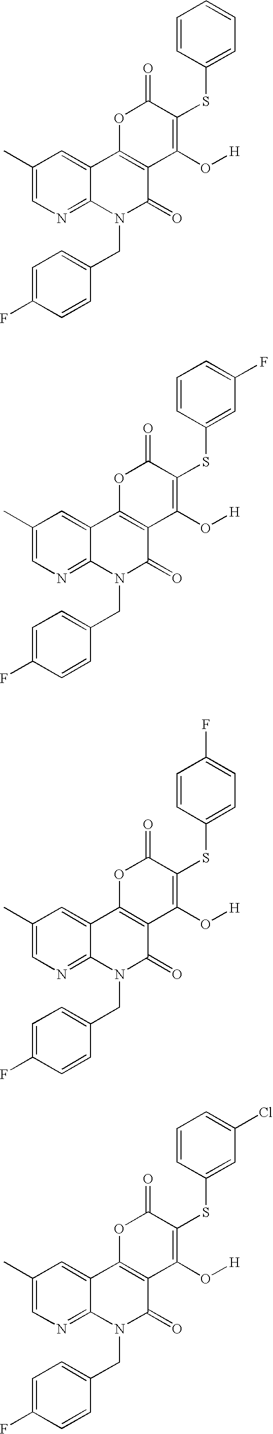 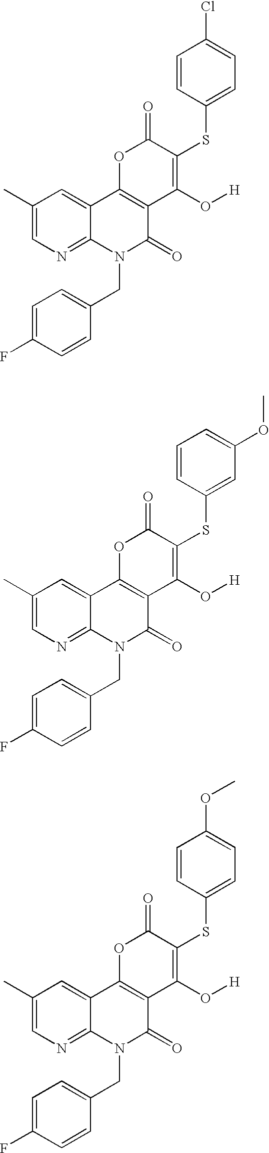 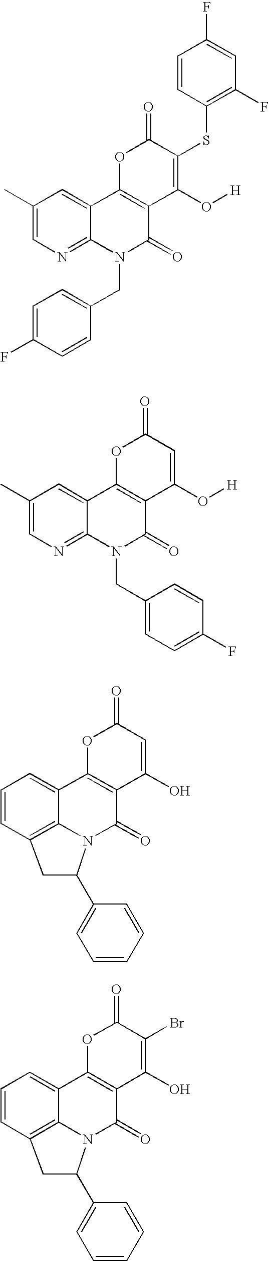 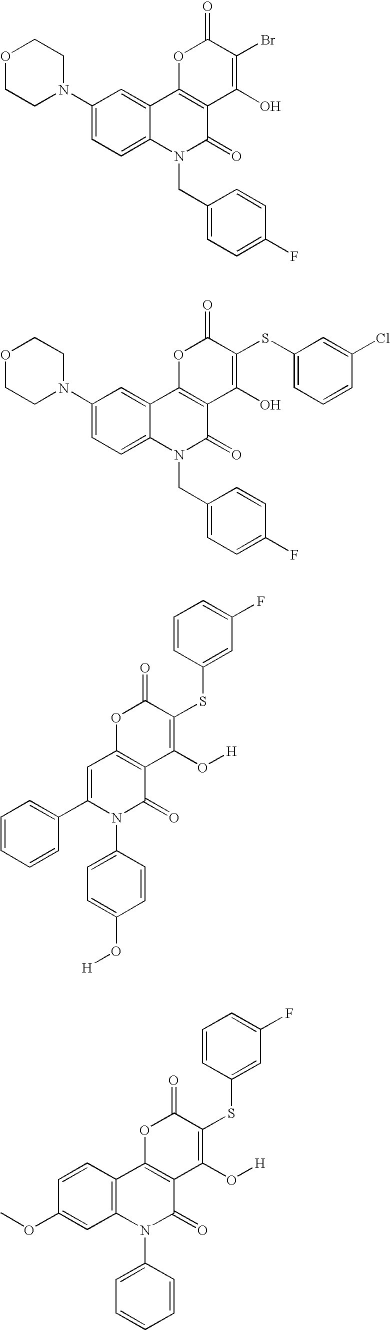 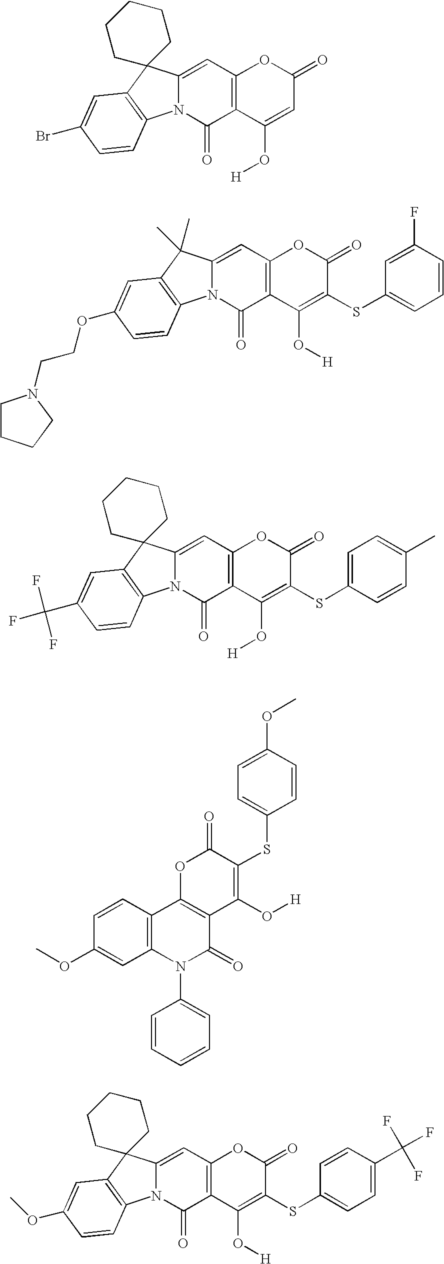 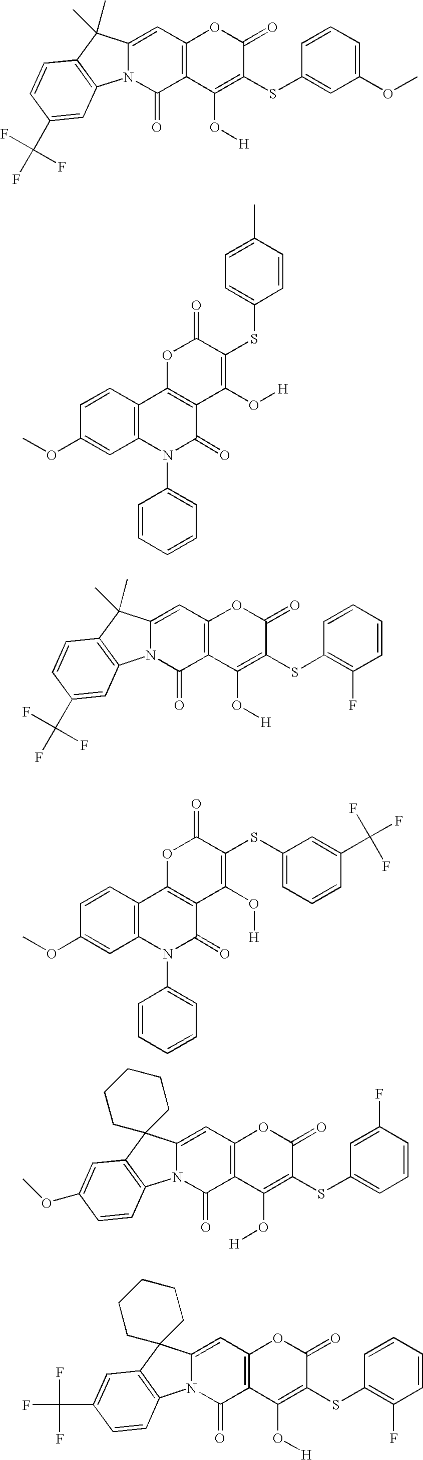 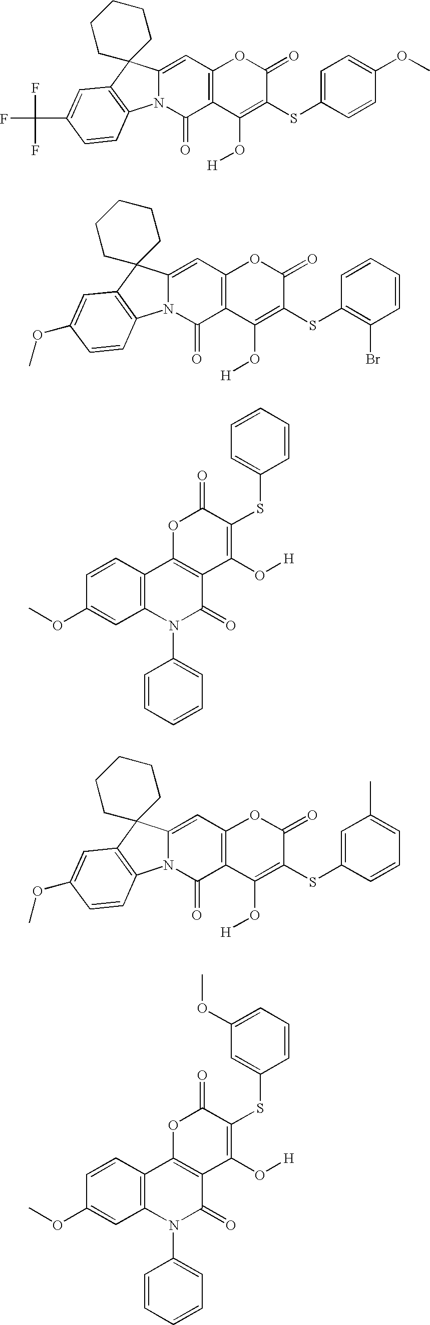 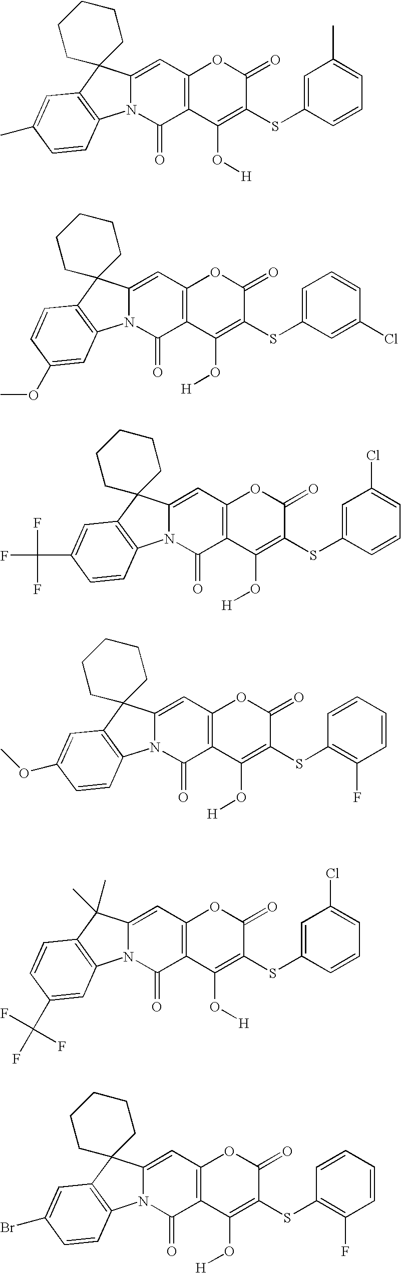 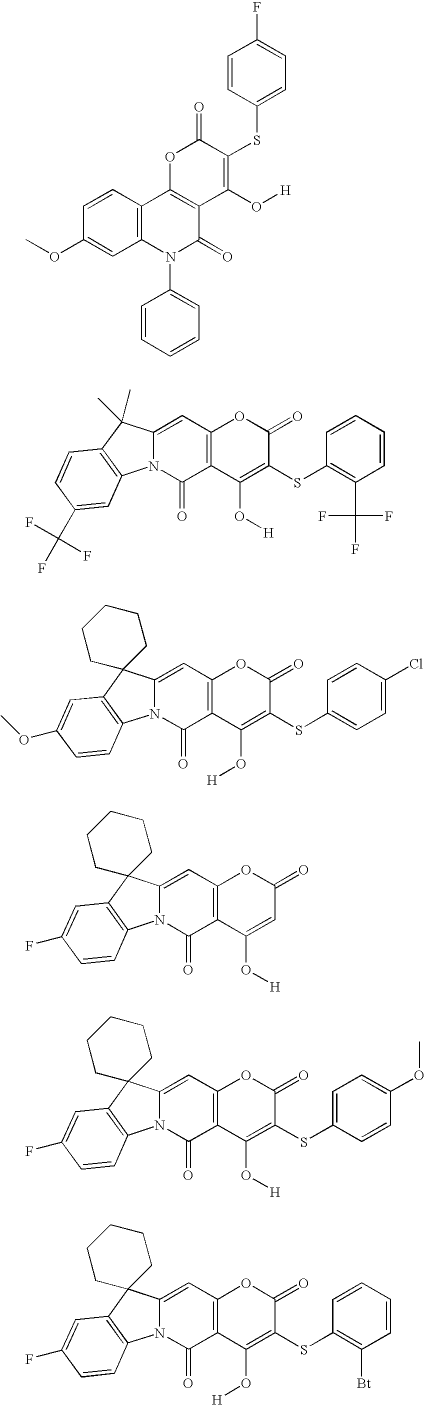 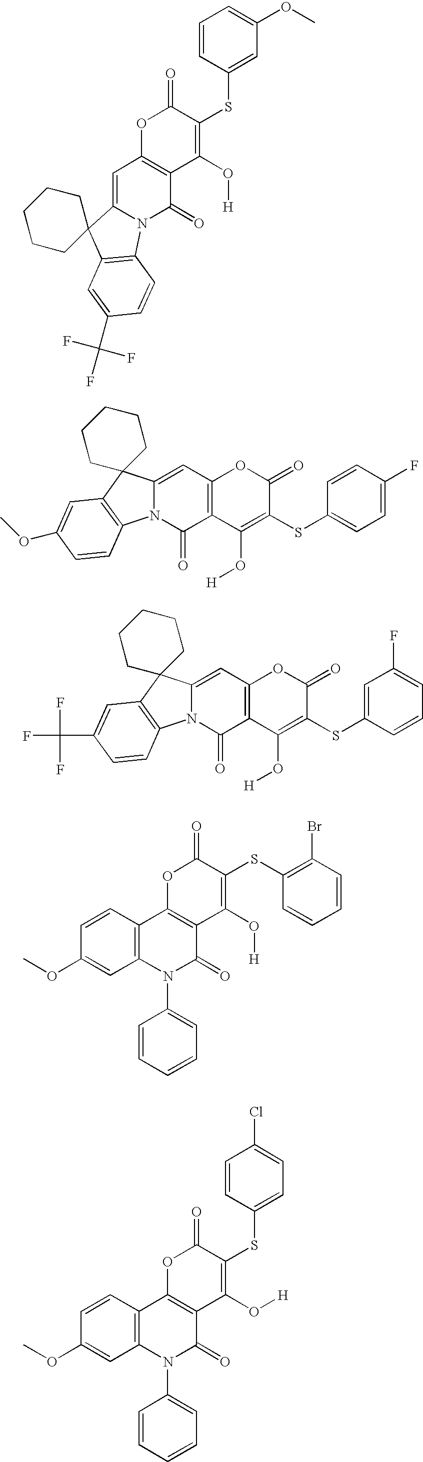 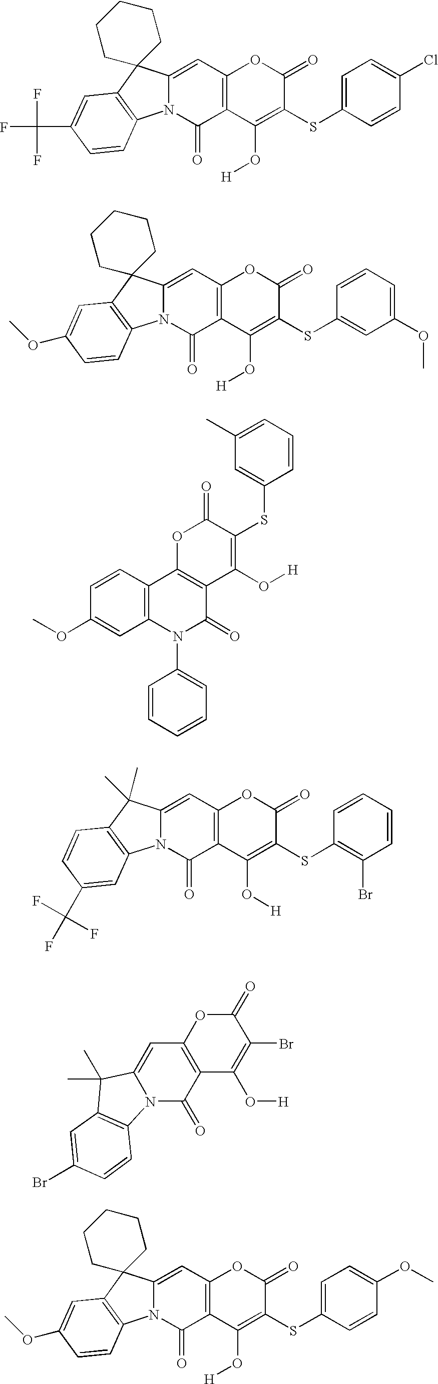 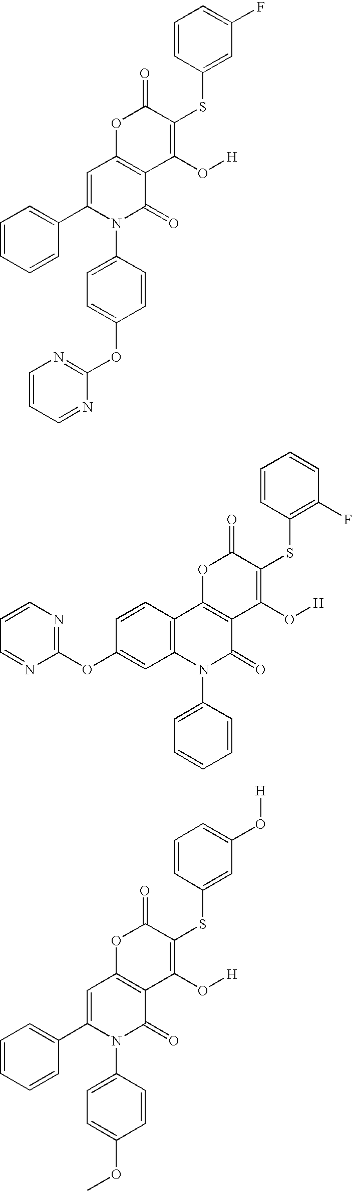 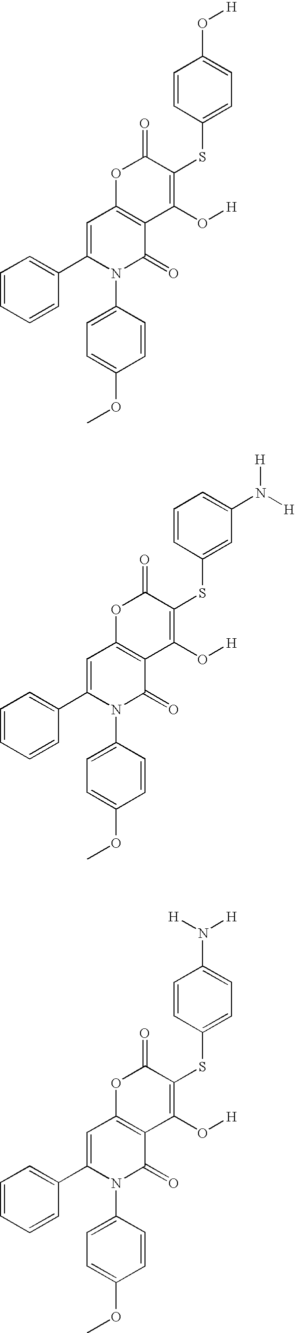 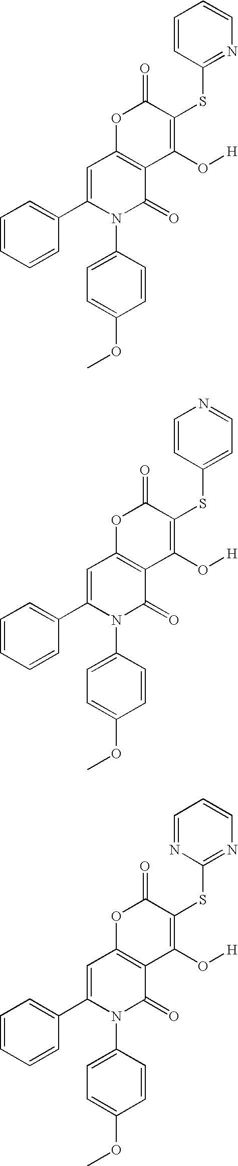 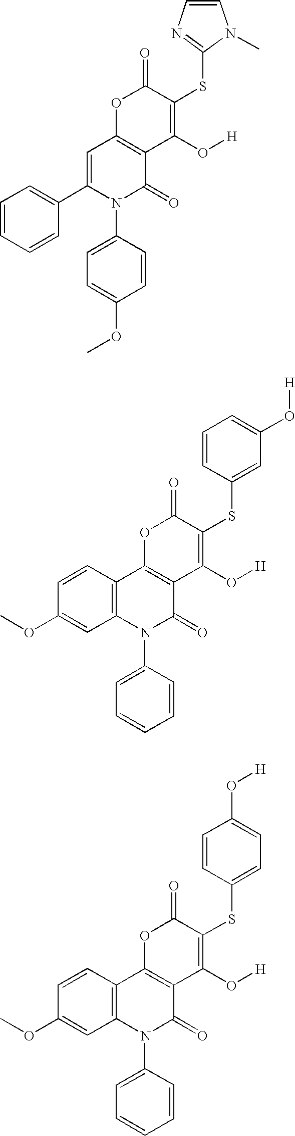 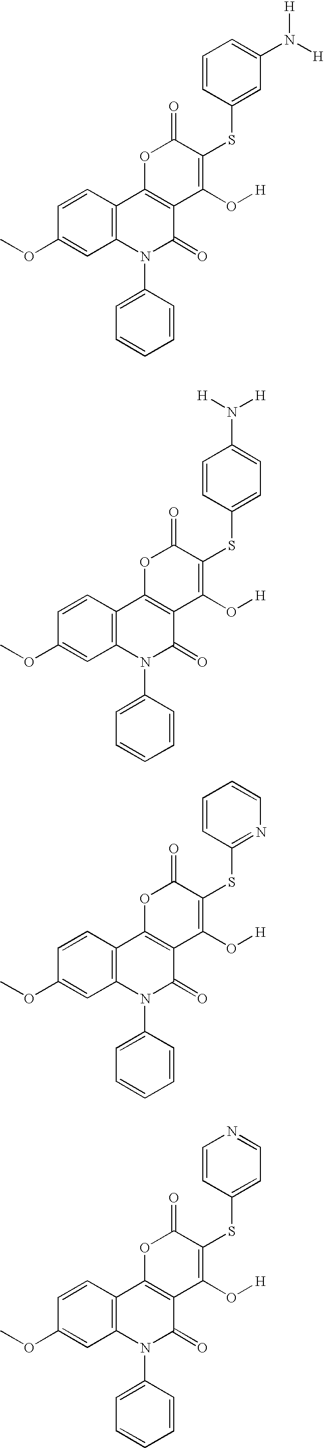 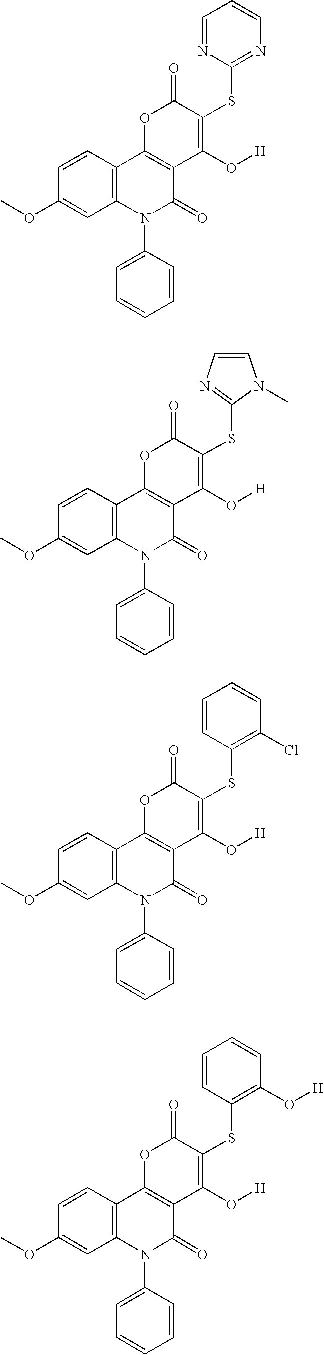 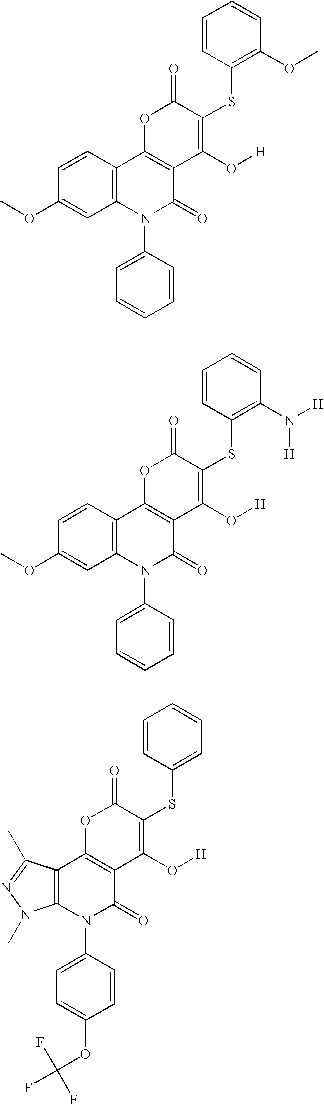 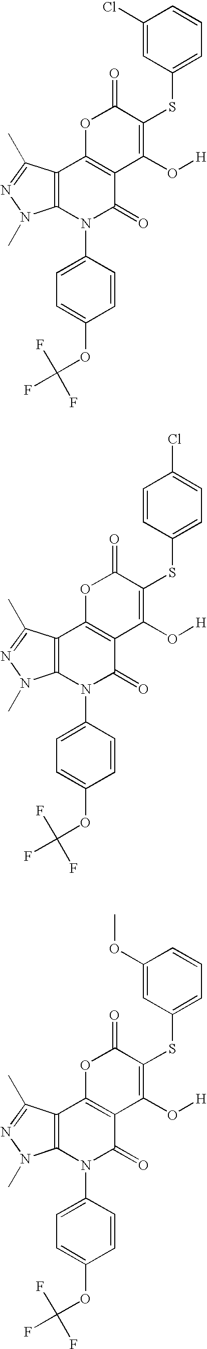 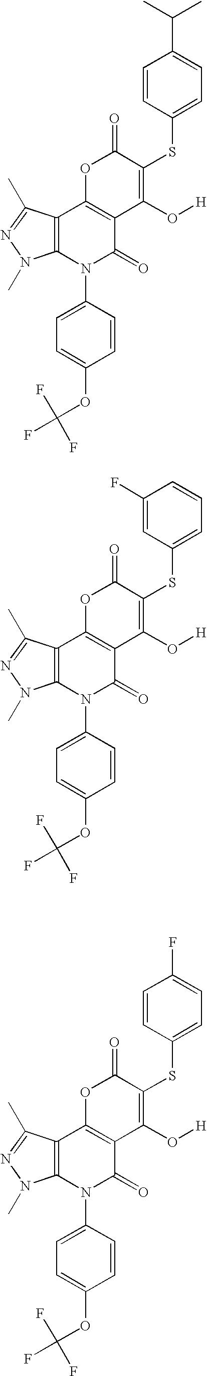 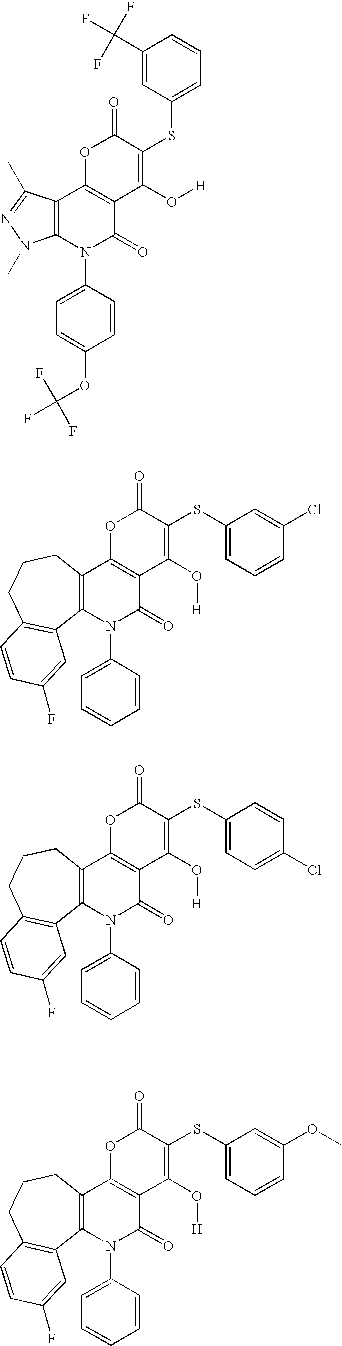 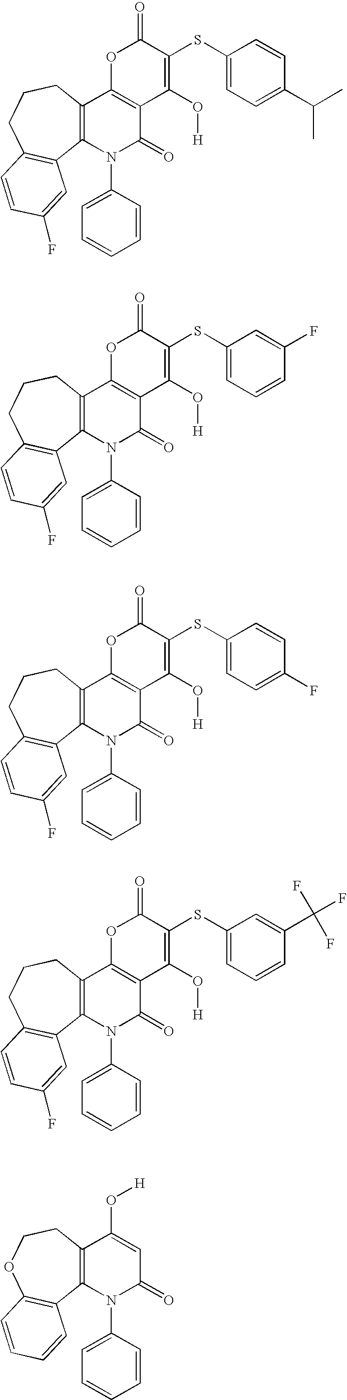 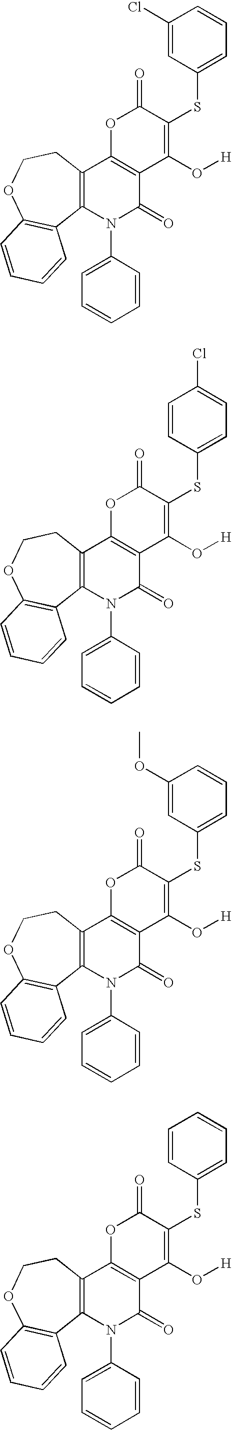 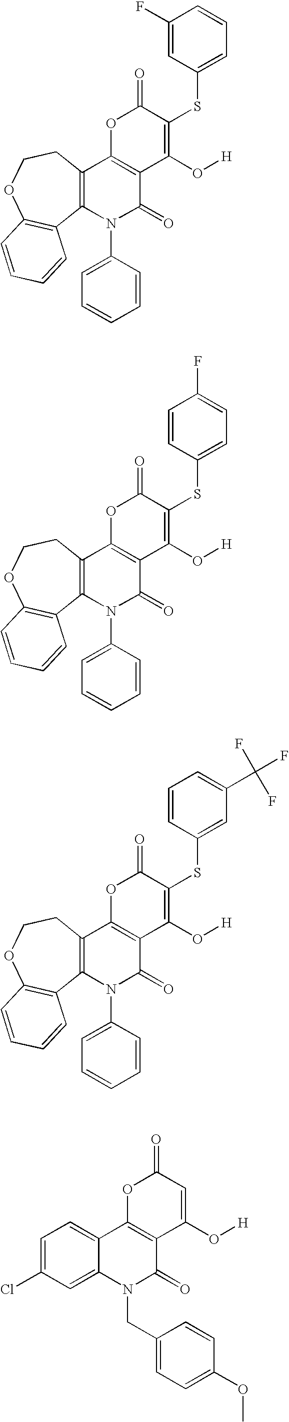 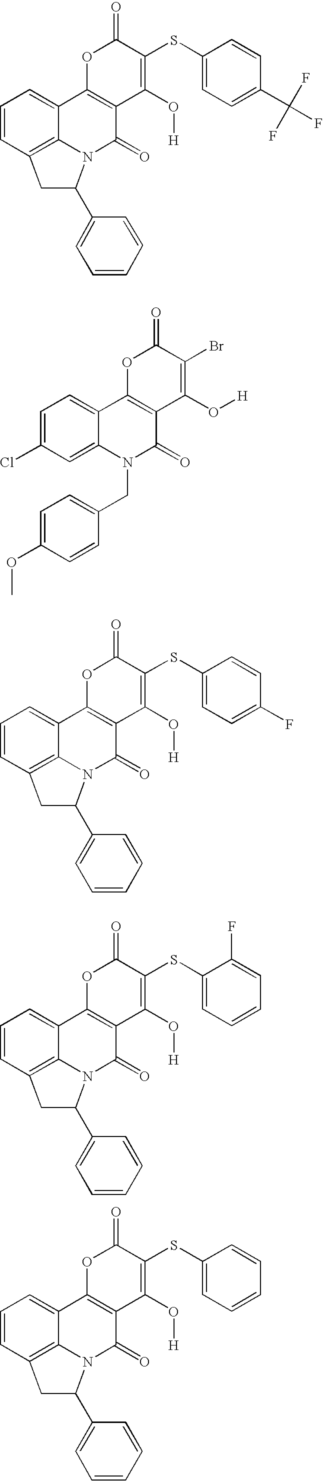 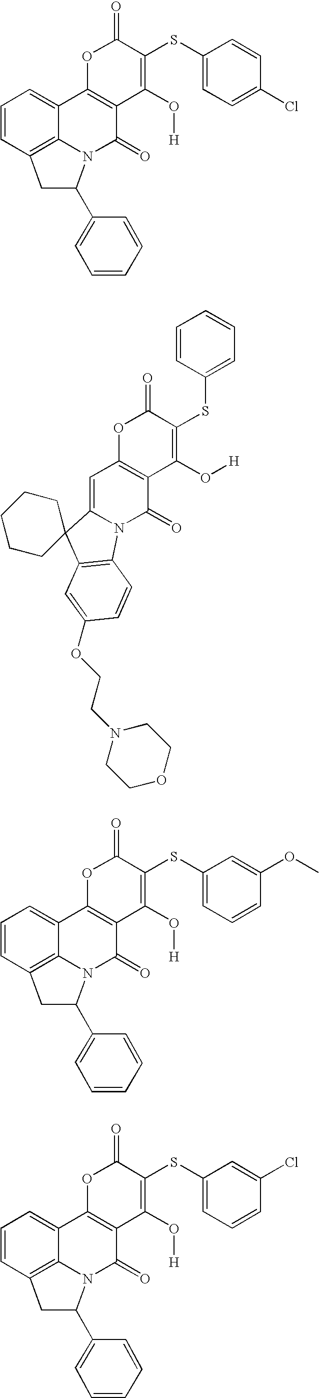 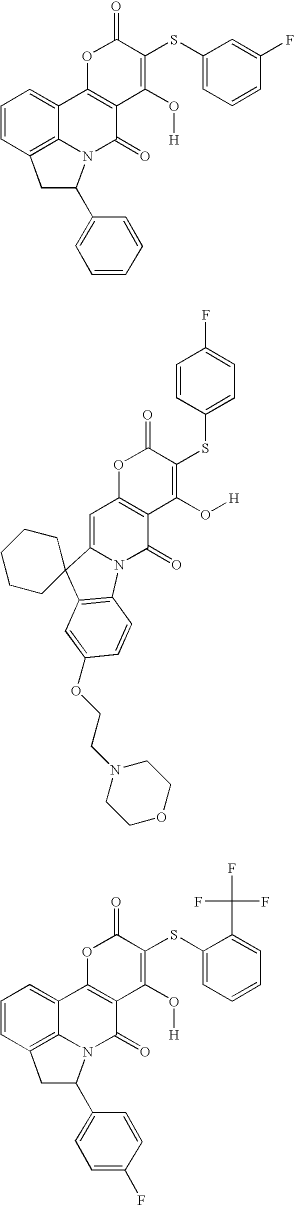 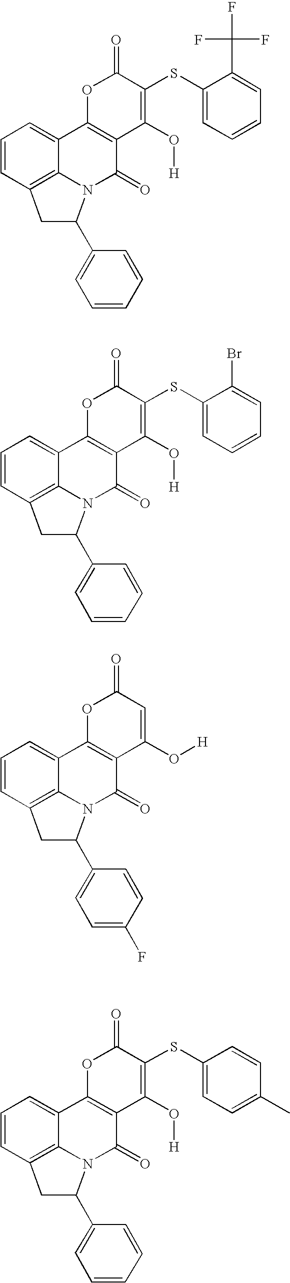 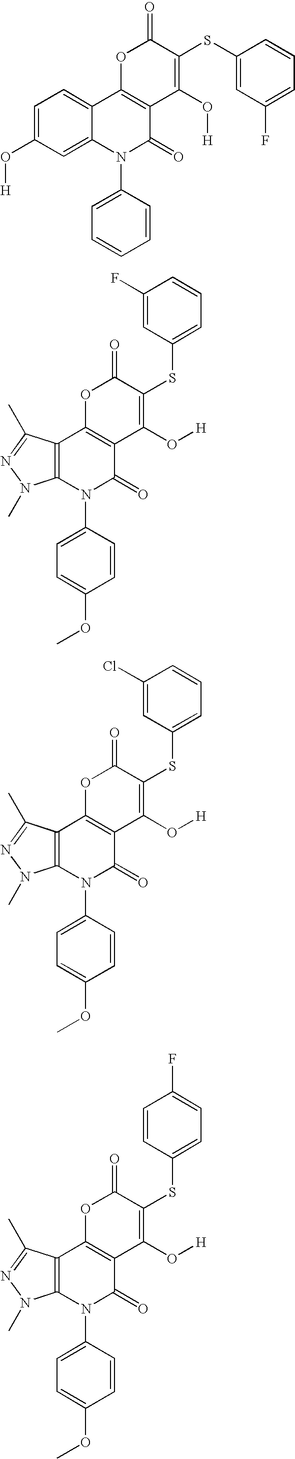 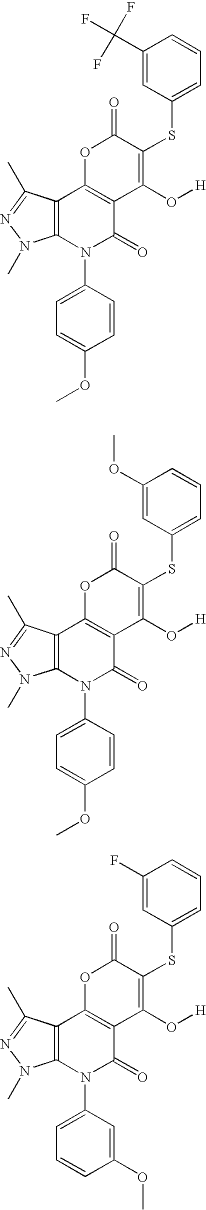 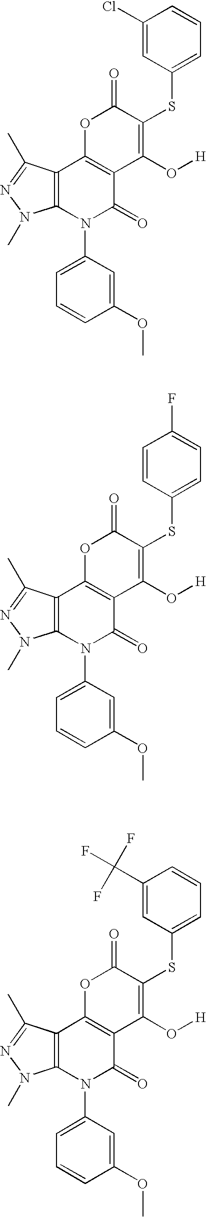 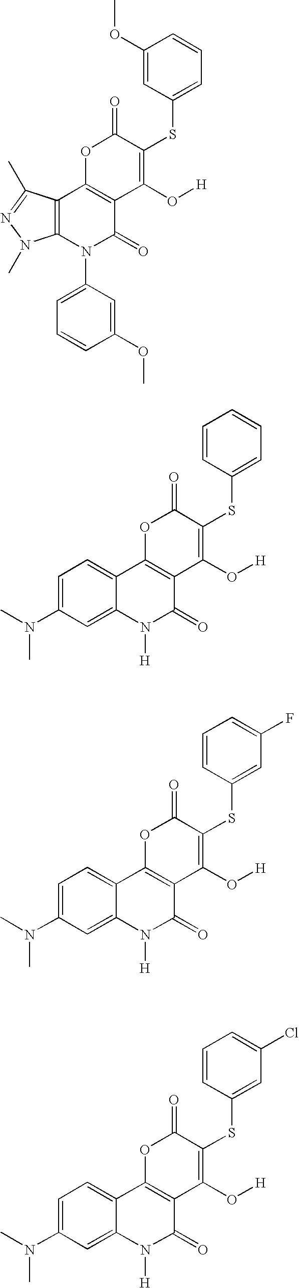 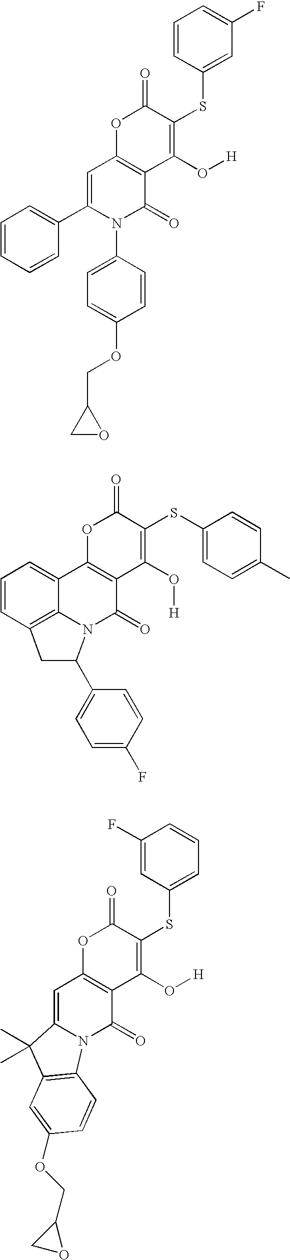 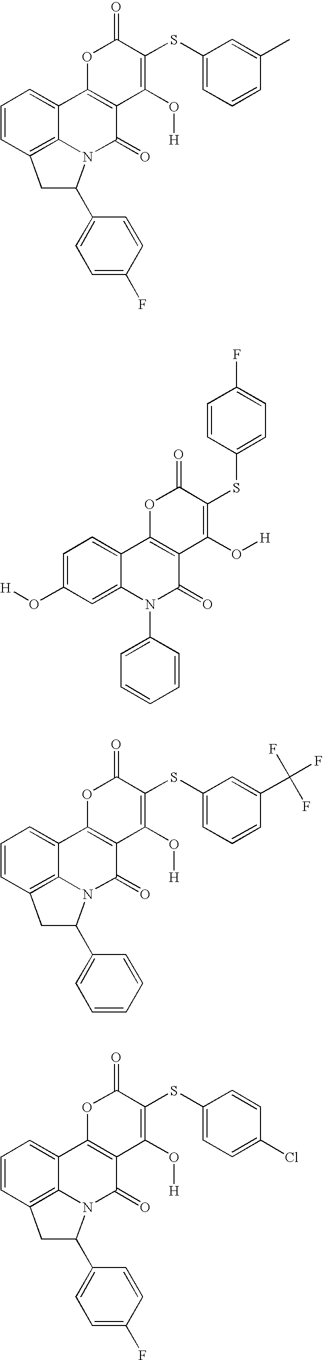 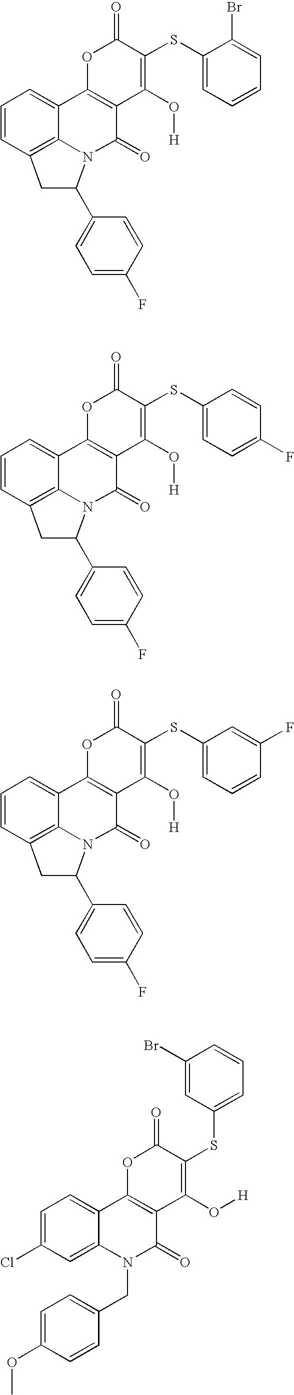 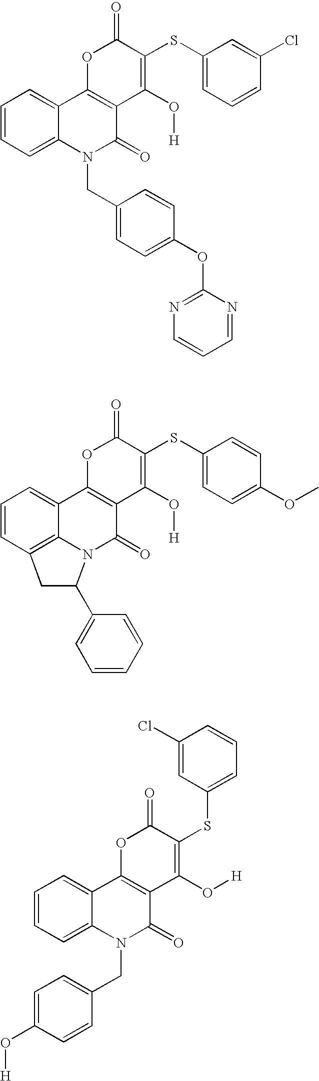 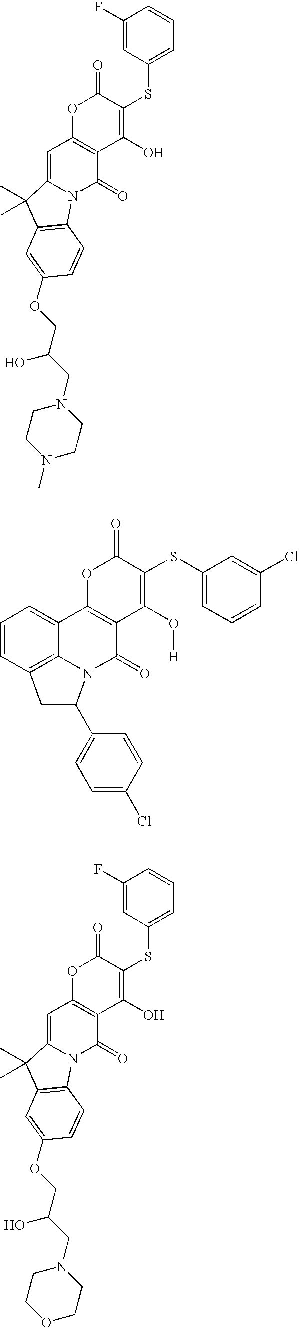 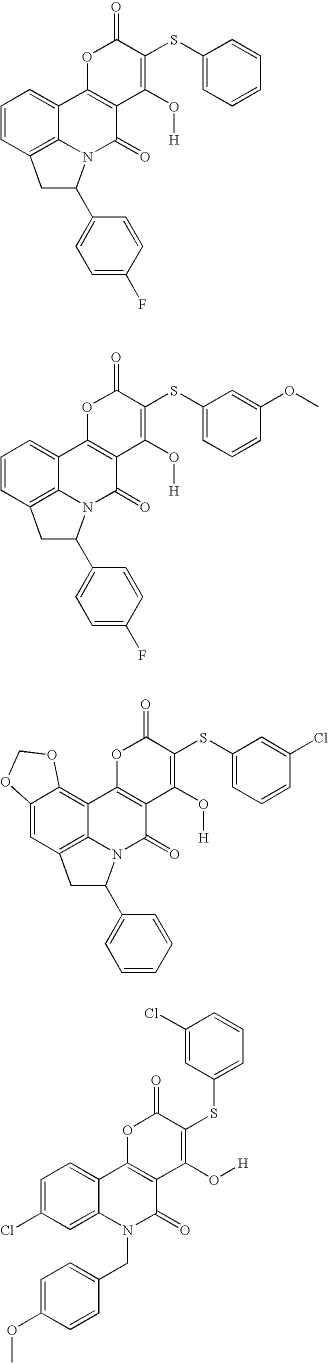 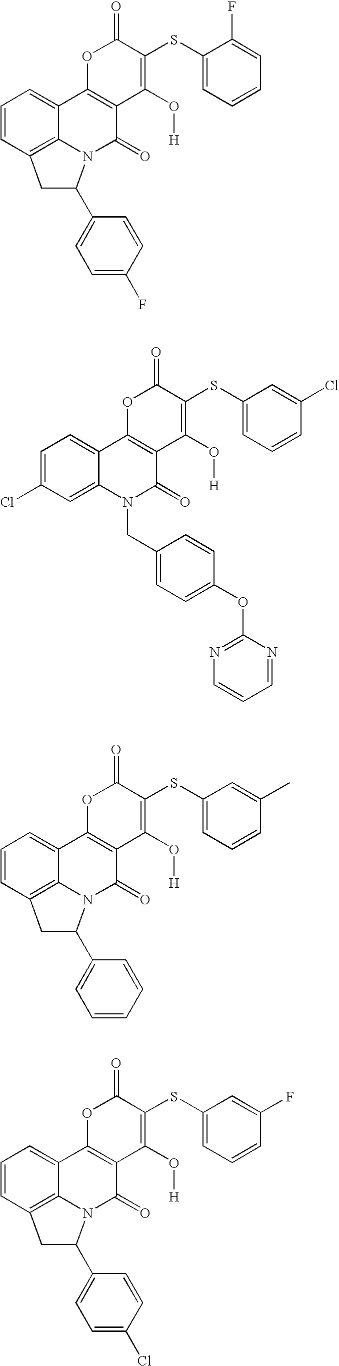 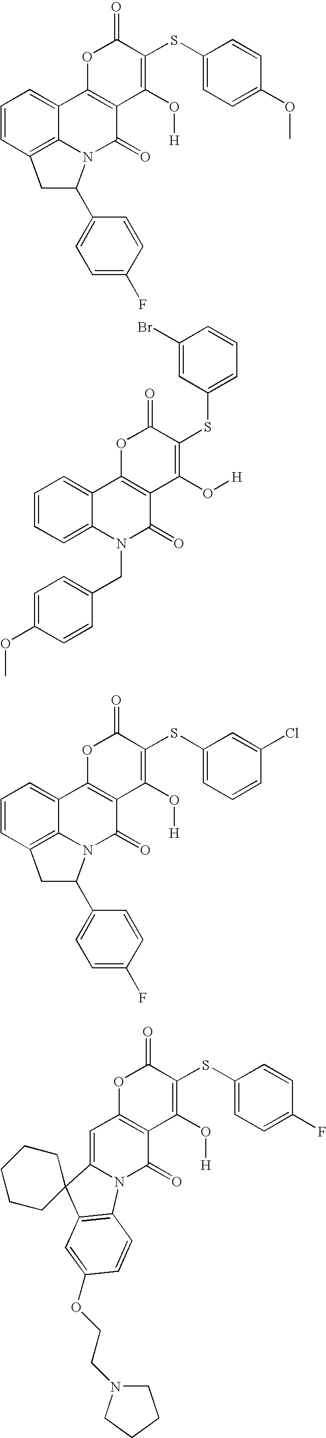 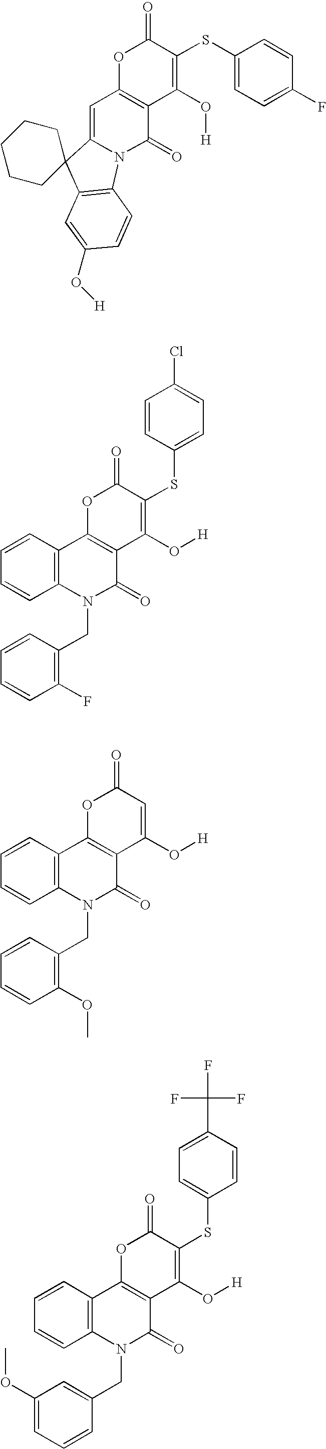 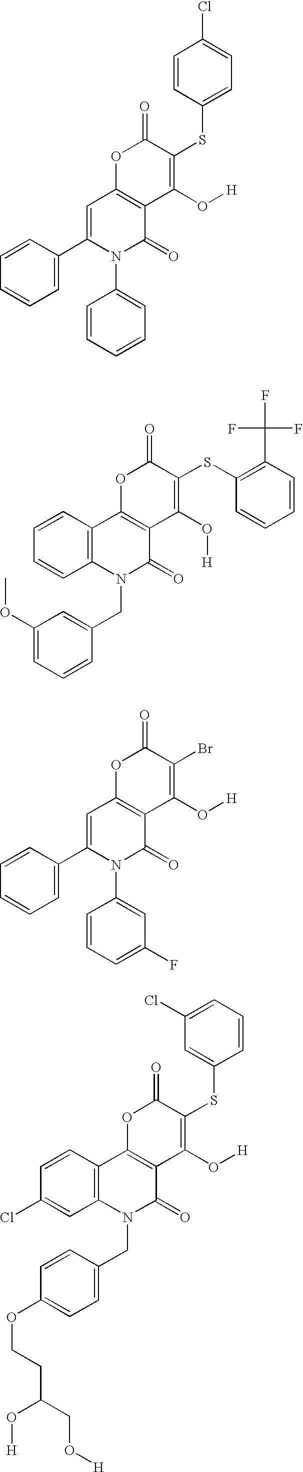 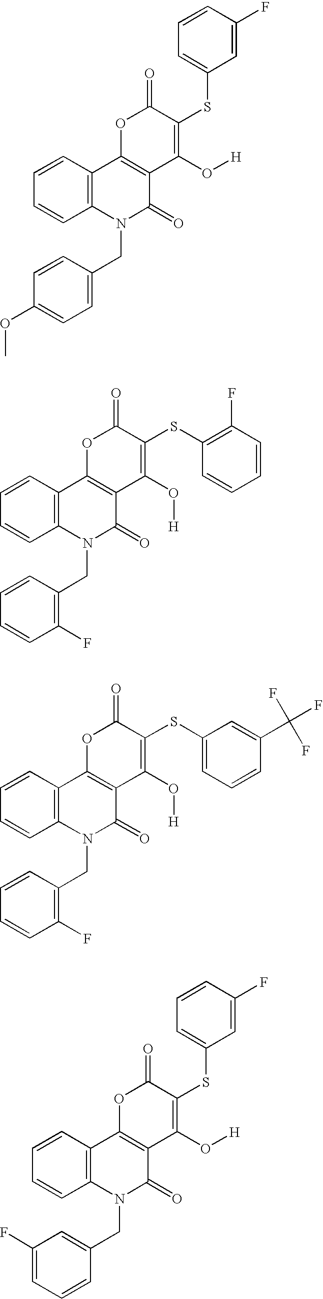 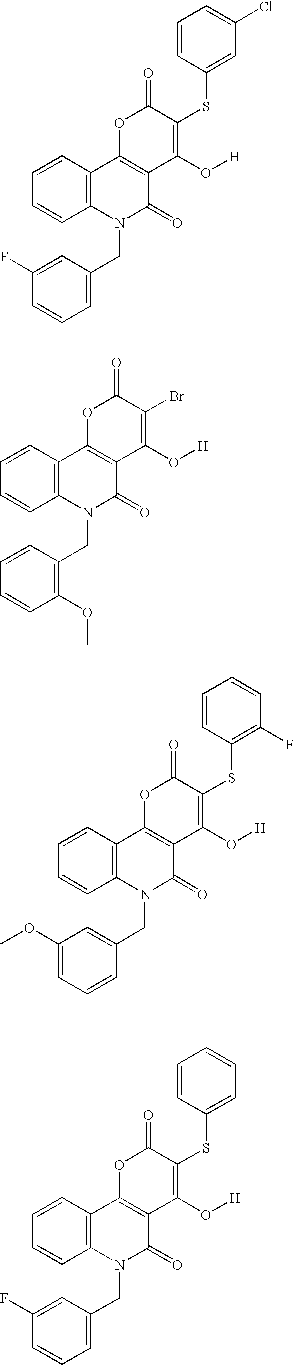 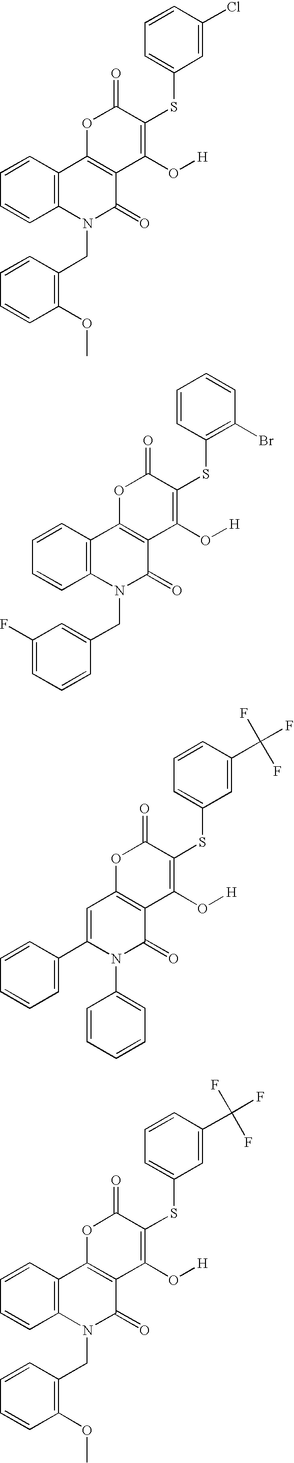 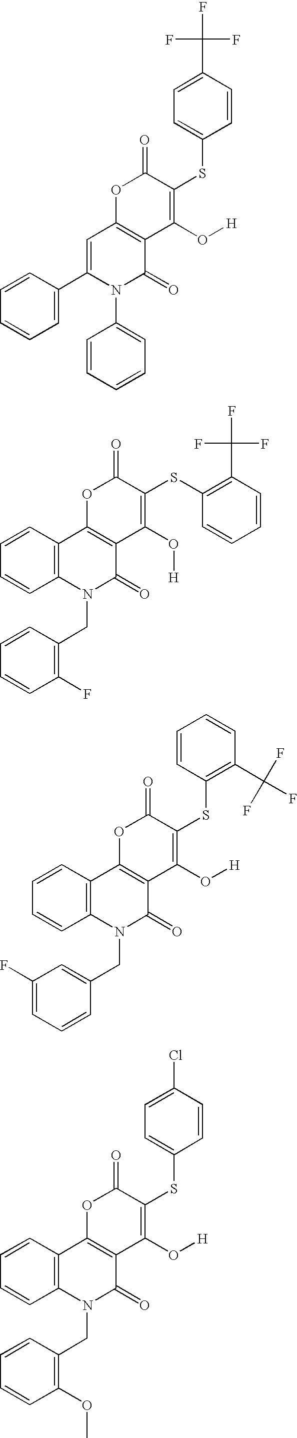 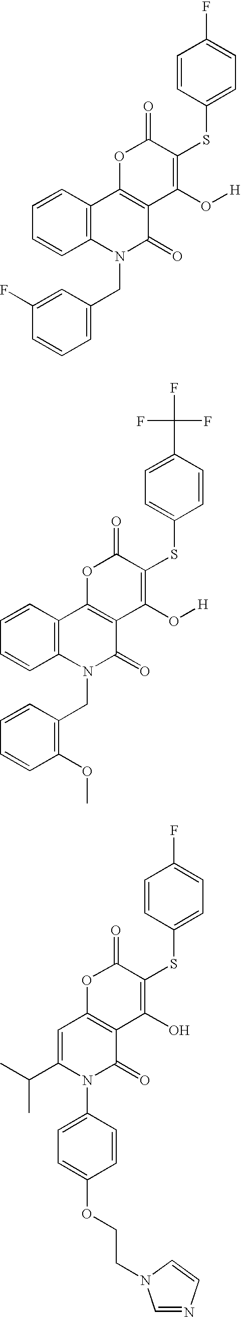 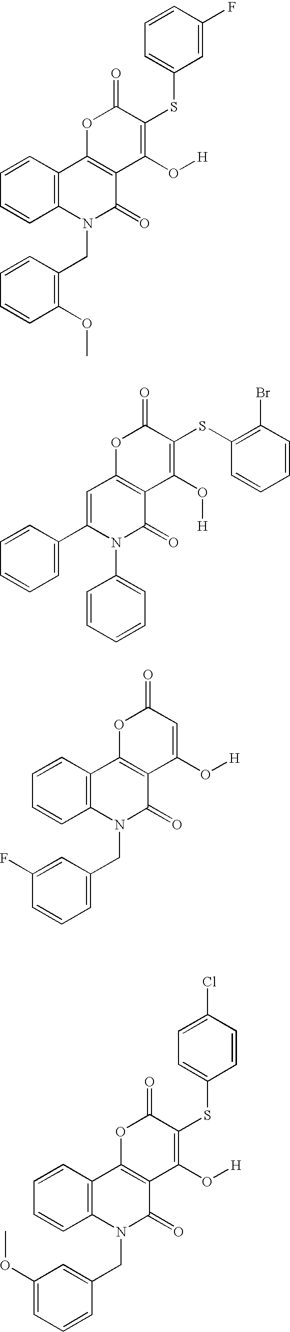 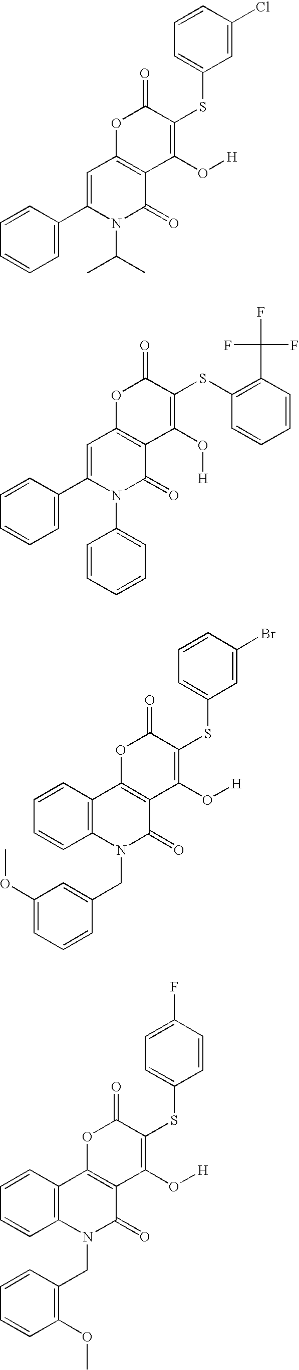 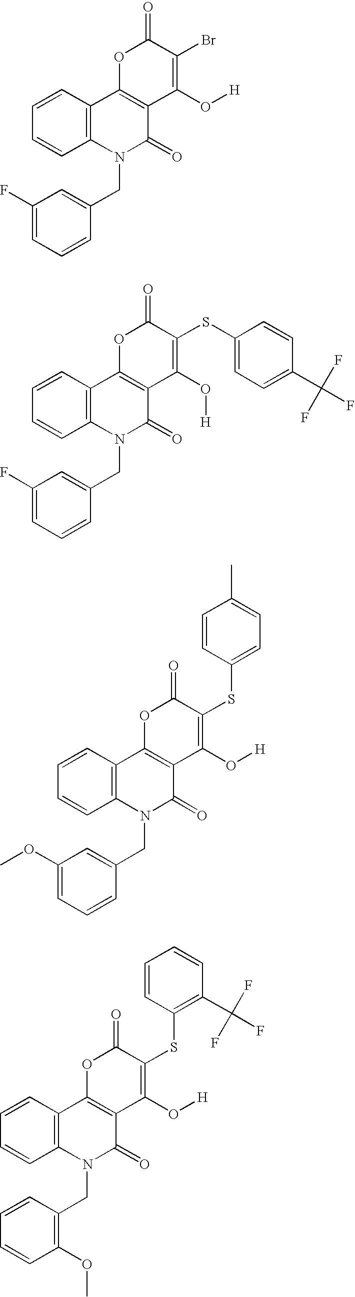 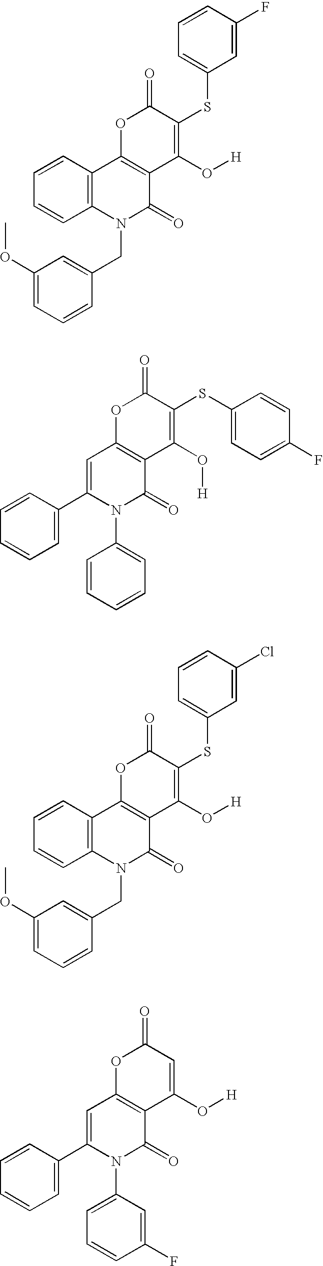 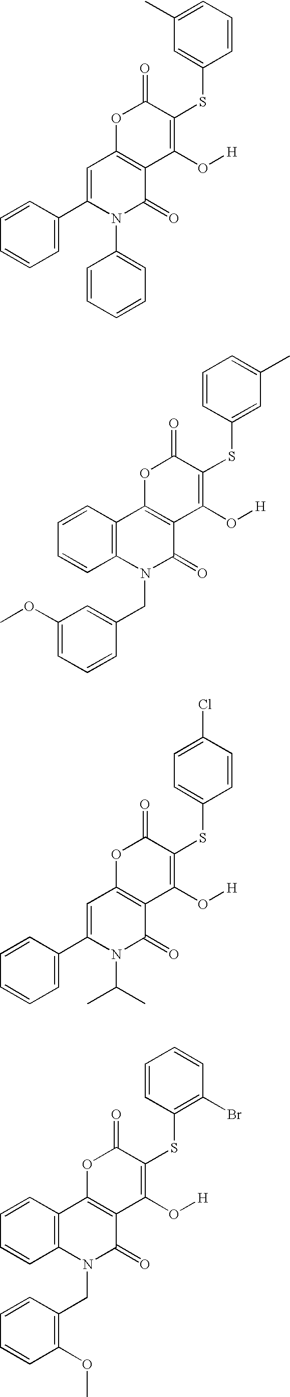 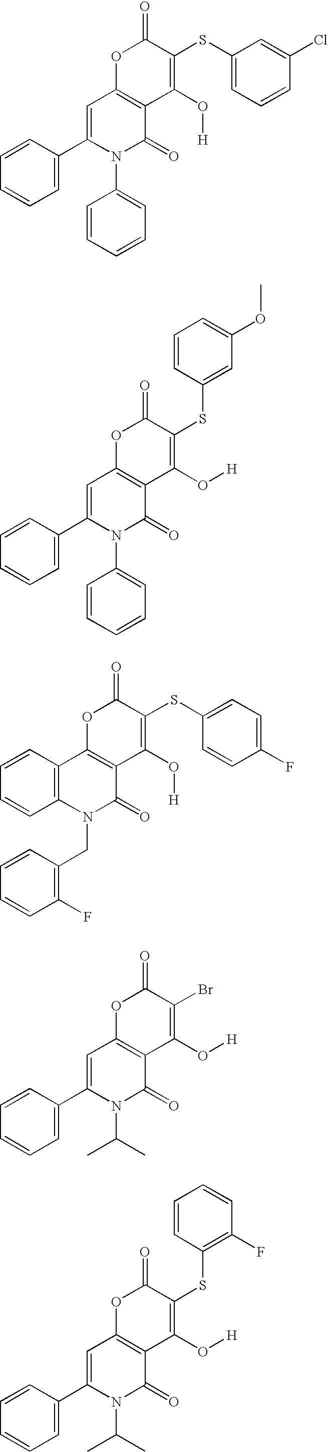 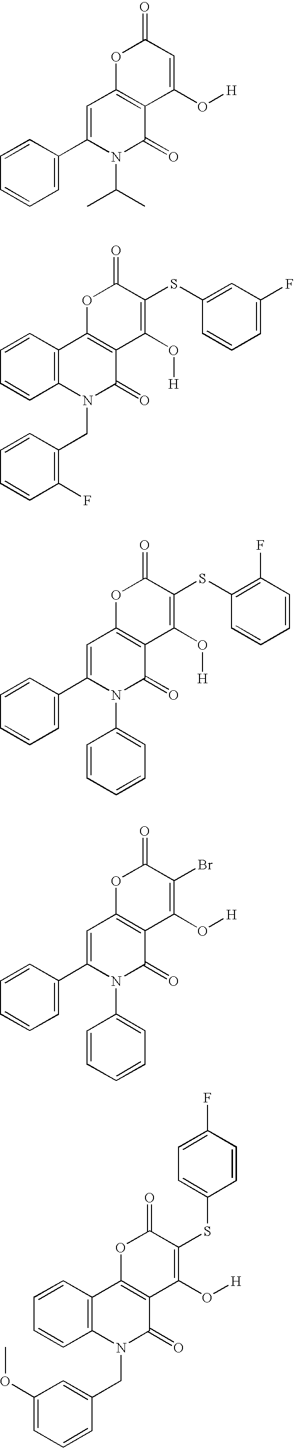 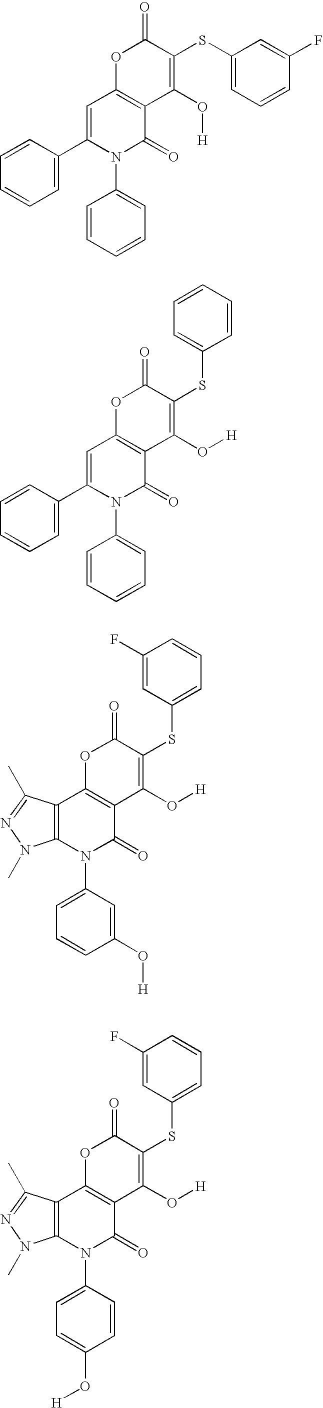 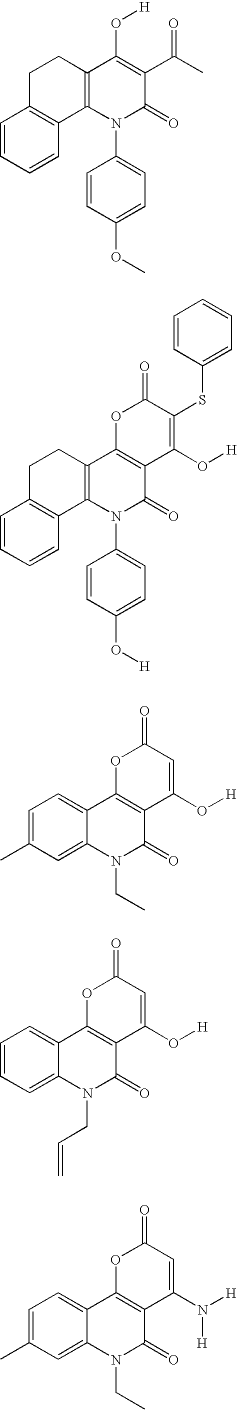 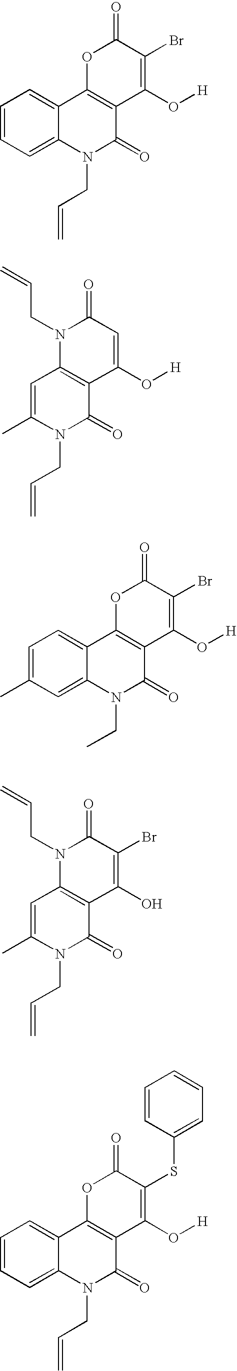 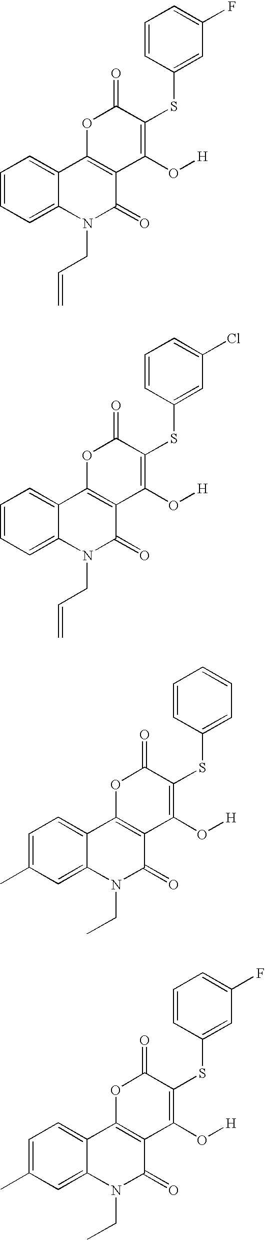 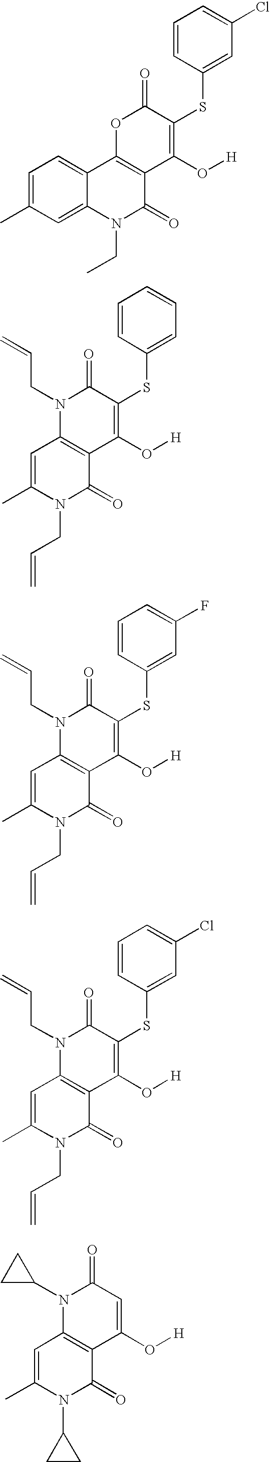 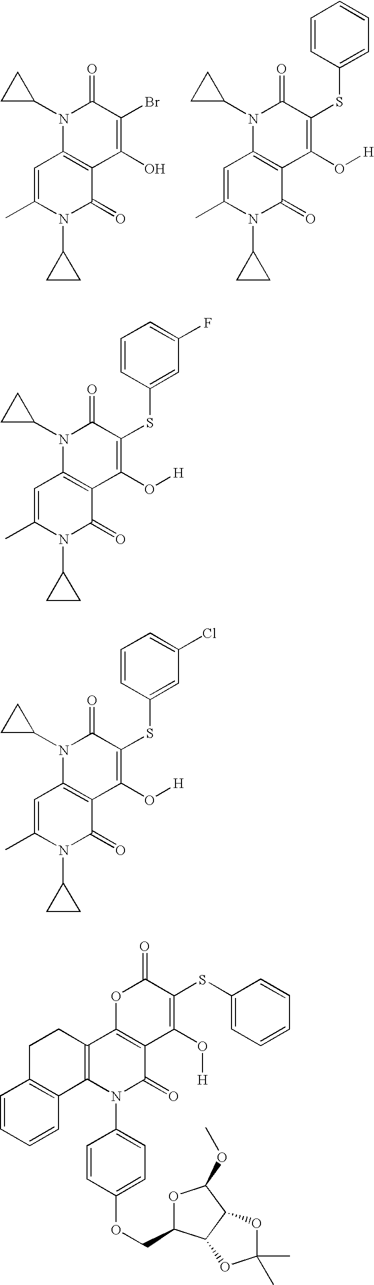 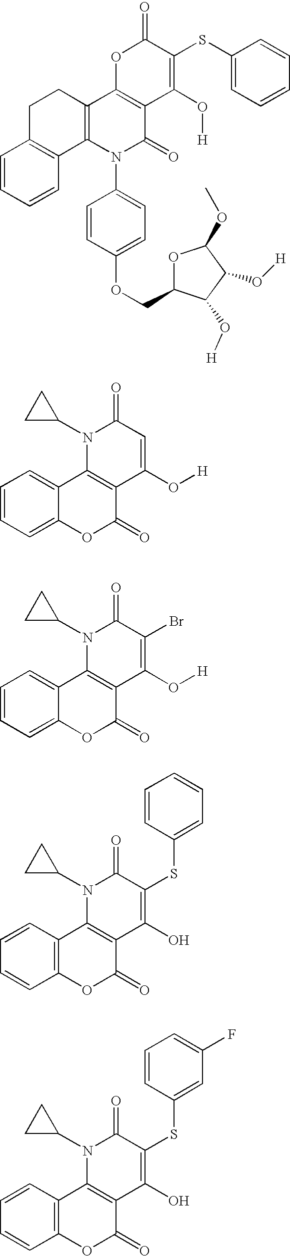 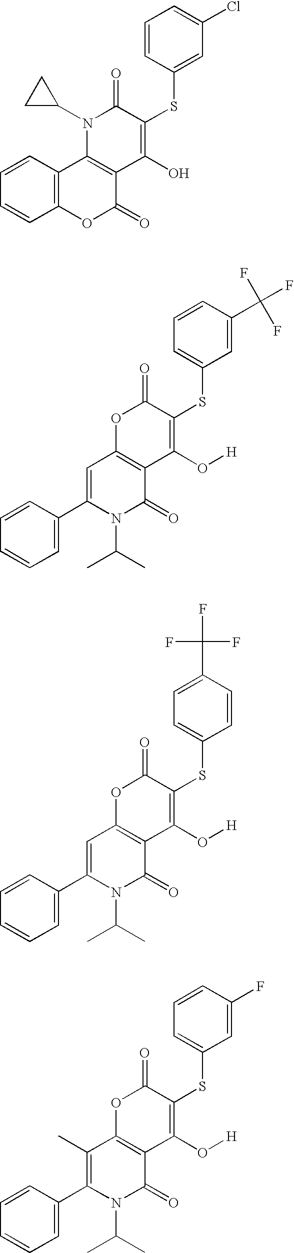 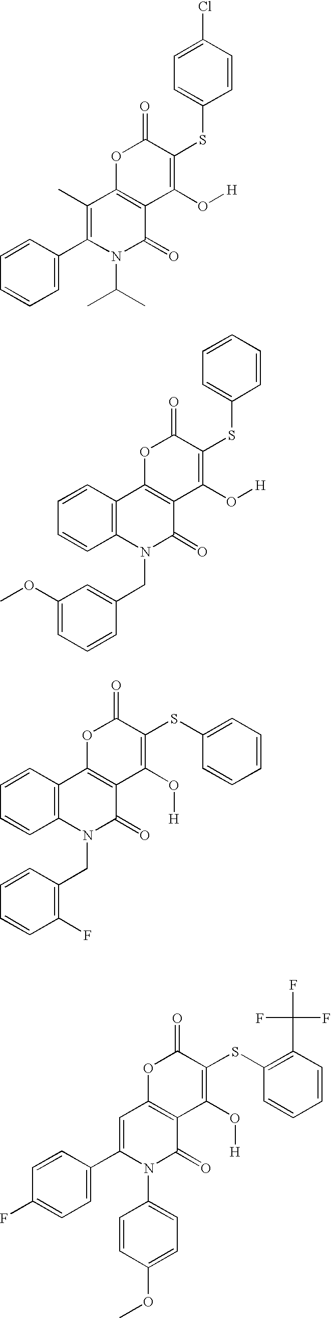 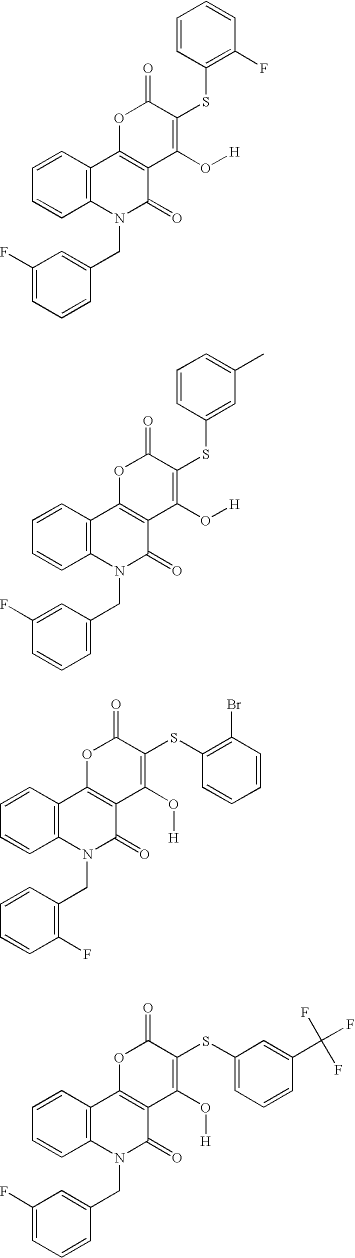 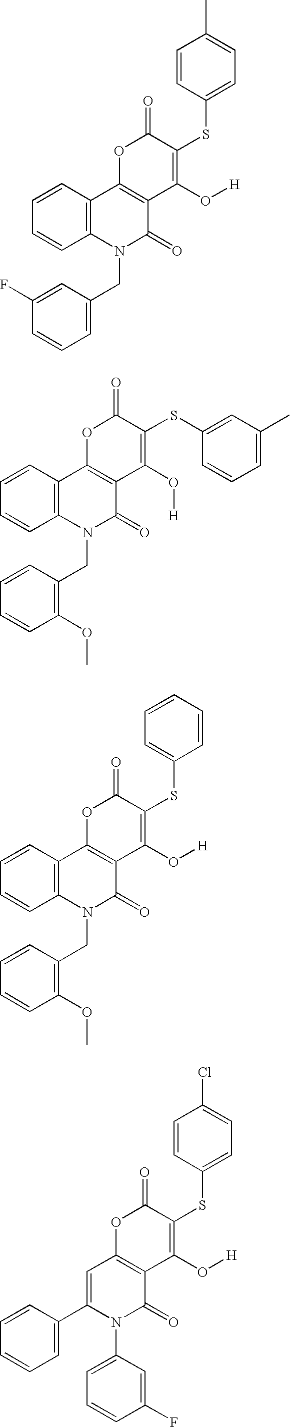 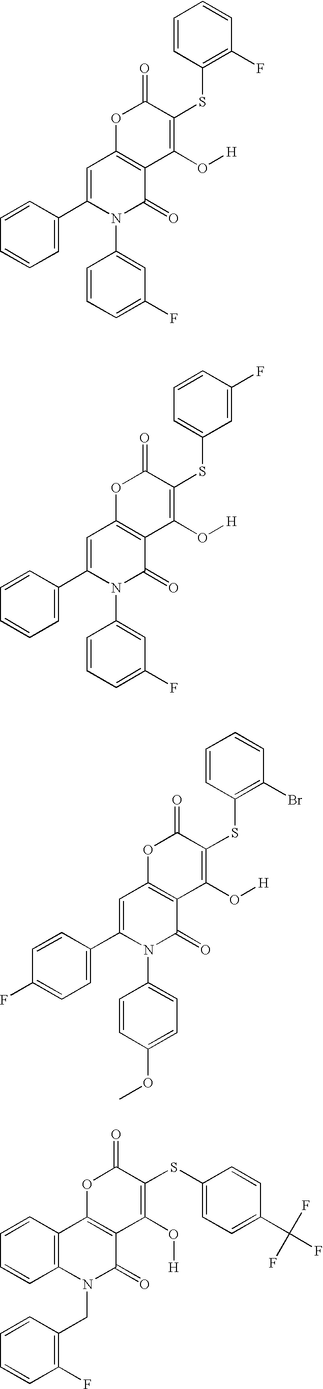 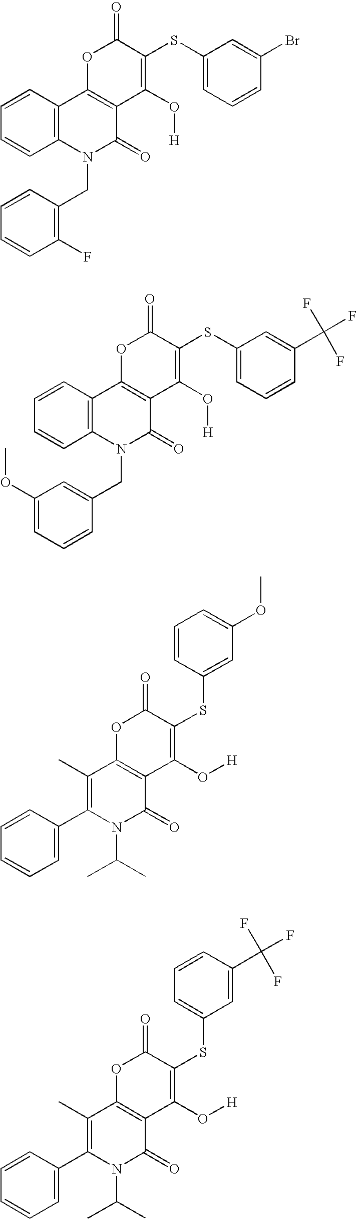 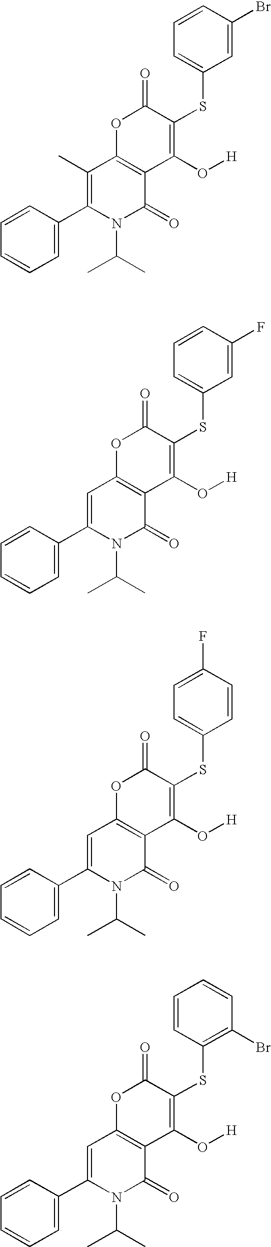 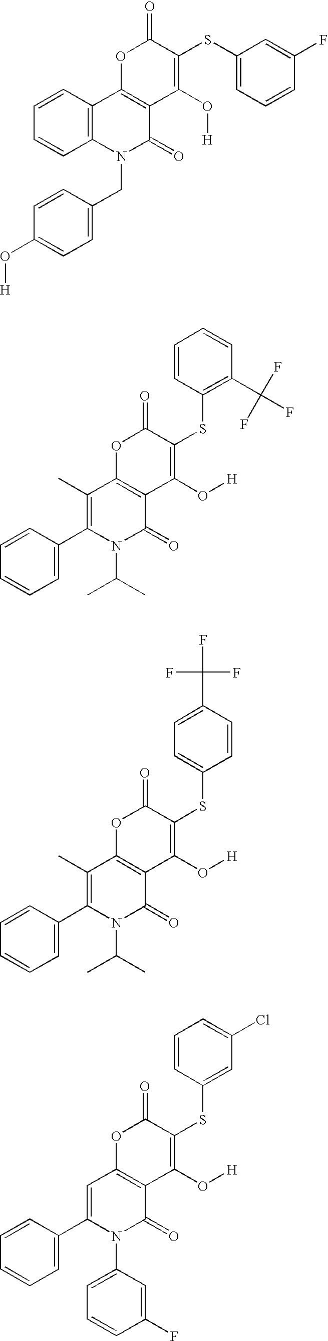 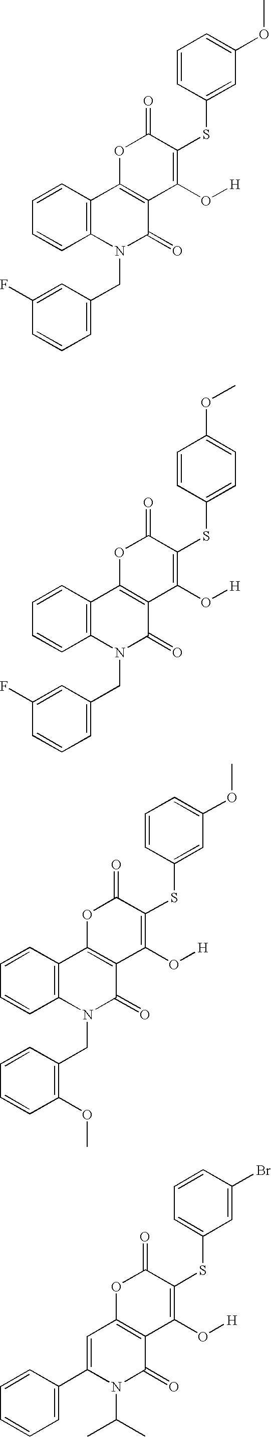 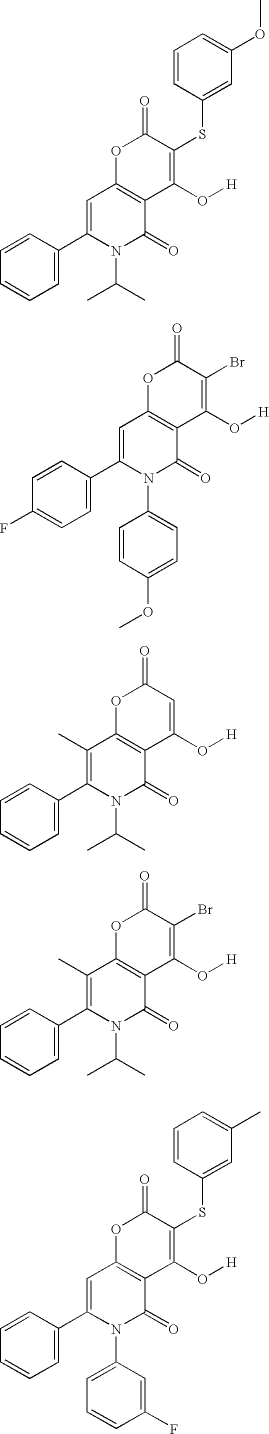 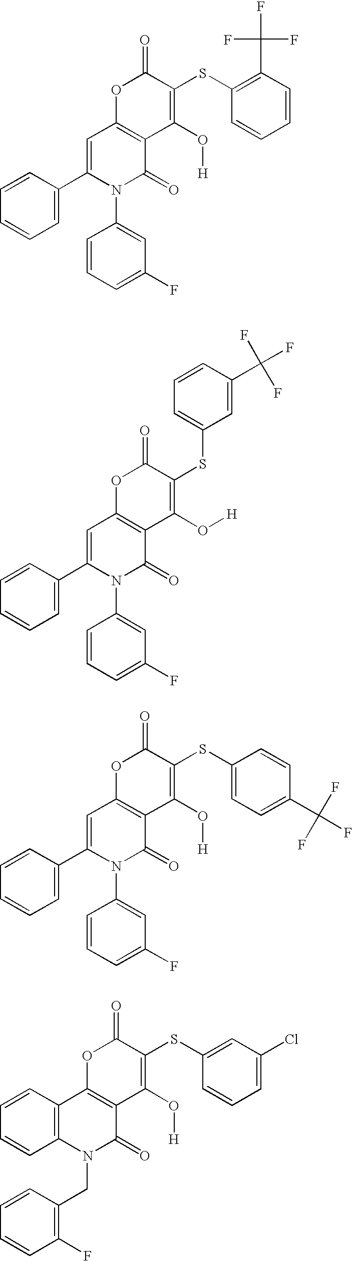 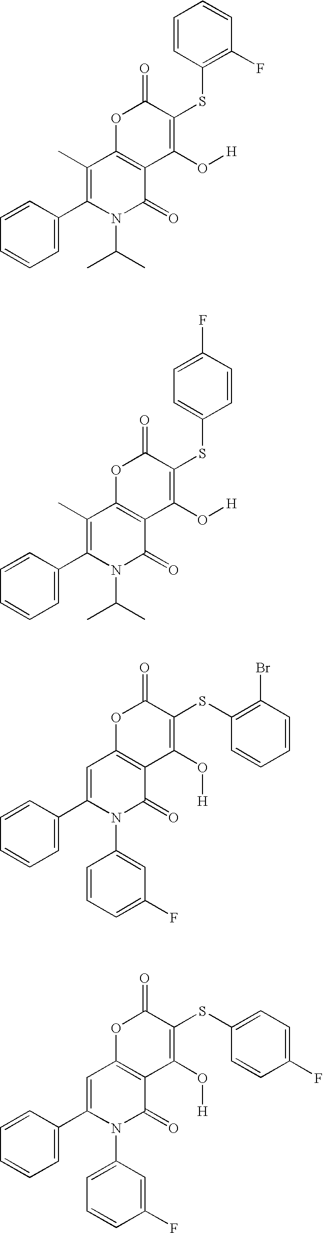 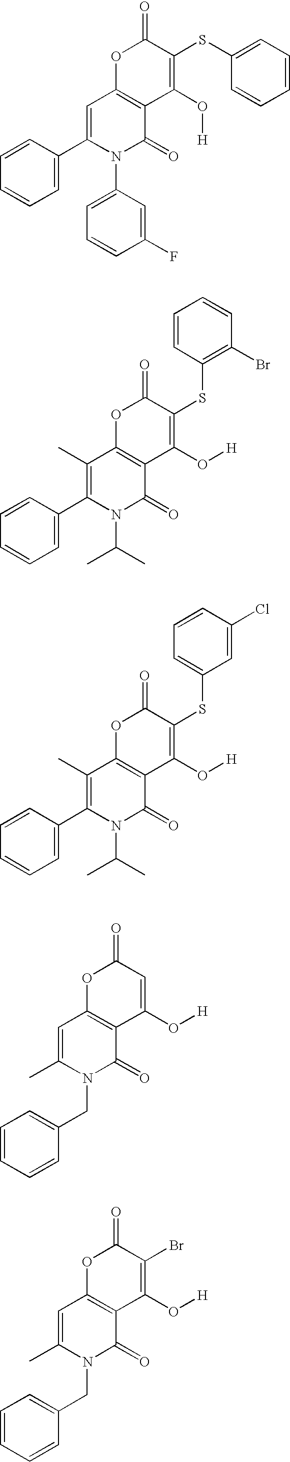 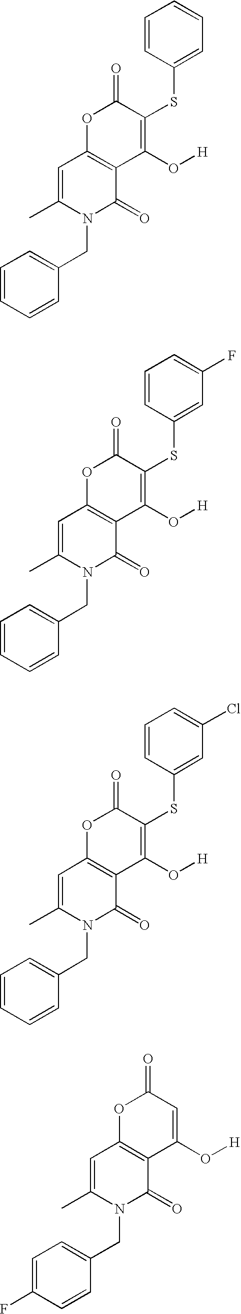 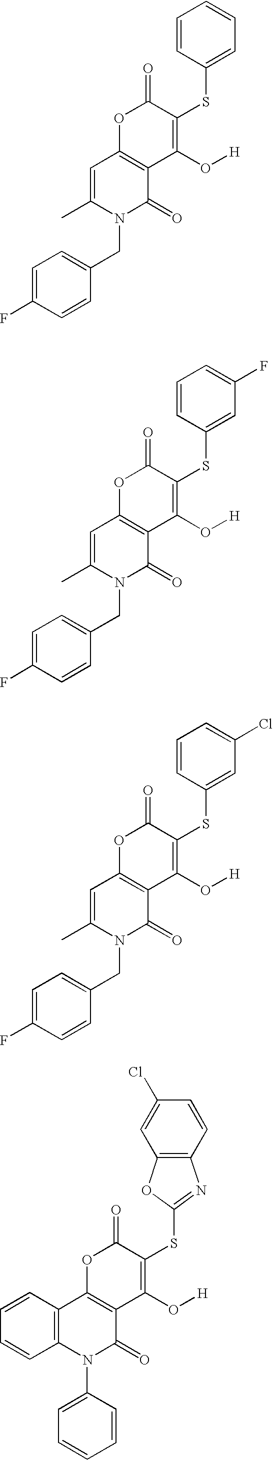 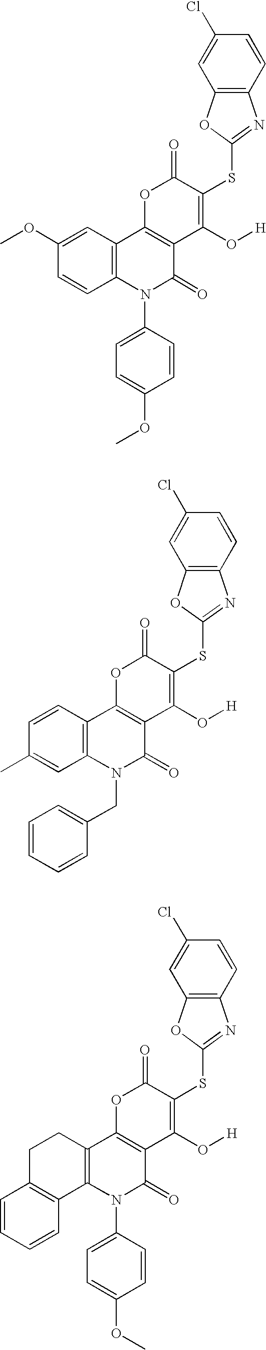 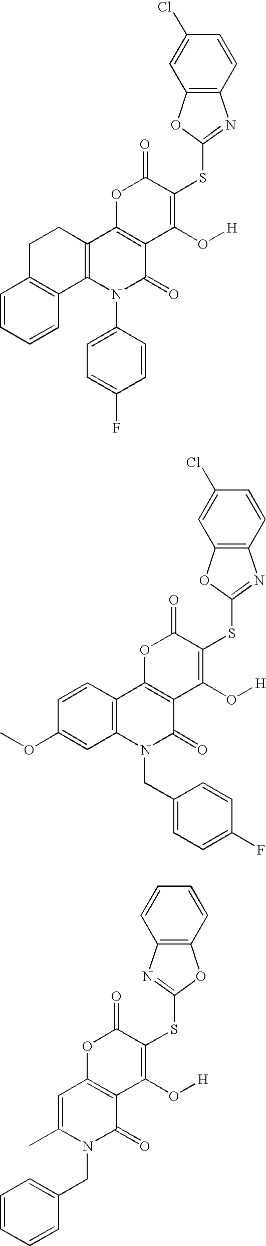 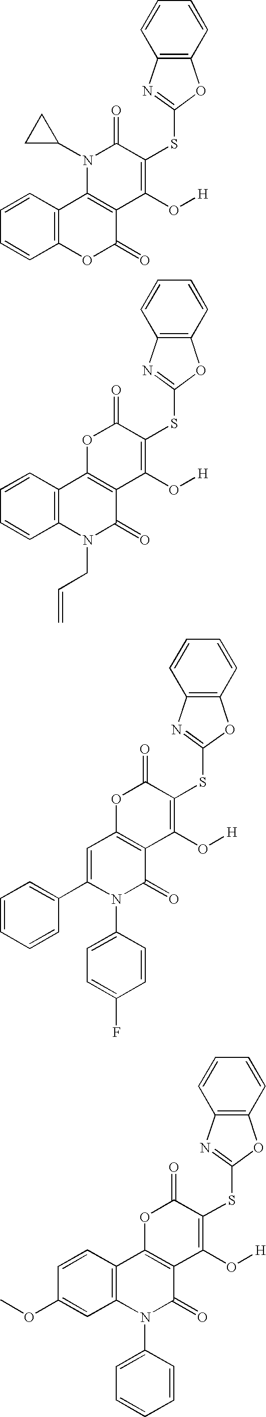 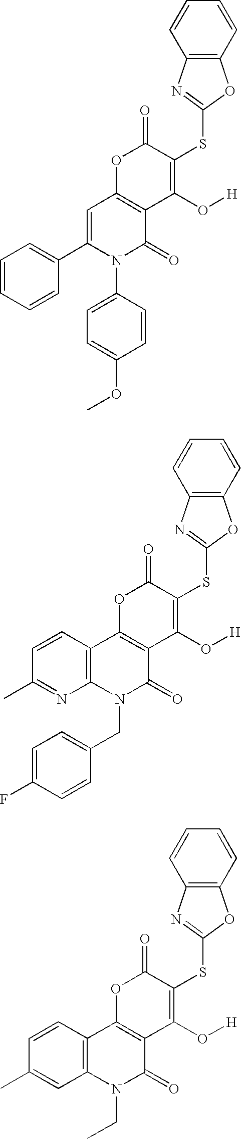 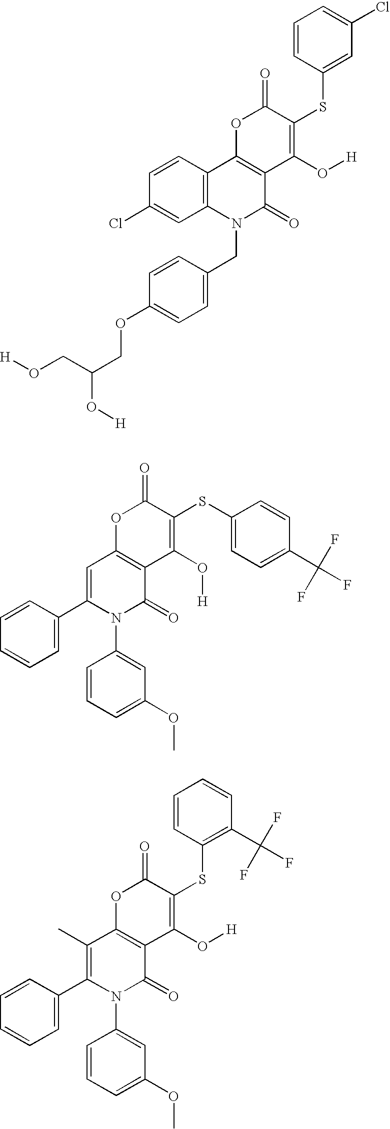 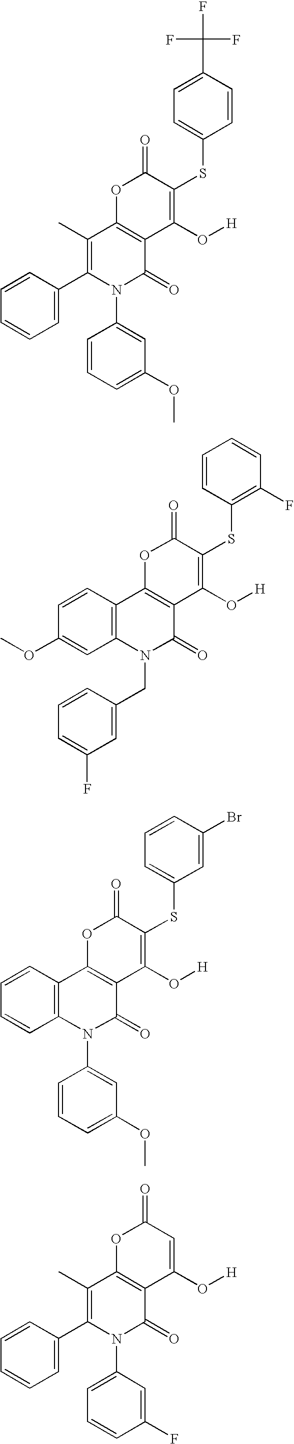 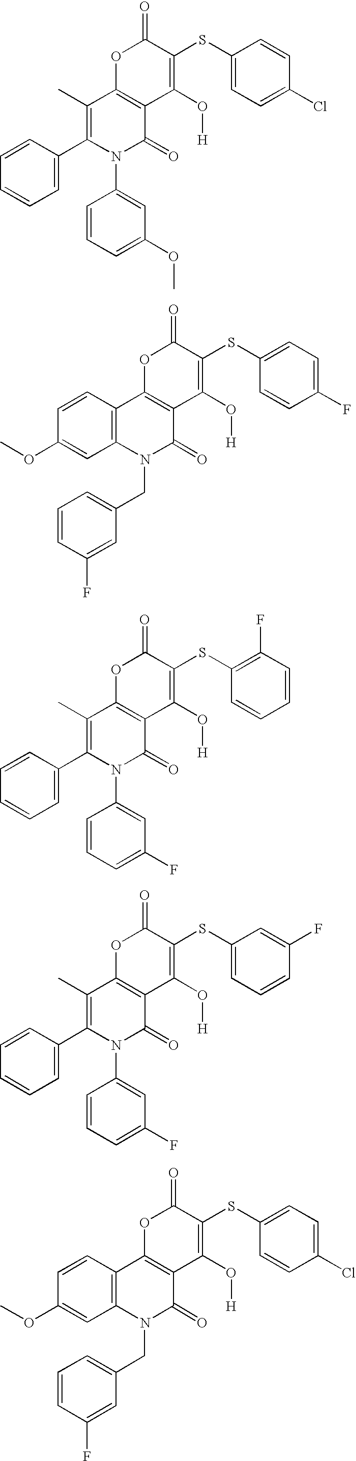 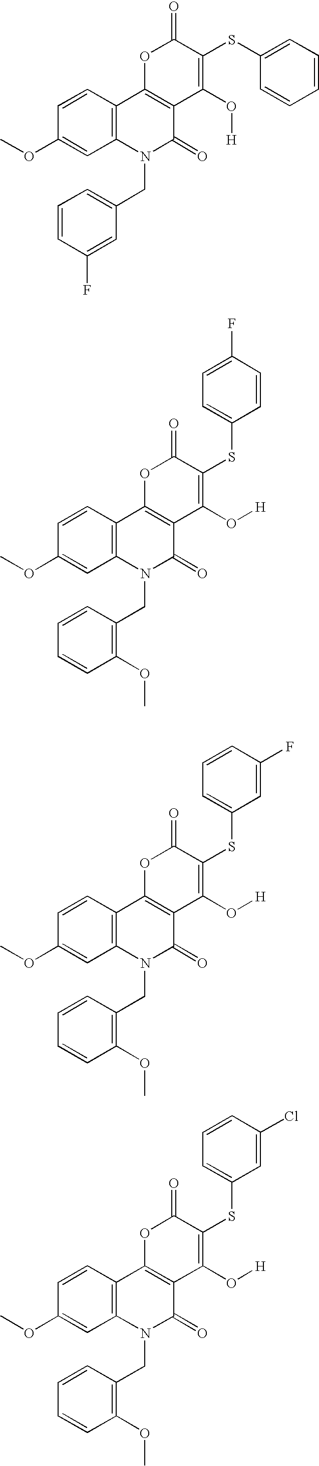 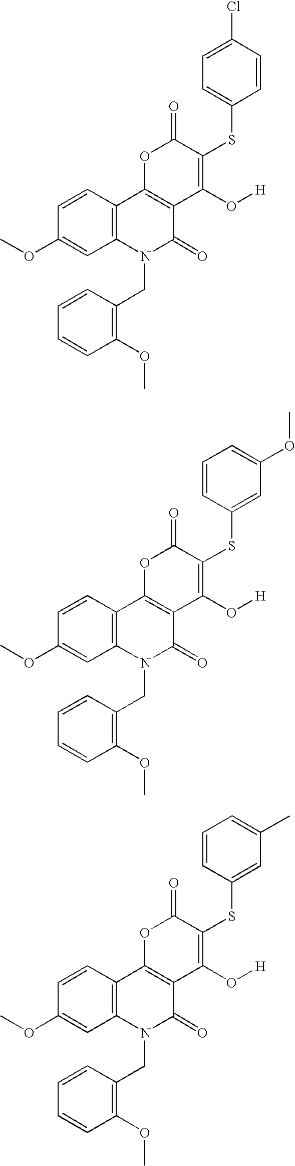 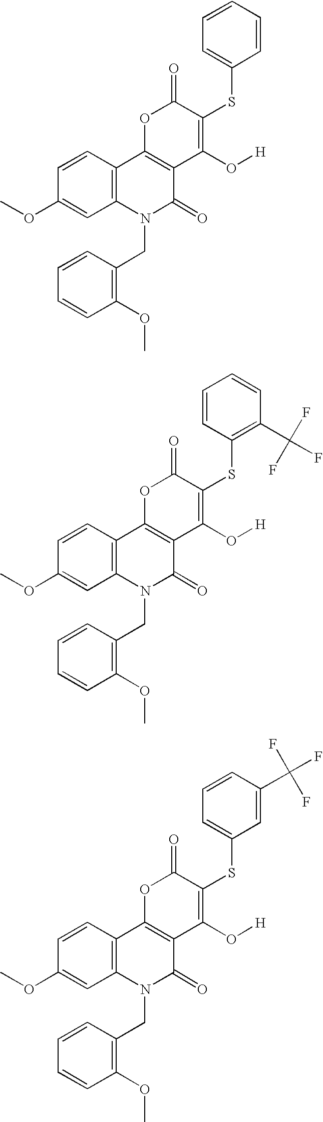 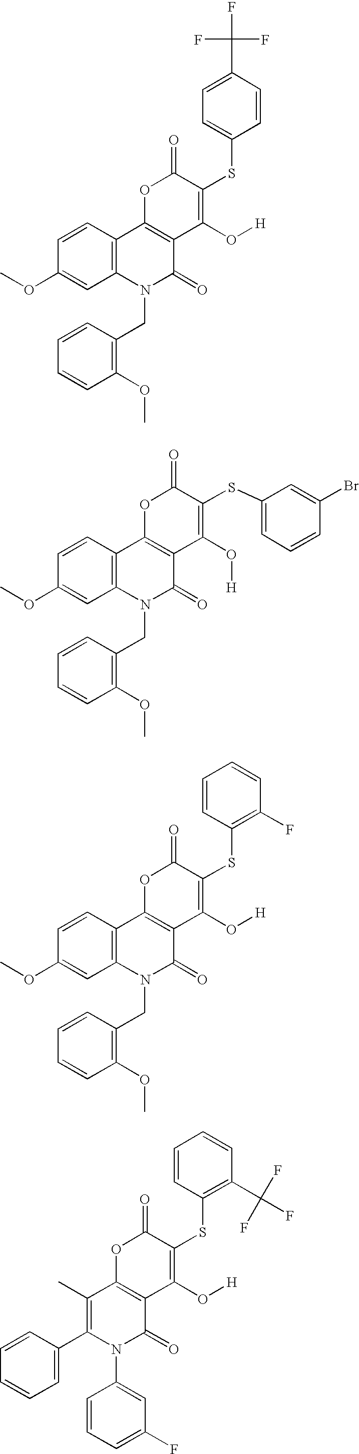 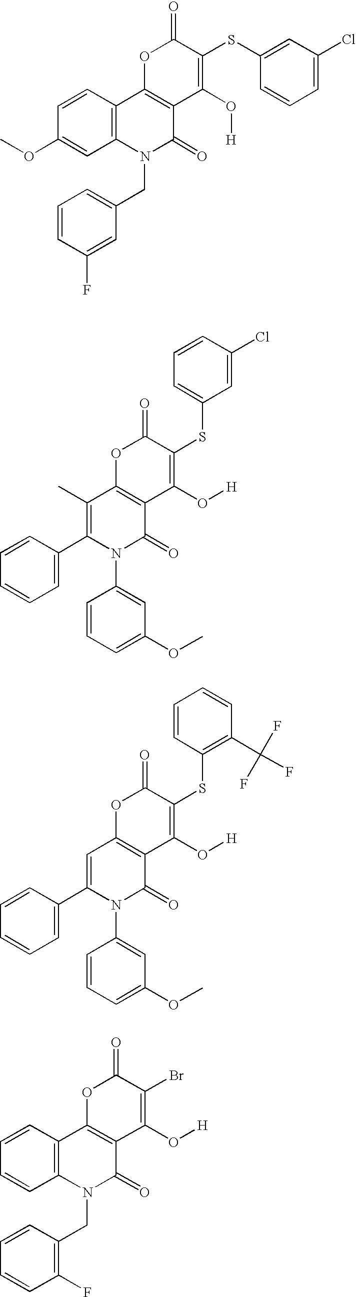 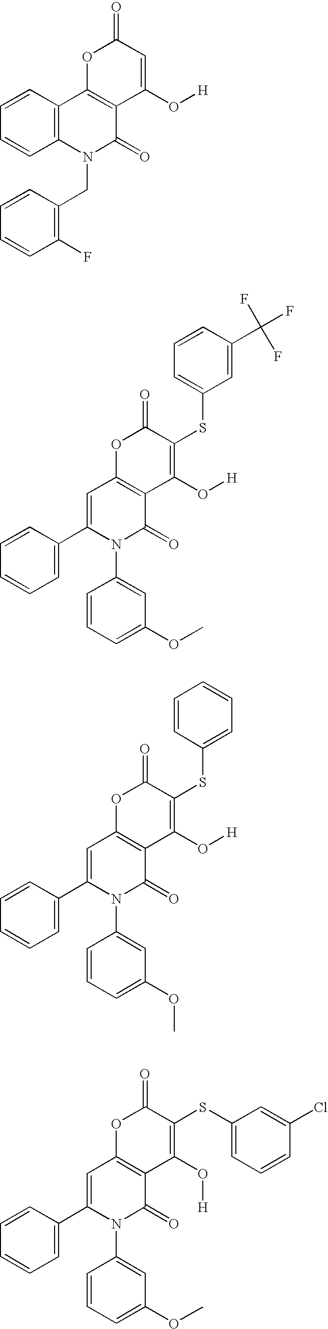 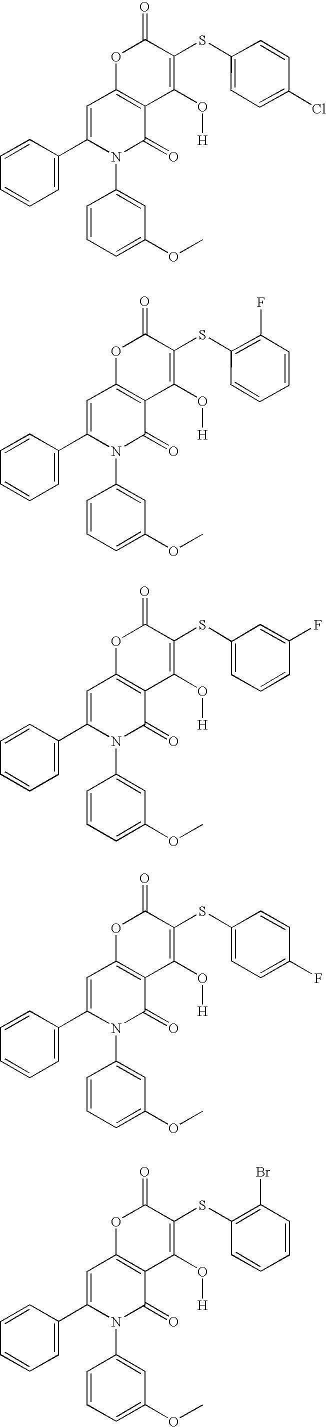 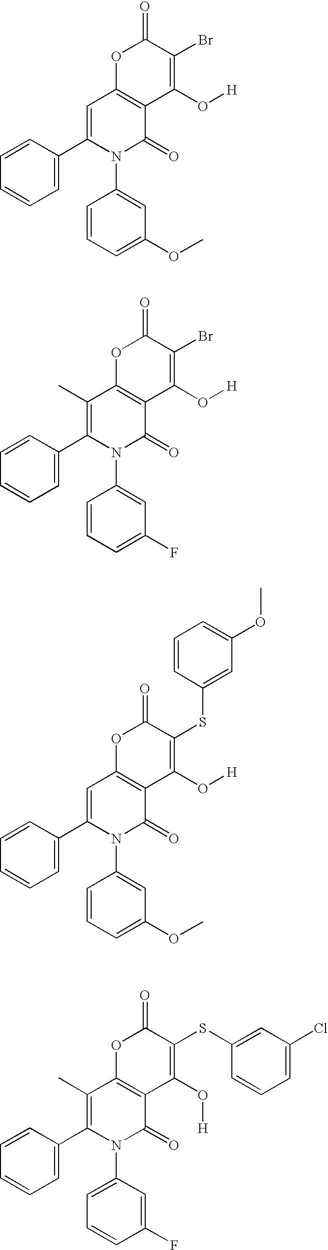 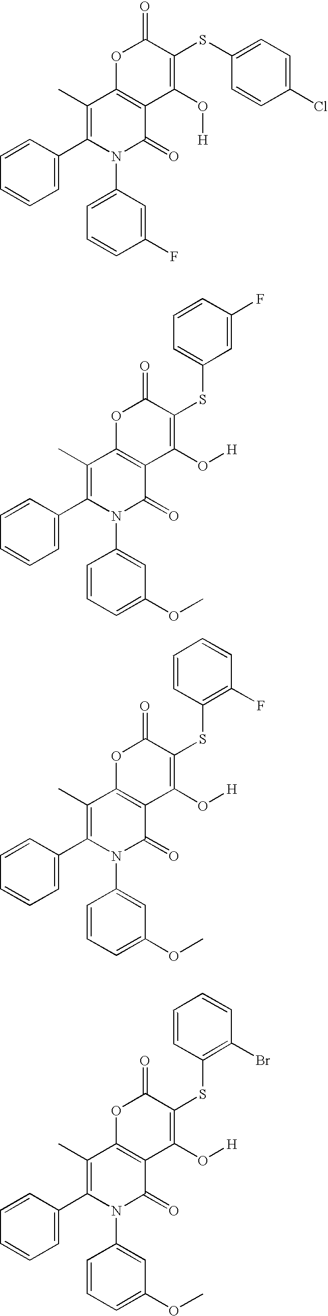 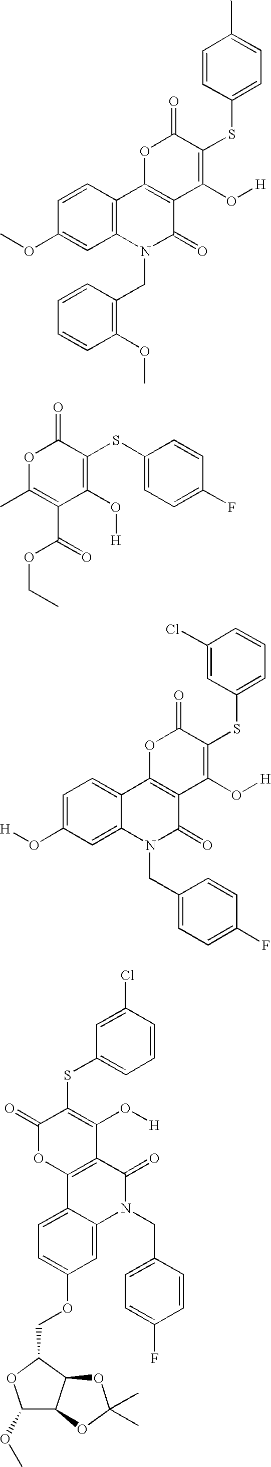 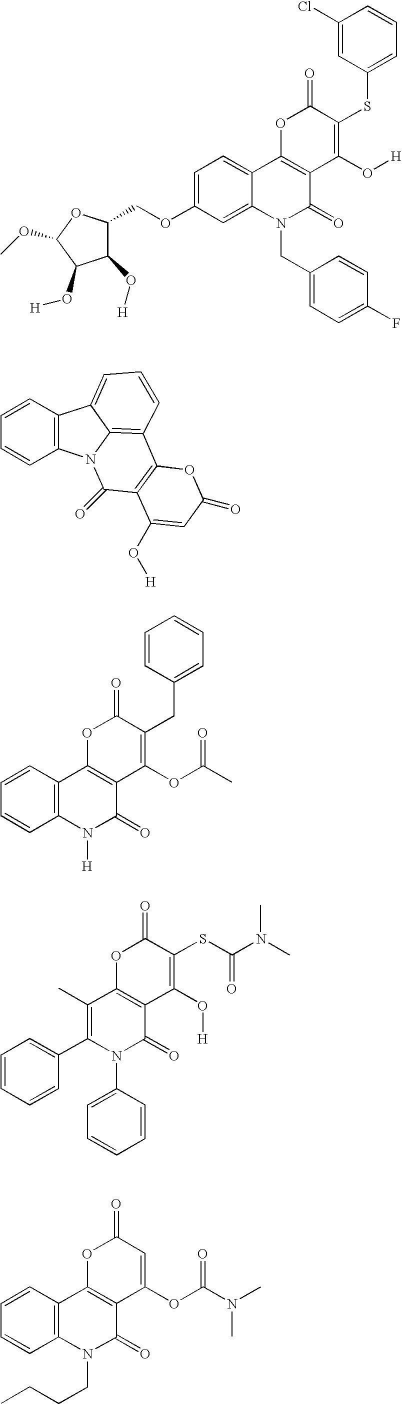 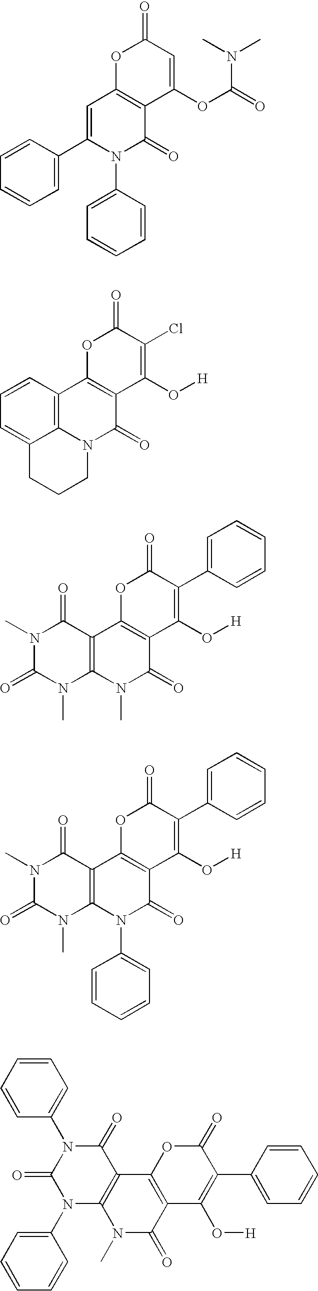 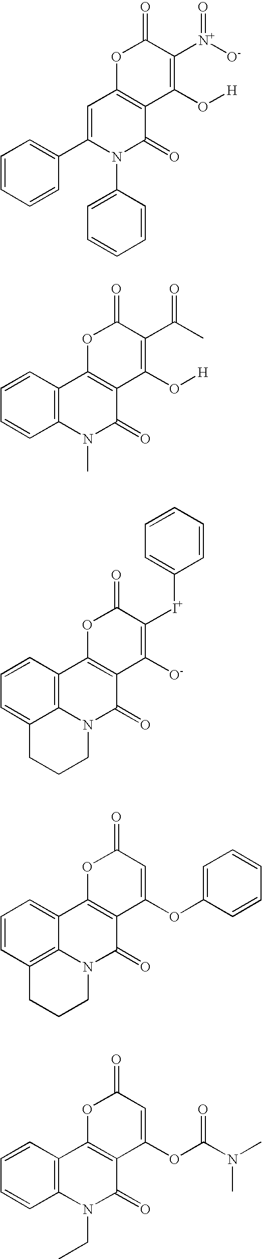 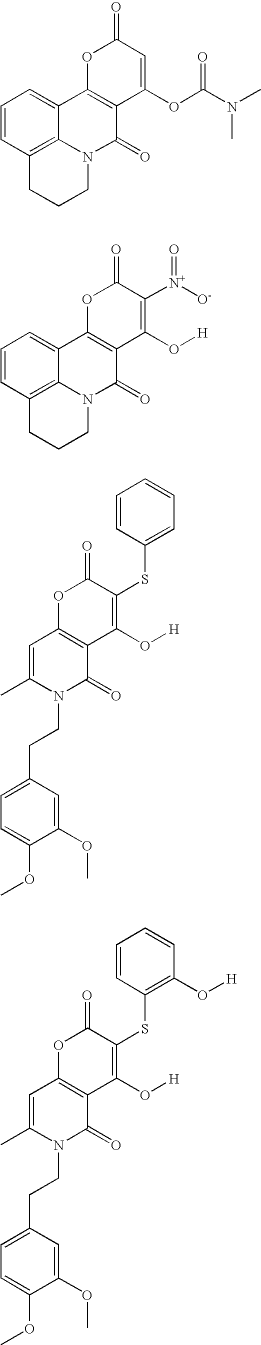 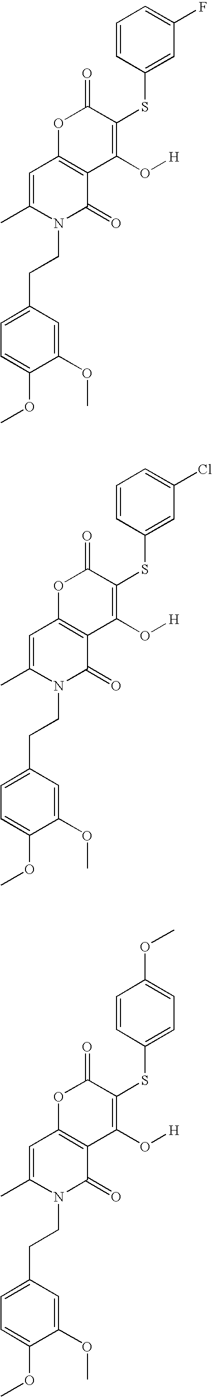 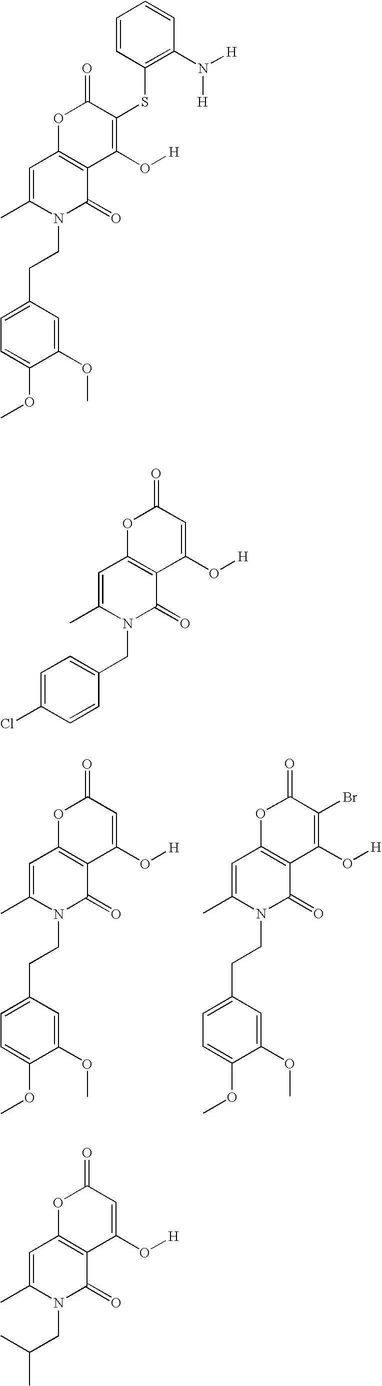 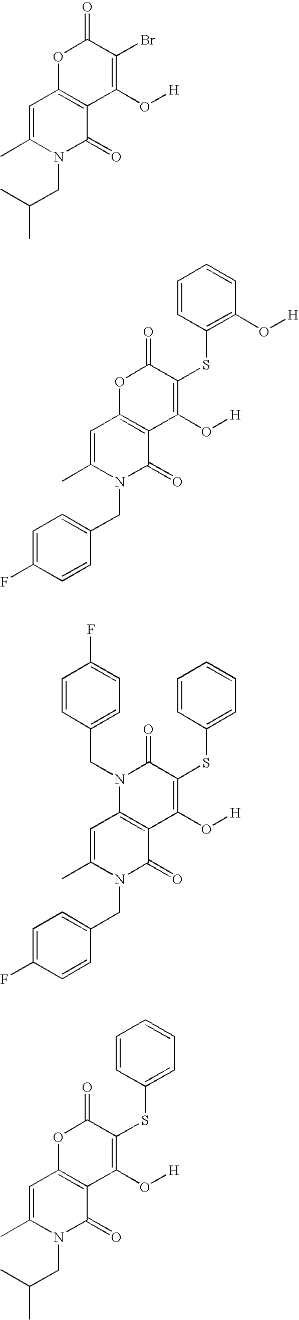 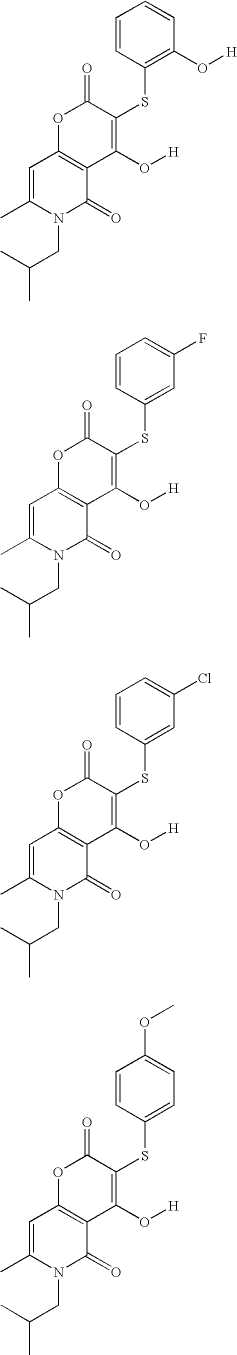 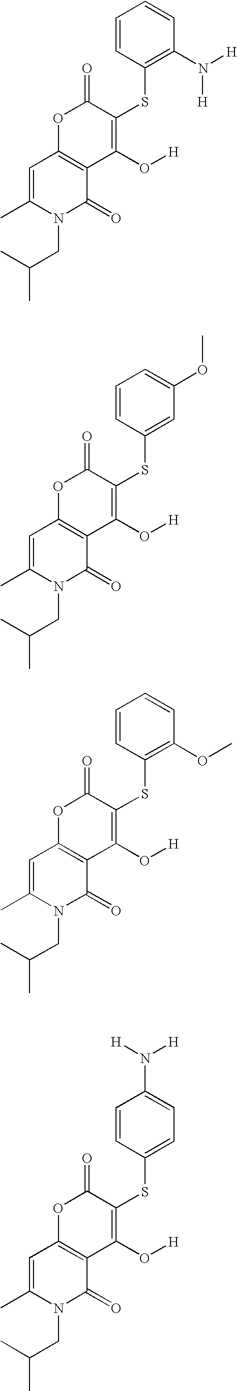 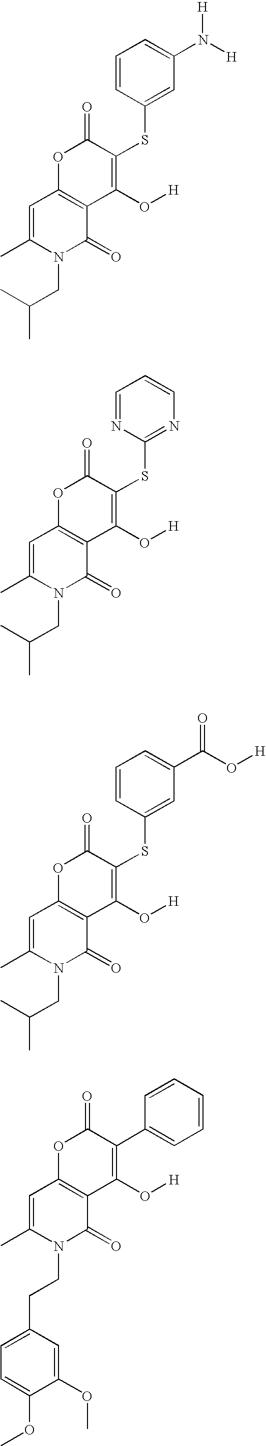 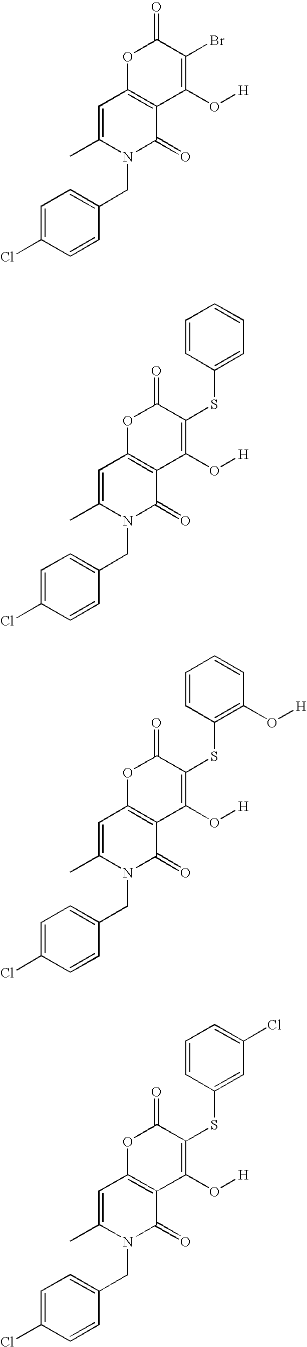 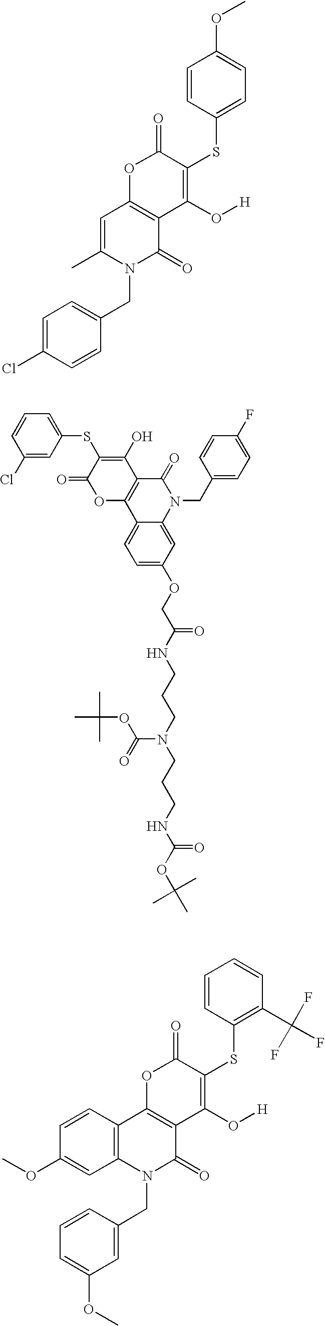 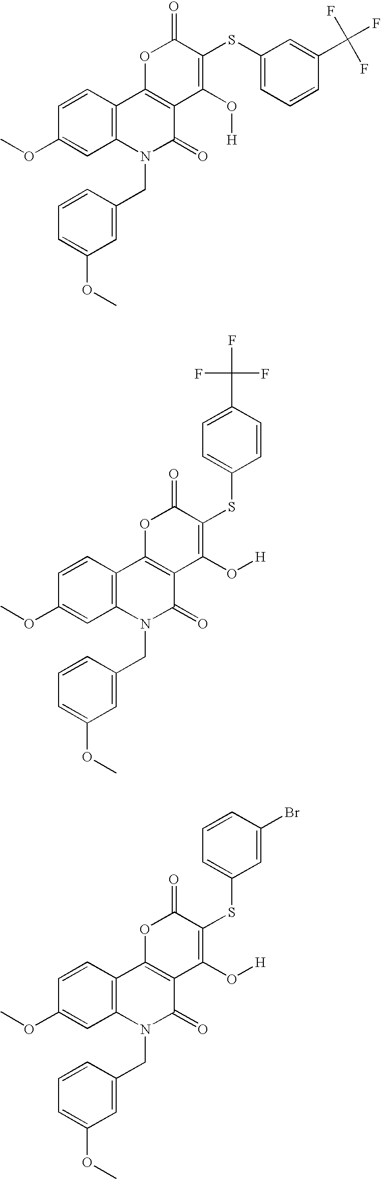 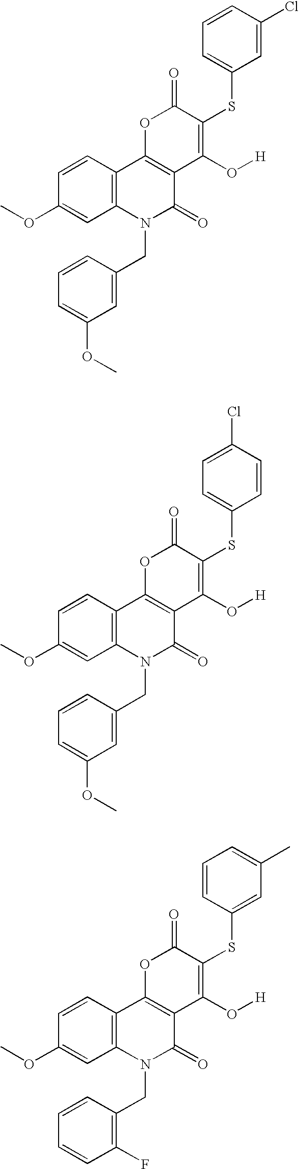 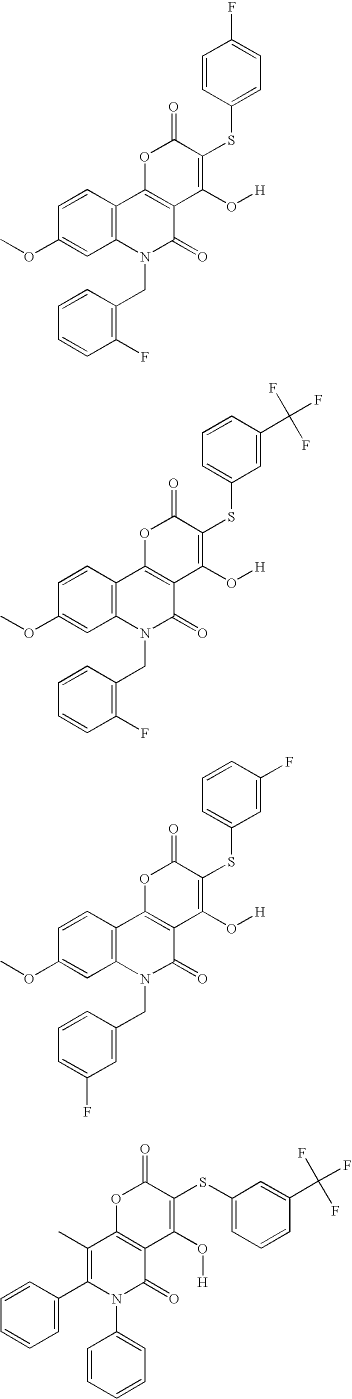 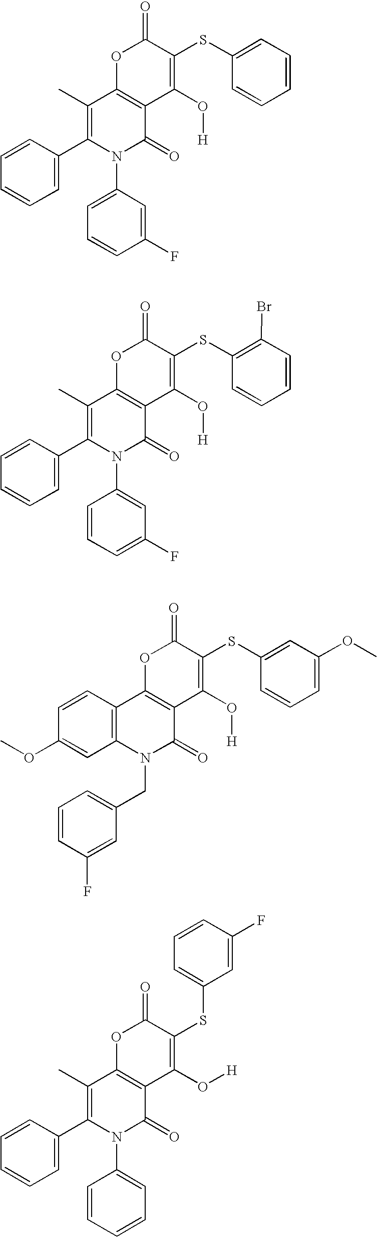 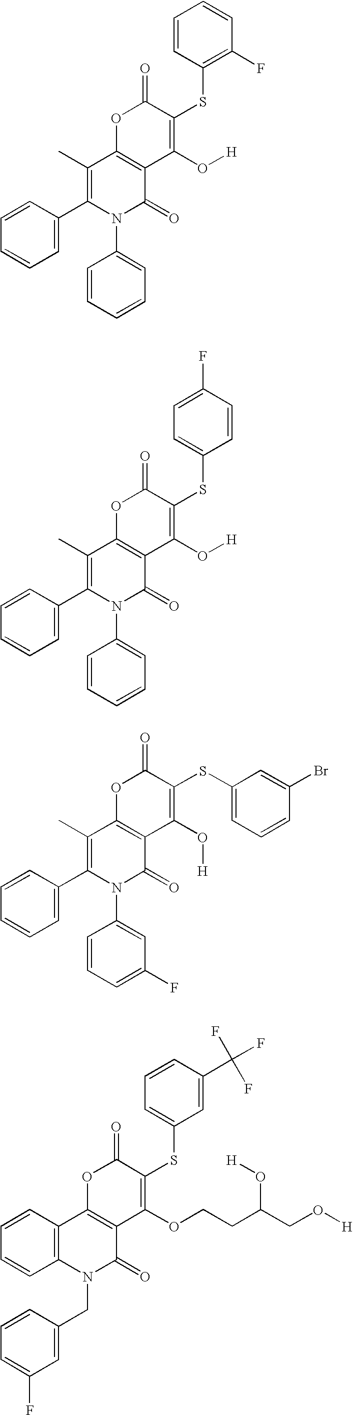 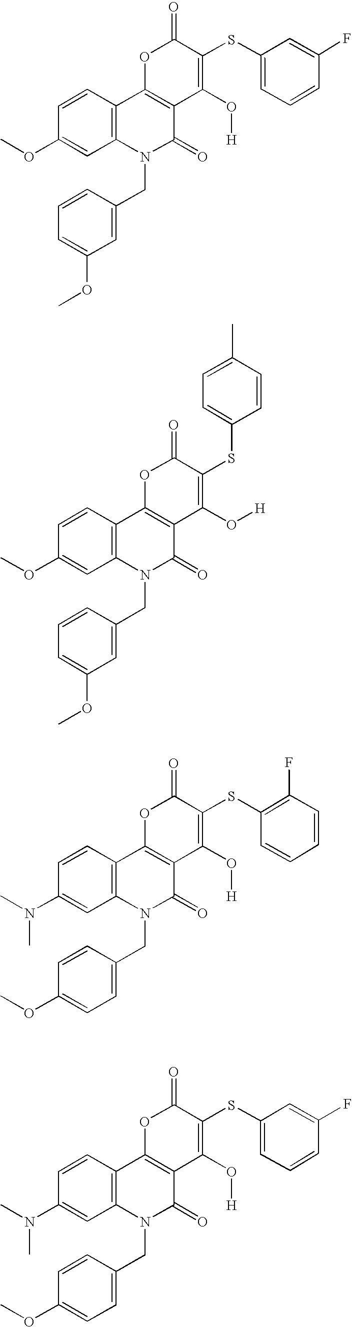 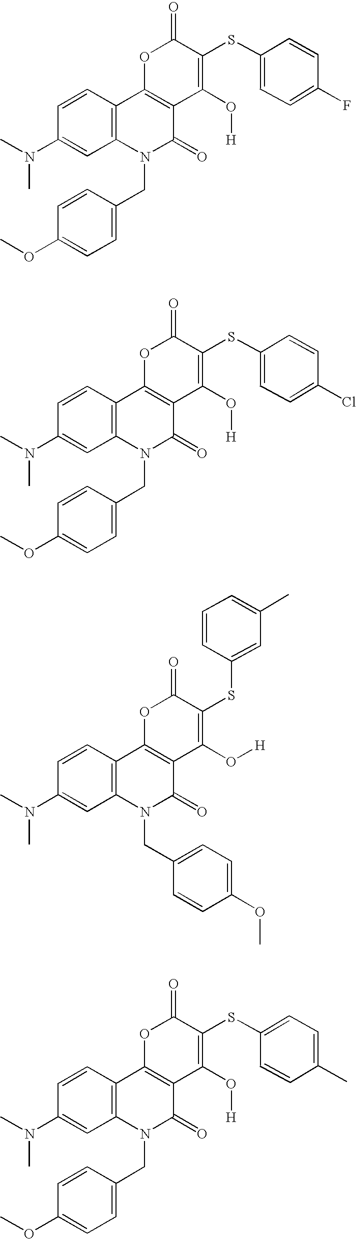 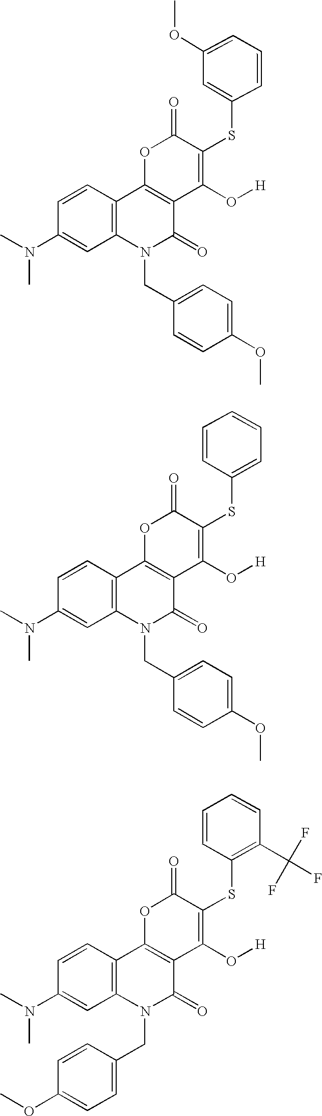 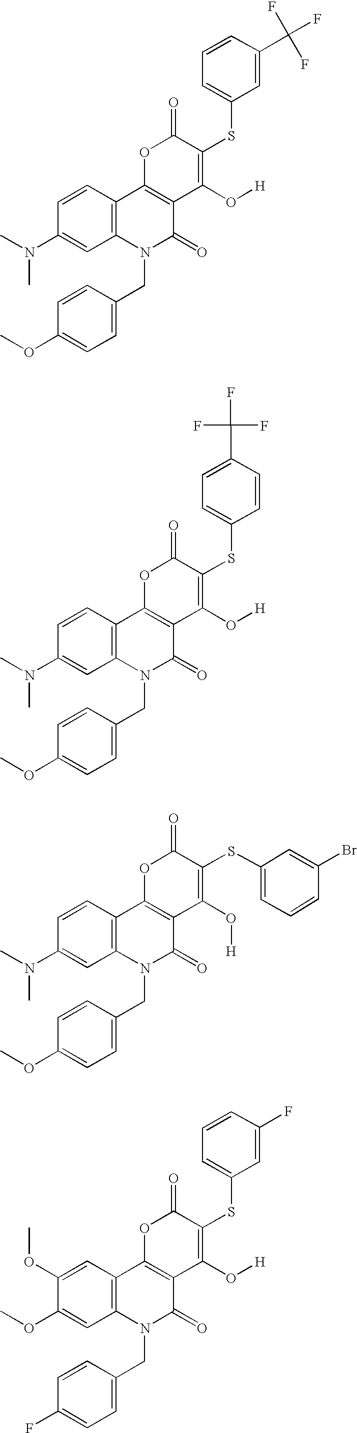 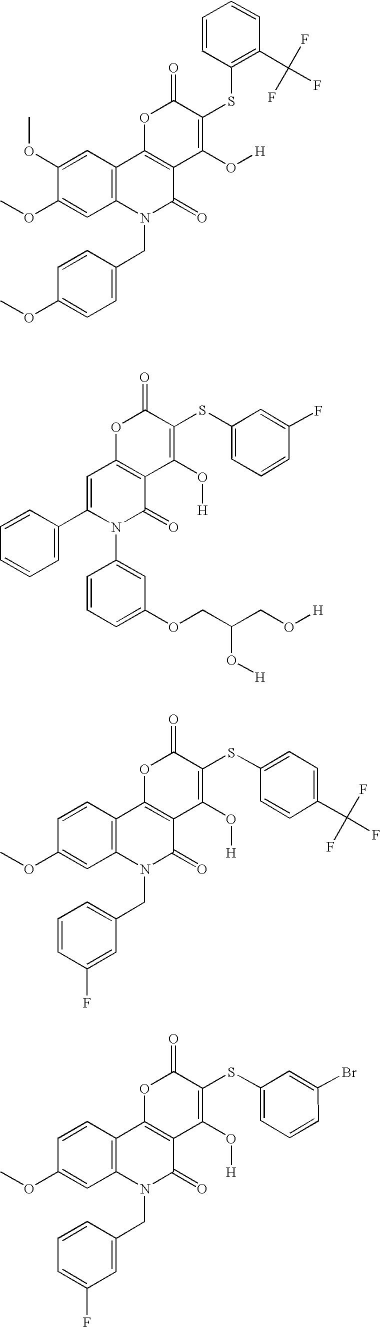 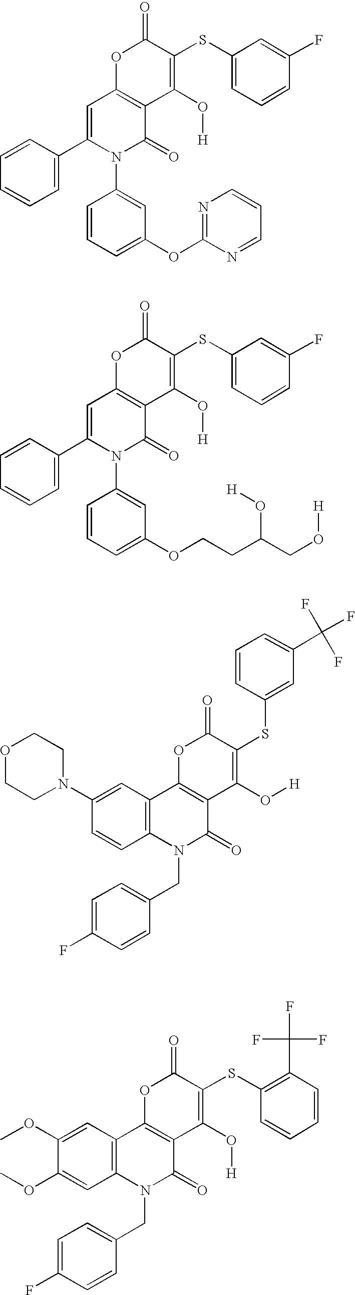 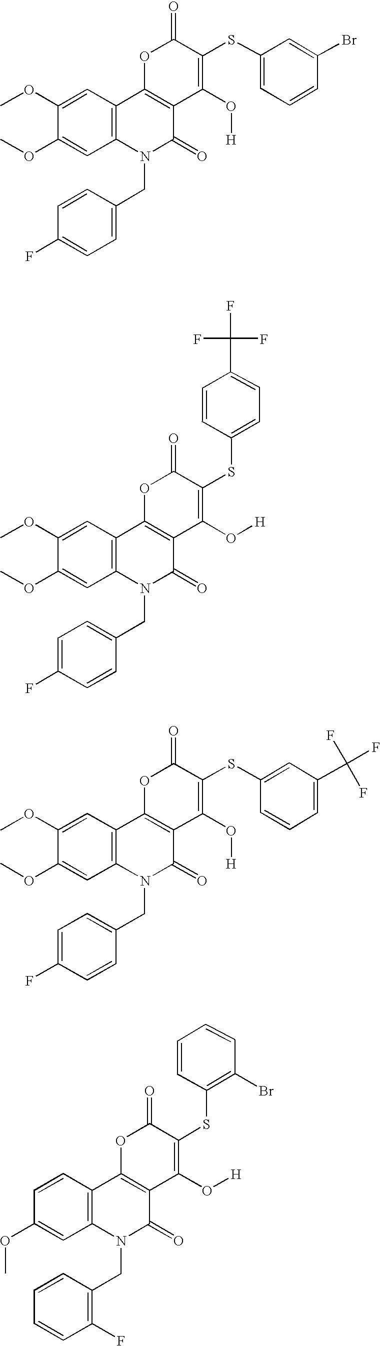 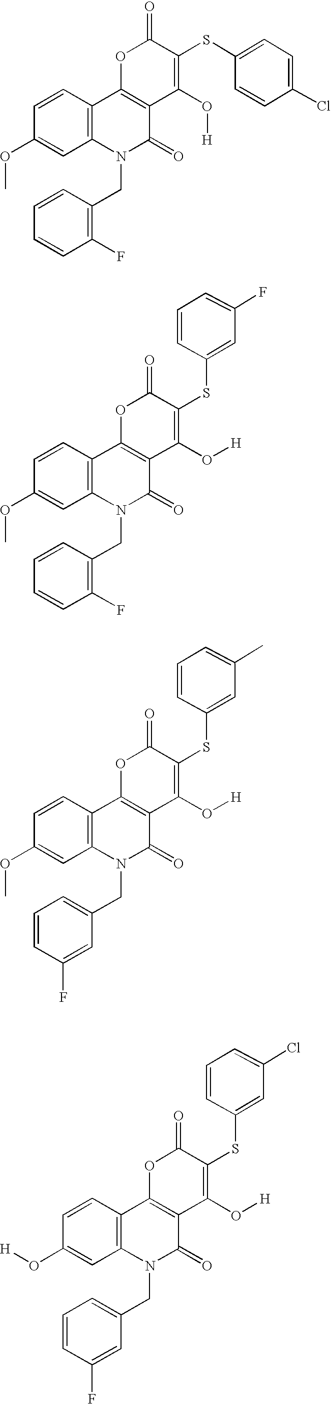 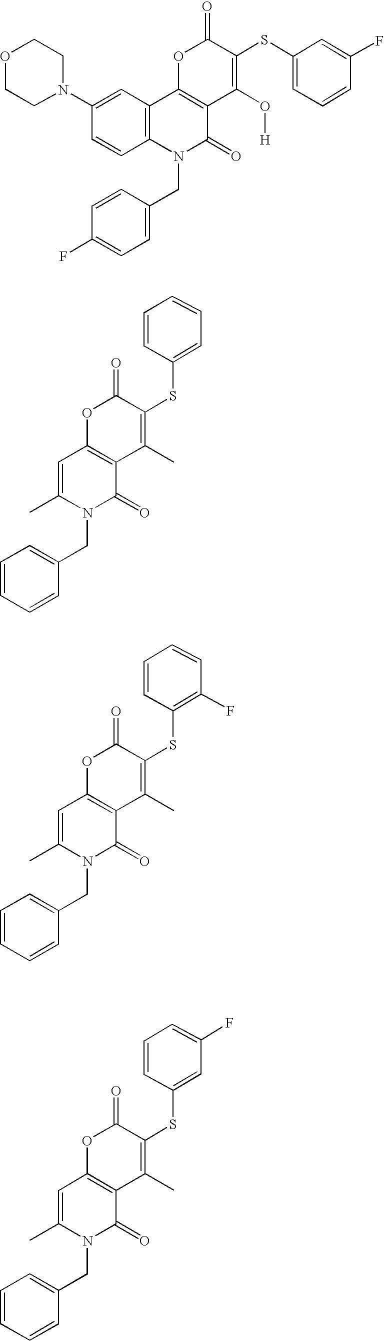 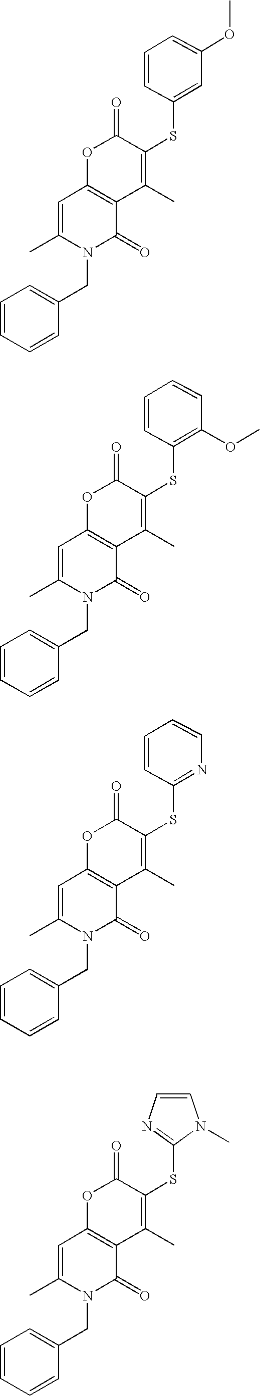 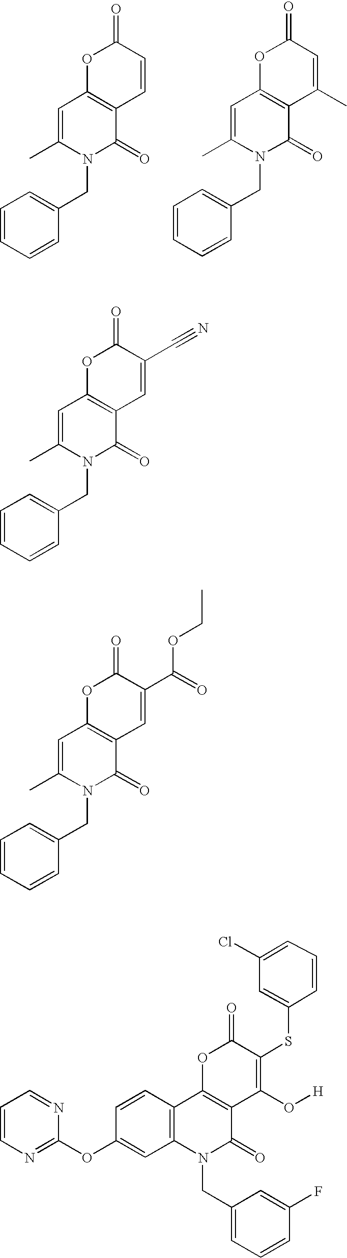 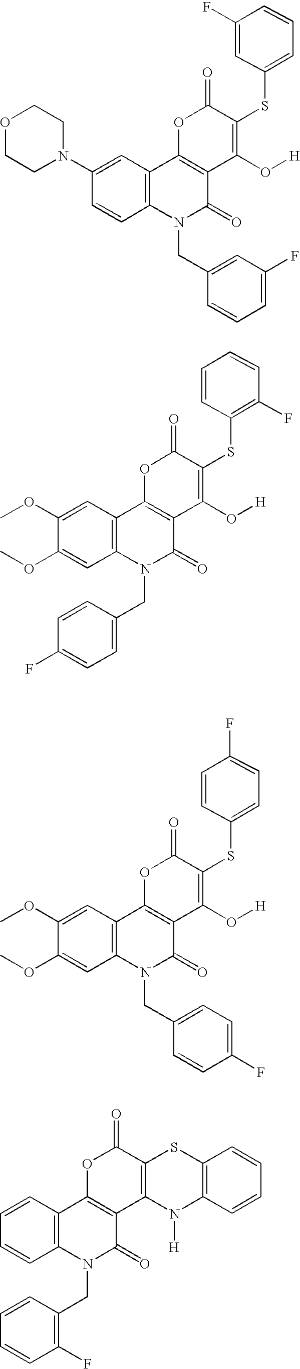 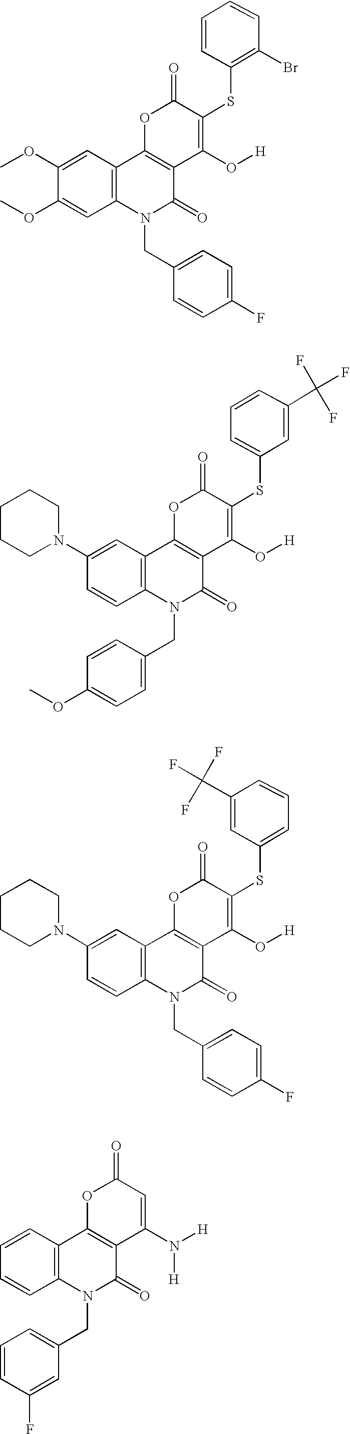 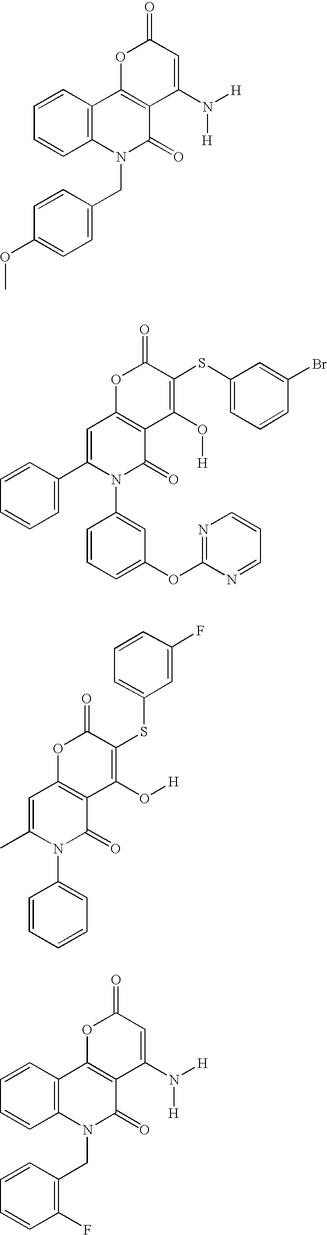 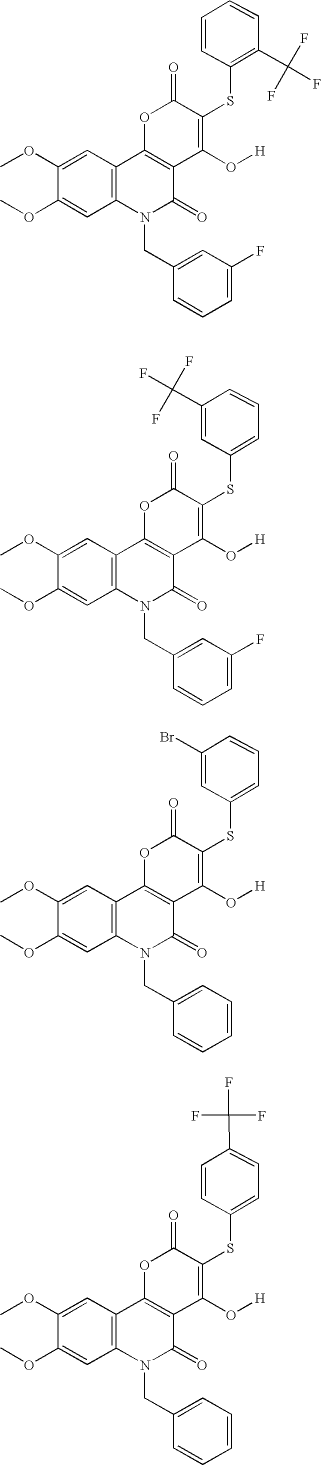 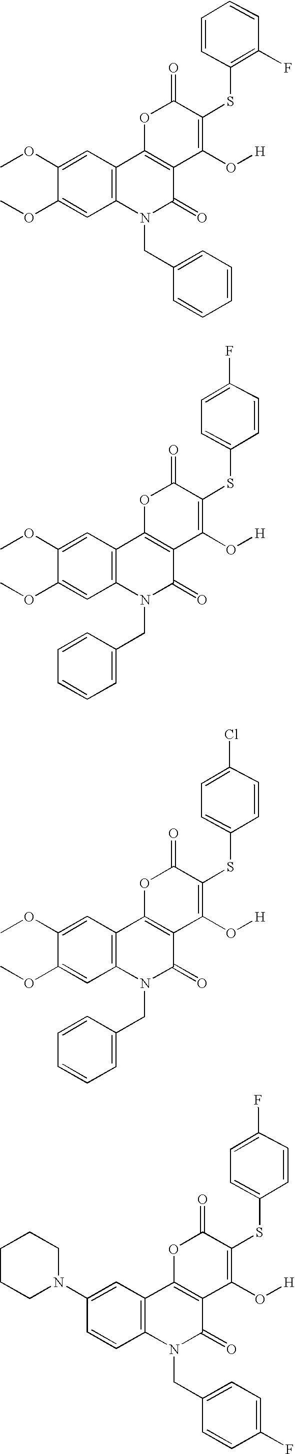 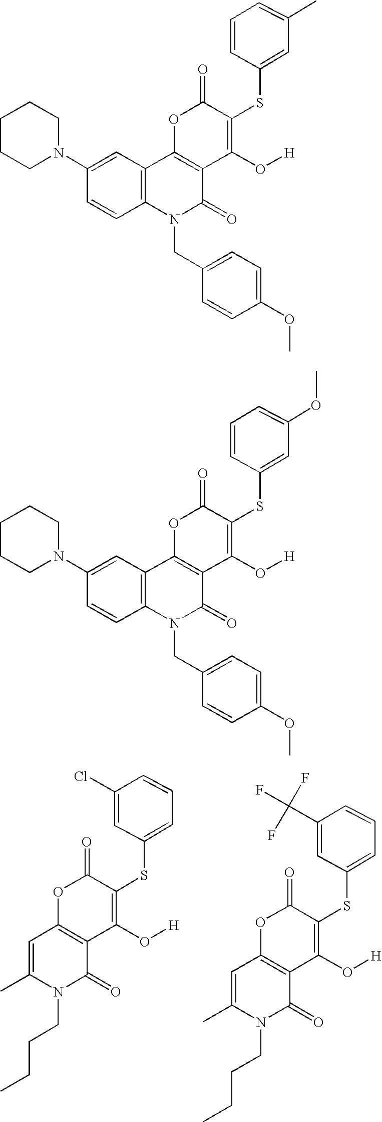 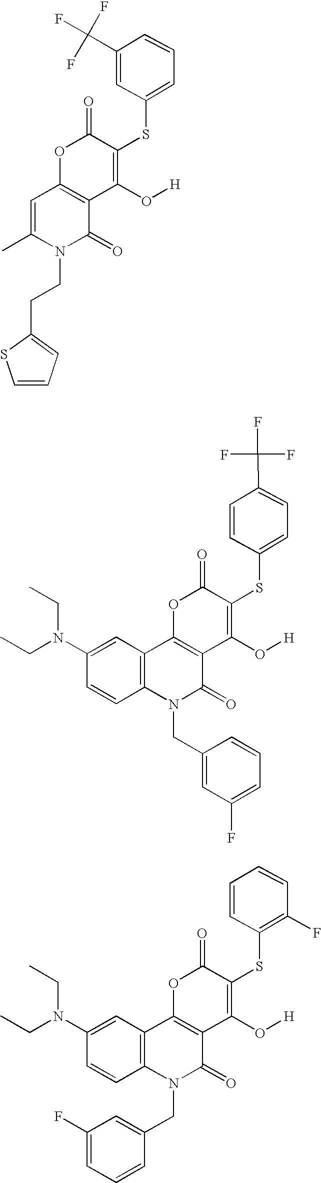 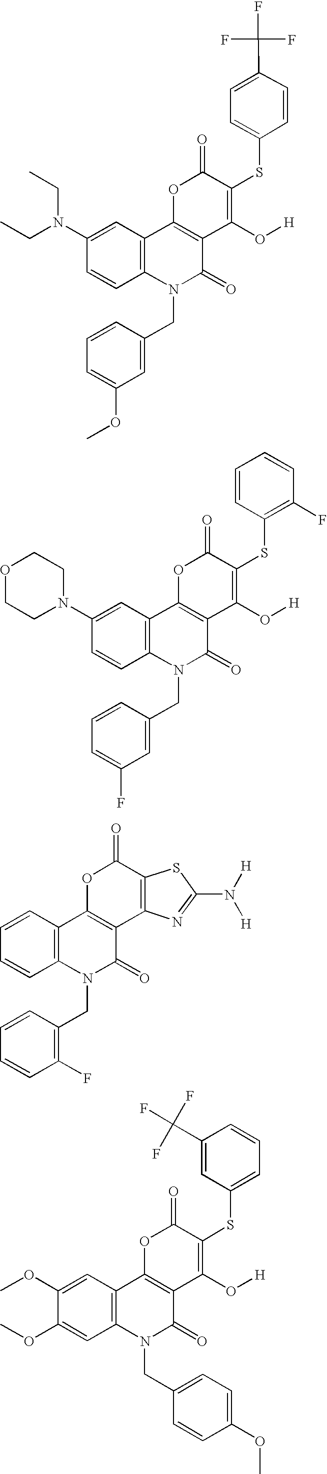 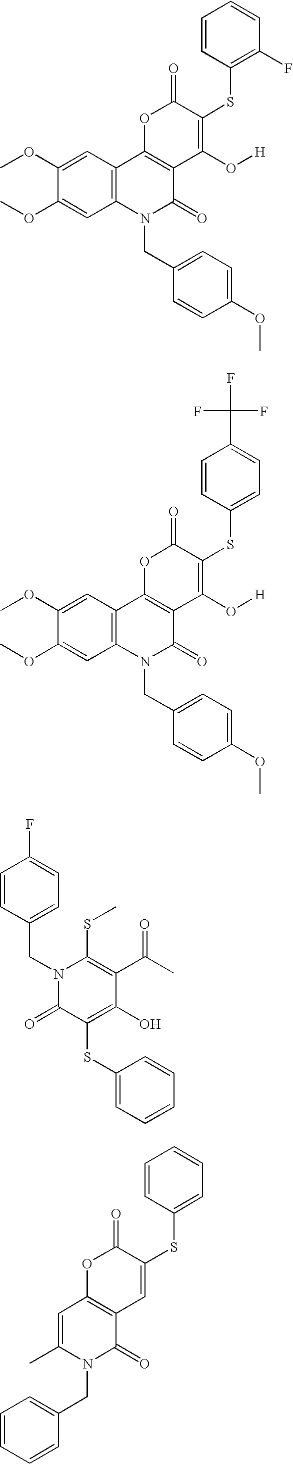 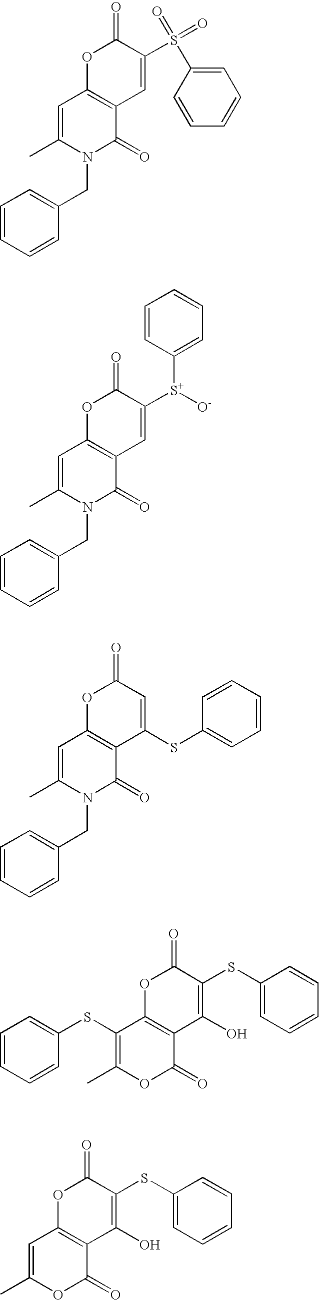 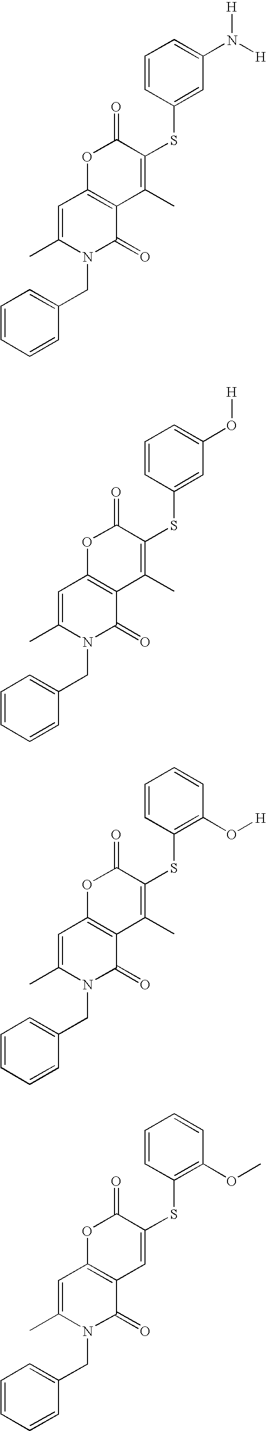 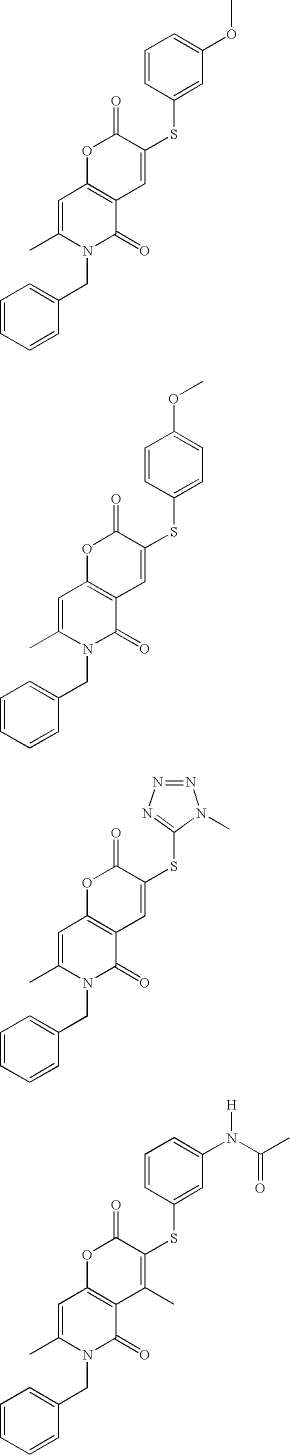 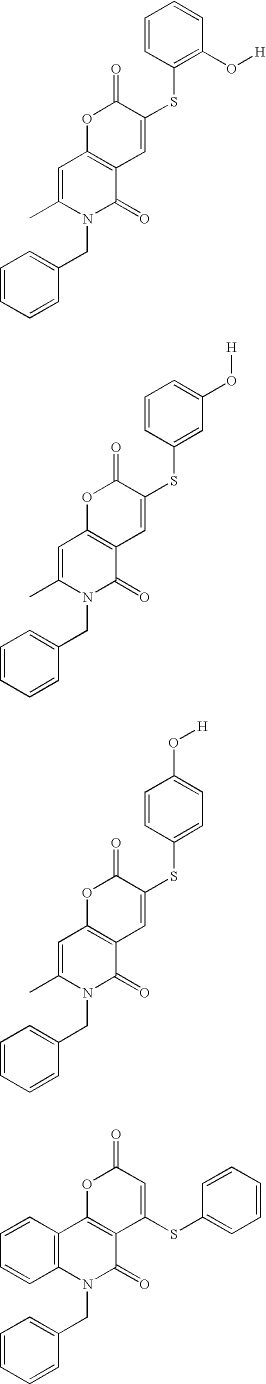 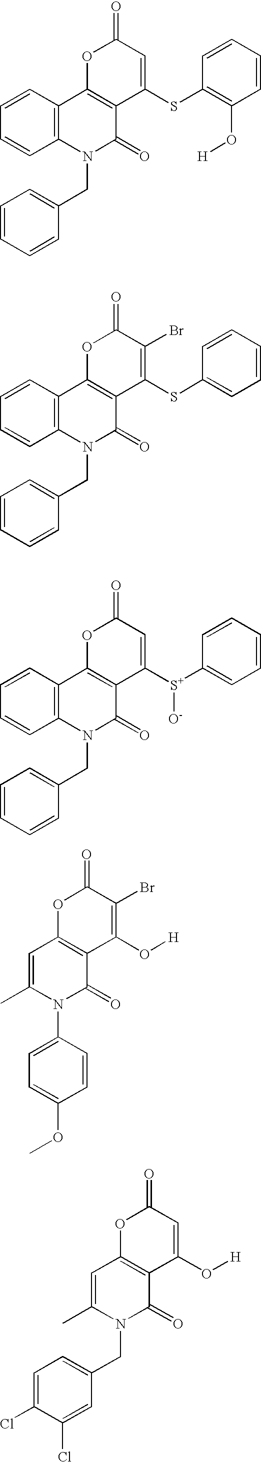 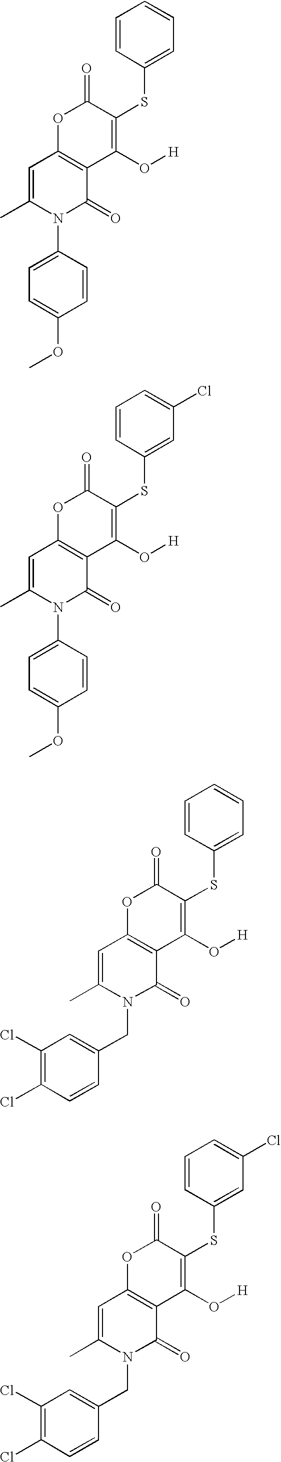 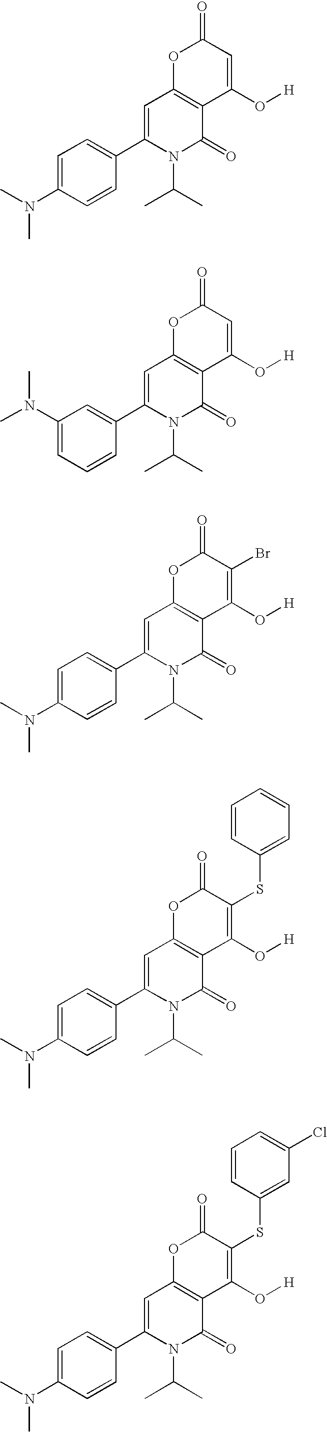 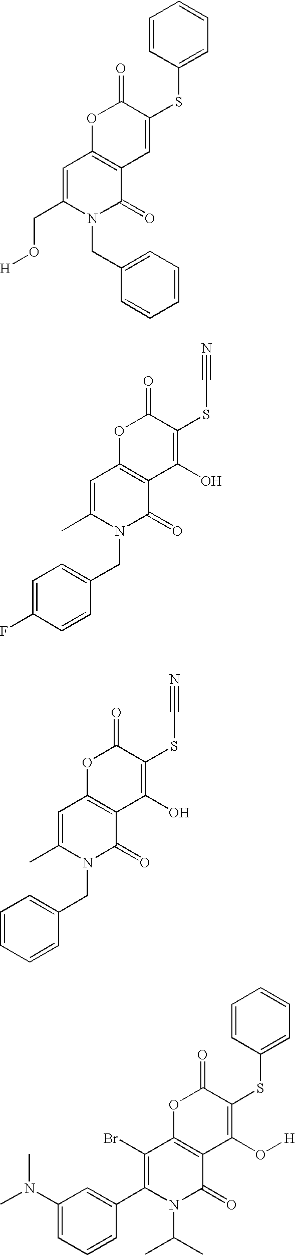 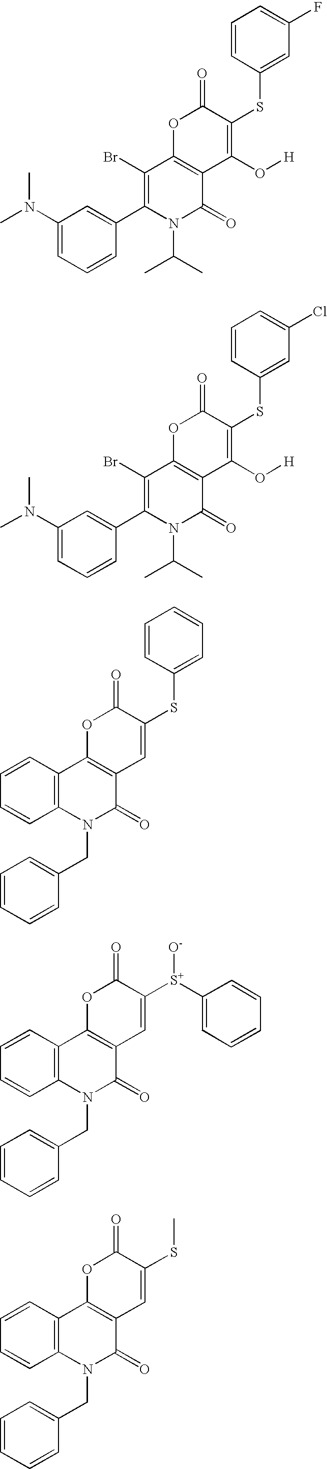 [chemical structure]
